(19) United States
(12) Patent Application Publication
(10) Pub. No.: US 2026/0347361 A1
(43) Pub. Date: Nov. 5, 2026

(54) LRRK2 INHIBITORS AND USES THEREOF

(71) Applicant: NEURON23, INC, South San Francisco, CA (US)

(72) Inventors: Ming Yu, South San Francisco, CA (US); Zhonghua Pei, South San Francisco, CA (US)

(21) Appl. No.: 19/106,830

(22) PCT Filed: Sep. 7, 2023
(86) PCT No.: PCT/US2023/032146

§ 371 Date: Feb. 26, 2025

Related U.S. Application Data
(60) Provisional application No. 63/404,742, filed on Sep. 8, 2022.

(51) Int. Cl. C07D 487/04; A61K 31/553; C07D 471/14; C07D 519/00

(52) U.S. Cl. CPC C07D 487/04; A61K 31/553; C07D 471/14; C07D 519/00

(57) ABSTRACT

The invention provides pyrazolo[1,5-a][1,3,5]benzotriazepinyl, pyrazolo[1,5-a]pyrido[3,4-f][1,3,5]triazepinyl and related tricyclic compounds that inhibit the activity of one or more kinases, more particularly the protein kinase leucine rich repeat kinase 2 (LRRK2). The inventive compounds find applications in the treatment of a variety of disorders, including cancer and neurodegenerative diseases such as Parkinson's disease.

### FIELD OF INVENTION

The present invention relates to compounds that are capable of inhibiting one or more kinases, more particularly, LRRK2. The compounds find applications in the treatment of a variety of disorders, including cancer and neurodegenerative diseases such as Parkinson's disease.

### BACKGROUND

A variety of medical conditions that affect millions of people are caused or exacerbated by unregulated activity of protein kinases. For example, aberrant kinase activity is associated with autoimmune diseases, inflammatory diseases, bone diseases, metabolic diseases, neurological and neurodegenerative diseases, cancer, cardiovascular diseases, allergies, asthma, Alzheimer's disease, Parkinson's disease, skin disorders, eye diseases, infectious diseases and hormone-related diseases. For many such disorders, however, no effective inhibitor or activator exists for the particular kinase that causes the disorder or its symptoms. Consequently, patients continue to suffer from an array of disorders due to the lack of a suitable drug for their conditions.

Parkinson's disease (PD) is a neurodegenerative disorder characterized by selective degeneration and cell death of dopaminergic neurons in the substantial nigra region of the brain. Parkinson's disease was generally considered to be sporadic and of unknown etiology. In the past few years, there has been an important development of the understanding of the genetic basis of this disease and associated pathogenic mechanisms. Parkinson's disease is a chronic, progressive motor system disorder that afflicts approximately one out of every 1000 people, with hereditary Parkinson's disease accounting for 5-10% of all of patients. Parkinson's disease is caused by progressive loss of mid-brain dopamine neurons, leaving patients with impaired ability to direct and control their movements. The primary Parkinson's disease symptoms are trembling, rigidity, slowness of movement, and impaired balance. Many Parkinson's disease patients also experience other symptoms such as emotional changes, memory loss, speech problems, and sleeping disorders.

Pathologically, the disease is characterized by loss of dopaminergic neurons with the consequent decrease in dopamine levels in the brain and by aggregation of the protein α-synuclein in the dopaminergic neurons. These aggregations called Lewy-bodies are composed of insoluble α-synuclein phosphorylated at serine-129 and ubiquitin. Current Parkinson's disease therapeutic intervention strategies aim at increasing the dopamine levels in areas innervated by dopaminergic neurons in the brain. Levadopa is a precursor of dopamine, and it is therapeutically used to increase dopamine levels. Carbidopa is an inhibitor of the enzyme aromatic-L-amino-acid decarboxylase also known as DOPA decarboxylase, and it is often co-administered with levadopa to increase the fraction of levadopa which reaches the clinically relevant regions in the brain.

### SUMMARY

Inhibitors of leucine rich repeat kinase 2 (LRRK2) are known to be effective for treatment of Parkinson's Disease (PD). The invention provides LRRK2 inhibitors for treatment of PD. In one aspect, the invention provides a compound of Formula (I):

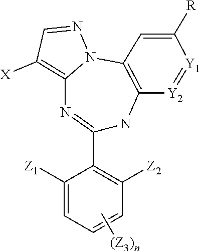

or an enantiomer, mixture of enantiomers, tautomer, or pharmaceutically acceptable salt thereof,
wherein: n is 1, 2, or 3;
Y₁ and Y₂ are independently N or CH;
Z₁, Z₂, and Z₃ are independently selected from H, —OH, halo, C₁-C₆ alkyl, C₁-C₆ heteroalkyl, haloalkyl, alkoxy, haloalkxoxy, —CH(OH)-alkyl, hydroxyalkyl, hydroxyalkoxy, amino, cyano, or morpholino;
X is H, —OH, halo, cyano, alkyl, haloalkyl, cyanoalkyl, or cycloalkyl with one or more heteroatoms, wherein the heteroatoms are selected from a group consisting of N, O, or S;
R is H, halo, substituted or unsubstituted aryl, substituted or unsubstituted C₁-C₆-alkyl, branched alkyl, alkenyl, alkynyl, haloalkyl, alkoxy, cycloalkoxy, haloalkoxy, -cyano, —CH₂-cycloalkyl, —CF₂-cycloalky, —CH(CH₃)-cycloalkyl, —CH₂-aryl, —CF₂-aryl, —CH(—CH₃)-aryl, C(═O)-alkyl, —C(═O)cycloalkyl, —C(═O)—NH-alkyl, —COOH (and esters and carboxamides thereof), C(═O)—NH—S(═O)₂alkyl, C(═O)—NH—S(═O)₂aryl, —C(═O)-morpholine, —C(═O)-heterocycles, —C(—CH₃)₂—OH, —CH₂—C(═O)—NH₂; -hydroxy, alkylhydroxy, alkyl-COOH (and esters and carboxamides thereof), substituted or unsubstituted alkylsulfonyl, arylsulfonyl, amino, —NHC(═O)alkyl, —N(alkyl)C(═O)alkyl, —NHC(═O)aryl, —N(alkyl)C(═O)aryl, —N(H)S(═O)₂alkyl, —N(H)S(═O)₂aryl, —N(alkyl)S(═O)₂alkyl, cyanoalkyl, haloalkyl, —S═(O)₂-alkyl, —S═(O)₂-cycloalkyl, —S═(O)₂-aryl, —S(═O)₂N(H)alkyl, substituted or unsubstituted morpholine, 3-7 membered heterocycle, any of which may have one or more substituents, 3-7 membered cycloalkyl or heterocycle, wherein the 3-7 membered cycloalkyl or heterocycle is fused with another 3-7 cycloalkyl or heterocycle, wherein the rings are spiro, bridged bicyclic, or spiro, wherein at least one heteroatom in the heterocycle rings are independently selected from O, S, and N. In certain embodiments, when Y₁ is CH and Y₂ is CH; R is not any of H, fluoro, chloro, or methyl.

In another embodiment, in the compound of Formula (I), Y₁ is N and Y₂ is CH.

In another embodiment, in the compound of Formula (I), Y₁ is CH and Y₂ is N.

In another embodiment, in the compound of Formula (I), Z₁ and Z₂ are each fluoro and Z₃ is H.

In another embodiment, in the compound of Formula (I), Z₁ and Z₂ are fluoro and chloro respectively, and Z₃ is H.

In another embodiment, in the compound of Formula (I), Z₁, Z₂, and Z₃ are fluoro.

In another embodiment, in the compound of Formula (I), R is selected from a group consisting of: chloro, hydrogen, bromo, fluoro, methyl, methylcyano, and morpholine.

In another embodiment, in the compound of Formula (I), X is H, bromo, chloro, fluoro, or methyl.

In another embodiment, in the compound of Formula (I), R is selected from a group consisting of substituted or unsubstituted morpholines, amides, piperazines, alkyls, azetidine, heterocycle, or heteroaryl.

In another embodiment, in the compound of Formula (I), R is not any of H, fluoro, chloro, or methyl.

In another embodiment, in the compound of Formula (I), X is not any of H, fluoro, chloro, or cyano.

In another embodiment, in the compound of Formula (I), R is selected from a group consisting of: H, bromo, amide, ester, carboxylic acid,

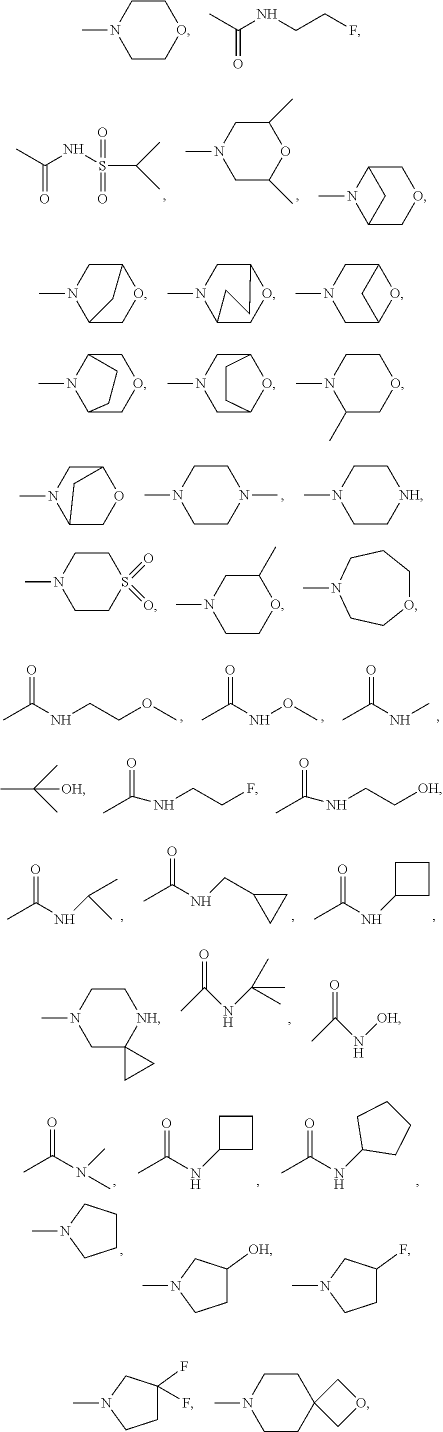

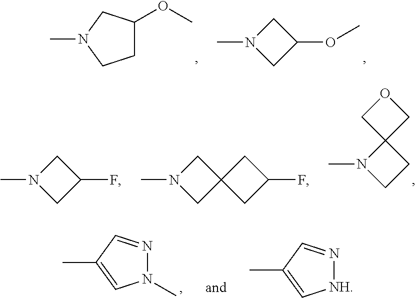

In another embodiment, one or more hydrogen atoms of the compound of Formula (I) are replaced by deuterium.

In another embodiment, in the compound of Formula (I), the compound is selected from a group consisting of

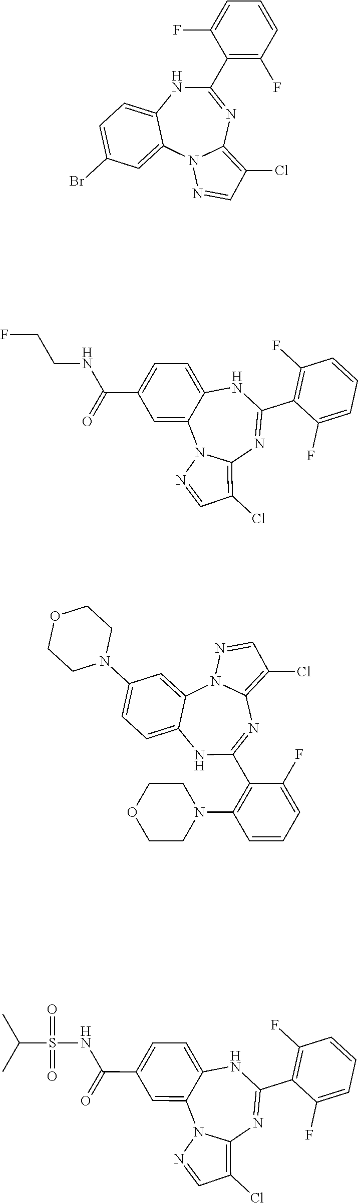 [SMILES: Fc1cccc(F)c1C1=Nc2c(Cl)cnn2-c2cc(Br)ccc2N1 ; O=C(NCCF)c1ccc2c(c1)-n1ncc(Cl)c1N=C(c1c(F)cccc1F)N2 ; Fc1cccc(N2CCOCC2)c1C1=Nc2c(Cl)cnn2-c2cc(N3CCOCC3)ccc2N1 ; CC(C)S(=O)(=O)NC(=O)c1ccc2c(c1)-n1ncc(Cl)c1N=C(c1c(F)cccc1F)N2]

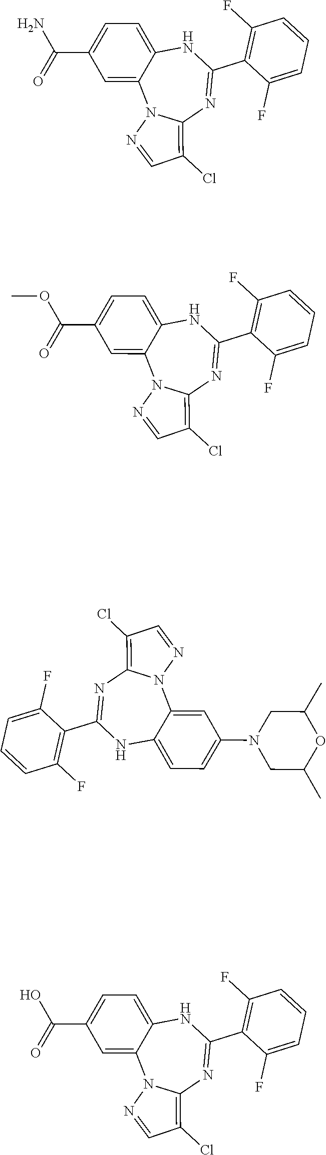 [SMILES: NC(=O)c1ccc2c(c1)-n1ncc(Cl)c1N=C(c1c(F)cccc1F)N2 ; COC(=O)c1ccc2c(c1)-n1ncc(Cl)c1N=C(c1c(F)cccc1F)N2 ; CC1CN(c2ccc3c(c2)-n2ncc(Cl)c2N=C(c2c(F)cccc2F)N3)CC(C)O1 ; O=C(O)c1ccc2c(c1)-n1ncc(Cl)c1N=C(c1c(F)cccc1F)N2]

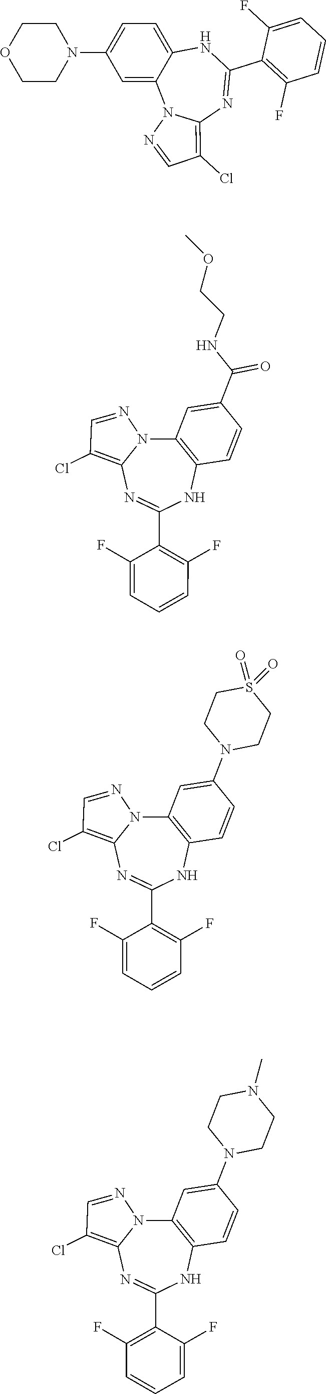 [SMILES: Fc1cccc(F)c1C1=Nc2c(Cl)cnn2-c2cc(N3CCOCC3)ccc2N1 ; COCCNC(=O)c1ccc2c(c1)-n1ncc(Cl)c1N=C(c1c(F)cccc1F)N2 ; O=S1(=O)CCN(c2ccc3c(c2)-n2ncc(Cl)c2N=C(c2c(F)cccc2F)N3)CC1 ; CN1CCN(c2ccc3c(c2)-n2ncc(Cl)c2N=C(c2c(F)cccc2F)N3)CC1]

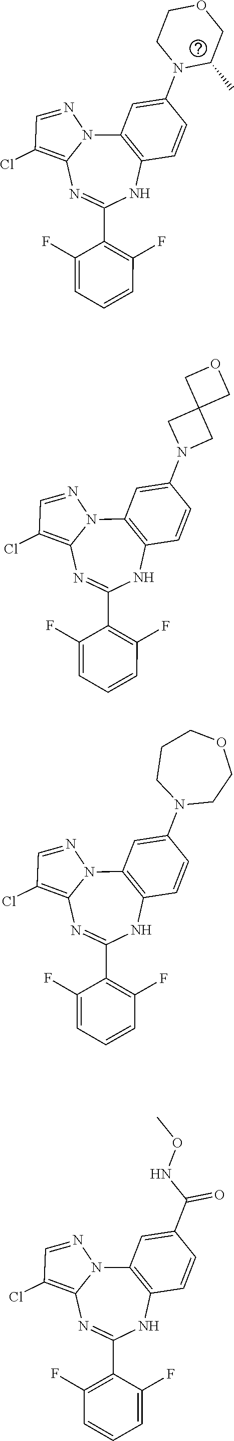 [SMILES: C[C@H]1COCCN1c1ccc2c(c1)-n1ncc(Cl)c1N=C(c1c(F)cccc1F)N2 ; Fc1cccc(F)c1C1=Nc2c(Cl)cnn2-c2cc(N3CC4(COC4)C3)ccc2N1 ; Fc1cccc(F)c1C1=Nc2c(Cl)cnn2-c2cc(N3CCCOCC3)ccc2N1 ; CONC(=O)c1ccc2c(c1)-n1ncc(Cl)c1N=C(c1c(F)cccc1F)N2]

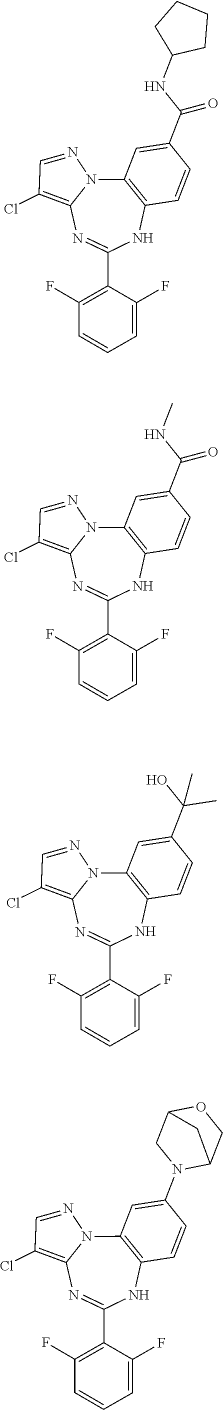 [SMILES: O=C(NC1CCCC1)c1ccc2c(c1)-n1ncc(Cl)c1N=C(c1c(F)cccc1F)N2 ; CNC(=O)c1ccc2c(c1)-n1ncc(Cl)c1N=C(c1c(F)cccc1F)N2 ; CC(C)(O)c1ccc2c(c1)-n1ncc(Cl)c1N=C(c1c(F)cccc1F)N2 ; Fc1cccc(F)c1C1=Nc2c(Cl)cnn2-c2cc(N3CC4CC3CO4)ccc2N1]

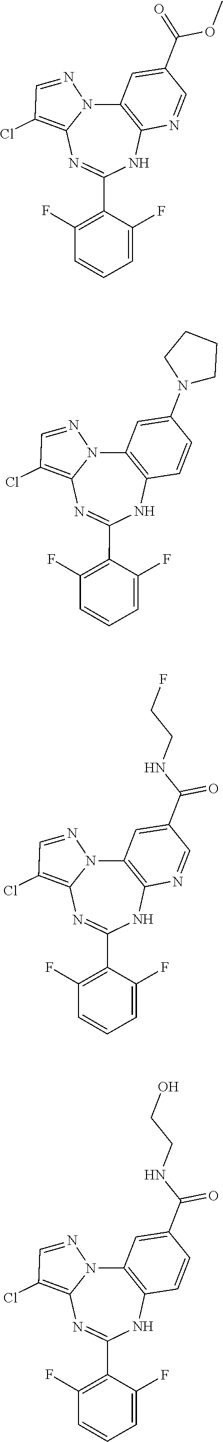 [SMILES: COC(=O)c1cnc2c(c1)-n1ncc(Cl)c1N=C(c1c(F)cccc1F)N2 ; Fc1cccc(F)c1C1=Nc2c(Cl)cnn2-c2cc(N3CCCC3)ccc2N1 ; O=C(NCCF)c1cnc2c(c1)-n1ncc(Cl)c1N=C(c1c(F)cccc1F)N2 ; O=C(NCCO)c1ccc2c(c1)-n1ncc(Cl)c1N=C(c1c(F)cccc1F)N2]

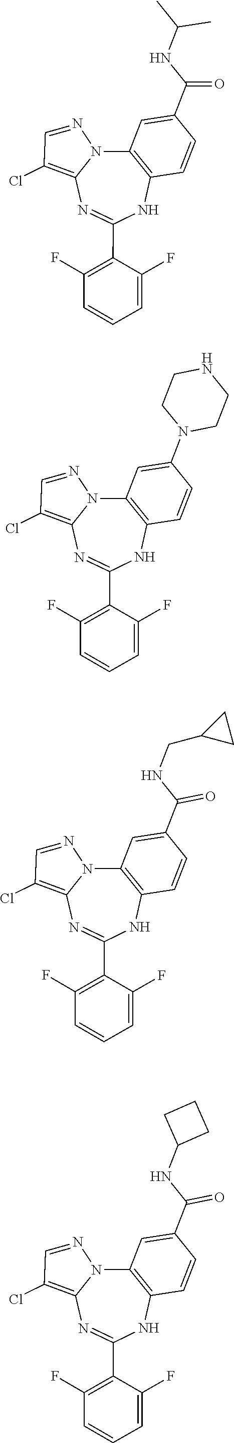 [SMILES: CC(C)NC(=O)c1ccc2c(c1)-n1ncc(Cl)c1N=C(c1c(F)cccc1F)N2 ; Fc1cccc(F)c1C1=Nc2c(Cl)cnn2-c2cc(N3CCNCC3)ccc2N1 ; O=C(NCC1CC1)c1ccc2c(c1)-n1ncc(Cl)c1N=C(c1c(F)cccc1F)N2 ; O=C(NC1CCC1)c1ccc2c(c1)-n1ncc(Cl)c1N=C(c1c(F)cccc1F)N2]

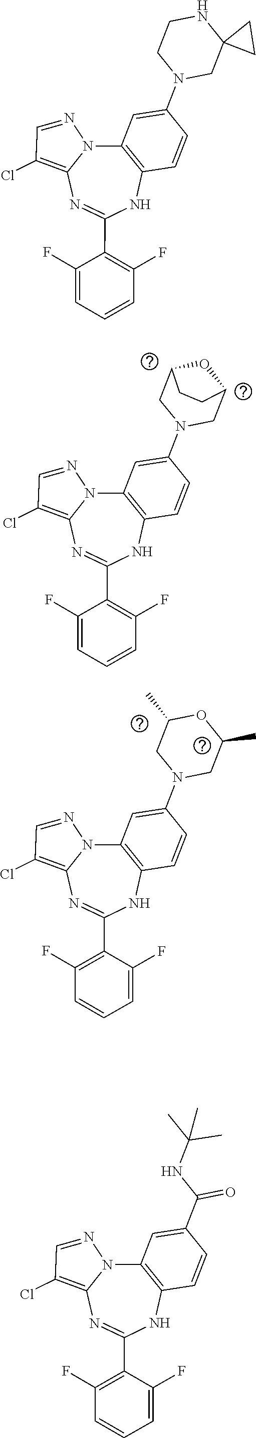

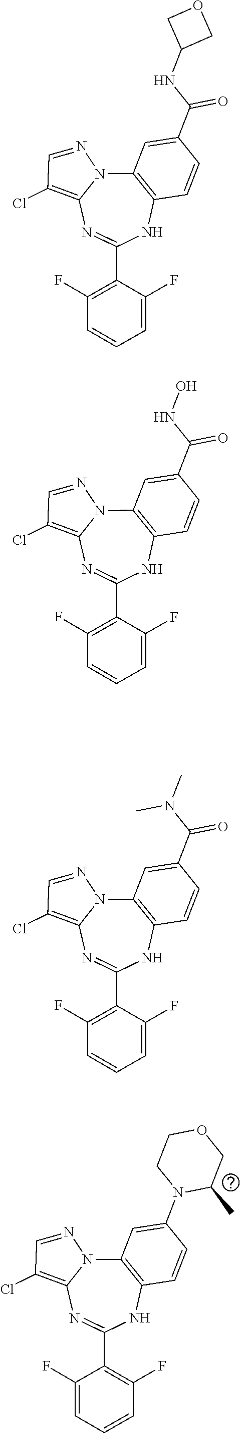 [SMILES: O=C(NC1COC1)c1ccc2c(c1)-n1ncc(Cl)c1N=C(c1c(F)cccc1F)N2 ; O=C(NO)c1ccc2c(c1)-n1ncc(Cl)c1N=C(c1c(F)cccc1F)N2 ; CN(C)C(=O)c1ccc2c(c1)-n1ncc(Cl)c1N=C(c1c(F)cccc1F)N2 ; C[C@@H]1COCCN1c1ccc2c(c1)-n1ncc(Cl)c1N=C(c1c(F)cccc1F)N2]

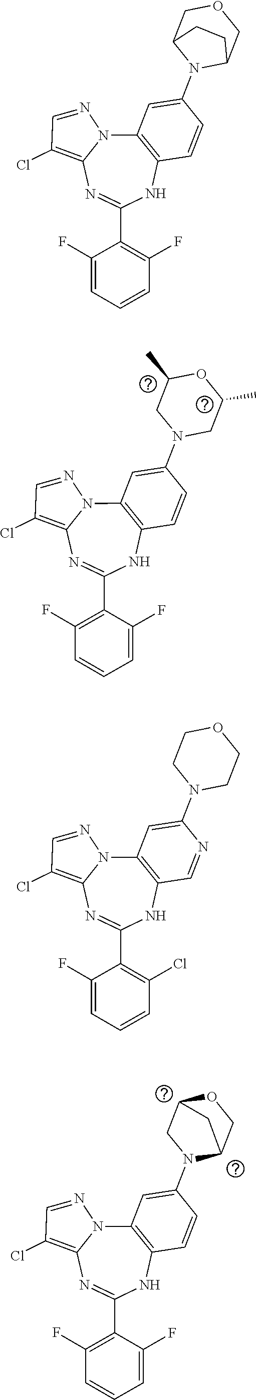 [SMILES: Fc1cccc(F)c1C1=Nc2c(Cl)cnn2-c2cc(N3C4CCC3COC4)ccc2N1 ; C[C@@H]1CN(c2ccc3c(c2)-n2ncc(Cl)c2N=C(c2c(F)cccc2F)N3)C[C@@H](C)O1 ; Fc1cccc(Cl)c1C1=Nc2c(Cl)cnn2-c2cc(N3CCOCC3)ncc2N1 ; Fc1cccc(F)c1C1=Nc2c(Cl)cnn2-c2cc(N3C[C@@H]4C[C@H]3CO4)ccc2N1]

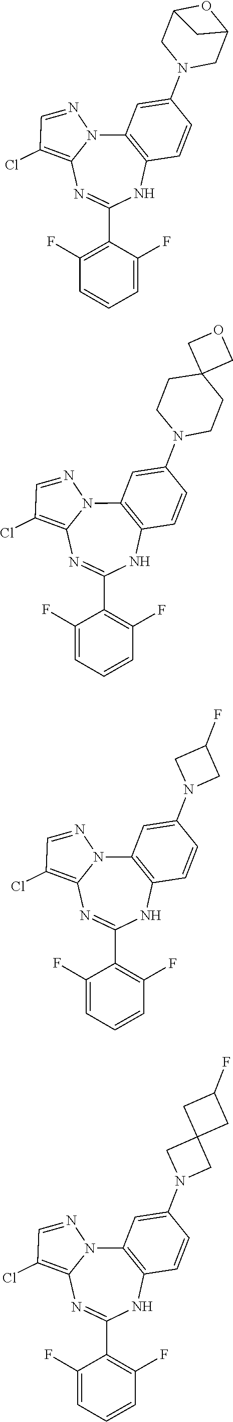 [SMILES: Fc1cccc(F)c1C1=Nc2c(Cl)cnn2-c2cc(N3CC4CC(C3)O4)ccc2N1 ; Fc1cccc(F)c1C1=Nc2c(Cl)cnn2-c2cc(N3CCC4(CC3)COC4)ccc2N1 ; Fc1cccc(F)c1C1=Nc2c(Cl)cnn2-c2cc(N3CC(F)C3)ccc2N1 ; Fc1cccc(F)c1C1=Nc2c(Cl)cnn2-c2cc(N3CC4(CC(F)C4)C3)ccc2N1]

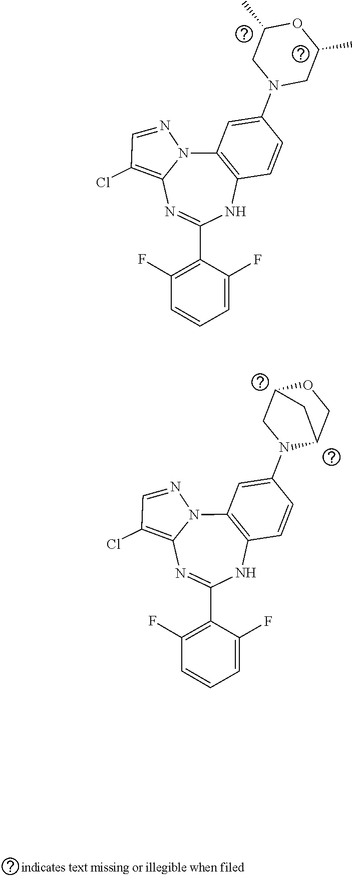 [SMILES: C[C@@H]1CN(c2ccc3c(c2)-n2ncc(Cl)c2N=C(c2c(F)cccc2F)N3)C[C@H](C)O1 ; Fc1cccc(F)c1C1=Nc2c(Cl)cnn2-c2cc(N3C[C@H]4C[C@@H]3CO4)ccc2N1]

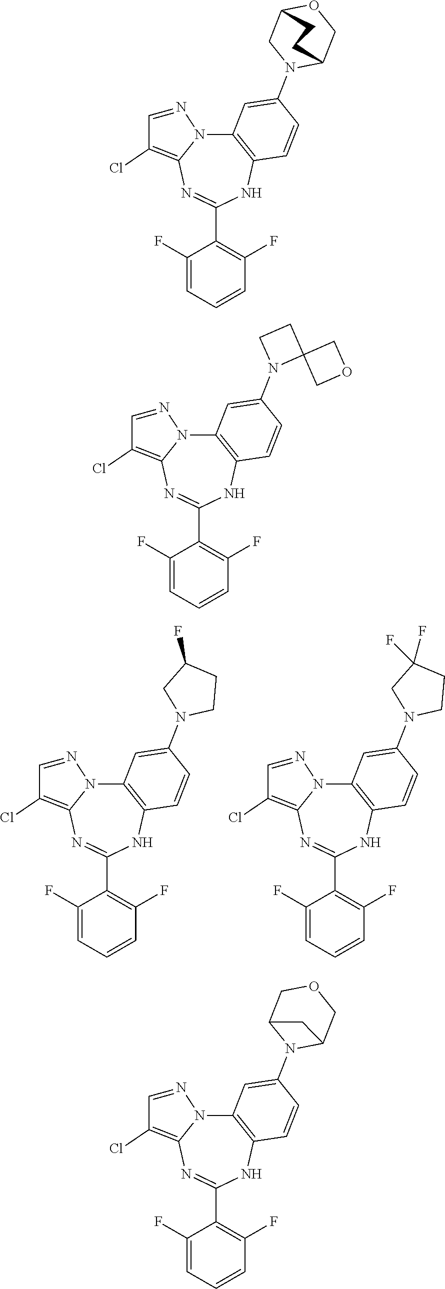

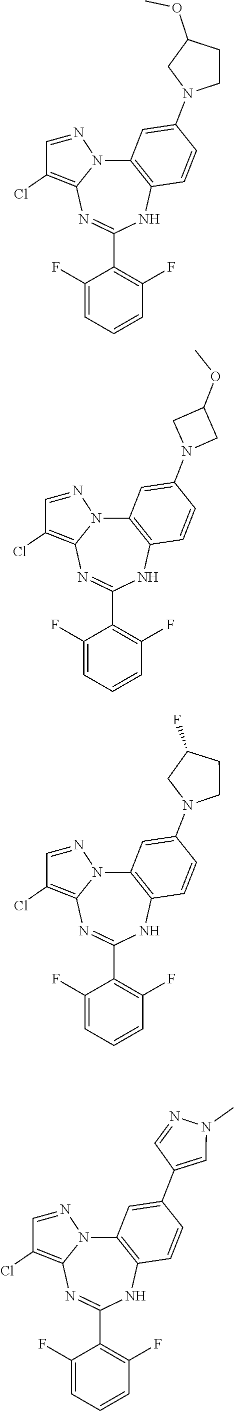 [SMILES: COC1CCN(c2ccc3c(c2)-n2ncc(Cl)c2N=C(c2c(F)cccc2F)N3)C1 ; COC1CN(c2ccc3c(c2)-n2ncc(Cl)c2N=C(c2c(F)cccc2F)N3)C1 ; Fc1cccc(F)c1C1=Nc2c(Cl)cnn2-c2cc(N3CC[C@@H](F)C3)ccc2N1 ; Cn1cc(-c2ccc3c(c2)-n2ncc(Cl)c2N=C(c2c(F)cccc2F)N3)cn1]

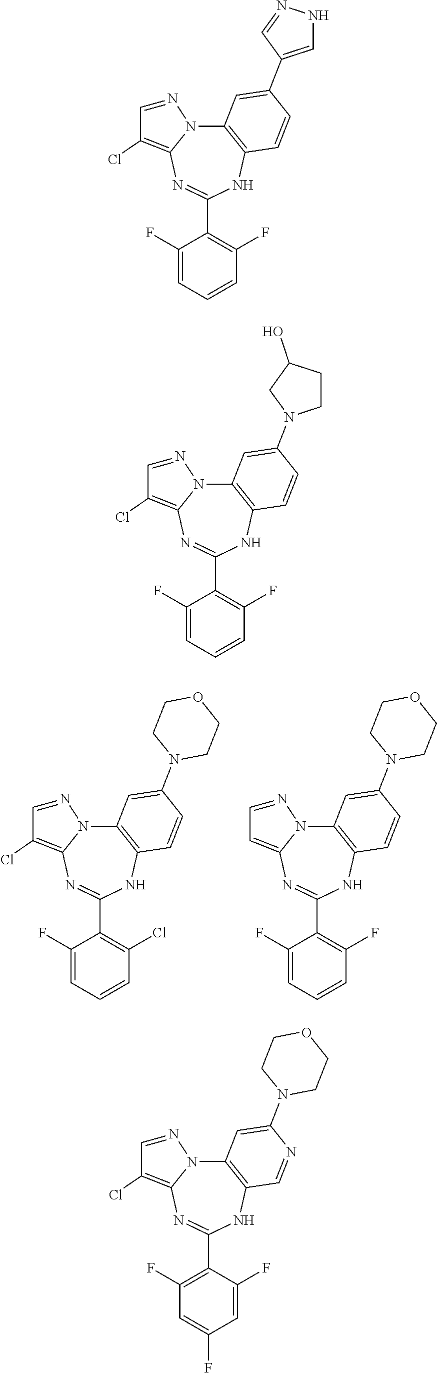

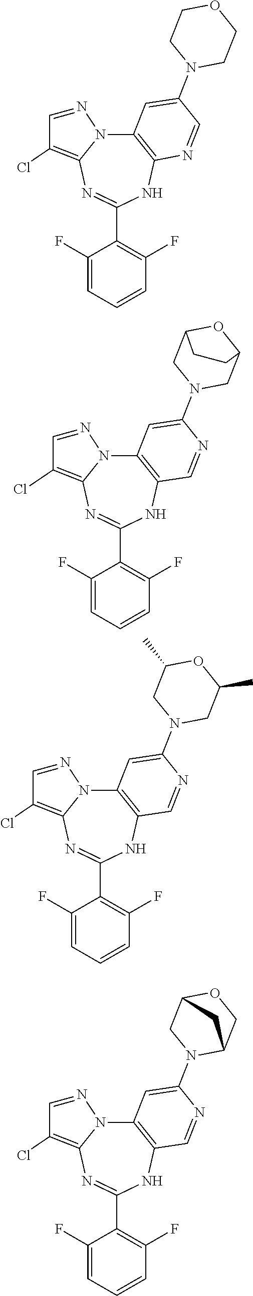

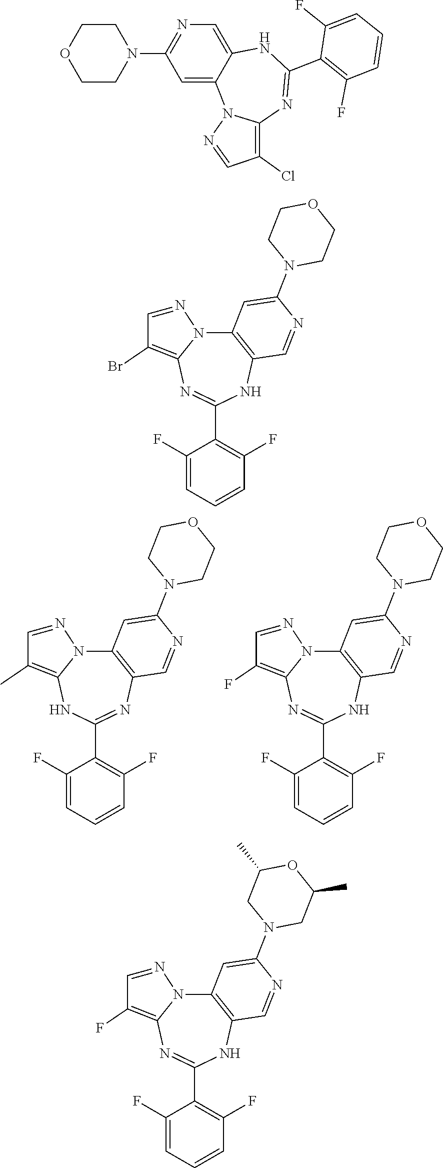

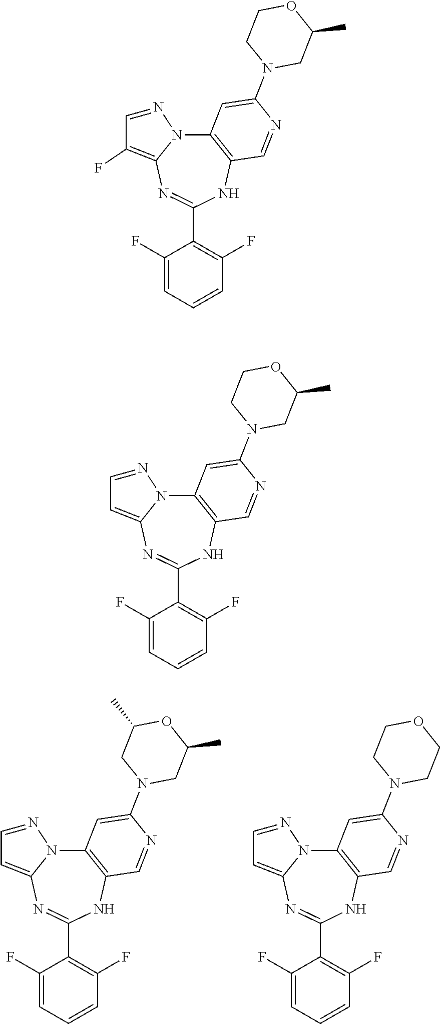 [SMILES: C[C@H]1CN(c2cc3c(cn2)NC(c2c(F)cccc2F)=Nc2c(F)cnn2-3)CCO1 ; C[C@H]1CN(c2cc3c(cn2)NC(c2c(F)cccc2F)=Nc2ccnn2-3)CCO1 ; C[C@H]1CN(c2cc3c(cn2)NC(c2c(F)cccc2F)=Nc2ccnn2-3)C[C@H](C)O1 ; Fc1cccc(F)c1C1=Nc2ccnn2-c2cc(N3CCOCC3)ncc2N1]

In one embodiment, the invention provides a pharmaceutical composition comprising a compound of the above Formula (I) and pharmaceutically acceptable salts thereof together with a pharmaceutically acceptable excipient.

In one embodiment, the invention provides compounds of the above Formula (I) and pharmaceutically acceptable salts thereof for use in therapy.

In one embodiment, the invention provides compounds of the above Formula (I) and pharmaceutically acceptable salts thereof for use in a method for the treatment of a disease associated with LRRK2.

In one embodiment, the invention relates to the use of a compound of the above Formula (I) and pharmaceutically acceptable salts thereof in the manufacture of a medicament for use in the treatment of a disease associated with LRRK2.

In one embodiment, the invention relates to a method for the treatment of a disease associated with LRRK2, including PD, the method comprising the administration of a therapeutically effective amount of a compound of the above formula A and pharmaceutically acceptable salts thereof to a patient in need thereof.

In another aspect, the invention provides methods of modulating the activity of a kinase by contacting cells containing a kinase with one or more compounds of the invention, such as any of those described above. The compound may inhibit activity of the kinase. The compound may increase activity of the kinase. The kinase may be LRRK2.

In embodiments of the use, the condition treated by the compounds of the invention is an autoimmune disease, inflammatory disease, bone disease, metabolic disease, neurological or neurodegenerative disease, cancer, cardiovascular disease, allergies, asthma, Alzheimer's disease, Parkinson's disease, skin disorder, eye disease, infectious disease, or hormone-related disease.

### DETAILED DESCRIPTION

### Chemical definitions

The expression alkyl refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”, also referred to herein as “lower alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), isobutyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently optionally substituted, i.e., unsubstituted (an “unsubstituted alkyl”) or substituted (a “substituted alkyl”) with one or more substituents; e.g., from 1 to 5 substituents, 1 to 3 substituents, or 1 substituent. In certain embodiments, the alkyl group is unsubstituted C₁₋₁₀ alkyl (e.g., —CH₃). In certain embodiments, the alkyl group is substituted C₁₋₁₀ alkyl. Common alkyl abbreviations include Me (—CH₃), Et (—CH₂CH₃), iPr (—CH(CH₃)₂), nPr (—CH₂CH₂CH₃), n-Bu (—CH₂CH₂CH₂CH₃), or i-Bu (—CH₂CH(CH₃)₂).

The expression heteroalkyl refers to an alkyl group, as defined herein, which further comprises 1 or more (e.g., 1, 2, 3, or 4) heteroatoms (e.g., oxygen, sulfur, nitrogen, boron, silicon, phosphorus) within the parent chain, wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkyl group refers to a saturated group having from 1 to 10 carbon atoms and 1, 2, 3, or 4 heteroatoms (“heteroC₁₋₁₀ alkyl”). In some embodiments, a heteroalkyl group is a saturated group having 1 to 9 carbon atoms and 1, 2, 3, or 4 heteroatoms (“heteroC₁₋₉ alkyl”). In some embodiments, a heteroalkyl group is a saturated group having 1 to 8 carbon atoms and 1, 2, 3, or 4 heteroatoms (“heteroC₁₋₈ alkyl”). In some embodiments, a heteroalkyl group is a saturated group having 1 to 7 carbon atoms and 1, 2, 3, or 4 heteroatoms (“heteroC₁₋₇ alkyl”). In some embodiments, a heteroalkyl group is a group having 1 to 6 carbon atoms and 1, 2, or 3 heteroatoms (“heteroC₁₋₆ alkyl”). In some embodiments, a heteroalkyl group is a saturated group having 1 to 5 carbon atoms and 1 or 2 heteroatoms (“heteroC₁₋₁₀ alkyl”). In some embodiments, a heteroalkyl group is a saturated group having 1 to 4 carbon atoms and lor 2 heteroatoms (“heteroC₁₋₄ alkyl”). In some embodiments, a heteroalkyl group is a saturated group having 1 to 3 carbon atoms and 1 heteroatom (“heteroC₁₋₃ alkyl”). In some embodiments, a heteroalkyl group is a saturated group having 1 to 2 carbon atoms and 1 heteroatom (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms (“heteroC₂₋₆ alkyl”).

The expression alkenyl refers to a radical of a straight-chain or branched hydrocarbon group having from 2 to 20 carbon atoms, one or more carbon-carbon double bonds (e.g., 1, 2, 3, or 4 carbon-carbon double bonds) (“C₂₋₂₀ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₉), and the like. Unless otherwise specified, each instance of an alkenyl group is independently optionally substituted, i.e., unsubstituted (an “unsubstituted alkenyl”) or substituted (a “substituted alkenyl”) with one or more substituents e.g., from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₁₀ alkenyl.

The term “heteroalkenyl,” as used herein, refers to an alkenyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkenyl group refers to a group having from 2 to 10 carbon atoms, at least one double bond, and 1, 2, 3, or 4 heteroatoms (“heteroC₂₋₁₀ alkenyl”). In some embodiments, a heteroalkenyl group has 2 to 9 carbon atoms at least one double bond, and 1, 2, 3, or 4 heteroatoms (“heteroC₂₋₉ alkenyl”). In some embodiments, a heteroalkenyl group has 2 to 8 carbon atoms, at least one double bond, and 1, 2, 3, or 4 heteroatoms (“heteroC₂₋₈ alkenyl”). In some embodiments, a heteroalkenyl group has 2 to 7 carbon atoms, at least one double bond, and 1, 2, 3, or 4 heteroatoms (“heteroC₂₋₇ alkenyl”). In some embodiments, a heteroalkenyl group has 2 to 6 carbon atoms, at least one double bond, and 1, 2, or 3 heteroatoms (“heteroC₂₋₆ alkenyl”). In some embodiments, a heteroalkenyl group has 2 to 5 carbon atoms, at least one double bond, and 1 or 2 heteroatoms (“heteroC₂₋₅ alkenyl”). In some embodiments, a heteroalkenyl group has 2 to 4 carbon atoms, at least one double bond, and 1 or 2 heteroatoms (“heteroC₂₋₄ alkenyl”). In some embodiments, a heteroalkenyl group has 2 to 3 carbon atoms, at least one double bond, and 1 heteroatom (“heteroC₂₋₃ alkenyl”). In some embodiments, a heteroalkenyl group has 2 to 6 carbon atoms, at least one double bond, and 1 or 2 heteroatoms (“heteroCC₂₋₆ alkenyl”).

The expression cycloalkyl refers to a saturated or partially unsaturated (for example, a cycloalkenyl group) cyclic group that contains one or more rings, e.g., 2 or 3 rings, and contains from 3 to 14 ring carbon atoms, such as from 3 to 10 (e.g., 3, 4, 5, 6 or 7) ring carbon atoms. The expression cycloalkyl refers furthermore to groups in which one or more hydrogen atoms have been replaced by fluorine, chlorine, bromine or iodine atoms or by OH, ═O, SH, ═S, NH₂, ═NH, N₃ or NO₂ groups, thus, for example, cyclic ketones such as, for example, cyclohexanone, 2-cyclohexenone or cyclopentanone. Further specific examples of cycloalkyl groups are a cyclopropyl, cyclobutyl, cyclopentyl, spiro[4,5]decanyl, norbornyl, cyclohexyl, cyclopentenyl, cyclohexadienyl, decalinyl, bicyclo[4.3.0]nonyl, tetraline, cyclopentylcyclohexyl, fluorocyclohexyl or cyclohex-2-enyl group.

The expression cycloheteroalkyl or heterocycle refers to a cycloalkyl group as defined above in which one or more (e.g., 1, 2, or 3) ring carbon atoms have been replaced by an oxygen, nitrogen, silicon, selenium, phosphorus or sulfur atom or a SO group or a SO₂ group. A cycloheteroalkyl or heterocycle group may have 1 or 2 rings containing from 3 to 10 (e.g., 3, 4, 5, 6 or 7) ring atoms (e.g., C, O, N or S). Cycloheteroalkyl or heterocycle groups include cycloheteroalkenyl or heterocycloalkenyl groups. The expression cycloheteroalkyl or heterocycle refers furthermore to groups that are substituted by fluorine, chlorine, bromine or iodine atoms or by OH, ═O, SH, ═S, NH₂, ═NH, N₃ or NO₂ groups. Examples are a piperidinyl, prolinyl, imidazolidinyl, piperazinyl, morpholinyl, urotro pinyl, pyrrolidinyl, tetrahydrothiophenyl, tetrahydropyranyl, tetrahydrofuryl or 2-pyrazolinyl group and also lactams, lactones, cyclic imides and cyclic anhydrides.

The expression alkylcycloalkyl refers to groups that contain both cycloalkyl and also alkyl, alkenyl or alkynyl groups in accordance with the above definitions, for example alkylcycloalkyl, cycloalkylalkyl, alkylcycloalkenyl, alkenylcycloalkyl and alkynylcycloalkyl groups. An alkylcycloalkyl group preferably contains a cycloalkyl group that contains one or two rings having from 3 to 10 (e.g., 3, 4, 5, 6 or 7) ring carbon atoms, and one or two alkyl or alkynyl groups having 1 or 2 to 6 carbon atoms.

The expression heteroalkylcycloalkyl refers to alkylcycloalkyl groups as defined above in which one or more (e.g., 1, 2 or 3) carbon atoms have been replaced by an oxygen, nitrogen, silicon, selenium, phosphorus or sulfur atom or a SO group or a SO₂ group. A heteroalkylcycloalkyl group preferably contains 1 or 2 rings having from 3 to 10 (e.g., 3, 4, 5, 6 or 7) ring atoms, and one or two alkyl, alkenyl, alkynyl or heteroalkyl groups having from 1 or 2 to 6 carbon atoms. Examples of such groups are alkylheterocycle, alkylheterocycloalkenyl, alkenylheterocycle, alkynylheterocycle, heteroalkylcycloalkyl, heteroalkylheterocycle and heteroalkylheterocycloalkenyl, the cyclic groups being saturated or mono-, di- or tri-unsaturated.

The expression aryl refers to an aromatic group that contains one or more rings, e.g., 2 or 3 rings, containing from 6 to 14 ring carbon atoms, such as from 6 to 10 ring carbon atoms. The expression aryl refers furthermore to groups that are substituted by fluorine, chlorine, bromine or iodine atoms or by CH₃, OH, SH, NH₂, N₃ or NO₂ groups. Examples are the phenyl, naphthyl, biphenyl, 2-fluorophenyl, anilinyl, 3-nitrophenyl or 4-hydroxyphenyl group.

The expression heteroaryl refers to an aromatic group that contains one or more rings, e.g., 2 or 3 rings, containing from 5 to 14 ring atoms, such as from 5 to 10 ring atoms, and contains one or more (e.g., 1, 2, 3 or 4) oxygen, nitrogen, phosphorus or sulfur ring atoms. The expression heteroaryl refers furthermore to groups that are substituted by fluorine, chlorine, bromine or iodine atoms or by CH₃, OH, SH, N₃, NH₂ or NO₂ groups. Examples are pyridyl (e.g. 4-pyridyl), imidazolyl (e.g. 2-imidazolyl), phenylpyrrolyl (e.g. 3-phenylpyrrolyl), thiazolyl, isothiazolyl, 1,2,3-triazolyl, 1,2,4-triazolyl, oxadiazolyl, thiadiazolyl, indolyl, indazolyl, tetrazolyl, pyrazinyl, pyrimidinyl, pyridazinyl, oxazolyl, isoxazolyl, triazolyl, tetrazolyl, isoxazolyl, indazolyl, indolyl, benzimidazolyl, benzoxazolyl, benzisoxazolyl, benzthiazolyl, pyridazinyl, quinolinyl, isoquinolinyl, pyrrolyl, purinyl, carbazolyl, acridinyl, pyrimidyl, 2,3′-bifuryl, pyrazolyl (e.g. 3-pyrazolyl) and isoquinolinyl groups.

The expression aralkyl refers to groups containing both aryl and also alkyl, alkenyl, alkynyl and/or cycloalkyl groups in accordance with the above definitions, such as, for example, arylalkyl, arylalkenyl, arylalkynyl, arylcycloalkyl, arylcycloalkenyl, alkylarylcycloalkyl and alkylarylcycloalkenyl groups. Specific examples of aralkyls are toluene, xylene, mesitylene, styrene, benzyl chloride, o-fluorotoluene, 1H-indene, tetraline, dihydronaphthalene, indanone, phenylcyclopentyl, cumene, cyclohexylphenyl, fluorene and indane. An aralkyl group preferably contains one or two aromatic ring systems containing from 6 to 10 carbon atoms and one or two alkyl, alkenyl and/or alkynyl groups containing from 1 or 2 to 6 carbon atoms and/or a cycloalkyl group containing 5 or 6 ring carbon atoms.

The expression heteroaralkyl refers to an aralkyl group as defined above in which one or more (e.g., 1, 2, 3 or 4) carbon atoms have been replaced by an oxygen, nitrogen, silicon, selenium, phosphorus, boron or sulfur atom, that is to say to groups containing both aryl or heteroaryl, respectively, and also alkyl, alkenyl, alkynyl and/or heteroalkyl and/or cycloalkyl and/or heterocycle groups in accordance with the above definitions. A heteroaralkyl group preferably contains one or two aromatic ring systems containing from 5 or 6 to 10 ring carbon atoms and one or two alkyl, alkenyl and/or alkynyl groups containing 1 or 2 to 6 carbon atoms and/or a cycloalkyl group containing 5 or 6 ring carbon atoms, wherein 1, 2, 3 or 4 of these carbon atoms have been replaced by oxygen, sulfur or nitrogen atoms.

Examples are arylheteroalkyl, arylheterocycle, arylheterocycloalkenyl, arylalkyl heterocycle, arylalkenylheterocycle, arylalkynylheterocycle, arylalkylhetero cycloalkenyl, heteroarylalkyl, heteroarylalkenyl, heteroarylalkynyl, heteroarylheteroalkyl, heteroarylcycloalkyl, heteroarylcycloalkenyl, heteroarylheterocycle, hetero arylheterocycloalkenyl, heteroarylalkylcycloalkyl, heteroarylalkylheterocycloalkenyl, hetero arylheteroalkylcycloalkyl, heteroarylheteroalkylcycloalkenyl and heteroarylheteroalkylhetero cycloalkyl groups, the cyclic groups being saturated or mono-, di- or tri-unsaturated. Specific examples are a tetrahydroisoquinolinyl, benzoyl, 2- or 3-ethylindolyl, 4-methylpyridino, 2-, 3- or 4-methoxyphenyl, 4-ethoxyphenyl, 2-, 3- or 4-carboxyphenylalkyl group.

As stated above, the expressions cycloalkyl, cycloheteroalkyl, heterocycle, alkylcycloalkyl, heteroalkylcycloalkyl, aryl, heteroaryl, aralkyl and heteroaralkyl also refer to groups that are substituted by fluorine, chlorine, bromine or iodine atoms or by CH₃, OH, ═O, SH, ═S, NH₂, ═NH, N₃ or NO₂ groups.

The expression carbocyclyl or carbocyclic refers to a radical of a non-aromatic cyclic hydrocarbon group having from 3 to 10 ring carbon atoms (“C₃₋₁₀ carbocyclyl”) and zero heteroatoms in the nonaromatic ring system. In some embodiments, a carbocyclyl group has 3 to 8 ring carbon atoms 10 (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (G), cyclooctenyl (G), bicyclo[2.2.1]heptanyl (C₇), bicyclo[2.2.2]octanyl (G), and the like. Exemplary C₃₋₁₀ carbocyclyl groups include, without 20 limitation, the aforementioned G-s carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

The expression heterocyclyl or heterocyclic refers to a radical of a 3- to 14-membered non-aromatic ring system having ring carbon atoms and 1 to 4 ring heteroatoms, wherein each heteroatom is independently selected from nitrogen, oxygen, sulfur, boron, phosphorus, and silicon (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1,4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups 5 containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

The expression optionally substituted means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction.

Heteroatoms, such as nitrogen, may have substituents, such as any suitable substituent described herein which satisfies the valencies of the heteroatoms and results in the formation of a stable moiety.

For example and without limitation, optional substituents include fluorine, chlorine, bromine, and iodine atoms and CF₃, CN, OH, ═O, SH, ═S, NH₂, ═NH, N₃ and NO₂ groups. Optional substituents also include C₁-C₁₀ alkyl, C₂-C₁₀ alkenyl, C₁-C₁₀ heteroalkyl, C₃-C₁₆ cycloalkyl, C₂-C₁₇ heterocycle, C₄-C₂₀ alkylcycloalkyl, C₂-C₁₉ heteroalkylcycloalkyl, C₆-Cis aryl, C₁₋₁₇ heteroaryl, C₇-C₂₀ aralkyl or C₂-C₁₉ heteroaralkyl, C₁-C₆ alkyl, C₂-C₆ alkenyl, C₁-C₆ heteroalkyl, C₃-C₁₀ cycloalkyl, C₂-C₉ heterocycle, C₇-C₁₂ alkylcycloalkyl, C₂-C₁₁ heteroalkylcycloalkyl, C₆-C₁₀ aryl, C₁-C₉ heteroaryl, C₇-C₁₂ aralkyl, C₂-C₁₁ heteroaralkyl, and C₁-C₁₀ haloalkyl groups.

Exemplary substituents are F, Cl, Br, OH, SH, ═O, NH₂, amino, C₁₋₄ alkyl, C₁₋₄ heteroalkyl cyclopropyl, SF₅, NO, NO₂.

Other exemplary substituents are F, Cl, Br, OH, SH, ═O, NH₂, C₁₋₄ alkyl (e.g. methyl, ethyl, t-butyl), NMe₂, CONH₂, CH₂NMe₂, NHSO₂Me, C(CH₃)₂CN, COMe, OMe, SMe, COOMe, COOEt, CH₂COOH, OCH₂COOH, COOH, SOMe, SO₂Me, cyclopropyl, SO₂NH₂, SO₂NHMe, SO₂CH₂CH₂OH, NHCH₂CH₂OH, CH₂CH₂OCH₃, SF₅, SO₂NMe₂, NO, NO₂, OCF₃, SO₂CF₃, CN or CF₃.

Other exemplary substituents are F, Cl, Br, Me, OMe, CN or CF₃.

The term halogen preferably refers to F, Cl, Br or I.

When an aryl, heteroaryl, cycloalkyl, alkylcycloalkyl, heteroalkylcycloalkyl, heterocycle, aralkyl or heteroaralkyl group contains more than one ring, these rings may be bonded to each other via a single or double bond or these rings may be annulated.

According to certain embodiments, all alkyl, alkenyl, alkynyl, heteroalkyl, aryl, heteroaryl, cycloalkyl, heterocycle, alkylcycloalkyl, heteroalkylcycloalkyl, aralkyl and heteroaralkyl groups described herein may optionally be substituted.

Other optional substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{CC}, —C(O)R^{aa}, —CO₂H, —CHO, —C(OR^{cc})₂, —CO₂R^{aa}, —OC(O)R^{aa}, —OCO₂R^{aa}, —C(O)N(Rb)₂, —C(O)N(R^{aa})(R^{bb}), —OC(O)N(R^{bb})₂, —NR^{bb}C(O)R^{aa}, —NR^{bb}CO₂R^{aa}, —NR^{bb}C(O)N(R^{bb})₂, —C(NR^{bb})R^{aa}, —C(NR^{bb})OR^{aa}, —OC(NR^{bb})R^{aa}, —OC(NR^{bb})OR^{aa}, —C(NR^{bb})N(R^{bb})₂, —OC(NR^{bb})N(R^{bb})₂, —NR^{bb}C(NR^{bb})N(R^{bb})₂, —C(O)NR^{bb}SO₂R^{aa}, —NR^{bb}SO₂R^{aa}, —SO₂N(R^{bb})₂, —SO₂R^{aa}, —SO₂R^{aa}, —SO₂OR^{aa}, —OSO₂R^{aa}, —S(O)R^{aa}, e.g., —S(O)R^{aa}, —OS(O)R^{aa}, —Si(R^{aa})₃, —OSi(R^{aa})₃ —C(S)N(R^{bb})₂, —C(O)SR^{aa}, —C(S)SR^{aa}, —SC(S)SR^{aa}, —SC(O)SR^{aa}, —OC(O)SR^{aa}, —SC(O)OR^{aa}, —SC(O)R^{aa}, —P(O)₂R^{aa}, —OP(O)₂R^{aa}, —P(O)(R^{aa})₂, —OP(O)(R^{aa})₂, —OP(O)(OR^{cc})₂, —P(O)₂N(R^{bb})₂, —OP(O)₂N(R^{bb})₂, —P(O)(NR^{bb})₂, —OP(O)(NR^{bb})₂, —NR^{bb}P(O)(OR^{cc})₂, —NR^{bb}P(O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), C₁₋₁₀ alkyl, C₁₋₁₀ haloalkyl, C₂₋₁₀ alkenyl, C₃₋₁₀ carbocyclyl, 3-14 membered heterocyclyl, C₆₋₁₄ aryl, and 5-14 membered heteroaryl, wherein each alkyl, alkenyl, carbocyclyl, heterocyclyl, aryl, and heteroaryl is independently substituted with 0, 1, 2, 3, 4, or 5 R^{dd} groups; or two geminal hydrogens on a carbon atom are replaced with the group ═O, ═S, ═NN(R^{bb})₂, ═NR^{bb}C(O)R^{aa}, —NNR^{bb}C(O)OR^{aa}, ═NNR^{bb}S(O)₂R^{aa}, ═NR^{bb}, or ═NOR^{cc}; in which:
each instance of R^{aa} is, independently, selected from C₁₋₁₀ alkyl, C₁₋₁₀ heteroalkyl, C₁₋₁₀ haloalkyl, C₂₋₁₀ alkenyl, C₃₋₁₀ cycloalkyl, C₃₋₁₀ cycloheteroalkyl, C₃₋₁₀ cycloalkenyl, C₃₋₁₀ cycloheteroalkenyl, C₃₋₁₀ carbocyclyl, 3-14 membered heterocyclyl, C₆₋₁₄ aryl, and 5-14 membered heteroaryl, or two R^{aa} groups are joined to form a 3-14 membered cycloalkyl, 3-14 membered cycloheteroalkyl, 3-14 membered heterocyclyl or 5-14 membered heteroaryl ring, wherein each alkyl, heteroalkyl, alkenyl, cycloalkyl, cycloheteroalkyl, cycloalkenyl, cycloheteroalkenyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(O)R^{aa}, —C(O)N(R^{cc})₂, —CO₂R^{aa}, —SO₂R^{aa}, —C(NR^{cc})OR^{aa}, —C(NR^{cc})N(R^{cc})₂, —SO₂N(R^{cc})₂, —SO₂R^{cc}, —SO₂OR^{cc}, —SOR^{aa}, —C(S)N(R^{cc})₂, —C(O)SR^{cc}, —C(S)SR^{cc}, —P(O)₂R^{aa}, —P(O)(R^{aa})₂, —P(O)₂N(R^{cc})₂, —P(O)(NR^{cc})₂, C₁₋₁₀ alkyl, C₁₋₁₀ heteroalkyl, C₁₋₁₀ haloalkyl, C₂₋₁₀ alkenyl, C₃₋₁₀ cycloalkyl, C₃₋₁₀ cycloheteroalkyl, C₃₋₁₀ cycloalkenyl, C₃₋₁₀ cycloheteroalkenyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, heteroalkyl, alkenyl, cycloalkyl, cycloheteroalkyl, cycloalkenyl, cycloheteroalkenyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, C₁₋₁₀ alkyl, C₁₋₁₀ haloalkyl, C₂₋₁₀ alkenyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(Rn); CX~, —N(OR^{ee})R^{ff}, —SH, —SR^{ee}, —SSR^{ee}, —C(O)R^{ee}, —CO₂H, —CO₂R^{ee}, —OC(O)R^{ee}, —OCO₂R^{ee}, —C(O)N(R^{ff})₂, —OC(O)N(R^{ff})₂, —NR^{ff}C(O)R^{ee}, —NR^{ff}CO₂R^{ee}, —NR^{ff}C(O)N(R^{ff})₂, —C(NR^{ff})OR^{ee}, —OC(NR^{ff})R^{ee}, —OC(NR^{ff})OR^{ee}, —C(NR^{ff})N(R^{ff})₂, —OC(NR^{ff})N(R^{ff})₂, —NR^{ff}C(NR^{ff})N(R^{ff})₂, —NR^{ff}SO₂R^{ee}, —SO₂R^{ee}, —SO₂N(R^{ff})₂, —SO₂R^{ee}, —SO₂OR^{ee}, —OSO₂R^{ee}, —S(O)R^{ee}, e.g., —S(O)R^{cc}, —Si(R^{ee})₃, —OSi(R^{ee})₃, —C(S)N(R^{ff})₂, —C(O)SR^{ee}, —C(S)SR^{ee}, —SC(S)SR^{ee}, —P(O)₂R^{ee}, —P(O)(R^{ee})₂, —OP(O)(R^{ee})₂, —OP(O)(OR^{ee})₂, C₁₋₆ alkyl, C₁₋₆ heteroalkyl, C₁₋₆ haloalkyl, C₂₋₆ alkenyl, C₃₋₁₀ carbocyclyl, 3-10 membered heterocyclyl, C₆₋₁₀ aryl, 5-10 membered heteroaryl, wherein each alkyl, alkenyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;
each instance of R^{ee} is, independently, selected from C₁₋₆ alkyl, C₁₋₆ haloalkyl, C₂₋₆ alkenyl, C₃₋₁₀ carbocyclyl, 3-10 membered heterocyclyl, C₆₋₁₀ aryl, 5-10 membered heteroaryl, wherein each alkyl, alkenyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₆ alkyl, C₁₋₆ haloalkyl, C₂₋₆ alkenyl, C₃₋₁₀ carbocyclyl, 3-10 membered heterocyclyl, C₆₋₁₀ aryl, 5-10 membered heteroaryl, or two R^{ff} groups are joined to form a 3-14 membered heterocyclyl or 5-14 membered heteroaryl ring, wherein each alkyl, alkenyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁻X⁻, —NH₂(C₁₋₆ alkyl)⁺X⁻-MR⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(O)(C₁₋₆ alkyl), —CO₂H, —CO₂(C₁₋₆ alkyl), —OC(O)(C₁₋₆ alkyl), —OCO₂(C₁₋₆ alkyl), —C(O)NH₂, —C(O)N(C₁₋₆ alkyl)₂, —OC(O)NH(C₁₋₆ alkyl), —NHC(O)(C₁₋₆ alkyl), —N(C₁₋₆ alkyl)C(O)(C₁₋₆ alkyl), —NHCO₂(C₁₋₆ alkyl), —NHC(O)N(C₁₋₆ alkyl)₂, —NHC(O)NH(C₁₋₆ alkyl), —NHC(O)NH₂, —C(NH)O(C₁₋₆ alkyl), —OC(NH)(C₁₋₆ alkyl), —OC(NH)OC₁₋₆ alkyl, —C(NH)N(C₁₋₆ alkyl)₂, —C(NH)NH(C₁₋₆ alkyl), —C(NH)NH₂, —OC(NH)N(C₁₋₆ alkyl)₂, —OC(NH)NH(C₁₋₆ alkyl), —OC(NH)NH₂, —NHC(NH)N(C₁₋₆ alkyl)₂, —NHC(NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂₀C₁₋₆ alkyl, —OSO₂C₁₋₆ alkyl, —SOC₁₋₆ alkyl, —Si(C₁₋₆ alkyl)₃, —OSi(C₁₋₆ alkyl)₃ —C(S)N(C₁₋₆ alkyl)₂, C(S)NH(C₁₋₆ alkyl), C(S)N₁₂, —C(O)S(C₁₋₆ alkyl), —C(S)SC₁₋₆ alkyl, —SC(S)SC₁₋₆ alkyl, —P(O)₂(C₁₋₆ alkyl), —P(O)(C₁₋₆ alkyl)₂, —OP(O)(C₁₋₆ alkyl)₂, —OP(O)(OC₁₋₆ alkyl)₂, C₁₋₆ alkyl, C₁₋₆ haloalkyl, C₂₋₆ alkenyl, C₃₋₁₀ carbocyclyl, C₃₋₁₀ aryl, 3-10 membered heterocyclyl, 5-10 membered heteroaryl; or two geminal R^{gg} substituents can be joined to form ═O or ═S; wherein X⁻ is a counterion.

### Compounds:

In one aspect, the invention provides a compound of Formula (I).

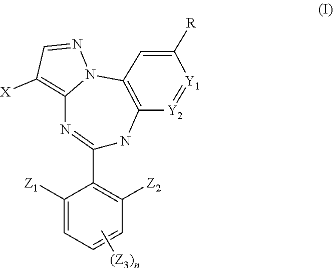

or an enantiomer, mixture of enantiomers, tautomer, or pharmaceutically acceptable salt thereof,
wherein: n is 1, 2, or 3;
Y₁ and Y₂ are independently N or CH;
Z₁, Z₂, and Z₃ are independently selected from H, —OH, halo, C₁-C₆ alkyl, C₁-C₆ heteroalkyl, haloalkyl, alkoxy, haloalkxoxy, —CH(OH)-alkyl, hydroxyalkyl, hydroxyalkoxy, amino, cyano, or morpholino;
X is H, —OH, halo, cyano, alkyl, haloalkyl, cyanoalkyl, or cycloalkyl with one or more heteroatoms, wherein the heteroatoms are selected from a group consisting of N, O, or S;
R is H, halo, substituted or unsubstituted aryl, substituted or unsubstituted C₁-C₆-alkyl, branched alkyl, alkenyl, alkynyl, haloalkyl, alkoxy, cycloalkoxy, haloalkoxy, -cyano, —CH₂-cycloalkyl, —CF₂-cycloalky, —CH(CH₃)-cycloalkyl, —CH₂-aryl, —CF₂-aryl, —CH(—CH₃)-aryl, C(═O)-alkyl, —C(═O)cycloalkyl, —C(═O)—NH-alkyl, —COOH (and esters and carboxamides thereof), C(═O)—NH—S(═O)₂alkyl, C(═O)—NH—S(═O)₂aryl, —C(═O)-morpholine, —C(═O)-heterocycles, —C(—CH₃)₂—OH, —CH₂—C(═O)—NH₂; -hydroxy, alkylhydroxy, alkyl-COOH (and esters and carboxamides thereof), substituted or unsubstituted alkylsulfonyl, arylsulfonyl, amino, —NHC(═O)alkyl, —N(alkyl)C(═O)alkyl, —NHC(═O)aryl, —N(alkyl)C(═O)aryl, —N(H)S(═O)₂alkyl, —N(H)S(═O)₂aryl, —N(alkyl)S(═O)₂alkyl, cyanoalkyl, haloalkyl, —S═(O)₂-alkyl, —S═(O)₂-cycloalkyl, —S═(O)₂-aryl, —S(═O)₂N(H)alkyl, substituted or unsubstituted morpholine, 3-7 membered heterocycle, any of which may have one or more substituents, 3-7 membered cycloalkyl or heterocycle, wherein the 3-7 membered cycloalkyl or heterocycle is fused with another 3-7 cycloalkyl or heterocycle, wherein the rings are spiro, bridged bicyclic, or spiro, wherein the at least one heteroatom in the heterocycle rings are independently selected from O, S, and N. In certain embodiments, when Y₁ is CH and Y₂ is CH; R is not any of H, fluoro, chloro, or methyl.

In another embodiment, in the compound of Formula (I), Y₁ is N and Y₂ is CH.

In another embodiment, in the compound of Formula (I), Y₁ is CH and Y₂ is N.

In another embodiment, in the compound of Formula (I), Z₁ and Z₂ are each fluoro and Z₃ is H.

In another embodiment, in the compound of Formula (I), Z₁ and Z₂ are fluoro and chloro respectively, and Z₃ is H.

In another embodiment, in the compound of Formula (I), Z₁, Z₂, and Z₃ are fluoro.

In another embodiment, in the compound of Formula (I), R is selected from a group consisting of: chloro, hydrogen, bromo, fluoro, methyl, methylcyano, and morpholine.

In another embodiment, in the compound of Formula (I), X is H, bromo, chloro, fluoro, or methyl.

In another embodiment, in the compound of Formula (I), R is selected from a group consisting of substituted or unsubstituted morpholines, amides, piperazines, alkyls, azetidine, heterocycle, or heteroaryl.

In another embodiment, in the compound of Formula (I), R is not any of H, fluoro, chloro, or methyl.

In another embodiment, in the compound of Formula (I), X is not any of H, fluoro, chloro, or cyano.

In another embodiment, in the compound of Formula (I), R is selected from a group consisting of: H, bromo, amide, ester, carboxylic acid,

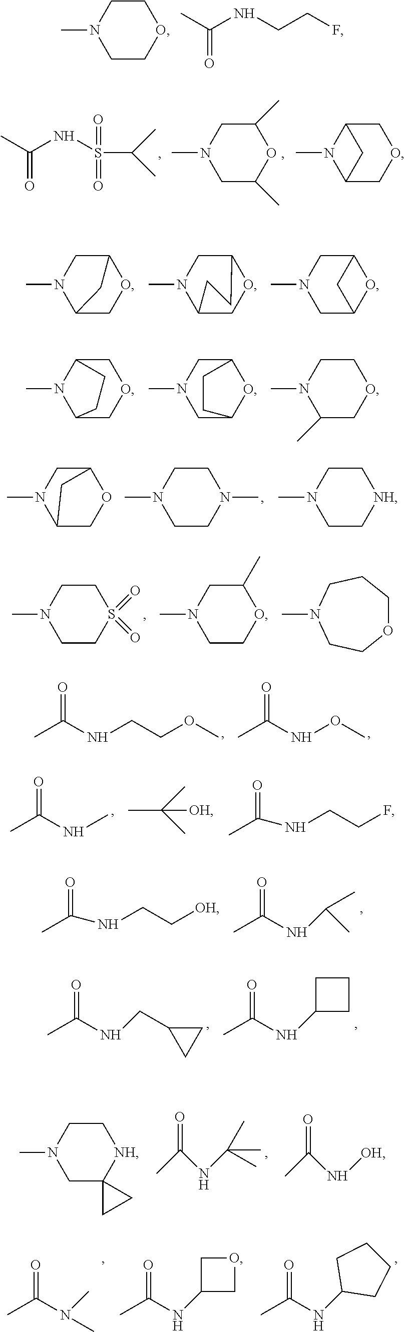

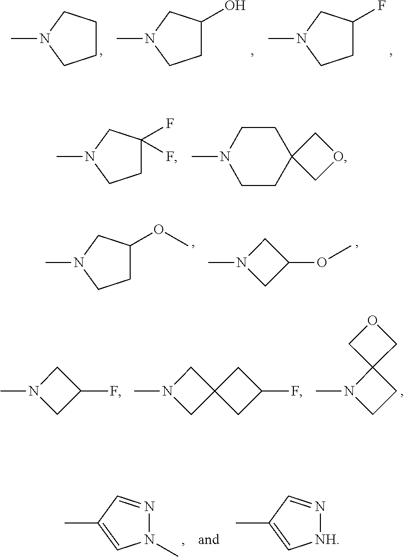

In another embodiment, in the compound of Formula (I), the compound is selected from a group consisting of:

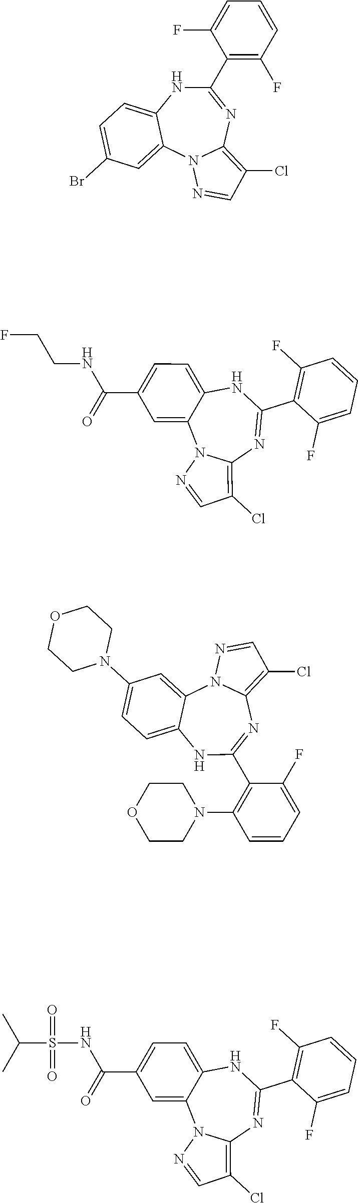 [SMILES: Fc1cccc(F)c1C1=Nc2c(Cl)cnn2-c2cc(Br)ccc2N1 ; O=C(NCCF)c1ccc2c(c1)-n1ncc(Cl)c1N=C(c1c(F)cccc1F)N2 ; Fc1cccc(N2CCOCC2)c1C1=Nc2c(Cl)cnn2-c2cc(N3CCOCC3)ccc2N1 ; CC(C)S(=O)(=O)NC(=O)c1ccc2c(c1)-n1ncc(Cl)c1N=C(c1c(F)cccc1F)N2]

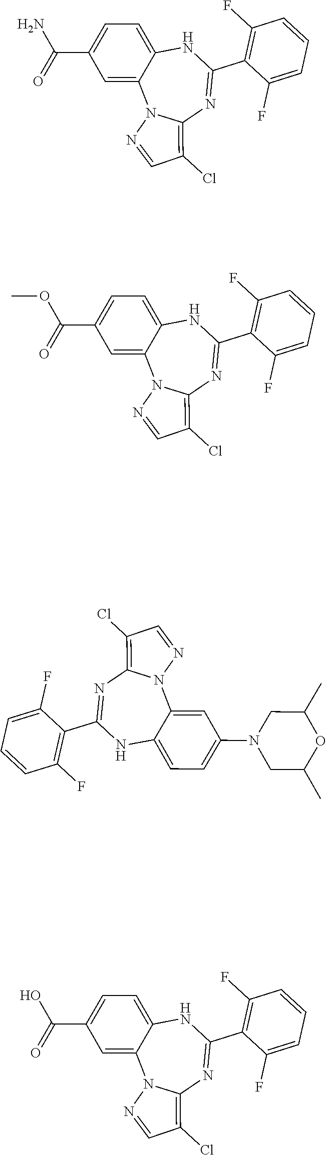 [SMILES: NC(=O)c1ccc2c(c1)-n1ncc(Cl)c1N=C(c1c(F)cccc1F)N2 ; COC(=O)c1ccc2c(c1)-n1ncc(Cl)c1N=C(c1c(F)cccc1F)N2 ; CC1CN(c2ccc3c(c2)-n2ncc(Cl)c2N=C(c2c(F)cccc2F)N3)CC(C)O1 ; O=C(O)c1ccc2c(c1)-n1ncc(Cl)c1N=C(c1c(F)cccc1F)N2]

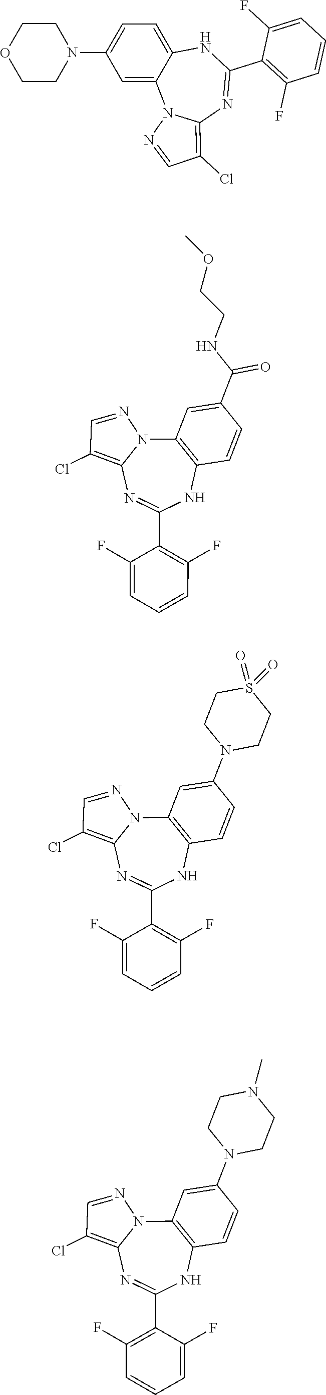 [SMILES: Fc1cccc(F)c1C1=Nc2c(Cl)cnn2-c2cc(N3CCOCC3)ccc2N1 ; COCCNC(=O)c1ccc2c(c1)-n1ncc(Cl)c1N=C(c1c(F)cccc1F)N2 ; O=S1(=O)CCN(c2ccc3c(c2)-n2ncc(Cl)c2N=C(c2c(F)cccc2F)N3)CC1 ; CN1CCN(c2ccc3c(c2)-n2ncc(Cl)c2N=C(c2c(F)cccc2F)N3)CC1]

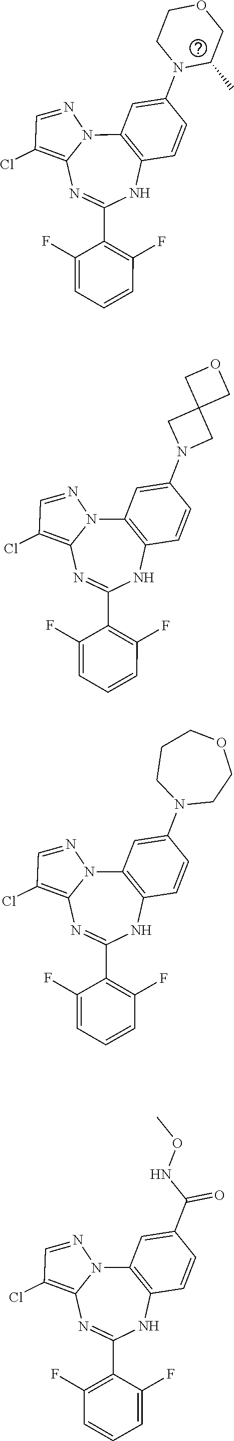 [SMILES: C[C@H]1COCCN1c1ccc2c(c1)-n1ncc(Cl)c1N=C(c1c(F)cccc1F)N2 ; Fc1cccc(F)c1C1=Nc2c(Cl)cnn2-c2cc(N3CC4(COC4)C3)ccc2N1 ; Fc1cccc(F)c1C1=Nc2c(Cl)cnn2-c2cc(N3CCCOCC3)ccc2N1 ; CONC(=O)c1ccc2c(c1)-n1ncc(Cl)c1N=C(c1c(F)cccc1F)N2]

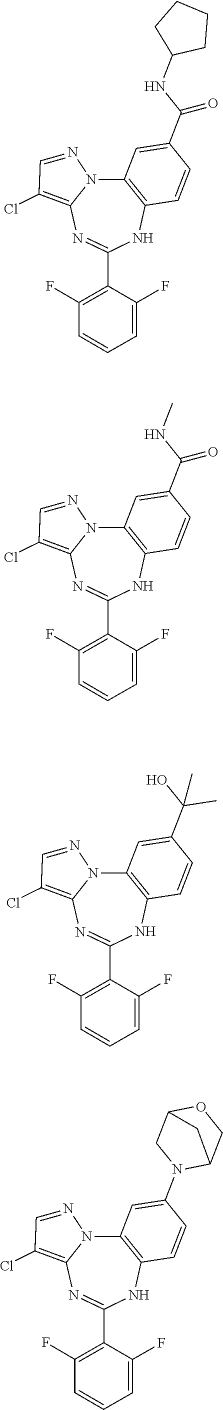 [SMILES: O=C(NC1CCCC1)c1ccc2c(c1)-n1ncc(Cl)c1N=C(c1c(F)cccc1F)N2 ; CNC(=O)c1ccc2c(c1)-n1ncc(Cl)c1N=C(c1c(F)cccc1F)N2 ; CC(C)(O)c1ccc2c(c1)-n1ncc(Cl)c1N=C(c1c(F)cccc1F)N2 ; Fc1cccc(F)c1C1=Nc2c(Cl)cnn2-c2cc(N3CC4CC3CO4)ccc2N1]

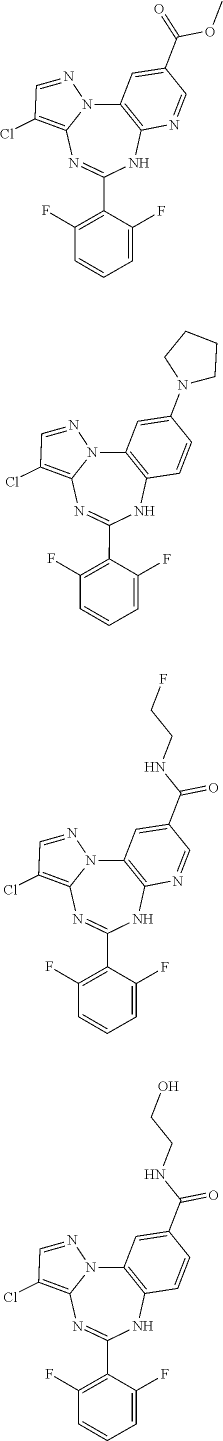 [SMILES: COC(=O)c1cnc2c(c1)-n1ncc(Cl)c1N=C(c1c(F)cccc1F)N2 ; Fc1cccc(F)c1C1=Nc2c(Cl)cnn2-c2cc(N3CCCC3)ccc2N1 ; O=C(NCCF)c1cnc2c(c1)-n1ncc(Cl)c1N=C(c1c(F)cccc1F)N2 ; O=C(NCCO)c1ccc2c(c1)-n1ncc(Cl)c1N=C(c1c(F)cccc1F)N2]

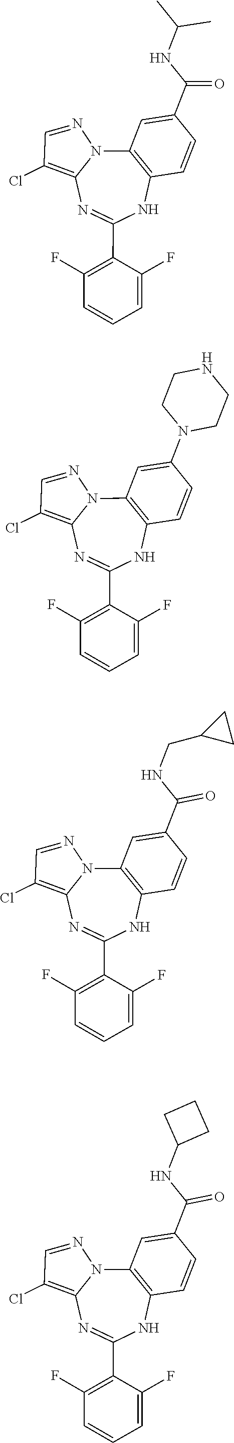 [SMILES: CC(C)NC(=O)c1ccc2c(c1)-n1ncc(Cl)c1N=C(c1c(F)cccc1F)N2 ; Fc1cccc(F)c1C1=Nc2c(Cl)cnn2-c2cc(N3CCNCC3)ccc2N1 ; O=C(NCC1CC1)c1ccc2c(c1)-n1ncc(Cl)c1N=C(c1c(F)cccc1F)N2 ; O=C(NC1CCC1)c1ccc2c(c1)-n1ncc(Cl)c1N=C(c1c(F)cccc1F)N2]

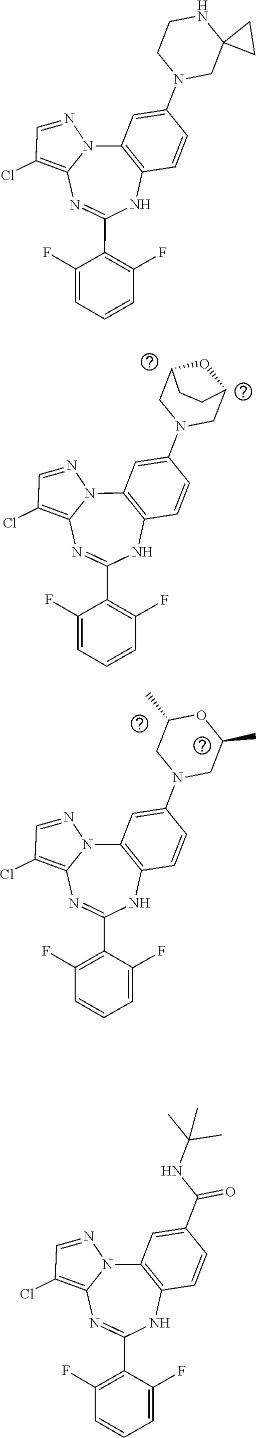

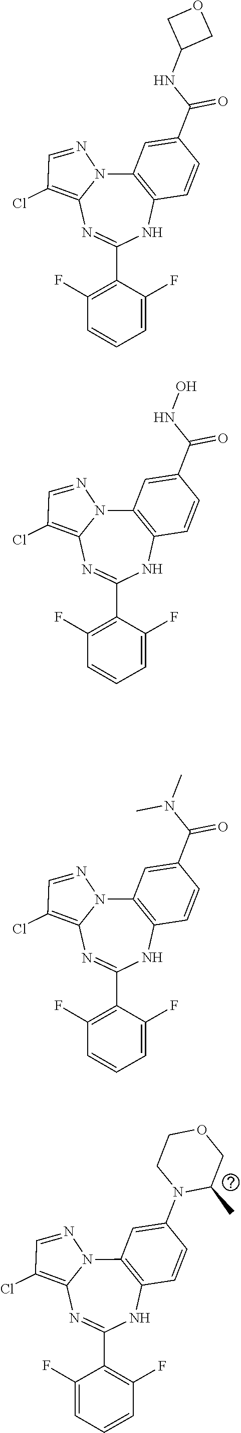 [SMILES: O=C(NC1COC1)c1ccc2c(c1)-n1ncc(Cl)c1N=C(c1c(F)cccc1F)N2 ; O=C(NO)c1ccc2c(c1)-n1ncc(Cl)c1N=C(c1c(F)cccc1F)N2 ; CN(C)C(=O)c1ccc2c(c1)-n1ncc(Cl)c1N=C(c1c(F)cccc1F)N2 ; C[C@@H]1COCCN1c1ccc2c(c1)-n1ncc(Cl)c1N=C(c1c(F)cccc1F)N2]

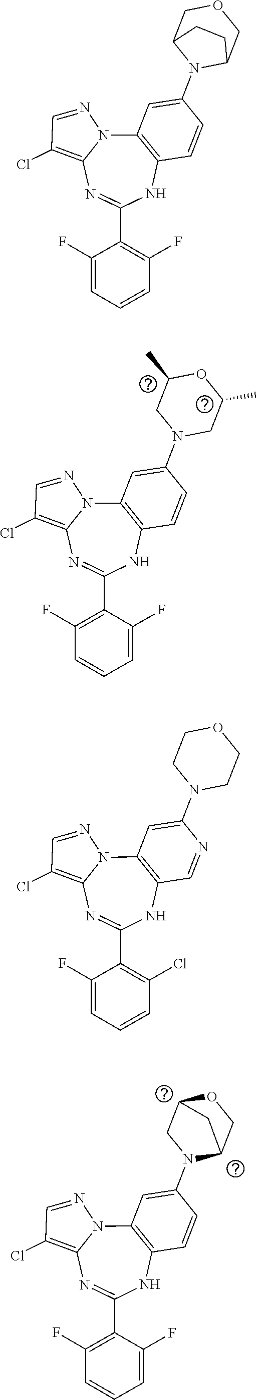 [SMILES: Fc1cccc(F)c1C1=Nc2c(Cl)cnn2-c2cc(N3C4CCC3COC4)ccc2N1 ; C[C@@H]1CN(c2ccc3c(c2)-n2ncc(Cl)c2N=C(c2c(F)cccc2F)N3)C[C@@H](C)O1 ; Fc1cccc(Cl)c1C1=Nc2c(Cl)cnn2-c2cc(N3CCOCC3)ncc2N1 ; Fc1cccc(F)c1C1=Nc2c(Cl)cnn2-c2cc(N3C[C@@H]4C[C@H]3CO4)ccc2N1]

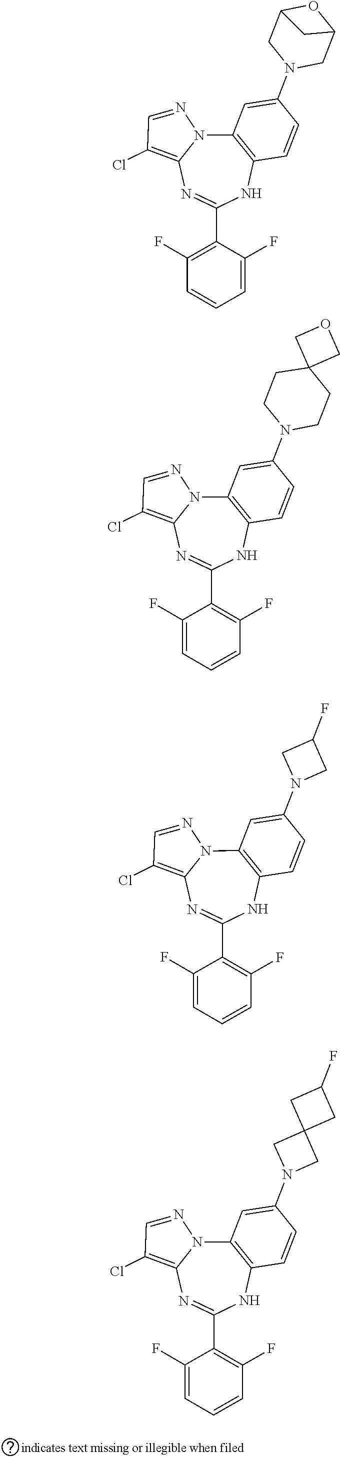 [SMILES: Fc1cccc(F)c1C1=Nc2c(Cl)cnn2-c2cc(N3CC4CC(C3)O4)ccc2N1 ; Fc1cccc(F)c1C1=Nc2c(Cl)cnn2-c2cc(N3CCC4(CC3)COC4)ccc2N1 ; Fc1cccc(F)c1C1=Nc2c(Cl)cnn2-c2cc(N3CC(F)C3)ccc2N1 ; Fc1cccc(F)c1C1=Nc2c(Cl)cnn2-c2cc(N3CC4(CC(F)C4)C3)ccc2N1]

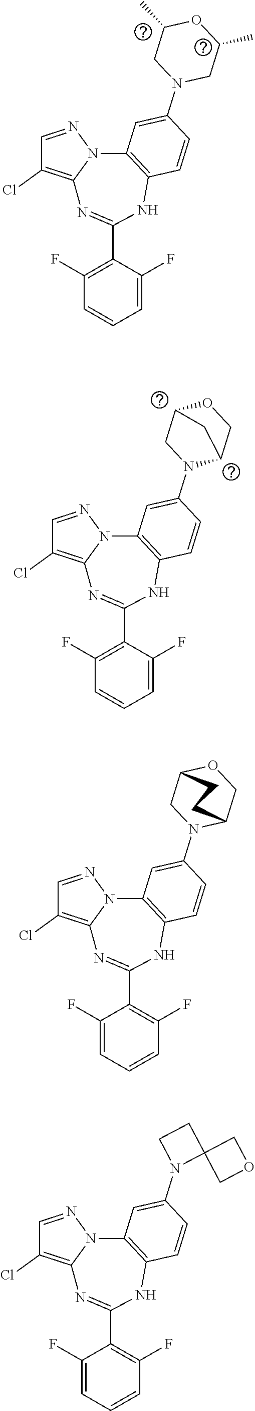

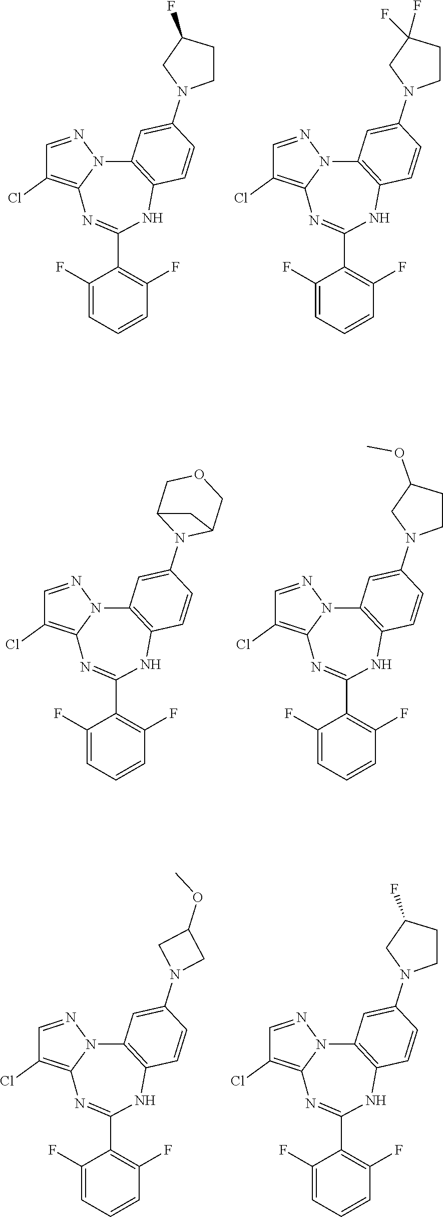 [SMILES: Fc1cccc(F)c1C1=Nc2c(Cl)cnn2-c2cc(N3CC[C@H](F)C3)ccc2N1 ; Fc1cccc(F)c1C1=Nc2c(Cl)cnn2-c2cc(N3CCC(F)(F)C3)ccc2N1 ; Fc1cccc(F)c1C1=Nc2c(Cl)cnn2-c2cc(N3C4COCC3C4)ccc2N1 ; COC1CCN(c2ccc3c(c2)-n2ncc(Cl)c2N=C(c2c(F)cccc2F)N3)C1 ; COC1CN(c2ccc3c(c2)-n2ncc(Cl)c2N=C(c2c(F)cccc2F)N3)C1 ; Fc1cccc(F)c1C1=Nc2c(Cl)cnn2-c2cc(N3CC[C@@H](F)C3)ccc2N1]

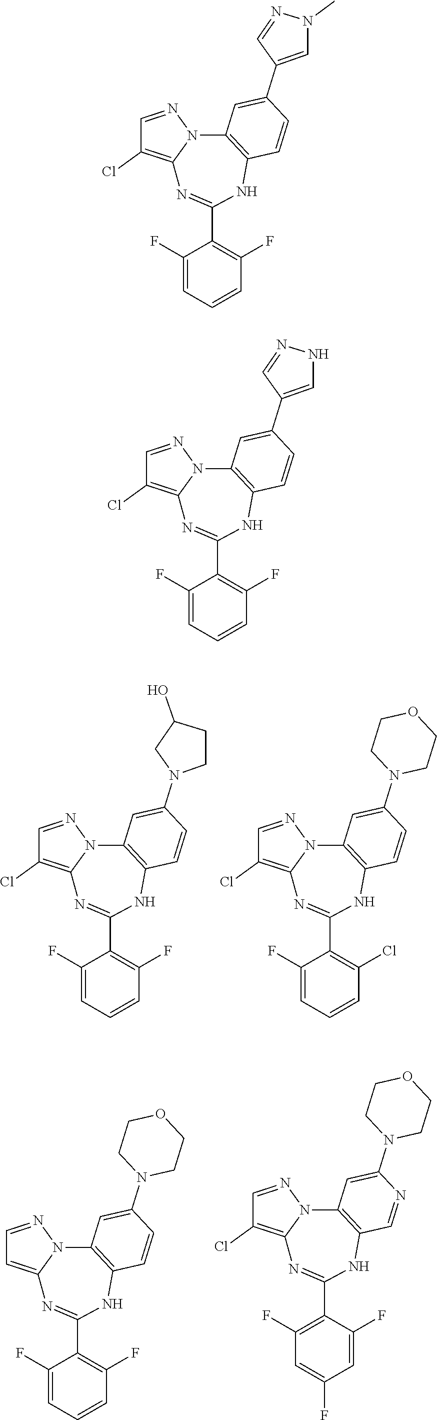 [SMILES: Cn1cc(-c2ccc3c(c2)-n2ncc(Cl)c2N=C(c2c(F)cccc2F)N3)cn1 ; Fc1cccc(F)c1C1=Nc2c(Cl)cnn2-c2cc(-c3cn[nH]c3)ccc2N1 ; OC1CCN(c2ccc3c(c2)-n2ncc(Cl)c2N=C(c2c(F)cccc2F)N3)C1 ; Fc1cccc(Cl)c1C1=Nc2c(Cl)cnn2-c2cc(N3CCOCC3)ccc2N1 ; Fc1cccc(F)c1C1=Nc2ccnn2-c2cc(N3CCOCC3)ccc2N1 ; Fc1cc(F)c(C2=Nc3c(Cl)cnn3-c3cc(N4CCOCC4)ncc3N2)c(F)c1]

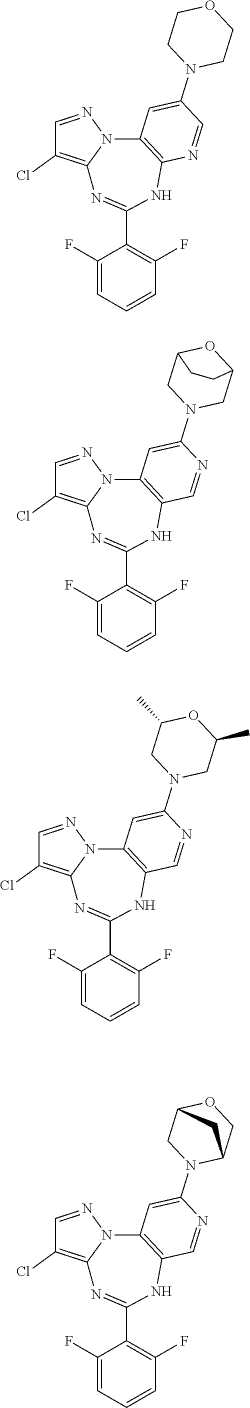 [SMILES: Fc1cccc(F)c1C1=Nc2c(Cl)cnn2-c2cc(N3CCOCC3)cnc2N1 ; Fc1cccc(F)c1C1=Nc2c(Cl)cnn2-c2cc(N3CC4CCC(C3)O4)ncc2N1 ; C[C@H]1CN(c2cc3c(cn2)NC(c2c(F)cccc2F)=Nc2c(Cl)cnn2-3)C[C@H](C)O1 ; Fc1cccc(F)c1C1=Nc2c(Cl)cnn2-c2cc(N3C[C@H]4C[C@@H]3CO4)ncc2N1]

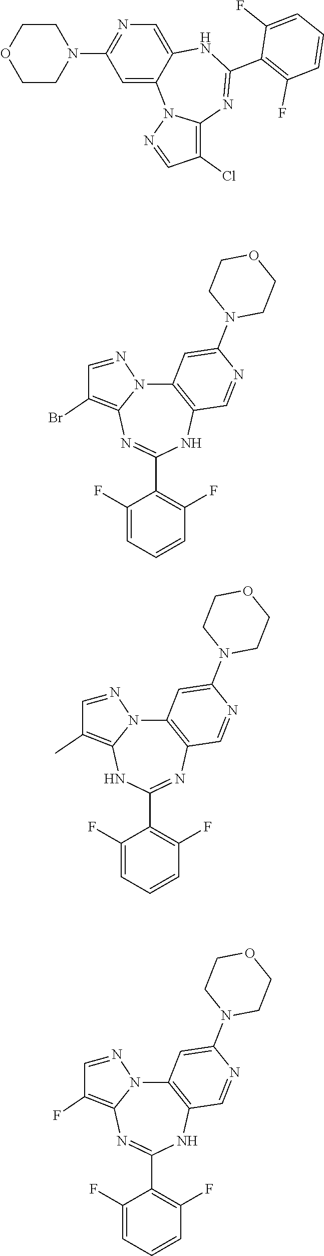 [SMILES: Fc1cccc(F)c1C1=Nc2c(Cl)cnn2-c2cc(N3CCOCC3)ncc2N1 ; Fc1cccc(F)c1C1=Nc2c(Br)cnn2-c2cc(N3CCOCC3)ncc2N1 ; Cc1cnn2c1NC(c1c(F)cccc1F)=Nc1cnc(N3CCOCC3)cc1-2 ; Fc1cccc(F)c1C1=Nc2c(F)cnn2-c2cc(N3CCOCC3)ncc2N1]

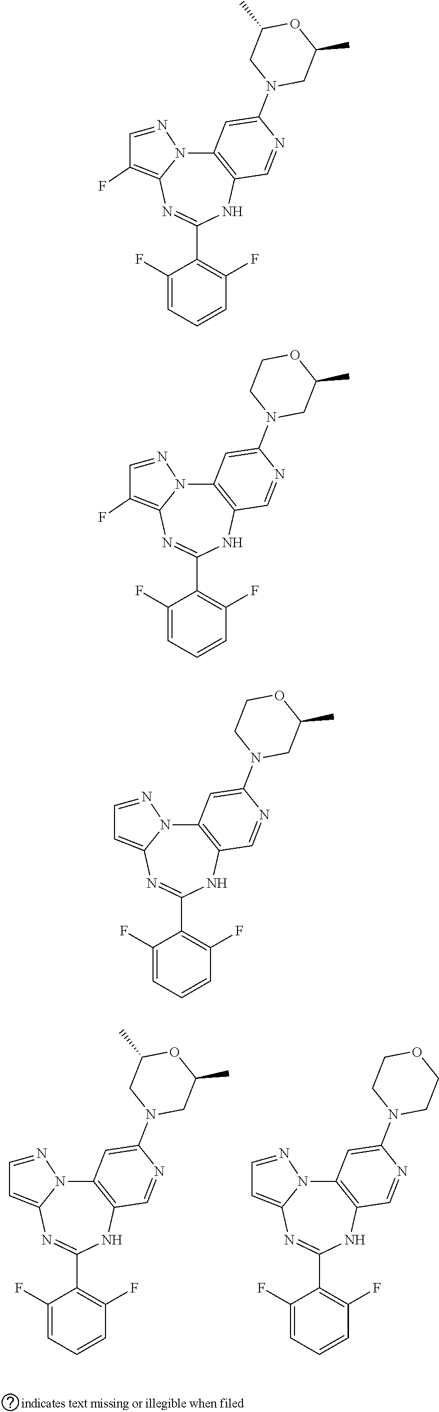 [SMILES: C[C@H]1CN(c2cc3c(cn2)NC(c2c(F)cccc2F)=Nc2c(F)cnn2-3)C[C@H](C)O1 ; C[C@H]1CN(c2cc3c(cn2)NC(c2c(F)cccc2F)=Nc2c(F)cnn2-3)CCO1 ; C[C@H]1CN(c2cc3c(cn2)NC(c2c(F)cccc2F)=Nc2ccnn2-3)CCO1 ; C[C@H]1CN(c2cc3c(cn2)NC(c2c(F)cccc2F)=Nc2ccnn2-3)C[C@H](C)O1 ; Fc1cccc(F)c1C1=Nc2ccnn2-c2cc(N3CCOCC3)ncc2N1]

### Pharmaceutical Compositions

The present invention provides pharmaceutical compositions containing one or more compounds described above, or a pharmaceutically acceptable ester, prodrug, hydrate, solvate or salt of such a compound, optionally in combination with a pharmaceutically acceptable carrier. The invention further provides such compounds for the preparation of a medicament for the treatment of one or more diseases mentioned herein.

A pharmaceutical composition may contain one or more compounds of the invention in a therapeutically effective amount. A therapeutically effective amount of a compound in accordance with this invention means an amount of compound that is effective to prevent, alleviate or ameliorate symptoms of disease or prolong the survival of the subject being treated. Determination of a therapeutically effective amount is within the skill in the art.

The therapeutically effective amount or dosage of a compound according to this invention can vary within wide limits and may be determined in a manner known in the art. Such dosage may be adjusted to the individual requirements in each particular case including the specific compound being administered, the route of administration, the condition being treated, as well as the patient being treated.

Compositions of the invention may include a vehicle for delivery of one or more compounds of the invention. For example, the composition may contain particles, such as nanoparticles, microparticles, liposomes, micelles, and virus particles.

Examples of pharmacologically acceptable salts of sufficiently basic compounds of the invention are salts of physiologically acceptable mineral acids like hydrochloric, hydrobromic, sulfuric and phosphoric acid; or salts of organic acids like methanesulfonic, p-toluenesulfonic, lactic, acetic, trifluoroacetic, citric, succinic, fumaric, maleic and salicylic acid. Further, a sufficiently acidic compound of the invention may form alkali or earth alkali metal salts, for example sodium, potassium, lithium, calcium or magnesium salts; ammonium salts; or organic base salts, for example methylamine, dimethylamine, trimethylamine, triethylamine, ethylenediamine, ethanolamine, choline hydroxide, meglumin, piperidine, morpholine, tris-(2-hydroxyethyl)amine, lysine or arginine salts; all of which are also further examples of salts of the invention. Compounds of the invention may be solvated, especially hydrated. The hydratization/hydration may occur during the process of production or as a consequence of the hygroscopic nature of the initially water free compounds of the invention. The solvates and/or hydrates may e.g. be present in solid or liquid form.

It should be appreciated that certain compounds of the invention may have tautomeric forms from which only one might be specifically mentioned or depicted in the following description, different geometrical isomers (which are usually denoted as cis/trans isomers or more generally as (E) and (Z) isomers) or different optical isomers as a result of one or more chiral carbon atoms (which are usually nomenclatured under the Cahn-Ingold-Prelog or R/S system). All these tautomeric forms, geometrical or optical isomers (as well as racemates and diastereomers) and polymorphous forms are included in the invention. Since the compounds of the invention may contain asymmetric C-atoms, they may be present either as achiral compounds, mixtures of diastereomers, mixtures of enantiomers or as optically pure compounds. The present invention comprises both all pure enantiomers and all pure diastereomers, and also the mixtures thereof in any mixing ratio.

According to a further embodiment of the present invention, one or more hydrogen atoms of the compounds of the present invention may be replaced by deuterium. Deuterium modification improves the metabolic properties of a drug with little or no change in its intrinsic pharmacology. Deuterium substitution at specific molecular positions improves metabolic stability, reduces formation of toxic metabolites and/or increases the formation of desired active metabolites. Accordingly, the present invention also encompasses the partially and fully deuterated compounds of the invention. The term hydrogen also encompasses deuterium.

The therapeutic use of compounds according to the invention, their pharmacologically acceptable salts, solvates and hydrates, respectively, as well as formulations and pharmaceutical compositions also lie within the scope of the present invention. The pharmaceutical compositions according to the present invention may comprise at least one compound of the invention as an active ingredient and, optionally, carrier substances and/or adjuvants.

The present invention also relates to prodrugs which are composed of a compound of the invention and at least one pharmacologically acceptable protective group which will be cleaved off under physiological conditions, such as an alkoxy-, arylalkyloxy-, acyl-, acyloxymethyl group (e.g. pivaloyloxymethyl), an 2-alkyl-, 2-aryl- or 2-arylalkyl oxycarbonyl-2-alkylidene ethyl group or an acyloxy group as defined herein, e.g. ethoxy, benzyloxy, acetyl or acetyloxy or, especially for a compound of the invention, carrying a hydroxy group (—OH): a sulfate, a phosphate (—OPO₃ or —OCH₂OPO₃) or an ester of an amino acid. For example, compositions may contain pro-drugs of the hydroxy group of a compound of the invention.

As used herein, the term pharmaceutically acceptable ester especially refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

The present invention also relates to a prodrug, a biohydrolyzable ester, a biohydrolyzable amide, a polymorph, tautomer, stereoisomer, metabolite, N-oxide, biohydrolyzable carbamate, biohydrolyzable ether, physiologically functional derivative, atropisomer, or in vivo-hydrolysable precursor, diastereomer or mixture of diastereomers, chemically protected form, affinity reagent, complex, chelate and a stereoisomer of the compounds of the invention.

As mentioned above, therapeutically useful agents that contain compounds of the invention, their solvates, salts or formulations are also comprised in the scope of the present invention. In general, compounds of the invention will be administered by using the known and acceptable modes known in the art, either alone or in combination with any other therapeutic agent.

For oral administration such therapeutically useful agents can be administered by one of the following routes: oral, e.g. as tablets, dragees, coated tablets, pills, semisolids, soft or hard capsules, for example soft and hard gelatin capsules, aqueous or oily solutions, emulsions, suspensions or syrups, parenteral including intravenous, intramuscular and subcutaneous injection, e.g. as an injectable solution or suspension, rectal as suppositories, by inhalation or insufflation, e.g. as a powder formulation, as microcrystals or as a spray (e.g. liquid aerosol), transdermal, for example via an transdermal delivery system (TDS) such as a plaster containing the active ingredient or intranasal. For the production of such tablets, pills, semisolids, coated tablets, dragees and hard, e.g. gelatin capsules, the therapeutically useful product may be mixed with pharmaceutically inert, inorganic or organic excipients as are e.g. lactose, sucrose, glucose, gelatine, malt, silica gel, starch or derivatives thereof, talc, stearinic acid or their salts, dried skim milk, and the like. For the production of soft capsules one may use excipients as are e.g. vegetable, petroleum, animal or synthetic oils, wax, fat, polyols. For the production of liquid solutions, emulsions or suspensions or syrups one may use as excipients e.g. water, alcohols, aqueous saline, aqueous dextrose, polyols, glycerin, lipids, phospholipids, cyclodextrins, vegetable, petroleum, animal or synthetic oils. Particularly useful are lipids, such as phospholipids (e.g., natural origin and/or with a particle size between 300 to 350 nm) in phosphate buffered saline (pH=7 to 8, e.g., 7.4). For suppositories one may use excipients as are e.g. vegetable, petroleum, animal or synthetic oils, wax, fat and polyols. For aerosol formulations one may use compressed gases suitable for this purpose, as are e.g. oxygen, nitrogen and carbon dioxide. The pharmaceutically useful agents may also contain additives for conservation, stabilization, e.g. UV stabilizers, emulsifiers, sweetener, aromatizers, salts to change the osmotic pressure, buffers, coating additives and antioxidants.

In general, in the case of oral or parenteral administration to adult humans weighing approximately 80 kg, a daily dosage of about 10 mg to about 10,000 mg, or from about 20 mg to about 1,000 mg, should be appropriate, although the upper limit may be exceeded when indicated. The daily dosage can be administered as a single dose or in divided doses, or for parenteral administration, it may be given as continuous infusion or subcutaneous injection.

### Methods of Making Compounds

The invention also provides methods of making compounds of the invention, such as those described above. Synthesis schemes for making specific compounds of Formula (I) are provided in the Examples below.

### Methods of Treating Conditions

The compounds and compositions of the invention modulate activity of one or more protein kinases. The compounds and compositions may inhibit, activate, or otherwise alter kinase activity. Consequently, the compounds and compositions may be used to diagnose, treat, or prevent a condition, such as a disease, disorder, or other condition for which modulation of kinase activity provides therapeutic benefit.

Diseases, disorders, and conditions that can be diagnosed and/or treated using compositions and methods of the invention include those associated with aberrant activity, e.g., increased activity or decreased activity, of one or more kinases. The disease, disorder, or condition may be associated with aberrant LRRK2 activity, such as Alzheimer's disease, Crohn's disease, inflammatory bowel disease, an inflammatory disease, leprosy, neurodegenerative diseases, a non-skin cancer, or Parkinson's disease, including familial Parkinson's disease, sporadic Parkinson's disease, late-onset Parkinson's disease (PD), and type 8 Parkinson's disease.

The disease, disorder, or condition may be or include a respiratory tract/obstructive airways disease or disorder, such as rhinorrhea, tracheal constriction, airway contraction, acute-, allergic, atrophic rhinitis or chronic rhinitis (such as rhinitis caseosa, hypertrophic rhinitis, rhinitis purulenta, rhinitis sicca), rhinitis medicamentosa, membranous rhinitis (including croupous, fibrinous and pseudomembranous rhinitis), scrofulous rhinitis, perennial allergic rhinitis, seasonal rhinitis (including rhinitis nervosa (hay fever) and vasomotor rhinitis), pollinosis, asthma (such as bronchial, atopic, allergic, intrinsic, extrinsic, exercise-induced, cold air-induced, occupational, bacterial infection-induced, and dust asthma particularly chronic or inveterate asthma (e.g. late asthma and airways hyper-responsiveness)), bronchitis (including chronic, acute, arachidic, catarrhal, croupus, phthinoid and eosinophilic bronchitis), cardiobronchitis, pneumoconiosis, chronic inflammatory disease of the lung which result in interstitial fibrosis, such as interstitial lung disease (ILD) (e.g., idiopathic pulmonary fibrosis, or ILD associated with rheumatoid arthritis, or other autoimmune conditions), acute lung injury (ALI), adult respiratory distress syndrome (ARDS), chronic obstructive pulmonary, airways or lung disease (CORD, COAD, COLD or COPD, such as irreversible COPD), chronic sinusitis, conjunctivitis (e.g. allergic conjunctivitis), cystic fibrosis, extrinsic allergic alveolitis (like farmer's lung and related diseases), fibroid lung, hypersensitivity lung diseases, hypersensitivity pneumonitis, idiopathic interstitial pneumonia, nasal congestion, nasal polyposis, otitis media, and cough (chronic cough associated with inflammation or iatrogenic induced), pleurisy, pulmonary congestion, emphysema, bronchiectasis, sarcoidosis, lung fibrosis, including cryptogenic fibrosing alveolitis, fibrosis complicating anti-neoplastic therapy and chronic infection, including tuberculosis and aspergillosis and other fungal infections, vasculitic and thrombotic disorders of the lung vasculature, and pulmonary hypertension, acute viral infection including the common cold, and infection due to respiratory syncytial virus, influenza, coronavirus (including SARS) and adenovirus, allergic bronchopulmonary mycosis, emphysema, diffuse panbronchiolitis, systemic anaphylaxis or hypersensitivity responses, drug allergies (e.g., to penicillin, cephalosporins), insect sting allergies, and food related allergies which may have effects remote from the gut (such as migraine, rhinitis and eczema), anaphylactic shock, or vascular spasms.

The disease, disorder, or condition may be or include a bone and joint related disease or disorder, such as osteoporosis, arthritis (including rheumatic, infectious, autoimmune, chronic, malignant), seronegative spondyloarthropathies (such as ankylosing spondylitis, rheumatoid spondylitis, psoriatic arthritis, enthesopathy, Bechet's disease, Marie-Strumpell arthritis, arthritis of inflammatory bowel disease, and Reiter's disease), systemic sclerosis, osteoarthritis, osteoarthrosis, both primary and secondary to e.g. congenital hip dysplasia, cervical and lumbar spondylitis, and low back and neck pain, Still's disease, reactive arthritis and undifferentiated spondarthropathy, septic arthritis and other infection-related arthropathies and bone disorders such as tuberculosis, including Pott's disease and Poncet's syndrome, acute and chronic crystal-induced synovitis including urate gout, calcium pyrophosphate deposition disease, and calcium apatite related tendon, bursar and synovial inflammation, primary and secondary Sjogren's syndrome, systemic sclerosis and limited scleroderma, mixed connective tissue disease, and undifferentiated connective tissue disease, inflammatory myopathies including, polymalgia rheumatica, juvenile arthritis including idiopathic inflammatory arthritides of whatever joint distribution and associated syndromes, other joint disease (such as intervertebral disc degeneration or temporomandibular joint degeneration), rheumatic fever and its systemic complications, vasculitides including giant cell arteritis, Takayasu's arteritis, polyarteritis nodosa, microscopic polyarteritis, and vasculitides to associated with viral infection, hypersensitivity reactions, cryoglobulins, paraproteins, low back pain, Familial Mediterranean fever, Muckle-Wells syndrome, and Familial Hibenian Fever, Kikuchi disease, drug-induced arthalgias, tendonititides, polychondritis, and myopathies, osteoporosis, osteomalacia like osteoporosis, osteopenia, osteogenesis imperfects, osteopetrosis, osteofibrosis, osteonecrosis, Paget's disease of bone, hypophosphatemia, Felty's syndrome, Still's disease, slack of artificial joint implant, sprain or strain of muscle or joint, tendinitis, fasciitis, periarthritis humeroscapularis, cervico-omo-brachial syndrome, or tenosynovitis.

The disease, disorder, or condition may be or include a skin or eye related disease or disorder, such as glaucoma, ocular hypertension, cataract, retinal detachment, psoriasis (including psoriasis vulgaris, pustular psoriasis, arthritic psoriasis, erythroderma psoriaticum), palmoplantar pustulosis, xerodoma, eczematous diseases (like atopic dermatitis, ultraviolet radiation dermatitis, contact dermatitis, and seborrheic dermatitis), phytodermatitis, photodermatitis, cutaneous eosinophilias, chronic skin ulcers, cutaneous lupus erythematosus, contact hypersensitivity/allergic contact dermatitis (including sensitivity to poison ivy, sumac, or oak), and eosinophilic folliculitis (Ofuji's disease), pruritus, drug eruptions, urticaria (acute or chronic, allergic or non-allergic), acne, erythema, dermatitis herpetiformis, scleroderma, vitiligo, lichen planus, lichen sclerosus et atrophica, pyodenna gangrenosum, skin sarcoid, pemphigus, ocular pemphigus, pemphigoid, epidermolysis bullosa, angioedema, vasculitides, toxic erythemas, cutaneous eosinophilias, alopecia areata, male-pattern baldness, Sweet's syndrome, Stevens-Johnson syndrome, Weber-Christian syndrome, erythema multiforme, cellulitis, both, infective and non infective, panniculitis, cutaneous Lymphomas, non-melanoma skin cancer and other dysplastic lesions, blepharitis, iritis, anterior and posterior uveitis, choroiditis, autoimmune, degenerative or inflammatory disorders affecting the retina, ophthalmitis including sympathetic ophthalmitis, sarcoidosis, xerosis infections including viral, fungal, and bacterial, allergic conjunctivitis, increased fibrosis, keloids, keloplasty, post surgical scars, epidermolysis bullosa, dry eye, ocular inflammation, allergic conjunctivitis, vernal conjunctivitis, vernal keratoconjunctivitis, and giant papillary conjunctivitis, ocular angiogenesis, cornea damage and scar, all forms of macular degeneration, macular edema, macular dystrophy, abnormal wound healing, scleritis, episcleritis, pachydermia, peripheral ulcerative keratitis, fungal keratitis, herpetic keratitis, invasive aspergillosis; conical cornea, dystorphia epithelialis comeae, or severe intraocular inflammation.

The disease, disorder, or condition may be or include a gastrointestinal tract and abdominal related disease or disorder, such as celiac/coeliac disease (e.g. celiac sprue), cholecystitis, enteritis (including infectious, ischemic, radiation, drug-induced, and eosinophilic gastroenteritis), eosinophilic esophagitis, eosinophilic gastrointestinal inflammation, allergen induced diarrhea, enteropathy associated with seronegative arthropathies, gastritis, autoimmune atrophic gastritis, ischemic bowel disease, inflammatory bowel disease (Crohn's disease and ulcerative colitis), colitis, Mooren's ulcer, irritable bowel syndrome, necrotizing enterocolitis, gut ischemia, glossitis, gingivitis, periodontitis, oesophagitis, including reflex, proctitis, fibrosis and cirrhosis of the liver, pancreatitis, both acute and chronic, pancreatic fibrosis, pancreatic sclerosis, pancreatolithiasis, hepatic cirrhosis, hepatitis (congestive, autoimmune, acute, fulminant, chronic, drug-induced, alcoholic, lupoid, steatohepatitis and chronic viral), fatty liver, primary biliary cirrhosis, hepatic porphyria, and gastrointestinal related allergic disorders, spastic colon, diverticulitis, gastroenteric bleeding, Behcet's disease; partial liver resection, acute liver necrosis (e.g. necrosis caused by toxins, viral hepatitis, shock or anoxia), or hemolytic uremic syndrome.

The disease, disorder, or condition may be or include a hematological disease or disorder, such as anemias, coagulation, myeloproliferative disorders, hemorrhagic disorders, leukopenia, eosinophilic disorders, leukemias (e.g. myelogenous, lymphomas, plasma cell dyscrasias, disorders of the spleen, Band's disease, hemophilia, purpura (including idiopathic thrombocytopenic purpura), or Wiskott-Aldrich syndrome.

The disease, disorder, or condition may be or include a metabolic disease or disorder, such as obesity, amyloidosis, disturbances of the amino and acid metabolism like branched chain disease, hyperaminoacidemia, hyperaminoaciduria, disturbances of the metabolism of urea, hyperammonemia, mucopolysaccharidoses e.g. Maroteaux-Lamy syndrome, storage disease like glycogen storage diseases and lipid storage diseases, glycogenosis I diseases like Cori's disease, malabsorption diseases like intestinal carbohydrate malabsorption, oligosaccharidase deficiency like maltase-, lactase-, sucrase-insufficiency, disorders of the metabolism of fructose, disorders of the metabolism of galactose, galactosaemia, disturbances of carbohydrate utilization like diabetes, hypoglycemia, disturbances of pyruvate metabolism, hypolipidemia, hypolipoproteinemia, hyperlipidemia, hyperlipoproteinemia, carnitine or carnitine acyltransferase deficiency, disturbances of the porphyrin metabolism, porphyrins, disturbances of the purine metabolism, lysosomal diseases, metabolic diseases of nerves and nervous systems like gangliosidoses, sphingolipidoses, sulfatidoses, leucodystrophies, or Lesch Nyhan syndrome.

The disease, disorder, or condition may be or include a cerebellar dysfunction or disturbance of brain metabolism, such as dementia, Alzheimer's disease, Huntington's chores, Parkinson's disease, Pick's disease, toxic encepha-lopathy, demyelinating neuropathies like inflammatory neuropathy, Guillain-Barre syndrome; Meniere's disease and radiculopathy, primary and secondary metabolic disorders associated with hormonal defects like any disorder stemming from either an hyperfunction or hypofunction of some hormone-secreting endocrine gland and any combination thereof. Sipple's syndrome, pituitary gland dysfunction and its effects on other endocrine glands, such as the thyroid, adrenals, ovaries, and testes, acromegaly, hyper- and hypothyroidism, euthyroid goiter, euthyroid sick syndrome, thyroiditis, and thyroid cancer, over or underproduction of the adrenal steroid hormones, adrenogenital syndrome, Cushing's syndrome, Addison's disease of the adrenal cortex, Addison's pernicious anemia, primary and secondary aldosteronism, diabetes insipidus, diabetes mellitus, carcinoid syndrome, disturbances caused by the dysfunction of the parathyroid glands, pancreatic islet cell dysfunction, diabetes, disturbances of the endocrine system of the female like estrogen deficiency, resistant ovary syndrome; muscle weakness, myotonia. Duchenne's and other muscular dystrophies, dystrophia myotonica of Steinert, mitochondrial myopathies like disturbances of the catabolic metabolism in the muscle, carbohydrate and lipid storage myopathies, glycogenoses, myoglobinuria, malignant hyperthermia, polymyalgia rheumatics, dermatomyositis, multiple myositis, primary myocardial disease, cardiomyopathy; disorders of the ectoderm, neurofibromatosis, scleroderma and polyar teritis, Louis-Bar syndrome, von Hippel-Lindau disease, Sturge-Weber syndrome, tuberous sclerosis, amyloidosis, porphyria; sexual dysfunction of the male and female; confused states and seizures due to inappropriate secretion of antidiuretic hormone from the pituitary gland, Liddle's syndrome, Bartter's syndrome, Fanconi's I syndrome, or renal electrolyte wasting.

The disease, disorder, or condition may be or include a transplant rejection related condition, such as acute and chronic allograft rejection following solid organ transplant, for example, transplantation of kidney, heart, liver, lung, and cornea, chronic graft versus host disease, skin graft rejection, and bone marrow transplant rejection, or immunosuppression.

The disease, disorder, or condition may be or include a genitourinary related condition, such as nephritis (interstitial, acute interstitial (allergic), and glomerulonephritis), nephrotic syndrome, cystitis including acute and chronic (interstitial) cystitis and Hunner's ulcer, acute and chronic urethritis, prostatitis, epididymitis, oophoritis, salpingitis, vulvo vaginitis, vulvovaginal candidiasis, Peyronie's disease, and erectile dysfunction, renal disease, renal fibrosis, nephropyelitis, secondary contracted kidney, steroid dependent and steroid-resistant nephrosis, or Goodpasture's syndrome.

The disease, disorder, or condition may be or include a CNS related disease or disorder, such as neurodegenerative diseases, Alzheimer's disease and other cementing disorders including CJD and nvCJD, amyloidosis, and other demyelinating syndromes, cerebral atherosclerosis and vasculitis, temporal arteritis, myasthenia gravis, acute and chronic so pain (acute, intermittent or persistent, whether of central or peripheral origin) including post-operative, visceral pain, headache, migraine, neuralgia (including trigeminal), atypical facial pain, joint and bone pain, pain arising from cancer and tumor invasion, neuropathic pain syndromes including diabetic, post-herpetic, and HIV-associated neuropathies, neurosarcoidosis, to brain injuries, cerebrovascular diseases and their consequences, Parkinson's disease, corticobasal degeneration, motor neuron disease, dementia, including ALS (Amyotrophic-lateral sclerosis), multiple sclerosis, traumatic brain injury, stroke, post-stroke, post-traumatic brain injury, and small-vessel cerebrovascular disease, dementias, vascular dementia, dementia with Lewy bodies, frontotemporal dementia and Parkinsonism linked 1 to chromosome 17, frontotemporal dementias, including Pick's disease, progressive supranuclear palsy, corticobasal degeneration, Huntington's disease, thalamic degeneration, HIV dementia, schizophrenia with dementia, and Korsakoffs psychosis, within the meaning of the definition are also considered to be CNS disorders central and peripheral nervous system complications of malignant, infectious or autoimmune processes, algesia, cerebral infarction, attack, cerebral ischemia, head injury, spinal cord injury, myelopathic muscular atrophy, Shy-Drager syndrome, Reye's syndrome, progressive multifocal leukoencephalopathy, normal pressure hydrocephalus, sclerosing panencephalitis, frontal lobe type dementia, acute anterior poliomyelitis (poliomyelitis), poliomyelitis neurosis, viral encephalitis, allergic encephalomyelitis, epileptic encephalopathies, Creutzfeldt-Jakob disease, Kuru disease, bovine spongiform encephalopathy (mad cow disease), scrapie, epilepsy, cerebral amyloid angiopathy, depression, mania, manic-depressive psychosis, hereditary cerebellar ataxia, peripheral neuropathy, Nasu-Hakola syndrome, or Machado-Joseph disease.

The disease, disorder, or condition may be or include an inflammatory or immunological disease or disorder, such as general inflammation (of the ocular, nasal, pulmonary, and gastrointestinal passages), mastocytosis/mast cell disorders (cutaneous, systemic, mast cell activation syndrome, and pediatric mast cell diseases), mastitis (mammary gland), vaginitis, vasculitis (e.g., necrotizing, cutaneous, and hypersensitivity vasculitis), Wegener granulamatosis, myyositis (including polymyositis, dermatomyositis), basophil related diseases including basophilic leukemia and basophilic leukocytosis, and eosinophil related diseases such as Churg-Strauss syndrome, eosinophilic granuloma, lupus erythematosus (such as, systemic lupus erythematosus, subacute cutaneous lupus erythematosus, and discoid lupus erythematosus), chronic thyroiditis, Hashimoto's thyroiditis, Grave's disease, type I diabetes, complications arising from diabetes mellitus, other immune disorders, eosinophilia fasciitis, hyper IgE syndrome, Addison's disease, antiphospholipid syndrome, immunodeficiency disease, acquired immune deficiency syndrome (AIDS), leprosy, Sezary syndrome, paraneoplastic syndromes, and other autoimmune disorders, fervescence, myositis, nervous diseases selected from multiple myositis, bursitis, Evans syndrome, leukotriene B4-mediated diseases, idiopathic hypoparathyroidism, nephrotic syndrome lupus, or immunosuppression.

The disease, disorder, or condition may be or include a cardiovascular disease or disorder, such as congestive heart failure, myocardial infarction, ischemic diseases of the heart, all kinds of atrial and ventricular arrhythmias, hypertension, cerebral trauma, occlusive vascular disease, stroke, cerebrovascular disorder, atherosclerosis, restenosis, affecting the coronary and peripheral is circulation, pericarditis, myocarditis, inflammatory and auto-immune cardiomyopathies including myocardial sarcoid, endocarditis, valvulitis, and aortitis including infective (e.g. syphilitic), hypertensive vascular diseases, peripheral vascular diseases, and atherosclerosis, vasculitides, disorders of the proximal and peripheral veins including phlebitis and thrombosis, including deep vein thrombosis and complications of varicose veins, aortic aneurism, periarteritis nodosa, cardiac fibrosis, post-myocardial infarction, idiopathic cardiomyopathy, or angioplasty.

The disease, disorder, or condition may be or include an oncological disease or disorder, such as common cancers (prostate, breast, lung, ovarian, pancreatic, bowel and colon, abdomen, stomach (and any other digestive system cancers), liver, pancreas, peritoneum, endocrine glands (adrenal, parathyroid, pituitary, testicles, ovary, thymus, thyroid), eye, head, neck, nervous system (central and peripheral), lymphatic system, blood, pelvic, skin, bone, soft tissue, spleen, thoracic, urogenital, and brain tumors), breast cancer, genitourinary cancer, lung cancer, gastrointestinal cancer, epidermoid cancer, melanoma, ovarian cancer, pancreas cancer, neuroblastoma, malignancies affecting the bone marrow (including the leukaemias) and lymphoproliferative systems, such as Hodgkin's and non-Hodgkin's lymphoma, B-cell lymphoma, follicular lymphoma, metastatic disease and tumor recurrences, and paraneoplastic syndromes, as well as hypergammaglobulinemia, lymphoproliferative diseases, disorders, and/or conditions, paraproteinemias, purpura (including idiopathic thrombocytopenic purpura), Waldenstron's Macroglobulinemia, Gaucher's Disease, histiocytosis, retinoblastoma and any other hyperproliferative disease, sarcomata, cachexia, tumor growth, tumor invasion, metastasis, AIDS-related lymphomas, malignant immunoproliferative diseases, multiple myeloma and malignant plasma cell neoplasms, lymphoid leukemia, acute or chronic myeloid leukemia, acute or chronic lymphocytic leukemia, monocytic leukemia, other leukemias of specified cell type, leukemia of unspecified cell type, other and unspecified malignant neoplasms of lymphoid, haematopoietic and related tissues, for example diffuse large cell lymphoma, T-cell lymphoma or cutaneous T-cell lymphoma). Myeloid cancer includes e.g. acute or chronic myeloid leukaemia, or keratoleukoma.

The disease, disorder, or condition may be or include another disease or disorder, such as pain, migraine, sleep disorders, fever, sepsis, idiopathic thrombocytopenia pupura, post-operative adhesions, flushing, ischemic/reperfusion injury in the heart, brain, peripheral limbs, bacterial infection, viral infection, fungal infection, thrombosis, endotoxin shock, septic shock, thermal regulation including fever, Raynaud's disease, gangrene, diseases requiring anti-coagulation therapy, congestive heart failure, mucus secretion disorders, pulmonary hypotension, prostanoid-induced smooth muscle contract associated with dysmenorrhea and premature labor, premature delivery, reperfusion injury, burn, thermal injury, hemorrhage or traumatic shock, menstrual pain, menstrual cramp, dysmenorrhea, periodontosis, rickettsial infectious disease, protozoal disease, reproduction disease, toothache, pain after tooth extraction, Herpes zoster, Herpes simplex, retroperitoneal fibrosis, or various radiation injuries.

In certain embodiments, the disease is selected from the group consisting of an inflammatory disease, an autoimmune disease, an allergic disorder, and an ocular disorder. In certain embodiments, the disease is selected from the group consisting of pruritus, eczema, asthma, rhinitis, dry eye, ocular inflammation, allergic conjunctivitis, vernal conjunctivitis, vernal keratoconjunctivitis, giant papillary conjunctivitis, fungal keratitis and uveitis.

The method may include modulating the activity of one or more kinases in a subject, such as any of the kinase described above. The method may include inhibiting a kinase. The method may include activating, e.g., stimulating or enhancing the activity of, a kinase. The method may include modulating activity of a single kinase or preferentially modulating activity of a specific kinase over others. The method may include modulating activity of multiple kinases or preferentially modulating activity of two more specific kinases over others.

The method may include providing a compound of the invention. The method may include providing multiple compounds of the invention.

The method may include contacting cells containing a kinase with one or more compounds of the invention. For example and without limitation, contacting a cell with a compound may include exposing a cell to a compound, e.g., in a formulation, such as any of those described above; delivering a compound inside a cell; providing a compound to a subject and allowing a cell in the subject to become exposed to the compound. Contacting may be performed in vivo or in vitro. In vitro contact may include exposure of cells or tissue isolated from a subject. The method may include contacting cells with a single compound of the invention. The method may include contact cells with multiple compounds of the invention.

The method may include administration of a composition to a subject. The compositions may be provided by any suitable route of administration. For example and without limitation, the compositions may be administered buccally, by injection, dermally, enterally, intraarterially, intravenously, intranasally, e.g., by inhalation, intraocularly, orally, parenterally, pulmonarily, rectally, subcutaneously, systemically, topically, e.g., to the skin or eye, transdermally, or with or on an implantable medical device (e.g., stent or drug-eluting stent or balloon equivalents).

The method may include using a composition of the invention to diagnose a disease, disorder, or condition in a subject. For example, a radiolabeled form of a compound may be used a tracer in positron emission tomography (PET) to identify anatomical locations of aberrant kinase activity. PET is known in the art and described in, for example, Wadsak Wolfgang, Mitterhauser Markus (2010), “Basics and principles of radiopharmaceuticals for PET/CT”, European Journal of Radiology, 73 (3): 461-469. doi:10.1016/j.ejrad.2009.12.022; Bailey, D. L; D. W. Townsend; P. E. Valk; M. N. Maisey (2005), Positron Emission Tomography: Basic Sciences. Secaucus, NJ: Springer-Verlag, ISBN 1-85233-798-2; and Carlson, Neil (Jan. 22, 2012). Physiology of Behavior. Methods and Strategies of Research, 11th edition, Pearson, p. 151, ISBN 0205239390, the contents of each of which are incorporated herein by reference. The invention may include administering one or more compositions of the invention for both diagnostic and therapeutic purposes.

### EXAMPLES

### General Synthetic Scheme

Compounds of this disclosure can be made by the methods depicted in the reaction schemes shown below. The starting materials and reagents used in preparing these compounds are either available from commercial suppliers such as Sigma-Aldrich Chemical Co., (Milwaukee, Wis.), Acros Organics, Bachem (Torrance, Calif.), Oakwood Chemicals, Matrix Chemicals, or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989).

Generic schemes are merely illustrative of some methods by which the compounds of this disclosure, and pharmaceutically acceptable salts thereof, can be synthesized, and various modifications to these schemes can be made and will be suggested to one skilled in the art reading this disclosure. The starting materials, the intermediates, and the final products of the reaction(s) may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78 °C to about 200 °C, such as from about 0 °C to about 125 °C and further such as at about room (or ambient) temperature, e.g., about 20 °C. The routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims.

Compounds of this invention can be synthesized by the methods depicted in the general reaction Schemes shown below.

The following Schemes (Scheme 1 and 2) represent general synthetic methods for some of the compounds in this invention and/or any intermediates required for preparing the compounds of the invention, i.e., compounds of formula (A) (B) (C) (D) (E) (F), wherein X, Y₁, Y₂, Z₁, Z₂, Z₃ are as defined above; R₁, R₂, R₃, R₄ and Y are either appropriate choices of, or, appropriate precursors of, R as defined above.

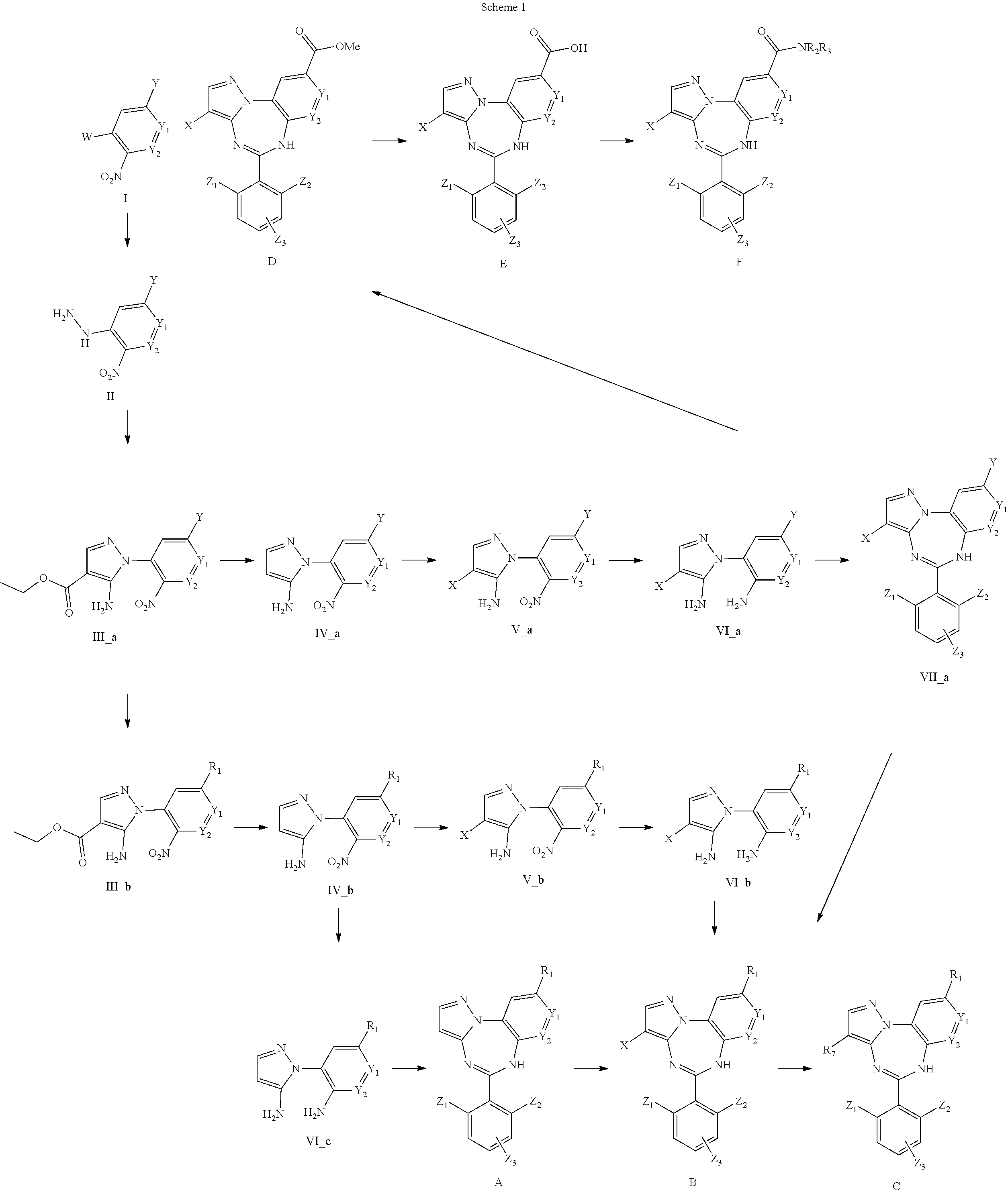

In Scheme 1, starting material I (W=halo) was purchased from commercial sources or prepared according to known literature methods. Treating I with hydrazine or hydrazine hydrate provided compound (II), which after cyclization with ethyl (Z)-2-cyano-3-ethoxy-prop-2-enoate afforded compound (IIIa). Decarboxylation of compound (II_a) led to formation of compound (IV_a), which upon halogenation provided compound (V_a). Reduction of the nitro group of compound (V_a) followed by cyclization of the resulting di-amino product (VI_a) led to the formation of tricyclic intermediate (VII_a). The resulting product VII_a can be further converted to compound of formula (B) via coupling reactions (for example, Buchwald-Hartwig or Suzuki coupling). Compound (B) could be further transformed to compound of formula (C) through various chemical transformations, for example, via hydrogenation, Suzuki coupling, cyanation when X=halo. Carboxylation of compound (VII_a) gave compound of formula (D), which after ester hydrolysis reaction afforded carboxylic acid compound (E). From compound (E), the corresponding amide products (F) could be made through conventional conditions with the corresponding amines.

Additionally, SnAr replacement reaction of compounds (III_a) could also lead to intermediate products (III_b), which after ester group decarboxylation provided compounds (IV_b). Halogenation of compound (IV_b) provided compound (V_b). Nitro group reduction of compound (V_b) generated di-amino intermediate compound (VI_b). Oxidative cyclization of VI_b with the corresponding aldehydes afforded compounds B, which could be further decorated as described above.

Reducing the nitro group of the compound (IV_b) provided compound (VI_c) which were further converted to compounds of formula (A) by a cyclization reaction. Direct halogenation of compound (A) gave the compound of formula (B).

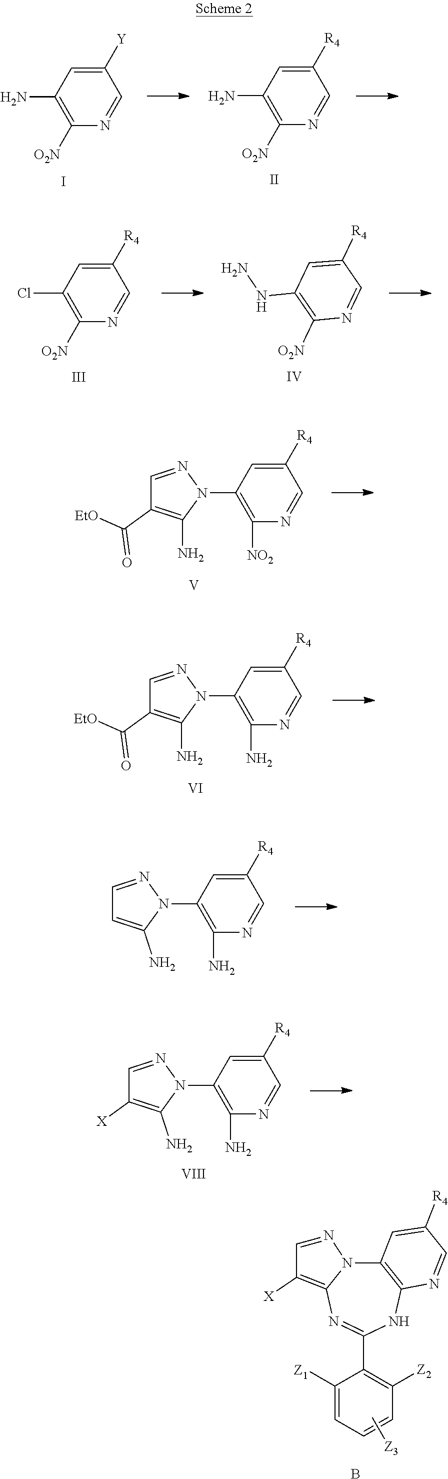

In Scheme 2, compound (I) was purchased from commercial sources or prepared according to well-known literature methods. Reacting compounds (I), wherein Y are an appropriate leaving groups, with corresponding replacement reagents provided intermediates II, which after Sandmeyer transformation provided chloro-compound (III). Treating compound (III) with hydrazine gave products (IV), which upon further reaction with ethyl (Z)-2-cyano-3-ethoxy-prop-2-enoate provided compound (V). Reducing the nitro group of compounds (V) provided the di-amino intermediate (VI), which after ester group decarboxylation led to precursors (VII).

Halogenation of compound (VII) afforded compound (VIII), which after treatment with appropriate aldehydes under oxidative conditions provided the desired compound of formula (B).

### SYNTHETIC EXAMPLES

The following preparations of intermediates, reference compounds, and/or compounds of Formula (I) are given to enable those skilled in the art to more clearly understand and to practice the present disclosure. They should not be considered as limiting the scope of the disclosure, but merely as being illustrative and representative thereof. Some common intermediates that had been made for the syntheses of the described examples are listed in Table 1 below.

TABLE 1

| | | MS Found |
|---|---|---|
| Intermediate | IUPAC | [M + H]⁺ |
| 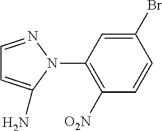 [SMILES: Nc1ccnn1-c1cc(Br)ccc1[N+](=O)[O-]] | 2-(5-bromo-2-nitro-phenyl) pyrazol-3-amine | 282.8 |
| Intermediate 1 | | |
| 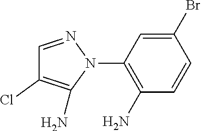 [SMILES: Nc1ccc(Br)cc1-n1ncc(Cl)c1N] | 2-(2-amino-5-bromo-phenyl)-4-chloro-pyrazol-3- amine | —^{a} |
| Intermediate 2 | | |
| 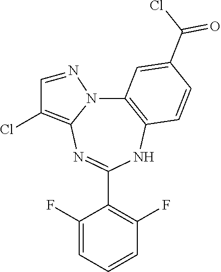 [SMILES: O=C(Cl)c1ccc2c(c1)-n1ncc(Cl)c1N=C(c1c(F)cccc1F)N2] | 3-chloro-5-(2,6-difluorophenyl)-6H-pyrazolo[1,5- a][1,3,5]benzotriazepine-9-carbonyl chloride | —^{a} |
| Intermediate 3 | | |
| 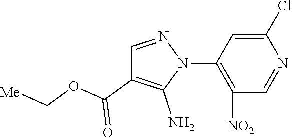 [SMILES: CCOC(=O)c1cnn(-c2cc(Cl)ncc2[N+](=O)[O-])c1N] | ethyl 5-amino-1-(2-chloro-5-nitropyridin-4-yl)- 1H-pyrazole-4-carboxylate | 311.9 |
| Intermediate 4 | | |
| 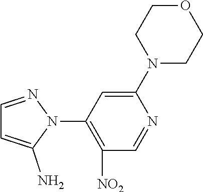 [SMILES: Nc1ccnn1-c1cc(N2CCOCC2)ncc1[N+](=O)[O-]] | 1-(2-morpholino-5-nitropyridin-4-yl)-1H-pyrazol- 5-amine | 290.9 |
| Intermediate 5 | | |
| 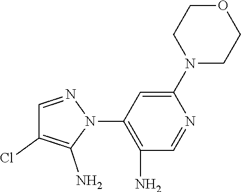 [SMILES: Nc1cnc(N2CCOCC2)cc1-n1ncc(Cl)c1N] | 4-(5-amino-4-chloro-1H-pyrazol-1-yl)-6- morpholinopyridin-3-amine | 295.1 |
| Intermediate 6 | | |
| 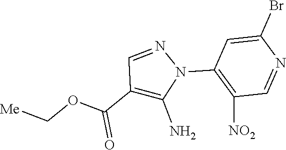 [SMILES: CCOC(=O)c1cnn(-c2cc(Br)ncc2[N+](=O)[O-])c1N] | ethyl 5-amino-1-(2-bromo-5-nitro-4- pyridyl)pyrazole-4-carboxylate | 355.9 |
| Intermediate 7 | | |
| 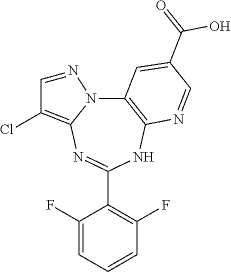 [SMILES: O=C(O)c1cnc2c(c1)-n1ncc(Cl)c1N=C(c1c(F)cccc1F)N2] | 5-Chloro-8-(2,6-difluorophenyl)-2,3,7,9,11- pentazatricyclo[8.4.0.02,6]tetradeca- 1(10),3,5,7,11,13-hexaene-13-carboxylic acid | 376.0 |
| Intermediate 8 | | |

ªLC-MS was not conducted.

### Reference 1

### Synthesis of 2-(5-bromo-2-nitro-phenyl) pyrazol-3-amine (Intermediate 1)

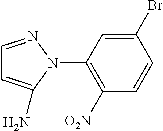 [SMILES: Nc1ccnn1-c1cc(Br)ccc1[N+](=O)[O-]]

### Step 1

To a 5 °C solution of 4-bromo-2-fluoro-1-nitro-benzene (40 g, 0.181 mol, 1.0 eq.) in EtOH (500 mL) was added N₂H₄—H₂O (20 g, 0.399 mol, 2.2 eq.) dropwise. The resulting mixture was stirred at 25 °C for 16 h. The precipitate was collected by filtration, washed with EtOH (50 mL×3), and treated with saturated aqueous NaHCO₃ solution till pH=9. The resulting mixture was extracted with EtOAc (500 mL×3). The combined organic layers were washed with brine (100 mL×3) and concentrated in vacuo to give (5-bromo-2-nitro-phenyl) hydrazine as a brick red solid (36.7 g, crude). ¹HNMR (400 MHz, CDCl₃) δ ppm 3.81 (br s, 2H), 6.69-6.86 (m, 1H), 7.81-8.02 (in, 211), 8.91 (br s, 1H).

### Step 2

A solution of (5-bromo-2-nitro-phenyl) hydrazine (36.7 g, 0.158 mol, 1.0 eq.) and ethyl (Z)-2-cyano-3-ethoxy-prop-2-enoate (32.1 g, 0.189 mol, 1.2 eq.) in EtOH (450 mL) was stirred at 80 °C for 0.5 h. The mixture was concentrated in vacuo to give a crude product of ethyl 5-amino-1-(5-bromo-2-nitro-phenyl) pyrazole-4-carboxylate as a red brick solid (60.6 g, crude). ¹HNMR (500 MHz, CDCl₃) δ ppm 1.43-1.48 (m, 3H), 4.32-4.39 (m, 2H), 7.89-7.98 (m, 1H), 8.01-8.06 (m, 1H), 8.11 (dd, J=8.93, 2.82 Hz, 1H), 9.41 (br s, 1H); LCMS (ESI) m/z 354.8, 356.9 [M+H]⁺.

### Step 3

Ethyl 5-amino-1-(5-bromo-2-nitro-phenyl) pyrazole-4-carboxylate (10 g, 28.1 mmol, 1.0 eq.) was dissolved in 85% H₃PO₄ (120 mL). The mixture was stirred at 170 °C for 2 h. The mixture was quenched by NaHCO₃, and adjusted to pH=9, and extracted with EtOAc (100 mL×3). The combined organic layers were washed with brine (150 mL×3) and concentrated in vacuo to provide 2-(5-bromo-2-nitro-phenyl) pyrazol-3-amine (Intermediate 1) as a brown solid (7.06 g, crude). ¹HNMR (500 MHz, CDCl₃) δ ppm 5.49 (d, J=1.98 Hz, 1H), 7.27 (s, 1H), 7.50 (dd, J=8.70, 2.14 Hz, 1H), 7.58-7.73 (m, 2H), LCMS (ESI) m/z 282.8, 284.8 [M+H]⁺.

### Reference 2

### Synthesis of 2-(5-bromo-2-nitro-phenyl) pyrazol-3-amine (Intermediate 2)

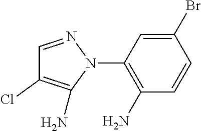 [SMILES: Nc1ccc(Br)cc1-n1ncc(Cl)c1N]

### Step 1

To a solution of 2-(5-Bromo-2-nitro-phenyl) pyrazol-3-amine (Intermediate 1) (7.06 g, 24.9 mmol, 1.0 eq.) in MeCN (80 mL) was added NCS (3.0 g, 22.4 mmol, 0.9 eq.). The resulting solution was stirred at 25 °C for 2 h. The mixture was concentrated in vacuo. The residue was purified by flash silica gel chromatography (ISCO®; 120 g SepaFlash® Silica Flash Column, Eluent of 0~35% EtOAc/petroleum ether gradient @ 50 mL/min), to provide 2-(5-bromo-2-nitro-phenyl)-4-chloro-pyrazol-3-amine as yellow solid (6 g, 75.8%). ¹HNMR (500 MHz, CDCl₃) δ ppm 7.51 (d, J=1.98 Hz, 1H), 7.53 (s, 1H), 7.58 (d, J=2.14 Hz, 1H), 7.68 (d, J=8.70 Hz, 1H); LCMS (ESI) m/z 318.9 [M+H]⁺.

### Step 2

To 2-(5-Bromo-2-nitro-phenyl)-4-chloro-pyrazol-3-amine (5.2 g. 16.3 mmol, 1.0 eq.) in EtOH/H₂O (30 mL, 1:1) were added Zn powder (5.35 g, 81.9 mmol, 5.0 eq.) and NH₄Cl (8.76 g, 0.164 mmol, 10.0 eq.). The resulting mixture was stirred at 50 °C for 2 h. After removal of the solid via filtration, the remaining solution was extracted with EtOAc (50 mL×3) and the combined organic layers were washed with brine (200 mL×3). Removal of the volatiles under reduced pressure provided the title product of 2-(2-amino-5-bromo-phenyl)-4-chloro-pyrazol-3-amine as a yellow solid (4.5 g, crude). ¹HNMR (400 MHz, CDCl₃) δ ppm 3.74 (br s, 4H), 6.65 (d, J=8.58 Hz, 1H), 7.22-7.39 (m, 2H), 7.47 (s, 1H).

### Reference 3

### Synthesis of 3-chloro-5-(2,6-difluorophenyl)-6H-pyrazolo[1,5-a][1,3,5]benzotriazepine-9-carbonyl chloride (Intermediate 3)

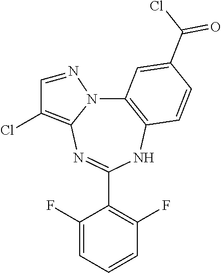 [SMILES: O=C(Cl)c1ccc2c(c1)-n1ncc(Cl)c1N=C(c1c(F)cccc1F)N2]

To a solution of 3-chloro-5-(2,6-difluorophenyl)-6H-pyrazolo[1,5-a][1,3,5]benzotriazepine-9-carboxylic acid (Example 40) (320 mg, 0.853 mmol, 1 eq) in CH₂Cl₂ (10 mL) was added (COCl)₂ (216 mg, 1.71 mmol, 2 eq) followed by DMF (3 drops). After stirring at 25 °C for 2 h, and resulting mixture was concentrated under reduced pressure to afford 3-chloro-5-(2,6-difluorophenyl)-6H-pyrazolo[1,5-a][1,3,5]benzotriazepine-9-carbonyl chloride as yellow solid (336 mg, 100%).

### Reference 4

### Synthesis of ethyl 5-amino-1-(2-chloro-5-nitropyridin-4-yl)-1H-pyrazole-4-carboxylate (Intermediate 4)

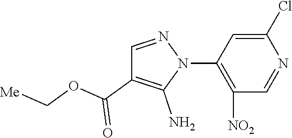 [SMILES: CCOC(=O)c1cnn(-c2cc(Cl)ncc2[N+](=O)[O-])c1N]

### Step 1

To a solution of 2,4-dichloro-5-nitro-pyridine (5 g, 25.9 mmol) in EtOH (100 mL) was added N₂H₄-H₂O (1.53 g, 25.9 mmol). The mixture was stirred at 80 °C for 6 h. The reaction mixture was concentrated under reduced pressure, and the remaining residue was purified by flash silica gel chromatography (ISCO®; 120 g SepaFlash® Silica Flash Column, petroleum ether/EtOAc with EtOAc from 0~30%, flow rate=80 mL/min, 254 nm) to provide (2-chloro-5-nitro-4-pyridyl)hydrazine as a yellow solid (7.3 g, 34.3%). LCMS (ESI) m/z 189.1 [M+H]⁺.

### Step 2

To a solution of (2-chloro-5-nitro-4-pyridyl)hydrazine (7.3 g, 38.7 mmol) in EtOH (150 mL) was added ethyl (E)-2-cyano-3-ethoxy-prop-2-enoate (6.55 g, 38.7 mmol). The mixture was stirred at 80 °C for 24 h. The reaction mixture was concentrated under reduced pressure, and the remaining residue was triturated with (petroleum ether/EtOAc=3:1, 80 mL) at 25 °C for 1 h to provide ethyl 5-amino-1-(2-chloro-5-nitro-4-pyridyl)pyrazole-4-carboxylate as a yellow solid (15 g, 72.9%). ¹HNMR (400 MHz, CDCl₃) δ ppm 1.38 (t, J=7.2 Hz, 3H), 4.25-4.40 (m, 2H), 5.45 (br s, 2H), 7.71 (s, 1H), 7.83 (s, 1H), 8.98 (s, 1H); LCMS (ESI) m/z 311.9 [M+H]⁺.

### Reference 5

### Synthesis of 1-(2-morpholino-5-nitropyridin-4-yl)-1H-pyrazol-5-amine (Intermediate 5)

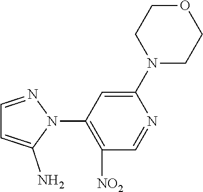 [SMILES: Nc1ccnn1-c1cc(N2CCOCC2)ncc1[N+](=O)[O-]]

### Step 1

To a solution of ethyl 5-amino-1-(2-chloro-5-nitro-4-pyridyl)pyrazole-4-carboxylate (Intermediate 4, 3.25 g, 10.4 mmol) in DCM (80 mL) were added TEA (3.17 g, 31.3 mmol) and morpholine (908 mg, 10.4 mmol). The mixture was stirred at 25 °C for 3 h and concentrated under reduced pressure. The remaining residue was purified by flash silica gel chromatography (ISCO®; 40 g SepaFlash® Silica Flash Column, petroleum ether/EtOAc with EtOAc from 0~50%, flow rate=60 mL/min, 254 nm) to provide ethyl 5-amino-1-(2-morpholino-5-nitro-4-pyridyl)pyrazole-4-carboxylate as a yellow solid (6.6 g, 87.3%). LCMS (ESI) m/z 363.0 [M+H]⁺.

### Step 2

A solution of ethyl 5-amino-1-(2-morpholino-5-nitro-4-pyridyl)pyrazole-4-carboxylate (2 g, 5.52 mmol) in H₃PO₄ (16 mL, 85 wt %) was stirred at 160 °C for 4 h. The reaction mixture was treated with H₂O (20 mL), neutralized with saturated aqueous NaHCO₃ solution to pH=7~8, and extracted with EtOAc (250 mL×3). The combined organic layers were washed with brine (50 mL), dried with anhydrous Na₂SO₄, filtered and concentrated under reduced pressure. The remaining residue was purified by flash silica gel chromatography (ISCO®; 12 g SepaFlash® Silica Flash Column, petroleum ether/EtOAc with EtOAc from 0~60%, flow rate=60 mL/min, 254 nm) to provide the title product of 2-(2-morpholino-5-nitro-4-pyridyl)pyrazol-3-amine as a yellow solid (2 g, 44.9%). LCMS (ESI) m/z 290.9 [M+H]⁺.

### Reference 6

### Synthesis of 4-(5-amino-4-chloro-1H-pyrazol-1-yl)-6-morpholinopyridin-3-amine (Intermediate 6)

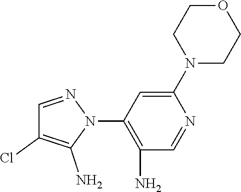 [SMILES: Nc1cnc(N2CCOCC2)cc1-n1ncc(Cl)c1N]

### Step 1

To a solution of 2-(2-morpholino-5-nitro-4-pyridyl)pyrazol-3-amine (Intermediate 5, 2 g, 6.89 mmol) in DCM (20 mL) was added NCS (920 mg, 6.89 mmol). The mixture was stirred at 25 °C for 2 h. The reaction mixture was treated with H₂O (20 mL) and extracted with EtOAc (30 mL×3). The combined organic layers were washed with brine (20 mL), dried with anhydrous Na₂SO₄, filtered and concentrated under reduced pressure. The remaining residue was purified by flash silica gel chromatography (ISCO®; 20 g SepaFlash® Silica Flash Column, petroleum ether/EtOAc with EtOAc from 0~70%, flow rate=50 mL/min, 254 nm) to provide 4-chloro-2-(2-morpholino-5-nitro-4-pyridyl)pyrazol-3-amine as a yellow solid (2.5 g, 94.9%). LCMS (ESI) m/z 324.9 [M+H]⁺.

### Step 2

To a solution of 4-chloro-2-(2-morpholino-5-nitro-4-pyridyl)pyrazol-3-amine (2.5 g, 6.47 mmol) in THF (30 mL) was added NiCl₂-6H₂O (3.07 g, 12.9 mmol) and NaBH₄ (1.22 g, 32.3 mmol) at 0 °C. The mixture was stirred at 0 °C for 1 h. After removal of the precipitate via filtration, the remaining solution was concentrated under reduced pressure. The remaining residue was purified by flash silica gel chromatography (ISCO®; 12 g SepaFlash® Silica Flash Column, petroleum ether/EtOAc with EtOAc from 0~70%, flow rate=50 mL/min, 254 nm) to provide the title product of 4-(5-amino-4-chloro-pyrazol-1-yl)-6-morpholino-pyridin-3-amine as a white solid (1.4 g, 73.4%). LCMS (ESI) m/z 295.1 [M+H]⁻.

### Reference 7

### Synthesis of ethyl 5-amino-1-(2-bromo-5-nitro-4-pyridyl)pyrazole-4-carboxylate (Intermediate 7)

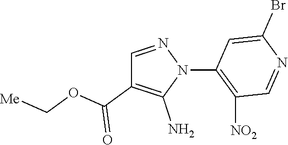 [SMILES: CCOC(=O)c1cnn(-c2cc(Br)ncc2[N+](=O)[O-])c1N]

### Step 1

A mixture of 2,4-dibromo-5-nitro-pyridine (6 g, 21.3 mmol), N₂H₄-H₂O (1.38 g, 23.4 mmol, 85% purity) in EtOH (250 mL) was degassed and purged with N₂ for 3 times. After stirring at 80 °C for 6 h under N₂ atmosphere, the reaction mixture was cooled to rt, treated with H₂O (30 mL), and extracted with EtOAc (30 mL×3). The combined organic layers were washed with brine (30 mL×2), dried with anhydrous Na₂SO₄, filtered. After removal of volatiles under reduced pressure, the remaining residue was purified by flash silica gel chromatography (ISCO®; 4 g SepaFlash® Silica Flash Column, petroleum ether/EtOAc with EtOAc from 0~35%, flow rate=30 mL/min, 254 nm) to provide (2-bromo-5-nitro-4-pyridyl)hydrazine as a yellow solid (13 g, 55.7%, 85% purity). LCMS (ESI) m/z 233.0 [M+H]⁺.

### Step 2

To a solution of ethyl (E)-2-cyano-3-ethoxy-prop-2-enoate (7.99 g, 47.2 mmol) in EtOH (100 mL) was added (2-bromo-5-nitro-4-pyridyl)hydrazine (10 g, 42.9 mmol). The mixture was stirred at 80 °C for 16 h and cooled to rt. The precipitate was collected via filtration, dried under reduced pressure, and triturated with petroleum ether at 25 °C for 1 h to provide the title compound as a yellow solid (8.8 g, 57.6%). LCMS (ESI) m/z 355.9 [M+H]⁺.

### Reference 8

### Synthesis of 5-Chloro-8-(2,6-difluorophenyl)-2,3,7,9,11-pentazatricyclo[8.4.0.02,6]tetradeca-1(10),3,5,7,11,13-hexaene-13-carboxylic acid (Intermediate 8)

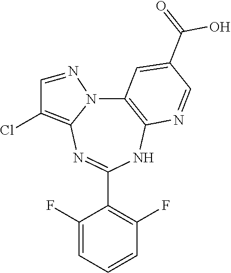 [SMILES: O=C(O)c1cnc2c(c1)-n1ncc(Cl)c1N=C(c1c(F)cccc1F)N2]

To a solution of methyl 5-chloro-8-(2,6-difluorophenyl)-2,3,7,9,11-pentazatricyclo[8.4.0.02,6]tetradeca-1(10),3,5,7,11,13-hexaene-13-carboxylate (Example 58, 45 mg, 0.115 mmol) in H₂O (1 mL) and THF (3 mL) was added LiOH (13.8 mg, 0.577 mmol). After stirring at 25 °C for 20 h, the resulting mixture was treated with HCl (2.0 N, 15 mL) and extracted with EtOAc (10 mL×3). The combined organic layers were washed with brine (20 mL). dried over anhydrous Na₂SO₄, filtered and concentrated under reduced pressure to provide crude product 5-chloro-8-(2,6-difluorophenyl)-2,3,7,9,11-pentazatricyclo[8.4.0.02,6]tetradeca-1(10),3,5,7,11,13-hexaene-13-carboxylic acid as a yellow solid (40 mg, crude). LCMS (ESI) m/z 376.0 [M+H]⁺

Representative compounds of Formula (I), or salts thereof, are disclosed in Table 2. Although Table 2 may show parent compound, or a specific salt of Formula (I), those skilled in the art will be able to recognize other salts, such as pharmaceutically acceptable salts, or the parent compound (wherein the “parent compound” is a compound without a salt moiety present), and of those compounds in Table 1.

Compounds disclosed in Table 2 were tested according to methods described in Example 75 and found to exhibit an LRRK2 IC₅₀ as indicated in the table. In Table 2, activity is provided as the following: “+++”=IC₅₀ less than 50 nM; “++”=IC₅₀ between 50 nM and 200 nM; “+”=IC₅₀> or =200 nM.

TABLE 2

| | | MS Found | LRRK2 |
|---|---|---|---|
| Compound Structure | IUPAC | [M + H]⁺ | IC₅₀ |
| 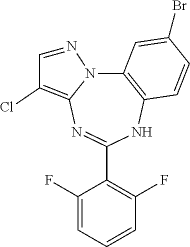 [SMILES: Fc1cccc(F)c1C1=Nc2c(Cl)cnn2-c2cc(Br)ccc2N1] | 9-bromo-3-chloro-5-(2,6- difluorophenyl)-6H-pyrazolo[1,5- a][1,3,5]benzotriazepine | 408.9 410.9 | +++ |
| 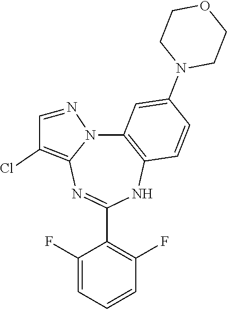 [SMILES: Fc1cccc(F)c1C1=Nc2c(Cl)cnn2-c2cc(N3CCOCC3)ccc2N1] | 4-[3-chloro-5-(2,6-difluorophenyl)-6H- pyrazolo[1,5-a][1,3,5]benzotriazepin-9- yl]morpholine | 416.1 | +++ |
| 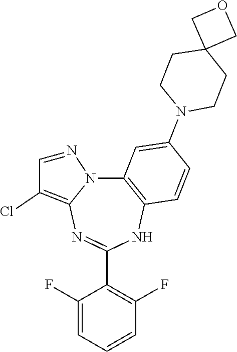 [SMILES: Fc1cccc(F)c1C1=Nc2c(Cl)cnn2-c2cc(N3CCC4(CC3)COC4)ccc2N1] | 7-[3-chloro-5-(2,6-difluorophenyl)-6H- pyrazolo[1,5-a][1,3,5]benzotriazepin-9- yl]-2-oxa-7-azaspiro[3.5]nonane | 456.1 | +++ |
| 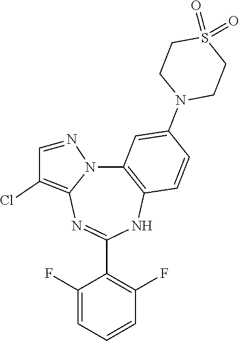 [SMILES: O=S1(=O)CCN(c2ccc3c(c2)-n2ncc(Cl)c2N=C(c2c(F)cccc2F)N3)CC1] | 4-[3-chloro-5-(2,6-difluorophenyl)-6H- pyrazolo[1,5-a][1,3,5]benzotriazepin-9- yl]morpholine | 464.2 | +++ |
| 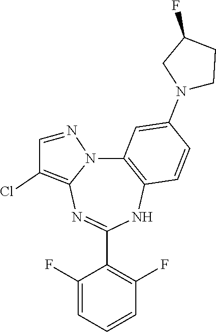 [SMILES: Fc1cccc(F)c1C1=Nc2c(Cl)cnn2-c2cc(N3CC[C@H](F)C3)ccc2N1] | 3-chloro-5-(2,6-difluorophenyl)-9- [(3S)-3-fluoropyrrolidin-1-yl]-6H- pyrazolo[1,5-a][1,3,5]benzotriazepine | 418.1 | +++ |
| 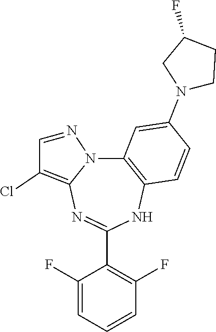 [SMILES: Fc1cccc(F)c1C1=Nc2c(Cl)cnn2-c2cc(N3CC[C@@H](F)C3)ccc2N1] | 3-chloro-5-(2,6-difluorophenyl)-9- [(3R)-3-fluoropyrrolidin-1-yl]-6H- pyrazolo[1,5-a][1,3,5]benzotriazepine | 418.1 | +++ |
| 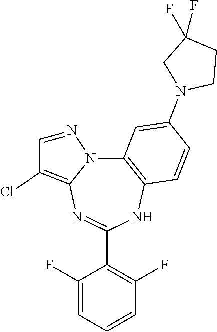 [SMILES: Fc1cccc(F)c1C1=Nc2c(Cl)cnn2-c2cc(N3CCC(F)(F)C3)ccc2N1] | 3-chloro-5-(2,6-difluorophenyl)-9-(3,3- difluoropyrrolidin-1-yl)-6H- pyrazolo[1,5-a][1,3,5]benzotriazepine | 436.1 | +++ |
| 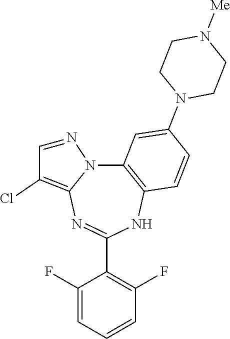 [SMILES: CN1CCN(c2ccc3c(c2)-n2ncc(Cl)c2N=C(c2c(F)cccc2F)N3)CC1] | 3-chloro-5-(2,6-difluorophenyl)-9-(4- methylpiperazin-1-yl)-6H-pyrazolo[1,5- a][1,3,5]benzotriazepine | 429.1 | +++ |
| 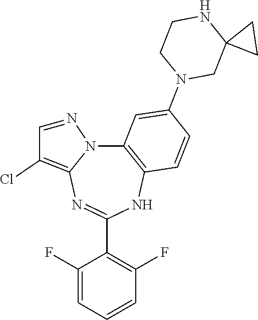 [SMILES: Fc1cccc(F)c1C1=Nc2c(Cl)cnn2-c2cc(N3CCNC4(CC4)C3)ccc2N1] | 3-chloro-9-(4,7-diazaspiro[2.5]octan-7- yl)-5-(2,6-difluorophenyl)-6H- pyrazolo[1,5-a][1,3,5]benzotriazepine | 441.1 | +++ |
| 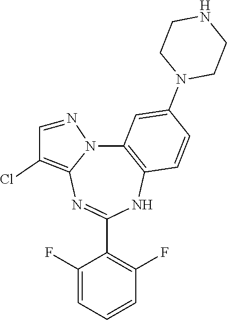 [SMILES: Fc1cccc(F)c1C1=Nc2c(Cl)cnn2-c2cc(N3CCNCC3)ccc2N1] | 3-chloro-5-(2,6-difluorophenyl)-9- piperazin-1-yl-6H-pyrazolo[1,5- a][1,3,5]benzotriazepine | 415.1 | +++ |
| 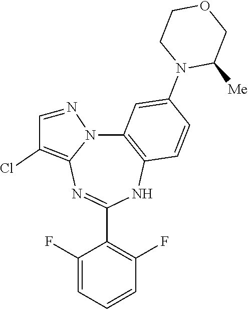 [SMILES: C[C@@H]1COCCN1c1ccc2c(c1)-n1ncc(Cl)c1N=C(c1c(F)cccc1F)N2] | (3R)-4-[3-chloro-5-(2,6- difluorophenyl)-6H-pyrazolo[1,5- a][1,3,5]benzotriazepin-9-yl]-3-methyl- morpholine | 430.1 | +++ |
| 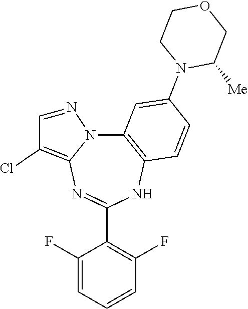 [SMILES: C[C@H]1COCCN1c1ccc2c(c1)-n1ncc(Cl)c1N=C(c1c(F)cccc1F)N2] | (3S)-4-[3-chloro-5-(2,6- difluorophenyl)-6H-pyrazolo[1,5- a][1,3,5]benzotriazepin-9-yl]-3-methyl- morpholine | 430.1 | +++ |
| 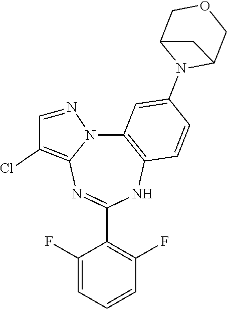 [SMILES: Fc1cccc(F)c1C1=Nc2c(Cl)cnn2-c2cc(N3C4COCC3C4)ccc2N1] | 6-[3-chloro-5-(2,6-difluorophenyl)-6H- pyrazolo[1,5-a][1,3,5]benzotriazepin-9- yl]-3-oxa-6-azabicyclo[3.1.1]heptane | 428.1 | +++ |
| 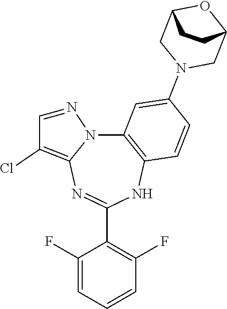 | (1R,5S)-3-[3-chloro-5-(2,6- difluorophenyl)-6H-pyrazolo[1,5- a][1,3,5]benzotriazepin-9-yl]-8-oxa-3- azabicyclo[3.2.1]octane | 442.1 | +++ |
| 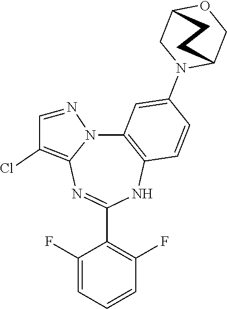 | (1R,4R)-5-[3-chloro-5-(2,6- difluorophenyl)-6H-pyrazolo[1,5- a][1,3,5]benzotriazepin-9-yl]-2-oxa-5- azabicyclo[2.2.2]octane | 442.1 | +++ |
| 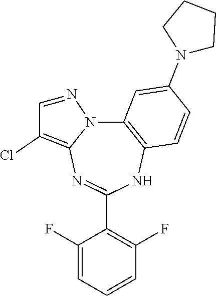 [SMILES: Fc1cccc(F)c1C1=Nc2c(Cl)cnn2-c2cc(N3CCCC3)ccc2N1] | 3-chloro-5-(2,6-difluorophenyl)-9- (pyrrolidin-1-yl)-6H- benzo[f]pyrazolo[1,5-a][1,3,5]triazepine | 400.1 | +++ |
| 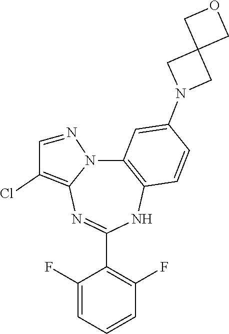 [SMILES: Fc1cccc(F)c1C1=Nc2c(Cl)cnn2-c2cc(N3CC4(COC4)C3)ccc2N1] | 6-[3-chloro-5-(2,6-difluorophenyl)-6H- pyrazolo[1,5-a][1,3,5]benzotriazepin-9- yl]-2-oxa-6-azaspiro[3.3]heptane | 428.1 | +++ |
| 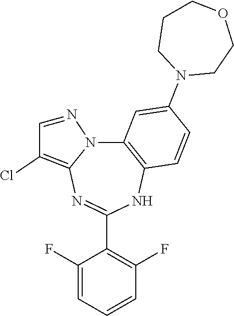 [SMILES: Fc1cccc(F)c1C1=Nc2c(Cl)cnn2-c2cc(N3CCCOCC3)ccc2N1] | 4-[3-chloro-5-(2,6-difluorophenyl)-6H- pyrazolo[1,5-a][1,3,5]benzotriazepin-9- yl]-1,4-oxazepane | 430.1 | +++ |
| 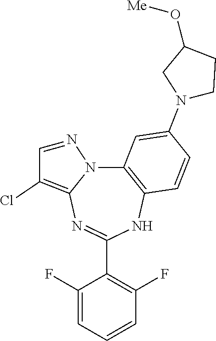 [SMILES: COC1CCN(c2ccc3c(c2)-n2ncc(Cl)c2N=C(c2c(F)cccc2F)N3)C1] | 3-chloro-5-(2,6-difluorophenyl)-9-(3- methoxypyrrolidin-1-yl)-6H- pyrazolo[1,5-a][1,3,5]benzotriazepine | 430.1 | +++ |
| (racemic mixture) | | | |
| 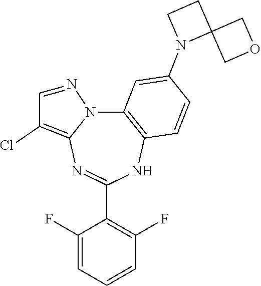 [SMILES: Fc1cccc(F)c1C1=Nc2c(Cl)cnn2-c2cc(N3CCC34COC4)ccc2N1] | 1-[3-chloro-5-(2,6-difluorophenyl)-6H- pyrazolo[1,5-a][1,3,5]benzotriazepin-9- yl]-6-oxa-1-azaspiro[3.3]heptane | 428.1 | +++ |
| 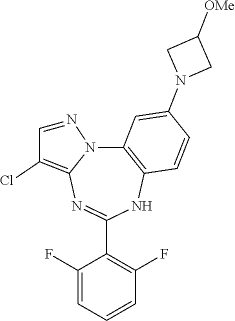 [SMILES: COC1CN(c2ccc3c(c2)-n2ncc(Cl)c2N=C(c2c(F)cccc2F)N3)C1] | 3-chloro-5-(2,6-difluorophenyl)-9-(3- methoxyazetidin-1-yl)-6H- pyrazolo[1,5-a][1,3,5]benzotriazepine | 416.1 | +++ |
| 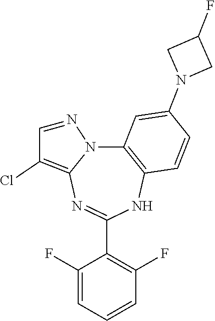 [SMILES: Fc1cccc(F)c1C1=Nc2c(Cl)cnn2-c2cc(N3CC(F)C3)ccc2N1] | 3-chloro-5-(2,6-difluorophenyl)-9-(3- fluoroazetidin-1-yl)-6H-pyrazolo[1,5- a][1,3,5]benzotriazepine | 403.9 | +++ |
| 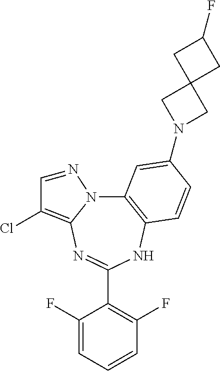 [SMILES: Fc1cccc(F)c1C1=Nc2c(Cl)cnn2-c2cc(N3CC4(CC(F)C4)C3)ccc2N1] | 3-chloro-5-(2,6-difluorophenyl)-9-(6- fluoro-2-azaspiro[3.3]heptan-2-yl)-6H- pyrazolo[1,5-a][1,3,5]benzotriazepine | 444.1 | +++ |
| 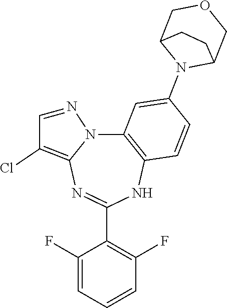 [SMILES: Fc1cccc(F)c1C1=Nc2c(Cl)cnn2-c2cc(N3C4CCC3COC4)ccc2N1] | 8-[3-chloro-5-(2,6-difluorophenyl)-6H- pyrazolo[1,5-a][1,3,5] benzotriazepin-9-yl]-3-oxa-8- azabicyclo[3.2.1]octane | 442.1 | +++ |
| 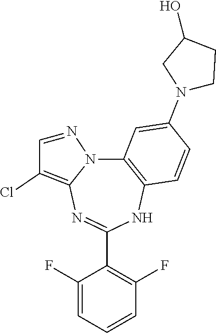 [SMILES: OC1CCN(c2ccc3c(c2)-n2ncc(Cl)c2N=C(c2c(F)cccc2F)N3)C1] | 1-[3-chloro-5-(2,6-difluorophenyl)-6H- pyrazolo[1,5-a][1,3,5]benzotriazepin-9- yl]pyrrolidin-3-ol | 416.1 | +++ |
| racemic mixture | | | |
| 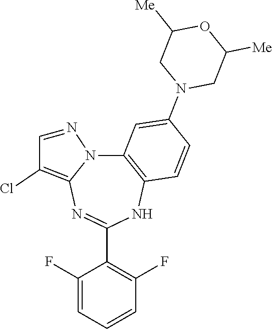 [SMILES: CC1CN(c2ccc3c(c2)-n2ncc(Cl)c2N=C(c2c(F)cccc2F)N3)CC(C)O1] | 4-[3-chloro-5-(2,6-difluorophenyl)-6H- pyrazolo[1,5-a][1,3,5]benzotriazepin-9- yl]-2,6-dimethyl-morpholine | 444.2 | +++ |
| 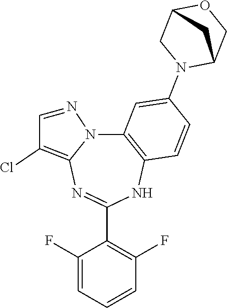 [SMILES: Fc1cccc(F)c1C1=Nc2c(Cl)cnn2-c2cc(N3C[C@H]4C[C@@H]3CO4)ccc2N1] | (1R,4R)-5-[3-chloro-5-(2,6- difluorophenyl)-6H-pyrazolo[1,5- a][1,3,5]benzotriazepin-9-yl]-2-oxa-5- azabicyclo[2.2.1]heptane | 427.8 | +++ |
| 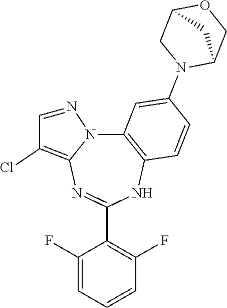 [SMILES: Fc1cccc(F)c1C1=Nc2c(Cl)cnn2-c2cc(N3C[C@@H]4C[C@H]3CO4)ccc2N1] | (1S,4S)-5-[3-chloro-5-(2,6- difluorophenyl)-6H-pyrazolo[1,5- a][1,3,5]benzotriazepin-9-yl]-2-oxa-5- azabicyclo[2.2.1]heptane | 427.8 | +++ |
| 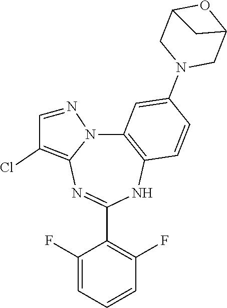 [SMILES: Fc1cccc(F)c1C1=Nc2c(Cl)cnn2-c2cc(N3CC4CC(C3)O4)ccc2N1] | 3-[3-chloro-5-(2,6-difluorophenyl)-6H- pyrazolo[1,5-a][1,3,5]benzotriazepin-9- yl]-6-oxa-3-azabicyclo[3.1.1]heptane | 428.1 | +++ |
| 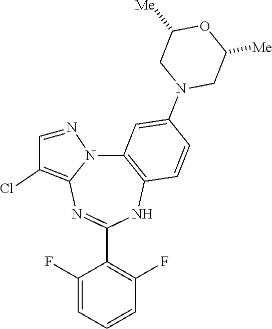 [SMILES: C[C@@H]1CN(c2ccc3c(c2)-n2ncc(Cl)c2N=C(c2c(F)cccc2F)N3)C[C@H](C)O1] | (2R,6S)-4-(3-chloro-5-(2,6- difluorophenyl)-6H- benzo[f]pyrazolo[1,5-a][1,3,5]triazepin- 9-yl)-2,6-dimethylmorpholine | 444.2 | ++ |
| 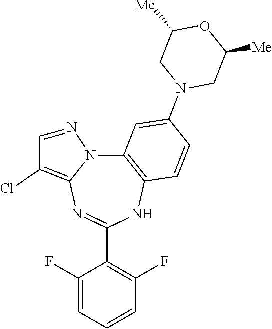 [SMILES: C[C@H]1CN(c2ccc3c(c2)-n2ncc(Cl)c2N=C(c2c(F)cccc2F)N3)C[C@H](C)O1] | (2S,6S)-4-(3-chloro-5-(2,6- difluorophenyl)-6H- benzo[f]pyrazolo[1,5-a][1,3,5]triazepin- 9-yl)-2,6-dimethylmorpholine | 443.8 | +++ |
| 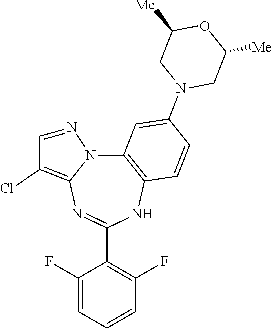 [SMILES: C[C@@H]1CN(c2ccc3c(c2)-n2ncc(Cl)c2N=C(c2c(F)cccc2F)N3)C[C@@H](C)O1] | (2R,6R)-4-(3-chloro-5-(2,6- difluorophenyl)-6H- benzo[f]pyrazolo[1,5-a][1,3,5]triazepin- 9-yl)-2,6-dimethylmorpholine | 443.8 | +++ |
| 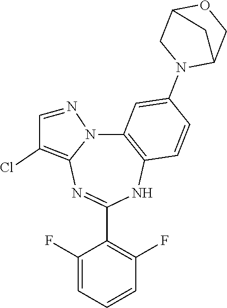 [SMILES: Fc1cccc(F)c1C1=Nc2c(Cl)cnn2-c2cc(N3CC4CC3CO4)ccc2N1] | 5-(3-chloro-5-(2,6-difluorophenyl)-6H- benzo[f]pyrazolo[1,5-a][1,3,5]triazepin- 9-yl)-2-oxa-5-azabicyclo[2.2.1]heptane | 428.1 | +++ |
| Racemic mixture | | | |
| 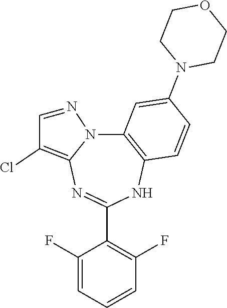 [SMILES: Fc1cccc(F)c1C1=Nc2c(Cl)cnn2-c2cc(N3CCOCC3)ccc2N1] | 4-[3-chloro-5-(2-chloro-6-fluoro- phenyl)-6H-pyrazolo[1,5- a][1,3,5]benzotriazepin-9- yl]morpholine | 432.1 | +++ |
| 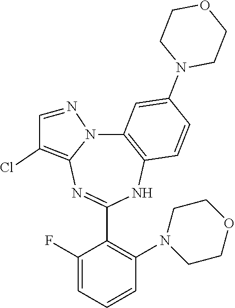 [SMILES: Fc1cccc(N2CCOCC2)c1C1=Nc2c(Cl)cnn2-c2cc(N3CCOCC3)ccc2N1] | 4-[3-chloro-5-(2-fluoro-6-morpholino- phenyl)-6H-pyrazolo[1,5- a][1,3,5]benzotriazepin-9- yl]morpholine | 483.2 | + |
| 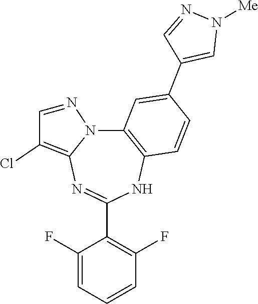 [SMILES: Cn1cc(-c2ccc3c(c2)-n2ncc(Cl)c2N=C(c2c(F)cccc2F)N3)cn1] | 3-chloro-5-(2,6-difluorophenyl)-9-(1- methylpyrazol-4-yl)-6H-pyrazolo[1,5- a][1,3,5]benzotriazepine | 411.1 | +++ |
| 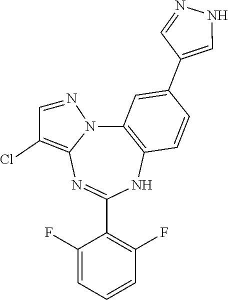 [SMILES: Fc1cccc(F)c1C1=Nc2c(Cl)cnn2-c2cc(-c3cn[nH]c3)ccc2N1] | 3-chloro-5-(2,6-difluorophenyl)-9-(1- methylpyrazol-4-yl)-6H-pyrazolo[1,5- a][1,3,5]benzotriazepine | 397.0 | +++ |
| 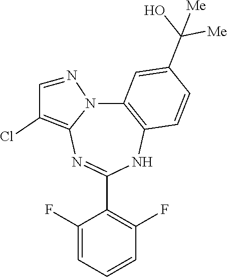 [SMILES: CC(C)(O)c1ccc2c(c1)-n1ncc(Cl)c1N=C(c1c(F)cccc1F)N2] | methyl 2-[3-chloro-5-(2,6- difluorophenyl)-6H-pyrazolo[1,5- a][1,3,5]benzotriazepin-9-yl]propan-2- ol | 389.0 | ++ |
| 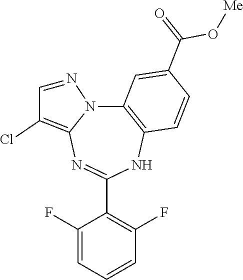 [SMILES: COC(=O)c1ccc2c(c1)-n1ncc(Cl)c1N=C(c1c(F)cccc1F)N2] | Methyl 3-chloro-5-(2,6- difluorophenyl)-6H-pyrazolo[1,5- a][1,3,5]benzotriazepine-9-carboxylate | 389.1 | +++ |
| 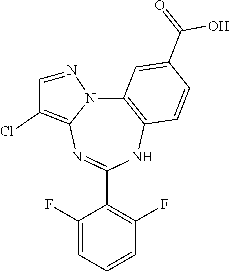 [SMILES: O=C(O)c1ccc2c(c1)-n1ncc(Cl)c1N=C(c1c(F)cccc1F)N2] | Methyl 3-chloro-5-(2,6- difluorophenyl)-6H-pyrazolo[1,5- a][1,3,5]benzotriazepine-9-carboxylic acid | 375.0 | + |
| 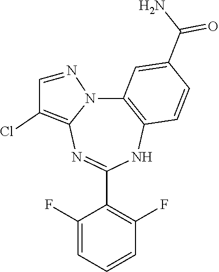 [SMILES: NC(=O)c1ccc2c(c1)-n1ncc(Cl)c1N=C(c1c(F)cccc1F)N2] | 3-chloro-5-(2,6-difluorophenyl)-6H- pyrazolo[1,5-a][1,3,5]benzotriazepine- 9-carboxamide | 374.0 | +++ |
| 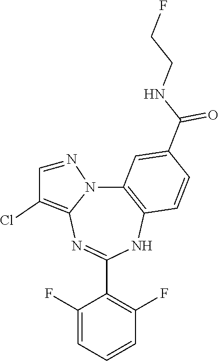 [SMILES: O=C(NCCF)c1ccc2c(c1)-n1ncc(Cl)c1N=C(c1c(F)cccc1F)N2] | 3-chloro-5-(2,6-difluorophenyl)-N-(2- fluoroethyl)-6H-pyrazolo[1,5- a][1,3,5]benzotriazepine-9-carboxamide | 420.1 | +++ |
| 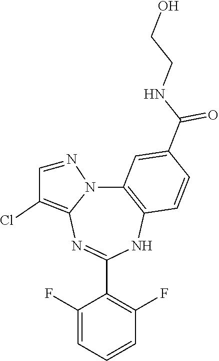 [SMILES: O=C(NCCO)c1ccc2c(c1)-n1ncc(Cl)c1N=C(c1c(F)cccc1F)N2] | 3-chloro-5-(2,6-difluorophenyl)-N-(2- hydroxyethyl)-6H-pyrazolo[1,5- a][1,3,5]benzotriazepine-9-carboxamide | 418.0 | +++ |
| 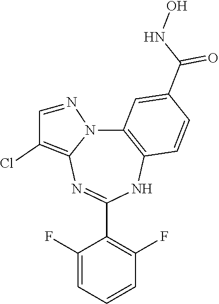 [SMILES: O=C(NO)c1ccc2c(c1)-n1ncc(Cl)c1N=C(c1c(F)cccc1F)N2] | 3-chloro-5-(2,6-difluorophenyl)-6H- pyrazolo[1,5-a][1,3,5]benzotriazepine- 9-carbohydroxamic acid | 390.0 | +++ |
| 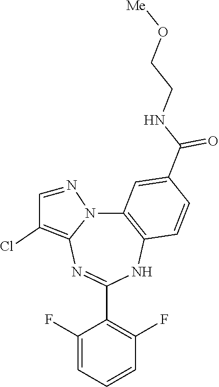 [SMILES: COCCNC(=O)c1ccc2c(c1)-n1ncc(Cl)c1N=C(c1c(F)cccc1F)N2] | 3-chloro-5-(2,6-difluorophenyl)-N-(2- methoxyethyl)-6H-pyrazolo[1,5- a][1,3,5]benzotriazepine-9-carboxamide | 432.1 | +++ |
| 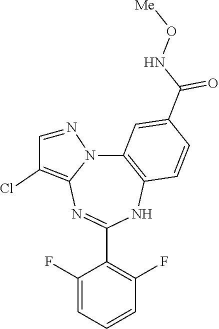 [SMILES: CONC(=O)c1ccc2c(c1)-n1ncc(Cl)c1N=C(c1c(F)cccc1F)N2] | 3-chloro-5-(2,6-difluorophenyl)-N- methoxy-6H-pyrazolo[1,5- a][1,3,5]benzotriazepine-9-carboxamide | 404.0 | +++ |
| 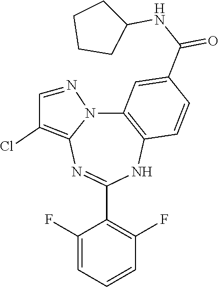 [SMILES: O=C(NC1CCCC1)c1ccc2c(c1)-n1ncc(Cl)c1N=C(c1c(F)cccc1F)N2] | 3-chloro-N-cyclopentyl-5-(2,6- difluorophenyl)-6H-pyrazolo[1,5- a][1,3,5]benzotriazepine-9-carboxamide | 442.0 | +++ |
| 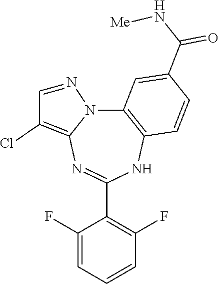 [SMILES: CNC(=O)c1ccc2c(c1)-n1ncc(Cl)c1N=C(c1c(F)cccc1F)N2] | 3-chloro-5-(2,6-difluorophenyl)-N- methyl-6H-pyrazolo[1,5- a][1,3,5]benzotriazepine-9-carboxamide | 387.9 | +++ |
| 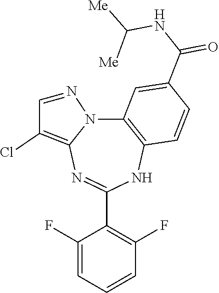 [SMILES: CC(C)NC(=O)c1ccc2c(c1)-n1ncc(Cl)c1N=C(c1c(F)cccc1F)N2] | 3-chloro-5-(2,6-difluorophenyl)-N- isopropyl-6H-pyrazolo[1,5- a][1,3,5]benzotriazepine-9-carboxamide | 416.0 | +++ |
| 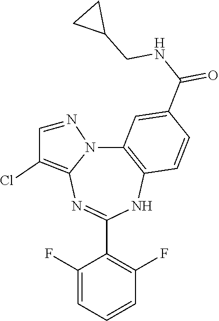 [SMILES: O=C(NCC1CC1)c1ccc2c(c1)-n1ncc(Cl)c1N=C(c1c(F)cccc1F)N2] | 3-chloro-N-(cyclopropylmethyl)-5-(2,6- difluorophenyl)-6H-pyrazolo[1,5- a][1,3,5]benzotriazepine-9-carboxamide | 428.0 | +++ |
| 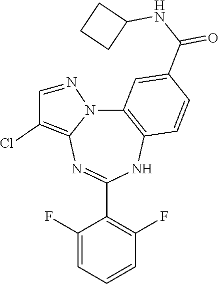 [SMILES: O=C(NC1CCC1)c1ccc2c(c1)-n1ncc(Cl)c1N=C(c1c(F)cccc1F)N2] | 3-chloro-N-cyclobutyl-5-(2,6- difluorophenyl)-6H-pyrazolo[1,5- a][1,3,5]benzotriazepine-9-carboxamide | 428.0 | +++ |
| 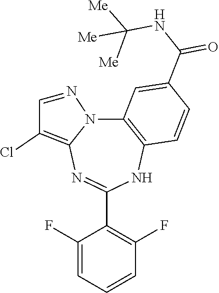 [SMILES: CC(C)(C)NC(=O)c1ccc2c(c1)-n1ncc(Cl)c1N=C(c1c(F)cccc1F)N2] | N-(tert-butyl)-3-chloro-5-(2,6- difluorophenyl)-6H- benzo[f]pyrazolo[1,5- a][1,3,5]triazepine-9-carboxamide | 430.1 | +++ |
| 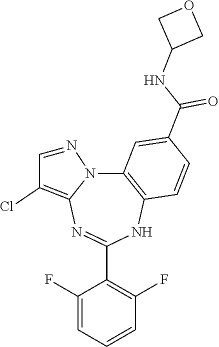 [SMILES: O=C(NC1COC1)c1ccc2c(c1)-n1ncc(Cl)c1N=C(c1c(F)cccc1F)N2] | 3-chloro-5-(2,6-difluorophenyl)-N- (oxetan-3-yl)-6H-benzo[f]pyrazolo[1,5- a] [1,3,5]triazepine-9-carboxamide | 430.0 | +++ |
| 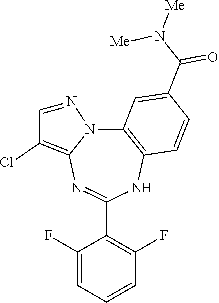 [SMILES: CN(C)C(=O)c1ccc2c(c1)-n1ncc(Cl)c1N=C(c1c(F)cccc1F)N2] | 3-chloro-5-(2,6-difluorophenyl)-N,N- dimethyl-6H-pyrazolo[1,5- a][1,3,5]benzotriazepine-9-carboxamide | 402.0 | ++ |
| 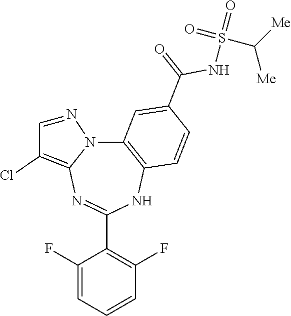 [SMILES: CC(C)S(=O)(=O)NC(=O)c1ccc2c(c1)-n1ncc(Cl)c1N=C(c1c(F)cccc1F)N2] | Methyl 3-chloro-5-(2,6- difluorophenyl)-N-isopropylsulfonyl- 6H-pyrazolo[1,5- a][1,3,5]benzotriazepine-9-carboxamide | 480.2 | + |
| 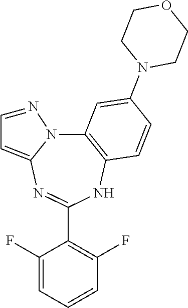 [SMILES: Fc1cccc(F)c1C1=Nc2ccnn2-c2cc(N3CCOCC3)ccc2N1] | 4-[5-(2,6-difluorophenyl)-6H- pyrazolo[1,5-a][1,3,5]benzotriazepin-9- yl]morpholine | 382.1 | +++ |
| 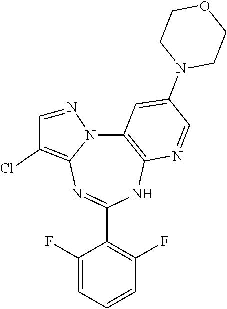 [SMILES: Fc1cccc(F)c1C1=Nc2c(Cl)cnn2-c2cc(N3CCOCC3)cnc2N1] | 4-[5-chloro-8-(2,6-difluorophenyl)- 2,3,7,9,11- pentazatricyclo[8.4.0.02,6]tetradeca- 1(10),3,5,11,13-pentaen-13- yl]morpholine | 417.0 | +++ |
| 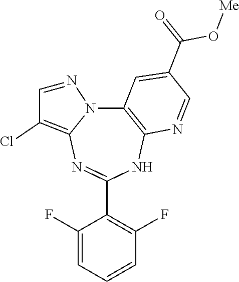 [SMILES: COC(=O)c1cnc2c(c1)-n1ncc(Cl)c1N=C(c1c(F)cccc1F)N2] | Methyl 5-chloro-8-(2,6- difluorophenyl)-2,3,7,9,11- pentazatricyclo[8.4.0.02,6]tetradeca- 1(10),3,5,7,11,13-hexaene-13- carboxylate | 390.0 | +++ |
| 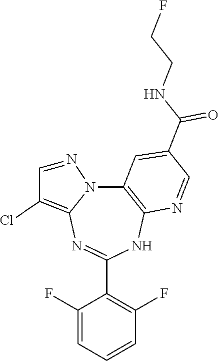 [SMILES: O=C(NCCF)c1cnc2c(c1)-n1ncc(Cl)c1N=C(c1c(F)cccc1F)N2] | 5-chloro-8-(2,6-difluorophenyl)-N-(2- fluoroethyl)-2,3,7,9,11- pentazatricyclo[8.4.0.02,6]tetradeca- 1(10),3,5,7,11,13-hexaene-13- carboxamide | 421.0 | +++ |
| 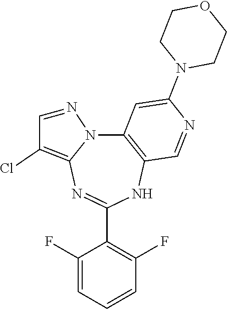 [SMILES: Fc1cccc(F)c1C1=Nc2c(Cl)cnn2-c2cc(N3CCOCC3)ncc2N1] | 4-(3-Chloro-5-(2,6-difluorophenyl)-6H- pyrazolo[1,5-a]pyrido[3,4- f][1,3,5]triazepin-9-yl)morpholine | 417.0 | +++ |
| 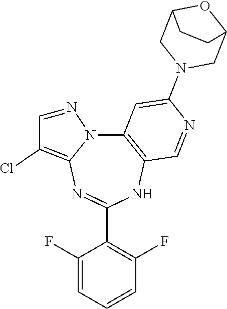 [SMILES: Fc1cccc(F)c1C1=Nc2c(Cl)cnn2-c2cc(N3CC4CCC(C3)O4)ncc2N1] | 3-(3-Chloro-5-(2,6-difluorophenyl)-6H- pyrazolo[1,5-a]pyrido[3,4- f][1,3,5]triazepin-9-yl)-8-oxa-3- azabicyclo[3.2.1]octne | 443.1 | +++ |
| 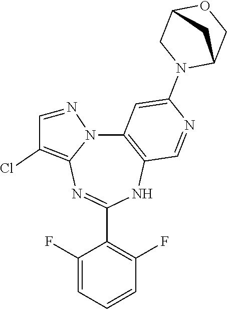 [SMILES: Fc1cccc(F)c1C1=Nc2c(Cl)cnn2-c2cc(N3C[C@H]4C[C@@H]3CO4)ncc2N1] | (1R,4R)-5-[5-chloro-8-(2,6- difluorophenyl)-2,3,7,9,12- pentazatricyclo[8.4.0.02,6]tetradeca- 1(10),3,5,7,11,13-hexaen-13-yl]-2-oxa- 5-azabicyclo[2.2.1]heptane | 429.0 | +++ |
| 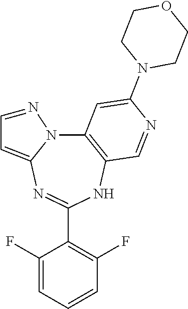 [SMILES: Fc1cccc(F)c1C1=Nc2ccnn2-c2cc(N3CCOCC3)ncc2N1] | 4-(5-(2,6-difluorophenyl)-6H- pyrazolo[1,5-a]pyrido[3,4- f][1,3,5]triazepin-9-yl)morpholine | 383.0 | +++ |
| 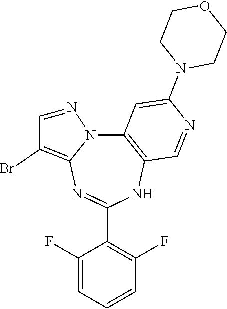 [SMILES: Fc1cccc(F)c1C1=Nc2c(Br)cnn2-c2cc(N3CCOCC3)ncc2N1] | 4-(3-bromo-5-(2,6-difluorophenyl)-6H- pyrazolo[1,5-a]pyrido[3,4- f][1,3,5]triazepin-9-yl)morpholine | 461.0 | +++ |
| 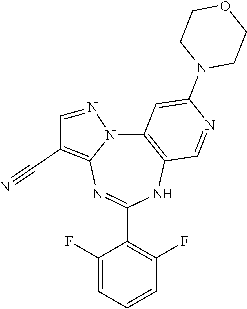 [SMILES: N#Cc1cnn2c1N=C(c1c(F)cccc1F)Nc1cnc(N3CCOCC3)cc1-2] | 5-(2,6-difluorophenyl)-9-morpholino- 6H-pyrazolo[1,5-a]pyrido[3,4- f][1,3,5]triazepine-3-carbonitrile | 408.1 | +++ |
| 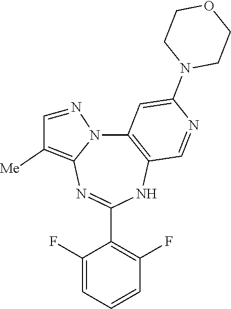 [SMILES: Cc1cnn2c1N=C(c1c(F)cccc1F)Nc1cnc(N3CCOCC3)cc1-2] | 4-(5-(2,6-difluorophenyl)-3-methyl-6H- pyrazolo[1,5-a]pyrido[3,4- f][1,3,5]triazepin-9-yl)morpholine | 397.0 | +++ |
| 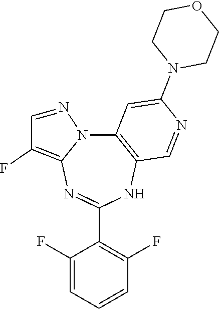 [SMILES: Fc1cccc(F)c1C1=Nc2c(F)cnn2-c2cc(N3CCOCC3)ncc2N1] | 4-(5-(2,6-difluorophenyl)-3-fluoro-6H- pyrazolo[1,5-a]pyrido[3,4- f][1,3,5]triazepin-9-yl)morpholine | 401.1 | +++ |
| 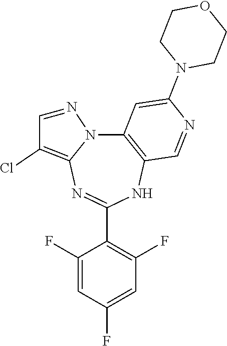 [SMILES: Fc1cc(F)c(C2=Nc3c(Cl)cnn3-c3cc(N4CCOCC4)ncc3N2)c(F)c1] | 4-(3-chloro-5-(2,4,6-trifluorophenyl)- 6H-pyrazolo[1,5-a]pyrido[3,4- f][1,3,5]triazepin-9-yl)morpholine | 435.0 | +++ |
| 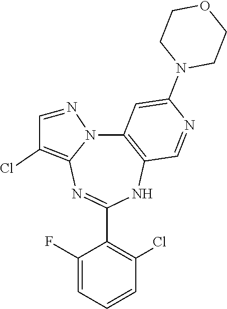 [SMILES: Fc1cccc(Cl)c1C1=Nc2c(Cl)cnn2-c2cc(N3CCOCC3)ncc2N1] | 4-(3-chloro-5-(2-chloro-6- fluorophenyl)-6H-pyrazolo[1,5- alpyrido[3,4-f][1,3,5]triazepin-9- yl)morpholine | 433.0 | +++ |
| 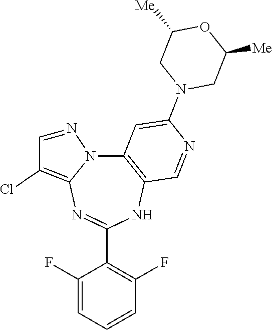 [SMILES: C[C@H]1CN(c2cc3c(cn2)NC(c2c(F)cccc2F)=Nc2c(Cl)cnn2-3)C[C@H](C)O1] | (2S,6S)-4-[5-chloro-8-(2,6- difluorophenyl)-2,3,7,9,12- pentazatricyclo[8.4.0.02,6]tetradeca- 1(10),3,5,7,11,13-hexaen-13-yl]-2,6- dimethyl-morpholine | 445.2 | +++ |
| 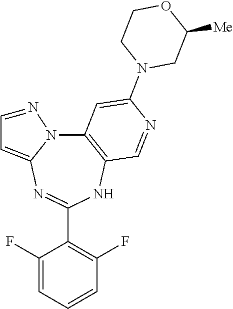 [SMILES: C[C@H]1CN(c2cc3c(cn2)NC(c2c(F)cccc2F)=Nc2ccnn2-3)CCO1] | (S)-4-(5-(2,6-difluorophenyl)-6H- pyrazolo[1,5-a]pyrido[3,4- f][1,3,5]triazepin-9-yl)-2- methylmorpholine | 397.0 | +++ |
| 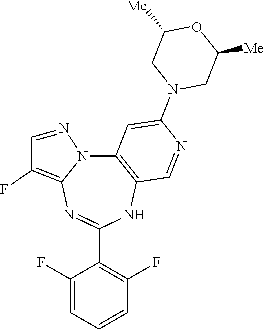 [SMILES: C[C@H]1CN(c2cc3c(cn2)NC(c2c(F)cccc2F)=Nc2c(F)cnn2-3)C[C@H](C)O1] | (2S,6S)-4-(5-(2,6-difluorophenyl)-3- fluoro-6H-pyrazolo[1,5-a]pyrido[3,4- f][1,3,5]triazepin-9-yl)-2,6- dimethylmorpholine | 429.1 | ++ |
| 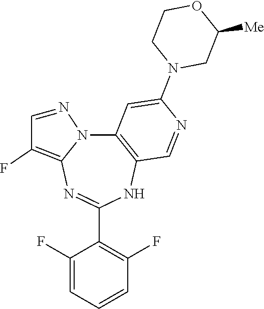 [SMILES: C[C@H]1CN(c2cc3c(cn2)NC(c2c(F)cccc2F)=Nc2c(F)cnn2-3)CCO1] | (S)-4-(5-(2,6-difluorophenyl)-3-fluoro- 6H-pyrazolo[1,5-a]pyrido[3,4- f][1,3,5]triazepin-9-yl)-2- methylmorpholine | 415.0 | +++ |
| 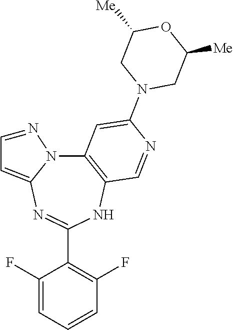 [SMILES: C[C@H]1CN(c2cc3c(cn2)NC(c2c(F)cccc2F)=Nc2ccnn2-3)C[C@H](C)O1] | (2S,6S)-4-(5-(2,6-difluorophenyl)-6H- pyrazolo[1,5-a]pyrido[3,4- f][1,3,5]triazepin-9-yl)-2,6- dimethylmorpholine | 411.2 | +++ |

### Example 1

### Synthesis of 9-bromo-3-chloro-5-(2,6-difluorophenyl)-6H-pyrazolo[1,5-a][1,3,5]benzotriazepine

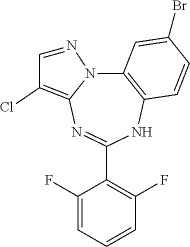 [SMILES: Fc1cccc(F)c1C1=Nc2c(Cl)cnn2-c2cc(Br)ccc2N1]

A solution of 2-(2-amino-5-bromo-phenyl)-4-chloro-pyrazol-3-amine (2.8 g, 9.74 mmol, 1.0 eq.) and 2,6-difluorobenzaldehyde (1.38 g, 9.74 mmol, 1.0 eq.) in t-BuOH (50 mL) was stirred at 50 °C for 15 min. To the reaction solution was added DDQ (2.21 g, 9.74 mmol, 1.0 eq.), and the resulting mixture was stirred at 50 °C for additional 30 min. The reaction mixture was cooled to rt, quenched with H₂O (50 mL), neutralized with aqueous NaHCO₃ solution to pH=9, and extracted with EtOAc (100 mL×3). The combined organic layers were concentrated under reduced pressure. The crude product was purified by flash chromatography (ISCO®; 80 g SepaFlash® Silica Flash Column, 0~40% EtOAc/petroleum, 30 mL/min, 254 mn) to afford 9-bromo-3-chloro-5-(2,6-difluorophenyl)-6H-pyrazolo[1,5-a][1,3,5]benzotriazepine as a brown solid (1.70 g, 42.6%). ¹HNMR (400 MHz, CDCl₃) δ ppm 6.75-6.77 (d, J=8.4, 1H), 7.24-7.28 (m, 3H), 7.59-7.61 (m, 1H), 7.72 (m, 2H), 9.73 (s, 1H); LCMS (ESI) m/z 408.9, 410.9 [M+H]⁺.

### Example 2

### Synthesis of 4-[3-chloro-5-(2,6-difluorophenyl)-6H-pyrazolo[1,5-a][1,3,5]benzotriazepin-9-yl]morpholine

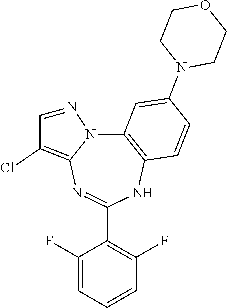 [SMILES: Fc1cccc(F)c1C1=Nc2c(Cl)cnn2-c2cc(N3CCOCC3)ccc2N1]

A mixture of 9-bromo-3-chloro-5-(2,6-difluorophenyl)-6H-pyrazolo[1,5-a][1,3,5]benzotriazepine (100 mg, 0.244 mmol, 1.0 eq.), NaO^{t}Bu (70.4 mg, 0.732 mmol), morpholine (23.4 mg, 0.269 mmol) and Pd(t-Bu₃P)₂ (12.5 mg, 24.4 mol, 1.0 eq.) in dioxane (3 mL) was stirred at 80 °C for 16 h under N₂ atmosphere. The reaction mixture quenched with water (2 mL), filtered and subjected to purification by reverse phase preparative HPLC (Prep-C18, 5 μM OBD column, 19×250 mm, waters; gradient elution of 0-40% MeCN in water over a 20 min period, where both water and MeCN contain 0.1% HCl acid, flow rate: 20 mL/min, detector UV wavelength: 254 nm), to provide the title compound as a yellow solid (4.6 mg, 4.47%). ¹HNMR (400 MHz, CD₃OD) δ ppm 3.27-3.32 (m, 4H), 3.86-3.92 (m, 4H), 6.68-6.69 (m, 2H), 7.18-7.26 (m, 3H) 7.53 (m, 1H). 7.62 (s, 1H), 9.41 (s, 1H); LCMS (ESI) m/z 416.1 [M+H]⁺.

### Example 3

### Synthesis of 7-[3-chloro-5-(2,6-difluorophenyl)-6H-pyrazolo[1,5-a][1,3,5]benzotriazepin-9-yl]-2-oxa-7-azaspiro[3.5]nonane

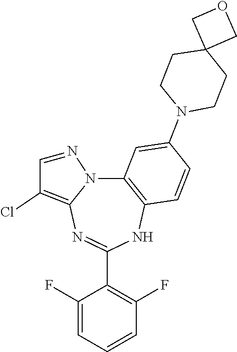 [SMILES: Fc1cccc(F)c1C1=Nc2c(Cl)cnn2-c2cc(N3CCC4(CC3)COC4)ccc2N1]

The title compound was made from 9-bromo-3-chloro-5-(2,6-difluorophenyl)-6H-pyrazolo[1,5-a][1,3,5]benzotriazepine (Example 1), following the synthetic procedure as described in the synthesis of 4-[3-chloro-5-(2,6-difluorophenyl)-6H-pyrazolo[1,5-a][1,3,5]benzotriazepin-9-yl]morpholine (Example 2), except that 2-oxa-7-azaspiro [3.5]nonane was used instead of morpholine, and the reaction was conducted in dioxane at 70 °C for 16 h. The crud product was purified by column chromatography (ISCO®; 4 g SepaFlash® Silica Flash Column, petroleum ether/EtOAc with EtOAc from 0~40.5%, 20 mL/min, 254 nm) to afford the title product as a yellow solid (25 mg, 44.5%). ¹HNMR (400 MHz, DMSO-d₆) δ ppm 1.89-1.80 (m, 4H), 3.03-2.97 (m, 4H), 4.32 (s, 4H), 6.66 (s, 2H), 7.18 (s, 1H), 7.23 (t. J=8.1 Hz, 2H), 7.64-7.52 (m, 2H), 9.39 (s, 1H); ¹⁹F NMR (377 MHz, DMSO-d₆) δ ppm −113.60; LCMS (ESI) m/z 456.1 [M+H]⁺.

### Example 4

### Synthesis of 4-[3-chloro-5-(2,6-difluorophenyl)-6H-pyrazolo[1,5-a][1,3,5]benzotriazepin-9-yl]morpholine

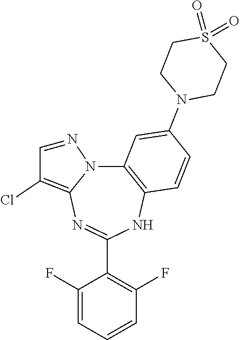 [SMILES: O=S1(=O)CCN(c2ccc3c(c2)-n2ncc(Cl)c2N=C(c2c(F)cccc2F)N3)CC1]

The title compound was made from 9-bromo-3-chloro-5-(2,6-difluorophenyl)-6H-pyrazolo[1,5-a][1,3,5]benzotriazepine (Example 1), following the synthetic procedure as described in the synthesis of 4-[3-chloro-5-(2,6-difluorophenyl)-6H-pyrazolo[1,5-a][1,3,5]benzotriazepin-9-yl]morpholine (Example 2), except that thiomorpholine 1,1-dioxide was used in the place of morpholine, and the reaction was conducted in dioxane at 70 °C for 16 h. The crude final product was purified by column chromatography (ISCO®; 4 g SepaFlash® Silica Flash Column, petroleum ether/EtOAc with EtOAc from 0-51%, 20 mL/min, 254 nm) to provide the title compound as a yellow solid (17.32 mg, 30.6%). ¹HNMR (400 MHz, DMSO-d₆) δ ppm 3.13 (br s, 4H), 3.69 (br s, 4H), 6.82-6.66 (m, 2H), 7.31-7.20 (m, 3H), 7.72-7.52 (m, 2H), 9.45 (s, 1H); ¹⁹F NMR (377 MHz, DMSO-d₆) δ=−113.56; LCMS (ESI) m/z 464.2 [M+H]⁻.

### Example 5

### Synthesis of 3-chloro-5-(2,6-difluorophenyl)-9-[(3S)-3-fluoropyrrolidin-1-yl]-6H-pyrazolo[1,5-a][1,3,5]benzotriazepine

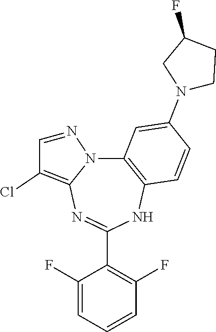 [SMILES: Fc1cccc(F)c1C1=Nc2c(Cl)cnn2-c2cc(N3CC[C@H](F)C3)ccc2N1]

The title compound was made from 9-bromo-3-chloro-5-(2,6-difluorophenyl)-6H-pyrazolo[1,5-a][1,3,5]benzotriazepine (Example 1), following the synthetic procedure as described in the synthesis of 4-[3-chloro-5-(2,6-difluorophenyl)-6H-pyrazolo[1,5-a][1,3,5]benzotriazepin-9-yl]morpholine (Example 2), except that (3S)-3-fluoropyrrolidine was used in the place of morpholine, and the reaction was conducted in dioxane at 70 °C for 16 h. The crude final product was purified b by prep-HPLC (column: 2_Phenomenex Gemini C18 75×40 mm×3 μm; mobile phase: [water (NH₄HCO₃)-MeCN]; B %: 53%-83%, 9.5 min) to provide the title compound as a yellow solid (21 mg, 40.1%). ¹HNMR (400 MHz, DMSO-d₆) δ ppm 2.28-2.13 (m, 2H), 3.35-3.24 (m, 2H), 3.55 (br dd, J=3.6, 12.1 Hz, 1H), 5.35 (br s, 0.5 H), 5.48 (br s, 0.5 H), 6.31 (dd, J=2.6, 8.8 Hz, 1H), 6.67 (d, J=8.8 Hz, 1H), 6.84 (d, J=2.6 Hz, 1H), 7.23 (t, J=8.1 Hz, 2H), 7.60-7.53 (m, 1H), 7.62-7.60 (m, 1H), 9.32 (s, 1H); ¹⁹F NMR (377 MHz, DMSO-d₆) δ ppm −113.61; LCMS (ESI) m/z 418.1 [M+H]⁺.

### Example 6

### Synthesis of 3-chloro-5-(2,6-difluorophenyl)-9-[(3R)-3-fluoropyrrolidin-1-yl]-6H-pyrazolo[1,5-a][1,3,5]benzotriazepine

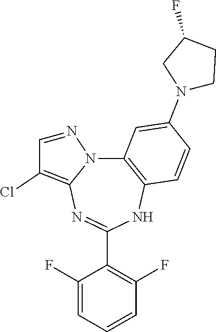 [SMILES: Fc1cccc(F)c1C1=Nc2c(Cl)cnn2-c2cc(N3CC[C@@H](F)C3)ccc2N1]

The title compound was made from 9-bromo-3-chloro-5-(2,6-difluorophenyl)-6H-pyrazolo[1,5-a][1,3,5]benzotriazepine (Example 1), following the synthetic procedure as described in the synthesis of 4-[3-chloro-5-(2,6-difluorophenyl)-6H-pyrazolo[1,5-a][1,3,5]benzotriazepin-9-yl]morpholine (Example 2), except that (3R)-3-fluoropyrrolidine was used in the place of morpholine, and the reaction was conducted in dioxane at 70 °C for 16 h. The crude final product was purified by the reverse phase HPLC (column: 2_Phenomenex Gemini C18 75×40 mm×3 m; mobile phase: [water (NH₄HCO₃)-ACN]; B %: 53%-83%, 9.5 min) to provide the title compound as a yellow solid (18 mg, 22.1%). ¹HNMR (400 MHz, DMSO-d₆) δ ppm 2.29-2.16 (m, 2H), 3.36-3.29 (m, 2H), 3.53-3.50 (m, 2H), 5.35 (br s, 0.5 H), 5.48 (br s, 0.5 H), 6.30 (br d, J=8.6 Hz, 1H), 6.67 (br d, J=8.4 Hz, 1H), 6.84 (br s, 1H), 7.23 (br t. J=8.0 Hz, 2H), 7.59-7.51 (m, 1H), 7.61 (br d, J=1.1 Hz, 1H), 9.31 (s. 1H); ¹⁹F NMR (377 MHz, DMSO-d₆) ppm −113.61; LCMS (ESI) m/z 418.1 [M+H]⁺.

### Example 7

### Synthesis of 3-chloro-5-(2,6-difluorophenyl)-9-(3,3-difluoropyrrolidin-1-yl)-6H-pyrazolo[1,5-a][1,3,5]benzotriazepine

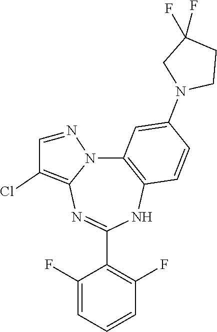 [SMILES: Fc1cccc(F)c1C1=Nc2c(Cl)cnn2-c2cc(N3CCC(F)(F)C3)ccc2N1]

The title compound was made from 9-bromo-3-chloro-5-(2,6-difluorophenyl)-6H-pyrazolo[1,5-a][1,3,5]benzotriazepine (Example 1), following the synthetic procedure as described in the synthesis of 4-[3-chloro-5-(2,6-difluorophenyl)-6H-pyrazolo[1,5-a][1,3,5]benzotriazepin-9-yl]morpholine (Example 2), except that 3,3-difluoropyrrolidine was used in the place of morpholine, and the reaction was conducted in dioxane at 70 °C for 16 h. The crude final product was purified by prep-HPLC ((column: 2_Phenomenex Gemini C18 75×40 mm×3 m; mobile phase: [water (NH₄HCO₃)-MeCN]; B %: 53%-83%, 9.5 min) to provide the title compound as a yellow solid (35 mg, 41.1%). ¹HNMR (400 MHz, CD₃OD) δ ppm 2.50 (tt, J=7.1, 14.1 Hz, 2H), 3.51 (t, J=7.2 Hz, 2H), 3.65 (t, J=13.2 Hz, 2H), 6.36 (dd, J=2.8, 8.6 Hz, 1H), 6.66 (d, J=8.8 Hz, 1H), 6.94 (d, J=2.8 Hz, 1H), 7.13-7.05 (m, 2H), 7.49 (s, 1H), 7.57-7.50 (m, 1H); ¹⁹F NMR (377 MHz, CD₃OD) δ ppm −100.71, −114.79; LCMS (ESI) m/z 436.1 [M+H]⁺.

### Example 8

### Synthesis of 3-chloro-5-(2,6-difluorophenyl)-9-(4-methylpiperazin-1-yl)-6H-pyrazolo[1,5-a][1,3,5]benzotriazepine

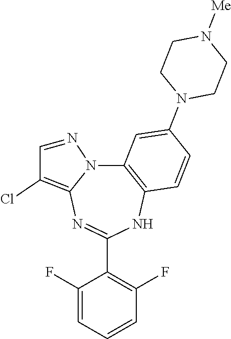 [SMILES: CN1CCN(c2ccc3c(c2)-n2ncc(Cl)c2N=C(c2c(F)cccc2F)N3)CC1]

The title compound was made from 9-bromo-3-chloro-5-(2,6-difluorophenyl)-6H-pyrazolo[1,5-a][1,3,5]benzotriazepine (Example 1), following the synthetic procedure as described in the synthesis of 4-[3-chloro-5-(2,6-difluorophenyl)-6H-pyrazolo[1,5-a][1,3,5]benzotriazepin-9-yl]morpholine (Example 2), except that 1-methylpiperazine was used in the place of morpholine, and the reaction was conducted in dioxane at 70 °C for 16 h. The crude final product was purified by flash chromatography (ISCO®; 4 g SepaFlash® Silica Flash Column, petroleum ether/EtOAc with EtOAc from 0~100%, 40 mL/min, 254 mn) to provide the title compound as a yellow solid (15 mg, 14.0%). ¹HNMR (400 MHz, CD₃OD) δ ppm 2.39 (s, 3H), 2.69-2.62 (m, 4H), 3.25-3.19 (m, 4H), 6.76-6.66 (m, 2H), 7.11 (t, J=8.2 Hz, 2H), 7.31 (d, J=2.5 Hz, 1H), 7.51 (s, 1H), 7.60-7.52 (m, 1H); LCMS (ESI) m/z 429.1 [M+H]⁺.

### Example 9

### Synthesis of 3-chloro-9-(4,7-diazaspiro[2.5]octan-7-yl)-5-(2,6-difluorophenyl)-6H-pyrazolo[1,5-a][1,3,5]benzotriazepine

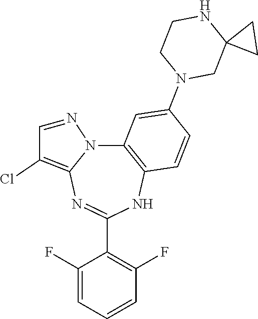 [SMILES: Fc1cccc(F)c1C1=Nc2c(Cl)cnn2-c2cc(N3CCNC4(CC4)C3)ccc2N1]

### Step 1

9-Bromo-3-chloro-5-(2,6-difluorophenyl)-6H-pyrazolo[1,5-a][1,3,5]benzotriazepine (Example 1) and tert-butyl 4, 7-diazaspiro [2.5]octane-4-carboxylate were used following the synthetic procedure as described in the synthesis of 4-[3-chloro-5-(2,6-difluorophenyl)-6H-pyrazolo[1,5-a][1,3,5]benzotriazepin-9-yl]morpholine (Example 2), except that the reaction was conducted in dioxane at 70 °C for 16 h. The reaction mixture was purified by column chromatography (ISCO®; 4 g SepaFlash® Silica Flash Column, petroleum ether/EtOAc with EtOAc from 0~15%, 20 mL/min, 254 nm) to provide tert-butyl 7-[3-chloro-5-(2,6-difluorophenyl)-6H-pyrazolo[1,5-a][1,3,5]benzotriazepin-9-yl]-4,7-diazaspiro[2.5]octane-4-carboxylate as a yellow solid (354 mg, 28.0%). LCMS (ESI) m/z 541.2 [M+H]⁺.

### Step 2

A mixture of tert-butyl 7-[3-chloro-5-(2,6-difluorophenyl)-6H-pyrazolo[1,5-a][1,3,5]benzotriazepin-9-yl]-4,7-diazaspiro[2.5]octane-4-carboxylate (59 mg, 0.109 mmol) in HCl/MeOH (2 mL) was stirred at 20 °C for 2 h. The reaction mixture was quenched with saturated aqueous NaHCO₃ solution (5 mL) and extracted with EtOAc (10 mL×3). The combined organic layers were washed with brine (5 mL), dried over Na₂SO₄, filtered and concentrated under reduced pressure to provide the title compound as a yellow solid (36 mg, 73.0%). ¹HNMR (400 MHz, DMSO-d₆) δ ppm 0.59-0.44 (m, 4H), 2.90-2.84 (m, 4H), 3.02-2.98 (m, 2H), 6.69-6.60 (m, 2H), 7.12 (d, J=2.5 Hz, 1H), 7.23 (t, J=8.2 Hz, 2 H), 7.60-7.54 (m, 1H), 7.61 (s, 1H), 8.16 (s, 1H), 9.37 (s, 1H); ¹⁹F NMR (377 MHz, DMSO-d₆) δ ppm −113.59; LCMS (ESI) m/z 441.1 [M+H]⁺.

### Example 10

### Synthesis of 3-chloro-5-(2,6-difluorophenyl)-9-piperazin-1-yl-6H-pyrazolo[1,5-a][1,3,5]benzotriazepine

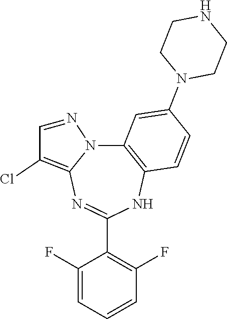 [SMILES: Fc1cccc(F)c1C1=Nc2c(Cl)cnn2-c2cc(N3CCNCC3)ccc2N1]

### Step 1

Compound 9-bromo-3-chloro-5-(2,6-difluorophenyl)-6H-pyrazolo[1,5-a][1,3,5]benzotriazepine (Example 1) was reacted with tert-butyl piperazine-1-carboxylate, following the synthetic procedure as described in the synthesis of 4-[3-chloro-5-(2,6-difluorophenyl)-6H-pyrazolo[1,5-a][1,3,5]benzotriazepin-9-yl]morpholine (Example 2), except that the reaction was conducted in dioxane at 70 °C for 16 h. The crude final product was purified by column chromatography (ISCO®; 4 g SepaFlash® Silica Flash Column, petroleum ether/EtOAc with EtOAc from 0-19.8%, 20 mL/min, 254 nm) to afford tert-butyl 4-(3-chloro-5-(2,6-difluorophenyl)-6H-benzo[f]pyrazolo[1,5-a][1,3,5]triazepin-9-yl)piperazine-1-carboxylate as a yellow solid (80 mg, 41.5%). LCMS (ESI) m/z 515.1 [M+H]⁺.

### Step 2

A solution of tert-butyl 4-(3-chloro-5-(2,6-difluorophenyl)-6H-benzo[f]pyrazolo[1,5-a][1,3,5]triazepin-9-yl)piperazine-1-carboxylate as a yellow solid (59 mg, 0.114 mmol) in HCl/MeOH (4 M, 0.5 mL) was stirred at 20 °C for 2 h. The reaction mixture was quenched with saturated aqueous NaHCO₃ solution (5 mL) and extracted with EtOAc (10 mL×3). The combined organic layers were washed with brine (5 mL), dried over Na₂SO₄, filtered and concentrated under reduced pressure. The crude product was purified by preparative TLC (silica, DCM/MeOH=10/1, 254 nm) to provide the title compound as a yellow solid (11 mg, 22.4%). ¹HNMR (400 MHz, DMSO-d₆) δ ppm 2.90 (br s, 4H), 3.04 (br s, 4H), 6.72-6.64 (m, 2H), 7.20-7.16 (m, 1H), 7.23 (t, J=8.2 Hz, 2H), 7.66-7.53 (m, 2H), 8.21 (s, 1H), 9.40 (s, 1H); ¹⁹F NMR (377 MHz, DMSO-d₆) δ ppm −113.59; LCMS (ESI) m/z 415.1 [M+H]⁺.

### Example 11

### Synthesis of (3R)-4-[3-chloro-5-(2,6-difluorophenyl)-6H-pyrazolo[1,5-a][1,3,5]benzotriazepin-9-yl]-3-methyl-morpholine

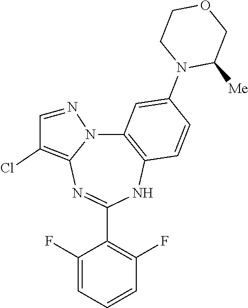 [SMILES: C[C@@H]1COCCN1c1ccc2c(c1)-n1ncc(Cl)c1N=C(c1c(F)cccc1F)N2]

The title compound was made from 9-bromo-3-chloro-5-(2,6-difluorophenyl)-6H-pyrazolo[1,5-a][1,3,5]benzotriazepine (Example 1), following the synthetic procedure as described in the synthesis of 4-[3-chloro-5-(2,6-difluorophenyl)-6H-pyrazolo[1,5-a][1,3,5]benzotriazepin-9-yl]morpholine (Example 2), except that (R)-3-methylmorpholine was used in the place of morpholine, and the reaction was conducted in dioxane at 70 °C for 16 h. The crude final product was purified by column chromatography (ISCO®; 4 g SepaFlash® Silica Flash Column, petroleum ether/EtOAc with EtOAc from 0~18.7%, 20 mL/min, 254 nm) to provide the title compound as a yellow solid (17 mg, 32.3%). ¹HNMR (400 MHz, DMSO-d₆) δ ppm 0.97 (d, J=6.4 Hz, 3H), 3.01-2.92 (m, 1H), 3.10-3.02 (m, 1H), 3.53 (br d, J=8.3 Hz, 2H), 3.65 (br d, J=8.4 Hz, 2H), 3.69 (br s, 1H), 3.76-3.68 (m, 1H), 3.72 (br d, J=6.5 Hz, 1H), 3.87 (br d, J=11.4 Hz, 1H), 6.66-6.60 (m, 1H), 6.73-6.67 (m, 1H), 7.14 (d, J=2.5 Hz, 1H), 7.24 (t, J=8.1 Hz, 2H), 7.61-7.53 (m, 1H), 7.65-7.61 (m, 1H), 9.41 (s, 1H); ¹⁹F NMR (377 MHz, DMSO-d₆) δ ppm −113.58; LCMS (ESI) m/z 430.1 [M+H]⁺.

### Example 12

### Synthesis of (3S)-4-[3-chloro-5-(2,6-difluorophenyl)-6H-pyrazolo[1,5-a][1,3,5]benzotriazepin-9-yl]-3-methyl-morpholine

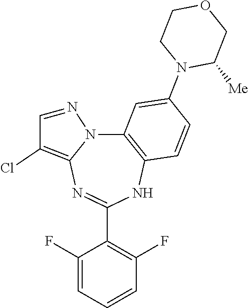 [SMILES: C[C@H]1COCCN1c1ccc2c(c1)-n1ncc(Cl)c1N=C(c1c(F)cccc1F)N2]

The title compound was made from 9-bromo-3-chloro-5-(2,6-difluorophenyl)-6H-pyrazolo[1,5-a][1,3,5]benzotriazepine (Example 1), following the synthetic procedure as described in the synthesis of 4-[3-chloro-5-(2,6-difluorophenyl)-6H-pyrazolo[1,5-a][1,3,5]benzotriazepin-9-yl]morpholine (Example 2), except that (3S)-3-methylmorpholine was used in the place of morpholine, and the reaction was conducted in dioxane at 70 °C for 16 h. The crude final product was purified by column chromatography (ISCO®; 4 g SepaFlash® Silica Flash Column, petroleum ether/EtOAc with EtOAc from 0~45%, 20 mL/min, 254 nm) to provide the title compound as a yellow solid (15 mg, 28.6%). ¹HNMR (400 MHz, DMSO-d₆) δ ppm 0.97 (d, J=6.3 Hz, 3H), 3.08-2.92 (m, 2H), 3.55-3.48 (m, 1H), 3.67-3.59 (m, 2H), 3.75-3.68 (m, 1H), 3.91-3.82 (m, 1H), 6.72-6.60 (m, 2H), 7.14 (d, J=2.5 Hz, 1H), 7.24 (t, J=8.2 Hz, 2H), 7.64-7.54 (m, 2H), 9.39 (s, 1H); ¹⁹F NMR (377 MHz, DMSO-d₆) δ ppm −113.56; LCMS (ESI) m/z 430.1 [M+H]⁺.

### Example 13

### Synthesis of 6-[3-chloro-5-(2,6-difluorophenyl)-6H-pyrazolo[1,5-a][1,3,5]benzotriazepin-9-yl]-3-oxa-6-azabicyclo[3.1.1]heptane

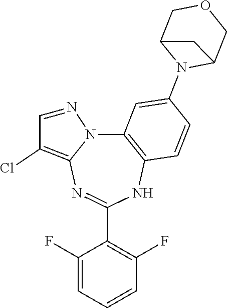 [SMILES: Fc1cccc(F)c1C1=Nc2c(Cl)cnn2-c2cc(N3C4COCC3C4)ccc2N1]

The title compound was made from 9-bromo-3-chloro-5-(2,6-difluorophenyl)-6H-pyrazolo[1,5-a][1,3,5]benzotriazepine (Example 1), following the synthetic procedure as described in the synthesis of 4-[3-chloro-5-(2,6-difluorophenyl)-6H-pyrazolo[1,5-a][1,3,5]benzotriazepin-9-yl]morpholine (Example 2), except that 3-oxa-6-azabicyclo [3.1.1]heptane was used in the place of morpholine, and the reaction was conducted in dioxane at 70 °C for 16 h. The crude final product was purified by prep-HPLC (column: 2_Phenomenex Gemini C18 75×40 mm×3 μm; mobile phase: [water (NH₄HCO₃)-MeCN]; B %: 44%-74%, 9.5 min) to provide the title compound as a yellow solid (12 mg, 14.2%). ¹HNMR (400 MHz, DMSO-d₆) δ ppm 1.79 (d, J=8.0 Hz, 1H), 2.63 (q, J=6.5 Hz, 1H), 3.60 (d, J=10.5 Hz, 2H), 4.10 (d, J=10.5 Hz, 2H), 4.20 (d, J=6.0 Hz, 2H), 6.31 (dd, J=2.5, 8.5 Hz, 1H), 6.68 (d, J=8.5 Hz, 1H), 6.78 (d, J=2.5 Hz, 1H), 7.24 (t, J=8.2 Hz, 2H), 7.59-7.54 (m, 1H), 7.61-7.59 (m, 1H), 9.37 (s, 1H); LCMS (ESI) m/z 428.1 [M+H]⁺.

### Example 14

### Synthesis of (1R,5S)-3-[3-chloro-5-(2,6-difluorophenyl)-6H-pyrazolo[1,5-a][1,3,5]benzotriazepin-9-yl]-8-oxa-3-azabicyclo[3.2.1]octane

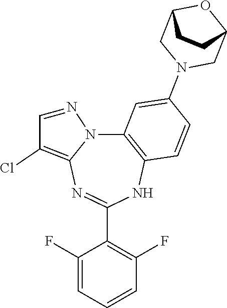

The title compound was made from 9-bromo-3-chloro-5-(2,6-difluorophenyl)-6H-pyrazolo[1,5-a][1,3,5]benzotriazepine (Example 1), following the synthetic procedure as described in the synthesis of 4-[3-chloro-5-(2,6-difluorophenyl)-6H-pyrazolo[1,5-a][1,3,5]benzotriazepin-9-yl]morpholine (Example 2), except that (1R,5S)-8-oxa-3-azabicyclo[3.2.1]octane was used in the place of morpholine, and the reaction was conducted in dioxane at 70 °C for 16 h. The crude final product was purified by column chromatography (ISCO®; 4 g SepaFlash® Silica Flash Column, petroleum ether/EtOAc with EtOAc from 0~21%, 20 mL/min, 254 nm) to provide the title compound as a yellow solid (25 mg, 28.2%). ¹HNMR (400 MHz, DMSO-d₆) δ ppm 1.80 (br s, 4H), 2.77 (br d, J=9.1 Hz, 2H), 3.28 (br d, J=10.8 Hz, 4H), 4.40 (br s, 2H), 6.58 (dd, J=2.7, 8.9 Hz, 1H), 6.71-6.64 (m, 1H), 7.08 (d, J=2.6 Hz, 1H), 7.24 (t, J=8.1 Hz, 2H), 7.65-7.53 (m, 2H), 9.37 (s, 1H); ¹⁹FNMR (377 MHz, DMSO-d₆) δ ppm −113.62; LCMS (ESI) m/z 442.1 [M+H]⁺.

### Example 15

### Synthesis of (1R,4R)-5-[3-chloro-5-(2,6-difluorophenyl)-6H-pyrazolo[1,5-a][1,3,5]benzotriazepin-9-yl]-2-oxa-5-azabicyclo[2.2.2]octane

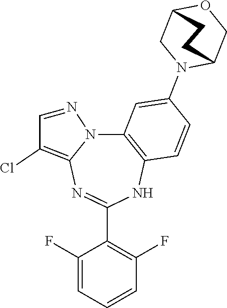

The title compound was made from 9-bromo-3-chloro-5-(2,6-difluorophenyl)-6H-pyrazolo[1,5-a][1,3,5]benzotriazepine (Example 1), following the synthetic procedure as described in the synthesis of 4-[3-chloro-5-(2,6-difluorophenyl)-6H-pyrazolo[1,5-a][1,3,5]benzotriazepin-9-yl]morpholine (Example 2), except that (1R,4R)-2-oxa-5-azabicyclo[2.2.2]octane was used in the place of morpholine, and the reaction was conducted in dioxane at 70 °C for 16 h. The crude final product was purified by prep-HPLC (Biotage®, Column: 2_Phenomenex Gemini C18 75×40 mm×3 m; mobile phase: [water (NH₄HCO₃)-MeCN]; B %: 60%-90%, 9.5 min) to provide the title compound as a yellow solid (13 mg, 23.98%). ¹HNMR (400 MHz, DMSO-d₆) δ ppm 1.66 (dt, J=4.3, 11.8 Hz, 1H), 1.93-1.81 (m, 2H), 1.98 (br d, J=10.8 Hz, 1H), 3.27 (br d, J=10.6 Hz, 2H), 3.60 (br d, J=10.4 Hz, 1H), 3.82 (br s, 1H), 3.98-3.89 (m, 3H), 6.40 (br d, J=8.9 Hz, 1H), 6.65 (br d, J=8.6 Hz, 1H), 6.90 (d, J=2.6 Hz, 1H), 7.22 (br t, J=8.0 Hz, 2H), 7.63-7.52 (m, 2H), 9.34 (br s, 1H); ¹⁹F NMR (377 MHz, DMSO-d₆) δ ppm −113.65; LCMS (ESI) m/z 442.1 [M+H]⁺.

### Example 16

### Synthesis of 3-chloro-5-(2,6-difluorophenyl)-9-(pyrrolidin-1-yl)-6H-benzo[f]pyrazolo[1,5-a][1,3,5]triazepine

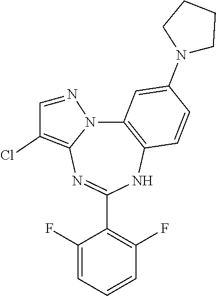 [SMILES: Fc1cccc(F)c1C1=Nc2c(Cl)cnn2-c2cc(N3CCCC3)ccc2N1]

The title compound was made from 9-bromo-3-chloro-5-(2,6-difluorophenyl)-6H-pyrazolo[1,5-a][1,3,5]benzotriazepine (Example 1), following the synthetic procedure as described in the synthesis of 4-[3-chloro-5-(2,6-difluorophenyl)-6H-pyrazolo[1,5-a][1,3,5]benzotriazepin-9-yl]morpholine (Example 2), except that pyrrolidine was used in the place of morpholine, and the reaction was conducted in dioxane at 70 °C for 16 h. The crude final product was purified by prep-HPLC (column: Waters Xbridge 15×25 mm×m; mobile phase: [water (FA)-MeCN]; B %: 56%-86%, 9.5 min) to provide the title compound as a yellow solid (5.31 mg, 10.8%). ¹HNMR (400 MHz, CD₃OD) δ ppm 2.02-2.06 (m, 4H), 3.29 (br s, 4H), 6.33 (br d, J=7.78 Hz, 1H), 6.63 (br d, J=8.78 Hz, 1H), 6.90 (d, J=2.00 Hz, 1H) 7.10 (t, J=8.16 Hz, 2H), 7.47-7.52 (m, 1H), 7.52-7.59 (m, 1H); LCMS (ESI) m/z 400.1 [M+H]⁺.

### Example 17

### Synthesis of 6-[3-chloro-5-(2,6-difluorophenyl)-6H-pyrazolo[1,5-a][1,3,5]benzotriazepin-9-yl]-2-oxa-6-azaspiro[3.3]heptane

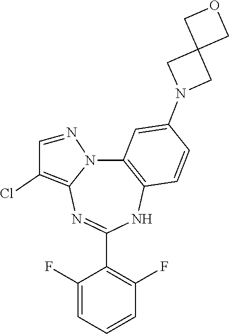 [SMILES: Fc1cccc(F)c1C1=Nc2c(Cl)cnn2-c2cc(N3CC4(COC4)C3)ccc2N1]

The title compound was made from 9-bromo-3-chloro-5-(2,6-difluorophenyl)-6H-pyrazolo[1,5-a][1,3,5]benzotriazepine (Example 1), following the synthetic procedure as described in the synthesis of 4-[3-chloro-5-(2,6-difluorophenyl)-6H-pyrazolo[1,5-a][1,3,5]benzotriazepin-9-yl]morpholine (Example 2), except that 2-oxa-6-azaspiro [3.3]heptane was used in the place of morpholine, and the reaction was conducted in dioxane at 70 °C for 16 h. The crude final product was purified by preparative TLC (silica, petroleum ether/EtOAc=1/1, 254 nm) to provide the title compound as a red solid (11 mg, 21.0%). ¹HNMR (400 MHz, DMSO-d₆) δ ppm 3.93 (s, 4H), 4.69 (s, 4H), 6.17 (dd, J=2.6, 8.7 Hz, 1H), 6.73-6.60 (m, 2H), 7.23 (t, J=8.0 Hz, 2H), 7.62-7.53 (m, 1H), 7.65-7.65 (m, 1H), 9.32 (s, 1H); ¹⁹F NMR (377 MHz, DMSO-d₆) δ ppm −113.61; LCMS (ESI) m/z 428.1 [M+H]⁻.

### Example 18

### Synthesis of 4-[3-chloro-5-(2,6-difluorophenyl)-6H-pyrazolo[1,5-a][1,3,5]benzotriazepin-9-yl]-1,4-oxazepane

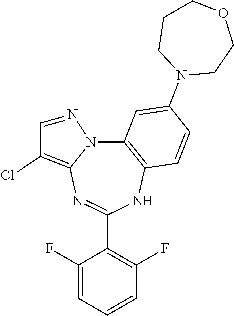 [SMILES: Fc1cccc(F)c1C1=Nc2c(Cl)cnn2-c2cc(N3CCCOCC3)ccc2N1]

The title compound was made from 9-bromo-3-chloro-5-(2,6-difluorophenyl)-6H-pyrazolo[1,5-a][1,3,5]benzotriazepine (Example 1), following the synthetic procedure as described in the synthesis of 4-[3-chloro-5-(2,6-difluorophenyl)-6H-pyrazolo[1,5-a][1,3,5]benzotriazepin-9-yl]morpholine (Example 2), except that 1, 4-oxazepane was used in the place of morpholine, and the reaction was conducted in dioxane at 70 °C for 16 h. The crude final product was purified by column chromatography (ISCO®; 4 g SepaFlash® Silica Flash Column, petroleum ether/EtOAc with EtOAc from 0~20%, 20 mL/min, 254 nm) to provide the title compound as a yellow solid (15 mg, 28.2%). ¹HNMR (400 MHz, DMSO-d₆) δ ppm 1.85 (quin, J=5.8 Hz, 2H), 3.52-3.47 (m, 5H), 3.58-3.54 (m, 3H), 3.70-3.65 (m, 2H), 6.47 (dd, J=2.9, 8.9 Hz, 1H), 6.64 (d, J=9.0 Hz, 1H), 6.99 (d, J=2.8 Hz, 1H), 7.22 (t, J=8.2 Hz, 2H), 7.58-7.53 (m, 1H), 7.59 (s, 1H), 9.30 (s, 1H); ¹⁹F NMR (376 MHz, DMSO-d₆) δ ppm −113.59; LCMS (ESI) m/z 430.1 [M+H]⁺.

### Example 19

### Synthesis of 3-chloro-5-(2,6-difluorophenyl)-9-(3-methoxypyrrolidin-1-yl)-6H-pyrazolo[1,5-a][1,3,5]benzotriazepine (racemic mixture)

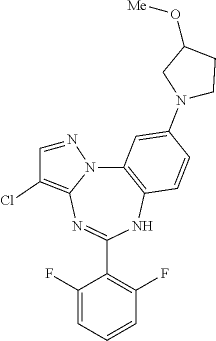 [SMILES: COC1CCN(c2ccc3c(c2)-n2ncc(Cl)c2N=C(c2c(F)cccc2F)N3)C1]

The title compound was made from 9-bromo-3-chloro-5-(2,6-difluorophenyl)-6H-pyrazolo[1,5-a][1,3,5]benzotriazepine (Example 1), following the synthetic procedure as described in the synthesis of 4-[3-chloro-5-(2,6-difluorophenyl)-6H-pyrazolo[1,5-a][1,3,5]benzotriazepin-9-yl]morpholine (Example 2), except that 3-methoxypyrrolidine (racemic mixture) was used in the place of morpholine, and the reaction was conducted in dioxane at 80 °C for 16 h. The crude final product was purified by prep-HPLC (column: 2_Phenomenex Gemini C18 75×40 mm×3 μm; mobile phase: [water (NH₄HCO₃)-MeCN]; B %: 50%-80%, 9.5 min) to provide the title compound as a yellow solid (24 mg, 28.6%). ¹HNMR (400 MHz, DMSO-d₆) δ ppm 2.06-2.00 (m, 2H), 3.23-3.15 (m, 2H), 3.21-3.16 (m, 1H), 3.25 (s, 4H), 4.05 (br d, J=2.5 Hz, 1H), 6.26 (dd, J=2.5, 8.8 Hz, 1H), 6.65 (d, J=8.8 Hz, 1H), 6.81 (d, J=2.8 Hz, 1H), 7.23 (t, J=8.0 Hz, 2H), 7.58-7.53 (m, 1H), 7.59 (s, 1H), 9.29 (s, 1H); ¹⁹F NMR (376 MHz, DMSO-d₆) δ ppm −113.60; LCMS (ESI) m/z 430.1 [M+H]⁺.

### Example 20

### Synthesis of 1-[3-chloro-5-(2,6-difluorophenyl)-6H-pyrazolo[1,5-a][1,3,5]benzotriazepin-9-yl]-6-oxa-1-azaspiro[3.3]heptane

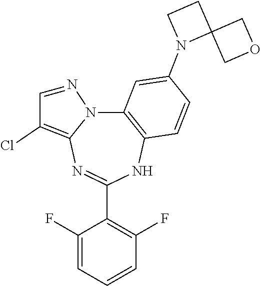 [SMILES: Fc1cccc(F)c1C1=Nc2c(Cl)cnn2-c2cc(N3CCC34COC4)ccc2N1]

The title compound was made from 9-bromo-3-chloro-5-(2,6-difluorophenyl)-6H-pyrazolo[1,5-a][1,3,5]benzotriazepine (Example 1), following the synthetic procedure as described in the synthesis of 4-[3-chloro-5-(2,6-difluorophenyl)-6H-pyrazolo[1,5-a][1,3,5]benzotriazepin-9-yl]morpholine (Example 2), except that 6-oxa-1-azaspiro [3.3]heptane was used in the place of morpholine, and the reaction was conducted in dioxane at 85 °C for 16 h. The crude final product was purified by column chromatography (ISCO®; 4 g SepaFlash® Silica Flash Column, petroleum ether/EtOAc with EtOAc from 0~24.5%, 20 mL/min, 254 nm) to provide the title compound as a yellow solid (20 mg, 23.8%). ¹HNMR (400 MHz, DMSO-d₆) δ ppm 2.48-2.43 (m, 2H), 3.58 (brt, J=6.9 Hz, 4H). 4.66 (d, J=7.9 Hz, 2H), 5.00 (d. J=7.9 Hz, 2H), 6.46 (dd, J=2.4, 8.6 Hz, 1H), 6.73 (d, J=8.6 Hz, 1H), 6.98 (d, J=2.4 Hz, 1H), 7.24 (br t, J=8.1 Hz, 2H), 7.61-7.54 (m, 1H), 7.62 (s, 1H), 9.38 (s, 1H); ¹⁹F NMR (377 MHz, DMSO-d₆) δ ppm −113.59; LCMS (ESI) m/z 428.1 [M+H]⁺.

### Example 21

### Synthesis of 3-chloro-5-(2,6-difluorophenyl)-9-(3-methoxyazetidin-1-yl)-6H-pyrazolo[1,5-a][1,3,5]benzotriazepine

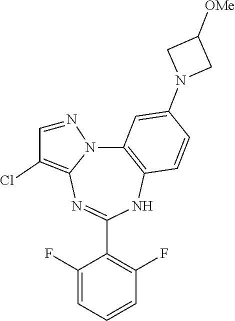 [SMILES: COC1CN(c2ccc3c(c2)-n2ncc(Cl)c2N=C(c2c(F)cccc2F)N3)C1]

The title compound was made from 9-bromo-3-chloro-5-(2,6-difluorophenyl)-6H-pyrazolo[1,5-a][1,3,5]benzotriazepine (Example 1), following the synthetic procedure as described in the synthesis of 4-[3-chloro-5-(2,6-difluorophenyl)-6H-pyrazolo[1,5-a][1,3,5]benzotriazepin-9-yl]morpholine (Example 2), except that 3-methoxyazetidine was used in the place of morpholine, and the reaction was conducted in dioxane at 75 °C for 16 h. The crude final product was purified by flash silica gel chromatography (ISCOR; 4 g SepaFlash® Silica Flash Column, petroleum ether/EtOAc with EtOAc from 0~20%, 20 mL/min, 254 nm) to provide the title compound as a yellow solid (37.3 mg, 36.5%). ¹HNMR (400 MHz, DMSO-d₆) (ppm 3.23 (s, 3H), 3.55 (dd, J=8.03, 4.27 Hz, 2H), 4.00 (t, J=7.15 Hz, 2H), 4.24-4.33 (m, 1H), 6.17 (dd, J=8.53, 2.76 Hz, 1H), 6.65 (d, J=8.53 Hz, 1H), 6.69 (d, J=2.76 Hz, 1H), 7.23 (t, J=8.16 Hz, 2H), 7.53-7.63 (m, 2H), 9.34 (s, 1H); ¹⁹F NMR (377 MHz, DMSO-d₆); LCMS (ESI) m/z 416.1 [M+H]⁺.

### Example 22

### Synthesis of 3-chloro-5-(2,6-difluorophenyl)-9-(3-fluoroazetidin-1-yl)-6H-pyrazolo[1,5-a][1,3,5]benzotriazepine

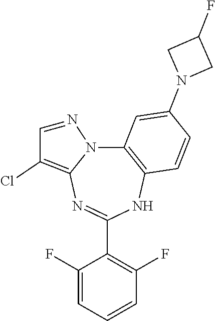 [SMILES: Fc1cccc(F)c1C1=Nc2c(Cl)cnn2-c2cc(N3CC(F)C3)ccc2N1]

The title compound was made from 9-bromo-3-chloro-5-(2,6-difluorophenyl)-6H-pyrazolo[1,5-a][1,3,5]benzotriazepine (Example 1), following the synthetic procedure as described in the synthesis of 4-[3-chloro-5-(2,6-difluorophenyl)-6H-pyrazolo[1,5-a][1,3,5]benzotriazepin-9-yl]morpholine (Example 2), except that 3-fluoroazetidine was used in the place of morpholine, and the reaction was conducted in dioxane at 75 °C for 16 h. The crude final product was purified by prep-HPLC (column: 2_Phenomenex Gemini C18 75×40 mm×3 μm; mobile phase: [water (NH₄HCO₃)-MeCN]; B %: 52%-82%, 9.5 min) to provide the title compound as a yellow solid (11 mg, 22.3%). ¹HNMR (400 MHz, CD₃OD) δ ppm 3.86 (dd, J=2.4, 9.2 Hz, 1H), 3.92 (dd, J=3.1, 9.7 Hz, 1H), 4.15-4.11 (m, 2H), 5.37-5.30 (m, 0.5 H), 5.36-5.30 (m, 0.5 H), 6.26 (dd, J=2.6, 8.7 Hz, 1H), 6.63 (d, J=8.5 Hz, 1H), 6.82 (d, J=2.5 Hz, 1H), 7.13-7.05 (m, 2H), 7.50-7.46 (m, 1H), 7.58-7.51 (m, 1H); ¹⁹F NMR (376 MHz, CD₃OD) δ ppm −114.84; LCMS (ESI) m/z 403.9 [M+H]⁺.

### Example 23

### Synthesis of 3-chloro-5-(2,6-difluorophenyl)-9-(6-fluoro-2-azaspiro[3.3]heptan-2-yl)-6H-pyrazolo[1,5-a][1,3,5]benzotriazepine

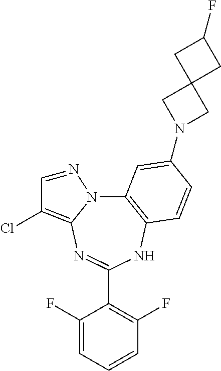 [SMILES: Fc1cccc(F)c1C1=Nc2c(Cl)cnn2-c2cc(N3CC4(CC(F)C4)C3)ccc2N1]

The title compound was made from 9-bromo-3-chloro-5-(2,6-difluorophenyl)-6H-pyrazolo[1,5-a][1,3,5]benzotriazepine (Example 1), following the synthetic procedure as described in the synthesis of 4-[3-chloro-5-(2,6-difluorophenyl)-6H-pyrazolo[1,5-a][1,3,5]benzotriazepin-9-yl]morpholine (Example 2), except that 6-fluoro-2-azaspiro [3.3]heptane was used in the place of morpholine, and the reaction was conducted in dioxane at 75 °C for 16 h. The crude final product was purified by preparative TLC (silica, petroleum ether/EtOAc=2/1, 254 nm) to provide the title compound as a yellow solid (12 mg, 20.8%). ¹HNMR (400 MHz, CD₃OD) δ ppm 2.46-2.33 (m, 2H), 2.69-2.59 (m, 2H), 3.82 (d, J=5.0 Hz, 4H), 5.07-4.99 (m, 1H), 6.21 (dd, J=2.6, 8.7 Hz, 1H), 6.60 (d, J=8.5 Hz, 1H), 6.78 (d, J=2.8 Hz, 1H), 7.08 (t, J=8.2 Hz, 2H), 7.50-7.44 (m, 1H), 7.57-7.50 (m, 1H); ¹⁹F NMR (377 MHz, CD₃OD) 5 ppm −114.83; LCMS (ESI) m/z 444.1 [M+H]⁺.

### Example 24

### Synthesis of 8-[3-chloro-5-(2, 6-difluorophenyl)-6H-pyrazolo [1,5-a][1, 3, 5]benzotriazepin-9-yl]-3-oxa-8-azabicyclo [3.2.1]octane

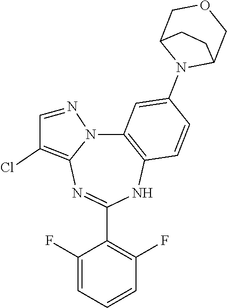 [SMILES: Fc1cccc(F)c1C1=Nc2c(Cl)cnn2-c2cc(N3C4CCC3COC4)ccc2N1]

The title compound was made from 9-bromo-3-chloro-5-(2,6-difluorophenyl)-6H-pyrazolo[1,5-a][1,3,5]benzotriazepine (Example 1), following the synthetic procedure as described in the synthesis of 4-[3-chloro-5-(2,6-difluorophenyl)-6H-pyrazolo[1,5-a][1,3,5]benzotriazepin-9-yl]morpholine (Example 2), except that 3-oxa-8-azabicyclo [3.2.1]octane was used in the place of morpholine, and the reaction was conducted in dioxane at 90 °C for 16 h. The crude final product was purified by the reverse phase prep-HPLC (Biotage®, Column: Welch Xtimate C18 100×40 mm×3 m; mobile phase: [water (HCl)-MeCN]; B %: 45%-75%, 8.5 min) to provide the title compound as a yellow solid (10 mg, 18.54%). ¹HNMR (400 MHz, CD₃OD) δ ppm 2.09-2.02 (m, 2H), 2.19-2.11 (m, 2H), 3.63 (br d, J=11.5 Hz, 2H), 3.90 (br d, J=11.3 Hz, 2H), 4.26 (br s, 2H), 6.87 (br s, 1H), 6.94-6.88 (m, 1H), 7.23 (t, J=8.4 Hz, 2H), 7.44 (br s, 1H), 7.65 (s, 1H), 7.77-7.68 (m, 1H); ¹⁹F NMR (377 MHz, CD₃OD) δ ppm −113.07; LCMS (ESI) m/z 442.1 [M+H]⁺.

### Example 25

### Synthesis of 1-[3-chloro-5-(2,6-difluorophenyl)-6H-pyrazolo[1,5-a][1,3,5]benzotriazepin-9-yl]pyrrolidin-3-ol (racemic mixture)

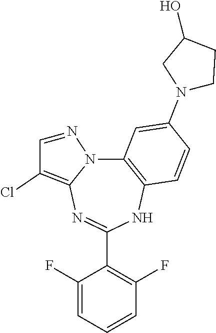 [SMILES: OC1CCN(c2ccc3c(c2)-n2ncc(Cl)c2N=C(c2c(F)cccc2F)N3)C1]

### Step 1

A mixture of 9-Bromo-3-chloro-5-(2,6-difluorophenyl)-6H-pyrazolo[1,5-a][1,3,5]benzotriazepine (300 mg, 0.732 mmol), 3-benzyloxypyrrolidine (157 mg, 0.732 mmol), t-BuONa (211 mg, 2.20 mmol) and Pd(t-Bu₃P)₂ (37.4 mg, 73.2 mol) were taken up into a microwave tube in dioxane (6 mL). The sealed tube was heated at 90 °C for 1 h by microwave. The solution was cooled to rt, treated with water (1 mL), filtered and concentrated under reduced pressure. The remaining residue was redissolved in DMSO (2 mL) and subjected to pre-HPLC purification (column: Boston Prime C18 150×30 mm×5 m; mobile phase: [water (ammonia hydroxide v/v)-MeCN]; B %: 54%-94%, 9 min) to give 9-(3-benzyloxypyrrolidin-1-yl)-3-chloro-5-(2,6-difluorophenyl)-6H-pyrazolo[1,5-a][1,3,5]benzotriazepine as a yellow solid (50 mg, 13.5%). ¹HNMR (400 MHz, DMSO-d₆) δ ppm 1.92-2.16 (m, 2H), 2.55-2.59 (m, 2H), 3.27 (br d, J=8.4 Hz, 2H), 3.40-3.76 (m, 1H), 4.26 (br d, J=16.1 Hz, 1H), 4.41-4.60 (m, 2H), 6.16-6.72 (m, 1H), 6.22-6.58 (m, 1H), 6.78-7.31 (m, 6H), 7.60-7.80 (m, 1H), 9.08-9.58 (m, 1H).

### Step 2

To a 25 °C mixture of 9-(3-benzyloxypyrrolidin-1-yl)-3-chloro-5-(2,6-difluorophenyl)-6H-pyrazolo[1,5-a][1,3,5]benzotriazepine (35 mg, 69.2 umol) in DCM (3 mL) under stirring was added BCl₃ (8.11 mg, 69.2 umol). The resulting mixture was stirred at rt under N₂ atmosphere for 16 h. The reaction solution was quenched with saturated aqueous NaHCO₃ solution till pH=8, and extracted with EtOAc (5 mL×2). The combined organic solutions were washed with brine (3 mL), dried over Na₂SO₄ and concentrated under reduced pressure. The resulting residue was purified by Pre-HPLC (column: Boston Green ODS 150×30 mm×5 μm; mobile phase: [water (HCl)-MeCN]; B %: 22%-62%, 9 min) to provide the title compound as a yellow solid (8 mg, 27.3%). ¹HNMR (400 MHz, CD₃OD) δ ppm 2.02-2.46 (m, 2H), 3.35-3.59 (m, 4H), 4.57-5.59 (m, 1H), 6.52-6.61 (m, 1H), 6.89-6.91 (m, 1H), 7.28-7.31 (m, 3H), 7.68 (s, 1H), 7.79-7.83 (m, 1H); LCMS (ESI) m/z 416.1 [M+H]⁺.

### Example 26

### Synthesis of 4-[3-chloro-5-(2,6-difluorophenyl)-6H-pyrazolo[1,5-a][1,3,5]benzotriazepin-9-yl]-2,6-dimethyl-morpholine

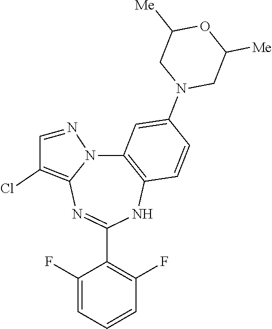 [SMILES: CC1CN(c2ccc3c(c2)-n2ncc(Cl)c2N=C(c2c(F)cccc2F)N3)CC(C)O1]

The title compound was made from 9-bromo-3-chloro-5-(2,6-difluorophenyl)-6H-pyrazolo[1,5-a][1,3,5]benzotriazepine (Example 1), following the synthetic procedure as described in the synthesis of 4-[3-chloro-5-(2,6-difluorophenyl)-6H-pyrazolo[1,5-a][1,3,5]benzotriazepin-9-yl]morpholine (Example 2), except that 2,6-di-methylmorpholine was used in the place of morpholine, and the reaction was conducted in dioxane at 60 °C for 16 h. The crude final product was purified by prep-HPLC (column: YMC-Actus Triart C18 150×30 mm×7 μm; mobile phase: [water (ammonia hydroxide v/v)-MeCN]; B %: 49%-89%, 9 min) to provide the title compound as a yellow solid (10.4 mg, 9.53%). ¹HNMR (500 MHz, CD₃OD) δ ppm 1.14-1.36 (m, 6H), 2.33 (t, J=11.14 Hz, 2H), 2.86 (m, 1H), 3.11 (m, 1H), 3.48 (br d, J=11.29 Hz, 1H), 3.66-3.85 (m, 2H), 4.02-4.22 (m, 1H), 6.59-6.75 (m, 2H), 7.09 (t, J=8.16 Hz, 2H), 7.20-7.31 (m, 1H), 7.45-7.55 (m, 2H); LCMS (ESI) m/z 444.2 [M+H]⁺.

### Example 27

### Synthesis of (1R,4R)-5-[3-chloro-5-(2,6-difluorophenyl)-6H-pyrazolo[1,5-a][1,3,5]benzotriazepin-9-yl]-2-oxa-5-azabicyclo[2.2.1]heptane

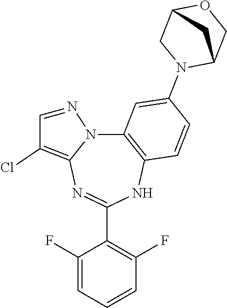 [SMILES: Fc1cccc(F)c1C1=Nc2c(Cl)cnn2-c2cc(N3C[C@H]4C[C@@H]3CO4)ccc2N1]

The title compound was made from 9-bromo-3-chloro-5-(2,6-difluorophenyl)-6H-pyrazolo[1,5-a][1,3,5]benzotriazepine (Example 1), following the synthetic procedure as described in the synthesis of 4-[3-chloro-5-(2,6-difluorophenyl)-6H-pyrazolo[1,5-a][1,3,5]benzotriazepin-9-yl]morpholine (Example 2), except that (1R,4R)-2-oxa-5-azabicyclo[2.2.1]heptane was used in the place of morpholine, and the reaction was conducted in dioxane at 75 °C for 24 h. The crude final product was purified by by prep-HPLC (column: Boston Green ODS 150×30 mm×5 μm; mobile phase: [water (HCl)-MeCN]; B %: 33%-73%, 9 min) to provide the title compound as a red solid (31.9 mg, 29.8%). ¹HNMR (500 MHz, DMSO-d₆) δ ppm 1.80-1.93 (m, 2H), 2.91 (d, J=9.46 Hz, 1H), 3.46 (d, J=8.54 Hz, 1H), 3.61-3.79 (m, 2H), 4.39-4.64 (m, 2H), 6.37 (dd, J=8.70, 2.59 Hz, 1H), 6.66 (d, J=8.54 Hz, 1H), 6.66 (d, J=8.54 Hz, 1H), 6.86 (d, J=2.75 Hz, 1H), 7.23 (t, J=8.09 Hz, 2H), 7.54-7.63 (m, 2H), 9.31 (s, 1H); LCMS (ESI) m/z 427.8 [M+H]⁺.

### Example 28

### Synthesis of (1S,4S)-5-[3-chloro-5-(2,6-difluorophenyl)-6H-pyrazolo[1,5-a][1,3,5]benzotriazepin-9-yl]-2-oxa-5-azabicyclo[2.2.1]heptane

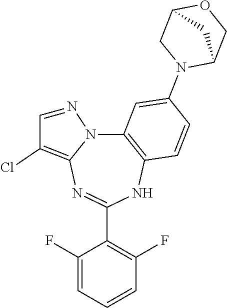 [SMILES: Fc1cccc(F)c1C1=Nc2c(Cl)cnn2-c2cc(N3C[C@@H]4C[C@H]3CO4)ccc2N1]

The title compound was made from 9-bromo-3-chloro-5-(2,6-difluorophenyl)-6H-pyrazolo[1,5-a][1,3,5]benzotriazepine (Example 1), following the synthetic procedure as described in the synthesis of 4-[3-chloro-5-(2,6-difluorophenyl)-6H-pyrazolo[1,5-a][1,3,5]benzotriazepin-9-yl]morpholine (Example 2), except that (1S,4S)-2-oxa-5-azabicyclo[2.2.1]heptane was used in the place of morpholine, and the reaction was conducted in dioxane at 75 °C for 24 h. The crude final product was purified by prep-HPLC (column: Boston Green ODS 150×30 mm×5 μm; mobile phase: [water (HCl)-MeCN];B %: 28%-68%, 9 min to provide the title compound as a red solid (14.0 mg 13.2%). ¹HNMR (500 MHz, DMSO-d₆) δ ppm 1.76-1.97 (m, 2H), 2.92 (br d, J=9.16 Hz, 1H), 3.60-3.78 (m. 2H), 4.44-4.58 (m, 2H), 6.37 (dd, J=8.54, 1.98 Hz, 1H), 6.66 (d, J=8.54 Hz, 1H), 6.86 (d, J=1.98 Hz, 1H), 7.23 (br t, J=8.09 Hz, 2H), 7.60 (s, 2H); LCMS (ESI) m/z: 427.8 [M+H]⁺.

### Example 29

### Synthesis of 3-[3-chloro-5-(2,6-difluorophenyl)-6H-pyrazolo [1,5-a][1,3,5]benzotriazepin-9-yl]-6-oxa-3-azabicyclo [3.1.1]heptane

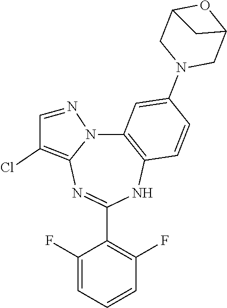 [SMILES: Fc1cccc(F)c1C1=Nc2c(Cl)cnn2-c2cc(N3CC4CC(C3)O4)ccc2N1]

The title compound was made from 9-bromo-3-chloro-5-(2,6-difluorophenyl)-6H-pyrazolo[1,5-a][1,3,5]benzotriazepine (Example 1), following the synthetic procedure as described in the synthesis of 4-[3-chloro-5-(2,6-difluorophenyl)-6H-pyrazolo[1,5-a][1,3,5]benzotriazepin-9-yl]morpholine (Example 2), except that 6-oxa-3-azabicyclo[3.1.1]heptane was used in the place of morpholine, and the reaction was conducted in dioxane at 70 °C for 24 h. The crude final product was purified by prep-HPLC (column: Welch Xtimate C18 150×25 mm×5 μm; mobile phase: [water (NH₄HCO₃)-MeCN]; B %: 40%-70%, 11 min) to provide the title compound as a yellow solid (14 mg. 13.4%). ¹HNMR (400 MHz, DMSO-d₆) δ ppm 1.89 (br s, 1H), 3.11 (br s, 6H). 4.68 (br s, 2H), 6.47 (br s, 1H), 6.71 (br s, 1H), 7.04 (br s, 1H), 7.23 (br s, 2H), 7.60 (br s, 2H), 9.32 (br s, 1H); LCMS (ESI) m/z: 428.1 [M+H]⁺.

### Example 30

### Synthesis of (2R,6S)-4-(3-chloro-5-(2,6-difluorophenyl)-6H-benzo[f]pyrazolo[1,5-a][1,3,5]triazepin-9-yl)-2,6-dimethylmorpholine

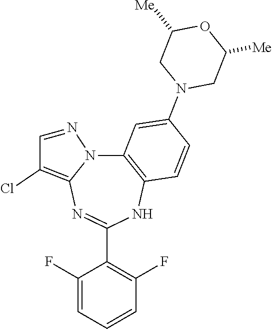 [SMILES: C[C@@H]1CN(c2ccc3c(c2)-n2ncc(Cl)c2N=C(c2c(F)cccc2F)N3)C[C@H](C)O1]

The title compound was made from 9-bromo-3-chloro-5-(2,6-difluorophenyl)-6H-pyrazolo[1,5-a][1,3,5]benzotriazepine (Example 1), following the synthetic procedure as described in the synthesis of 4-[3-chloro-5-(2,6-difluorophenyl)-6H-pyrazolo[1,5-a][1,3,5]benzotriazepin-9-yl]morpholine (Example 2), except that (2R,6S)-2,6-dimethylmorpholine was used in the place of morpholine, and the reaction was conducted in dioxane at 80 °C for 16 h. The crude final product was purified by prep-HPLC (YMC-Triart Prep C18 150×40 mm×7 μm; mobile phase: [water (NH₃H₂O+NH₄HCO₃)-MeCN]; B %: 50%-90%, 9 min) to provide the title compound as a yellow solid (1.9 mg, 1.75%). ¹HNMR (400 MHz, DMSO-d₆) δ ppm 0.79-0.80 (d, J=4.8 Hz, 6H). 3.14-3.15 (m, 2H), 3.35-3.53 (m, 2H), 3.70-3.72 (dd, J=2.4 Hz 2H), 6.75-6.79 (m, 2H), 7.24-7.27 (m, 1H), 7.27-7.31 (d, 1H), 7.58-7.60 (m, 1H), 9.64 (s, 1H). LCMS (ESI) m/z: 444.2 [M+H]⁺.

### Example 31

### Synthesis of (2S,6S)-4-(3-chloro-5-(2,6-difluorophenyl)-6H-benzo[f]pyrazolo[1,5-a][1,3,5]triazepin-9-yl)-2,6-dimethylmorpholine

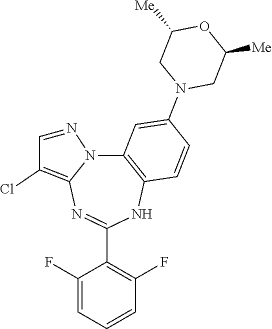 [SMILES: C[C@H]1CN(c2ccc3c(c2)-n2ncc(Cl)c2N=C(c2c(F)cccc2F)N3)C[C@H](C)O1]

The title compound was made from 9-bromo-3-chloro-5-(2,6-difluorophenyl)-6H-pyrazolo[1,5-a][1,3,5]benzotriazepine (Example 1), following the synthetic procedure as described in the synthesis of 4-[3-chloro-5-(2,6-difluorophenyl)-6H-pyrazolo[1,5-a][1,3,5]benzotriazepin-9-yl]morpholine (Example 2), except that (2S, 6S)-2,6-di-methylmorpholine was used in the place of morpholine, and the reaction was conducted in dioxane at 65 °C for 16 h. The crude final product was purified by prep-HPLC (YMC-Triart Prep C18 150×40 mm×7 μm; mobile phase: [water (NH₃H₂O+NH₄HCO₃)-MeCN]; B %: 50%-90%, 9 min) to provide the title compound as a yellow solid (33 mg, 30.45%). ¹HNMR (400 MHz, DMSO-d₆) δ ppm 1.19 (d, J=6.4 Hz, 6H), 2.74-2.76 (m, 2H), 3.08-3.11 (m, 2H), 4.02-4.03 (m, q H), 6.68-6.70 (m, 2H), 7.15-7.25 (m, 3H), 7.56-7.62 (m, 2H), 9.39 (s, 1H); LCMS (ESI) m/z: 443.8 [M+H]⁺.

### Example 32

### Synthesis of (2R,6R)-4-(3-chloro-5-(2,6-difluorophenyl)-6H-benzo[f]pyrazolo[1,5-a][1,3,5]triazepin-9-yl)-2,6-dimethylmorpholine

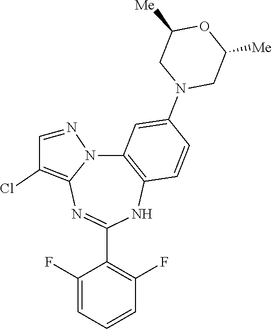 [SMILES: C[C@@H]1CN(c2ccc3c(c2)-n2ncc(Cl)c2N=C(c2c(F)cccc2F)N3)C[C@@H](C)O1]

The title compound was made from 9-bromo-3-chloro-5-(2,6-difluorophenyl)-6H-pyrazolo[1,5-a][1,3,5]benzotriazepine (Example 1), following the synthetic procedure as described in the synthesis of 4-[3-chloro-5-(2,6-difluorophenyl)-6H-pyrazolo[1,5-a][1,3,5]benzotriazepin-9-yl]morpholine (Example 2), except that (2R,6R)-2,6-di-methylmorpholine was used in the place of morpholine, and the reaction was conducted in dioxane at 65 °C for 16 h. The crude final product was purified by prep-HPLC (YMC-Triart Prep C18 150×40 mm×7 m; mobile phase: [water (NH₃H₂O+NH₄HCO₃)-MeCN]; B %: 50%-90%, 9 min) to provide the title compound as a yellow solid (16 mg, 14.77%). ¹HNMR (400 MHz, DMSO-d₆) (ppm 1.19 (d, J=6.0 Hz, 6H), 2.74-2.78 (m, 2H), 3.08-3.11 (m, 2H), 4.02-4.03 (m, 2H), 6.67-6.68 (m, 2H), 7.15-7.23 (m, 3H), 7.54-7.62 (m, 2H), 9.39 (s, 1H). LCMS (ESI) m/z: 443.8 [M+H]⁺.

### Example 33

### Synthesis of 5-(3-chloro-5-(2,6-difluorophenyl)-6H-benzo[f]pyrazolo[1,5-a][1,3,5]triazepin-9-yl)-2-oxa-5-azabicyclo[2.2.1]heptane (racemic mixture)

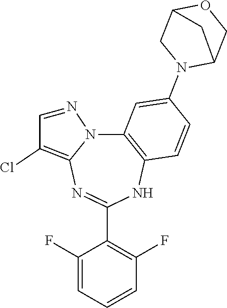 [SMILES: Fc1cccc(F)c1C1=Nc2c(Cl)cnn2-c2cc(N3CC4CC3CO4)ccc2N1]

The title compound was made from 9-bromo-3-chloro-5-(2,6-difluorophenyl)-6H-pyrazolo[1,5-a][1,3,5]benzotriazepine (Example 1), following the synthetic procedure as described in the synthesis of 4-[3-chloro-5-(2,6-difluorophenyl)-6H-pyrazolo[1,5-a][1,3,5]benzotriazepin-9-yl]morpholine (Example 2), except that 2-oxa-5-azabicyclo[2.2.1]heptane (racemic mixture) was used in the place of morpholine, and the reaction was conducted in dioxane at 65 °C for 16 h. The crude final product was purified by prep-HPLC (YMC-Triart Prep C18 150×40 mm×7 m; mobile phase: [water (NH₃H₂O+NH₄HCO₃)-MeCN]; B %: 50%-90%, 9 min) to provide the title compound as a yellow solid (18.5 mg, 17.7%). ¹HNMR (500 MHz, DMSO-d₆) δ ppm 1.80-1.91 (m, 2H), 2.92 (d, J=9.46 Hz, 1H), 3.42-3.46 (m, 1H), 4.44-4.59 (m, 2H), 6.37 (dd, J=8.70, 2.59 Hz, 1H), 6.66 (d, J=8.54 Hz, 1H), 6.86 (d, J=2.75 Hz, 1H), 7.24 (t, J=8.09 Hz, 2H), 7.58-7.61 (m, 2H), 9.32 (s, 1H); LCMS (ESI) m/z: 428.1 [M+H]⁺.

### Example 34

### Synthesis of 4-[3-chloro-5-(2-chloro-6-fluoro-phenyl)-6H-pyrazolo[1,5-a][1,3,5]benzotriazepin-9-yl]morpholine

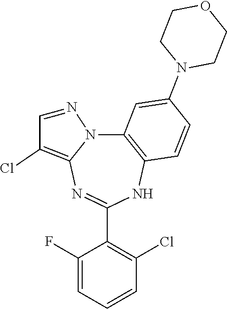 [SMILES: Fc1cccc(Cl)c1C1=Nc2c(Cl)cnn2-c2cc(N3CCOCC3)ccc2N1]

### Step 1

A solution of 2-(2-amino-5-bromo-phenyl)-4-chloro-pyrazol-3-amine (Intermediate 2) (5 g, 17.4 mmol) and 2-chloro-6-fluoro-benzaldehyde (2.76 g, 17.4 mmol) in t-BuOH (100 mL) was stirred at 50 °C for 15 min, followed by addition of DDQ (3.95 g, 17.4 mmol, 1 eq). The resulting mixture was allowed to stir at 50 °C for additional 30 min, cooled to rt, and concentrated under reduced pressure. The remaining residue was purified by flash silica gel chromatography (ISCO®; 80 g SepaFlash® Silica Flash Column, Eluent of 0~35% EtOAc/petroleum ether, flow rate: 50 mL/min) to afford 9-bromo-3-chloro-5-(2-chloro-6-fluoro-phenyl)-6H-pyrazolo[1,5-a][1,3,5]benzotriazepine (4 g, 54.0%) as a black brown solid. ¹HNMR (400 MHz, CDCl₃) δ ppm 4.13 (d, J=7.2 Hz, 1H), 6.11 (br s, 1H), 6.42 (d, J=8.3 Hz, 1H), 7.07-7.19 (m, 2H), 7.33-7.44 (m, 1H), 7.49 (s, 1H), 7.95 (d, J=2.2 Hz, 1H).

### Step 2

A mixture of 9-bromo-3-chloro-5-(2-chloro-6-fluoro-phenyl)-6H-pyrazolo[1,5-a][1,3,5]benzotriazepine (200 mg, 0.469 mmol), morpholine (45.0 mg, 0.516 mmol), tBuXPhos-Pd-G3 (37.3 mg, 46.9 mmol) and NaO^{t}Bu (135 mg, 1.41 mmol) in dioxane (2 mL) under N₂ atmosphere was stirred at 110 °C for 16 h. The mixture was cooled to rt, and concentrated under reduced pressure. The remaining residue was purified by preparative HPLC (column: Boston Green ODS 150×30 mm×5 m; mobile phase: [water (HCl)-MeCN]; B %: 35%-75%, 9 min, 254 nm) to afford the title compound as a yellow solid (14.8 mg, 6.5%). ¹HNMR (500 MHz, DMSO-d₆) δ ppm 3.02-3.07 (m, 4H), 3.68-3.77 (m, 4H), 6.70 (s, 2H), 7.21 (s, 1H), 7.37 (t, J=8.4 Hz, 1H), 7.45 (d, J=8.1 Hz, 1H), 7.55 (td, J=8.3, 6.2 Hz, 1H), 7.62 (s, 1H); LCMS (ESI) m/z 432.1 [M+H]⁺.

### Example 35

### Synthesis of 4-[3-chloro-5-(2-fluoro-6-morpholino-phenyl)-6H-pyrazolo[1,5-a][1,3,5]benzotriazepin-9-yl]morpholine

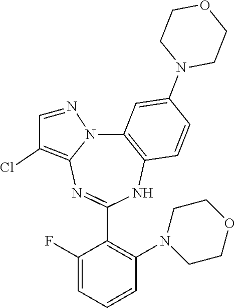 [SMILES: Fc1cccc(N2CCOCC2)c1C1=Nc2c(Cl)cnn2-c2cc(N3CCOCC3)ccc2N1]

To a solution of 9-bromo-3-chloro-5-(2,6-difluorophenyl)-6H-pyrazolo[1,5-a][1,3,5]benzotriazepine (70 mg, 0.17 mmol, 1 eq) and morpholine (14.9 mg, 0.17 mmol, 1 eq) in dioxane (2 mL) was added Cs₂CO₃ (167.0 mg, 0.51 mmol, 3 eq) and Xantphos-Pd-G3 (14.8 mg, 17.1 mol, 0.1 eq). The resulting mixture was stirred at 100 °C for 16 h, cooled to rt, filtered through a small pad of celite, and the celite was washed with dioxane (3 mL). The combined organic solutions were concentrated under reduced pressure and the remaining residue was purified by flash chromatography (ISCO®; 4 g SepaFlash® Silica Flash Column, petroleum ether/EtOAc with EtOAc from 0~100%, 20 mL/min, 254 mn) to afford the crude product. This crude product was further purified by preparative HPLC (column: Boston Prime C18 150×30 mm×5 μm; mobile phase: [water (FA)-MeCN]; B %: 27%-67%, 9 min) to provide the title product as a yellow solid (7.2 mg, 8.7%). ¹HNMR (400 MHz, DMSO-d₆) δ ppm 3.00-3.13 (m, 8H), 3.54-3.62 (m, 4H), 3.67-3.77 (m, 4H), 6.68-6.84 (m, 2H), 6.90-7.05 (m, 2H), 7.15 (d, J=2.4 Hz, 1H), 7.38-7.50 (m, 1H), 7.65 (s, 1H), 9.03 (s, 1H); LCMS (ESI) m/z 483.2 [M+H]⁺.

### Example 36

### Synthesis of 3-chloro-5-(2,6-difluorophenyl)-9-(1-methylpyrazol-4-yl)-6H-pyrazolo[1,5-a][1,3,5]benzotriazepine

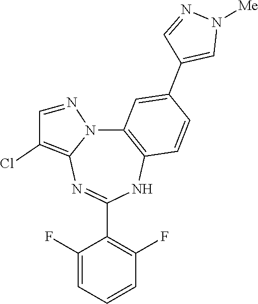 [SMILES: Cn1cc(-c2ccc3c(c2)-n2ncc(Cl)c2N=C(c2c(F)cccc2F)N3)cn1]

A mixture of 9-bromo-3-chloro-5-(2,6-difluorophenyl)-6H-pyrazolo[1,5-a][1,3,5]benzotriazepine (200 mg, 0.488 mmol, 1 eq), 1-methyl-4-(4,4,5,5-tetramethyl-1,3,2-dioxaborolan-2-yl)pyrazole (111 mg, 0.537 mmol, 1.1 eq), Pd₂(dba)₃ (44.7 mg, 48.8 mol, 0.1 eq), XPhos (46.5 mg, 97.6 mol, 0.2 eq), and K₃PO₄ (310 mg, 1.46 mmol, 3 eq) in dioxane (1 mL) and H₂O (0.5 mL) was degassed and purged with N₂ for 3 times. Then resulting mixture was stirred at 90 °C under N₂ atmosphere for 6 h, cooled to rt, diluted with H₂O (10 mL) and extracted with EtOAc (10 mL×3). The organic layers were combined, washed with brine (10 mL×2), dried with anhydrous Na₂SO₄, filtered and concentrated under reduced pressure. The remaining residue was purified by flash silica gel chromatography (ISCO®; 12 g SepaFlash® Silica Flash Column, Eluent of 0~11% petroleum ether/EtOAc @35 mL/min) to give the title compounds as a yellow solid (73.14 mg, 33.9%). ¹HNMR (400 MHz, DMSO-d₆) δ 3.84 (s, 3H), 6.81 (d, J=8.3 Hz, 1H), 7.30-7.23 (m, 3H), 7.71-7.67 (m, 1H), 7.65-7.55 (m, 1H), 7.80-7.75 (m, 2H), 8.11 (s, 1H), 9.62 (s, 1H); LCMS (ESI) m/z: 411.1 [M+H]⁺.

### Example 37

### Synthesis of 3-chloro-5-(2,6-difluorophenyl)-9-(1-methylpyrazol-4-yl)-6H-pyrazolo[1,5-a][1,3,5]benzotriazepine

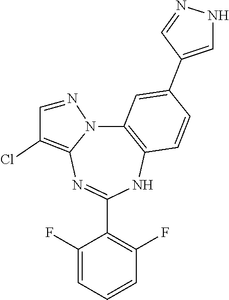 [SMILES: Fc1cccc(F)c1C1=Nc2c(Cl)cnn2-c2cc(-c3cn[nH]c3)ccc2N1]

The title compound was made from 9-bromo-3-chloro-5-(2,6-difluorophenyl)-6H-pyrazolo[1,5-a][1,3,5]benzotriazepine (Example 1), following the synthetic procedure as described in the synthesis of 3-chloro-5-(2,6-difluorophenyl)-9-(1-methylpyrazol-4-yl)-6H-pyrazolo[1,5-a][1,3,5]benzotriazepine (Example 36), except that tert-butyl 4-(4,4,5,5-tetramethyl-1,3,2-dioxaborolan-2-yl)pyrazole-1-carboxylate was used in the place of 1-methyl-4-(4,4,5,5-tetramethyl-1,3,2-dioxaborolan-2-yl)pyrazole, and the reaction was conducted in dioxane at 90 °C for 6 h. The crude final product was purified by flash silica gel chromatography (ISCO®; 12 g SepaFlash® Silica Flash Column, Eluent of 0~1% petroleum ether/EtOAc @35 mL/min) to provide the title compound as a yellow solid (17 mg, 8.7%). ¹HNMR (400 MHz, DMSO-d) a ppm 6.82 (d, J=8.3 Hz, 1H), 7.36-7.22 (m, 3H), 7.66-7.55 (m, 1H), 7.71 (s, 1H), 7.90-7.77 (m, 2H), 8.16 (s, 1H), 9.61 (s, 1H), 12.98 (br s, 1H); LCMS (ESI) m/z: 397.0 [M+H]⁺.

### Example 38

### Synthesis of Methyl 2-[3-chloro-5-(2,6-difluorophenyl)-6H-pyrazolo[1,5-a][1,3,5]benzotriazepin-9-yl]propan-2-ol

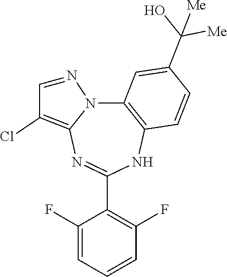 [SMILES: CC(C)(O)c1ccc2c(c1)-n1ncc(Cl)c1N=C(c1c(F)cccc1F)N2]

To a −78 °C solution of 9-bromo-3-chloro-5-(2,6-difluorophenyl)-6H-pyrazolo[1,5-a][1,3,5]benzotriazepine (100 mg, 0.244 mmol, 1 eq) in THF (4 mL) under N₂ atmosphere was added n-BuLi (2.5 M, 0.535 mmol, 2.2 eq). The resulting mixture was stirred at −78 °C for 30 min followed by addition of anhydrous acetone (70.9 mg, 1.22 mmol, 5 eq). After stirring at −78 °C for additional 3.0 h, the reaction mixture was allowed to warm to rt over 16 h and quenched with water (2 mL). After removal of volatile under reduced pressure, the remaining residue was purified by preparative HPLC (column: YMC-Actus Triart C18 150×30 mm×7 μm; mobile phase: [water (ammonia hydroxide v/v)-MeCN]; B %: 36%-76%, 9 min) to afford the title compound as yellow solid (0.95 mg, 1.0% yield). ¹HNMR (400 MHz, DMSO-d₆) δ ppm 1.38 (s, 6H), 5.08 (s, 1H), 6.76 (d, J=8.3 Hz, 1H), 7.12 (dd, J=8.3, 2.1 Hz, 1H), 7.26 (t, J=8.1 Hz, 2H), 7.56-7.69 (m, 2H), 7.78 (d, J=2.1 Hz, 1H), 9.54 (s, 1H); LCMS (ESI) m/z: 389.0 [M+H]⁺.

### Example 39

### Synthesis of Methyl 3-chloro-5-(2,6-difluorophenyl)-6H-pyrazolo[1,5-a][1,3,5]benzotriazepine-9-carboxylate

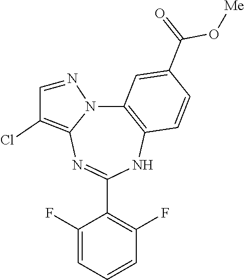 [SMILES: COC(=O)c1ccc2c(c1)-n1ncc(Cl)c1N=C(c1c(F)cccc1F)N2]

A solution of 9-bromo-3-chloro-5-(2,6-difluorophenyl)-6H-pyrazolo[1,5-a][1,3,5]benzotriazepine (1.0 g, 2.44 mmol, 1.0 eq), Et₃N (741 mg, 7.32 mmol, 3 eq) and Pd(dppf)Cl₂ (179 mg, 0.244 mmol, 0.1 eq) in DMF (50 mL) and MeOH (5 mL) was degassed and purged with CO for 3 times. The resulting mixture was stirred under CO (50 psi) at 80 °C for 16 h and cooled to rt. After removal of volatiles under reduced pressure, the remaining residue was purified by flash chromatography (ISCO®; 12 g SepaFlash® Silica Flash Column, petroleum ether/EtOAc with EtOAc from 0~35%, 20 mL/min, 254 nm) to afford the title compound as yellow solid (750 mg, 79.0%). ¹HNMR (400 MHz, DMSO-d₆) δ ppm 3.83 (s, 3H), 6.93 (d, J=8.3 Hz, 1H), 7.24-7.33 (m, 2H), 7.59-7.66 (m, 2H), 7.74 (s, 1H), 8.23 (d, J=2.1 Hz, 1H), 9.98 (s, 1H); LCMS (ESI) m/z 389.1 [M+H]⁺.

### Example 40

### Synthesis of Methyl 3-chloro-5-(2,6-difluorophenyl)-6H-pyrazolo[1,5-a][1,3,5]benzotriazepine-9-carboxylic acid

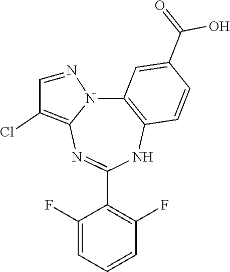 [SMILES: O=C(O)c1ccc2c(c1)-n1ncc(Cl)c1N=C(c1c(F)cccc1F)N2]

A mixture of methyl 3-chloro-5-(2,6-difluorophenyl)-6H-pyrazolo[1,5-a][1,3,5]benzotriazepine-9-carboxylate (Example 39, 750 mg, 1.93 mmol, 1 eq) and LiOH—H₂O (242.8 mg, 5.79 mmol, 3 eq) in H₂O/THF (1:1, 20 mL) was stirred at rt for 16 h. The organic solvent was removed under reduced pressure. The remaining mixture was adjusted to pH=3 with 1.0 N HCl, and extracted with EtOAc (20 mL×2). The combined organic layers were washed with water, brine, and concentrated to afford the title compound as yellow solid (650 mg, 89.9%). ¹HNMR (400 MHz, DMSO-d₆) δ ppm 6.91 (d, J=8.4 Hz, 1H), 7.27 (t, J=8.1 Hz, 2H), 7.55-7.67 (m, 2H), 7.73 (s, 1H), 8.21 (d, J=1.9 Hz, 1H), 9.93 (s, 1H); LCMS (ESI) m/z 375.0 [M+H]⁺.

### Example 41

### Synthesis of 3-chloro-5-(2,6-difluorophenyl)-6H-pyrazolo[1,5-a][1,3,5]benzotriazepine-9-carboxamide

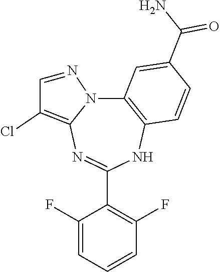 [SMILES: NC(=O)c1ccc2c(c1)-n1ncc(Cl)c1N=C(c1c(F)cccc1F)N2]

To a solution of 3-chloro-5-(2,6-difluorophenyl)-6H-pyrazolo[1,5-a][1,3,5]benzotriazepine-9-carboxylic acid (Example 40) (50 mg, 0.133 mmol, 1 eq) in DMF (2 mL) was added HATU (60.9 mg, 0.16 mmol, 1.2 eq), hunig base (51.7 mg, 0.40 mmol, 3 eq) and anhydrous NH₄Cl (21.4 mg, 0.40 mmol, 3 eq). The mixture was stirred at rt for 16 h. treated with water (1 mL), filtered through a small pad of celite. The filtrate was subjected to purification by preparative HPLC (column: Boston Prime C18 150×30 mm×5 μm; mobile phase: [water (ammonia hydroxide v/v)-MeCN]; B %: 19%-59%, 9 min) to afford the title product as yellow solid (26.7 mg, 53.5%). ¹HNMR (400 MHz, DMSO-d₆) δ ppm 6.85 (d, J=8.4 Hz, 1H), 7.22-7.36 (m, 3H), 7.55 (dd, J=8.3, 1.9 Hz, 1H), 7.58-7.66 (m, 1H), 7.73 (s, 1H), 7.94 (br s, 1H), 8.15 (d, J=1.9 Hz, 1H), 9.81 (br s, 1H); LCMS (ESI) m/z 374.0 [M+H]⁻.

### Example 42

### Synthesis of 3-chloro-5-(2,6-difluorophenyl)-N-(2-fluoroethyl)-6H-pyrazolo[1,5-a][1,3,5]benzotriazepine-9-carboxamide

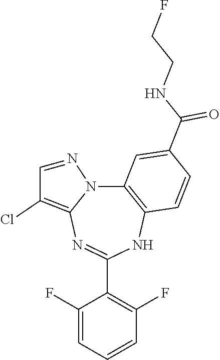 [SMILES: O=C(NCCF)c1ccc2c(c1)-n1ncc(Cl)c1N=C(c1c(F)cccc1F)N2]

The title compound was made from 3-chloro-5-(2,6-difluorophenyl)-6H-pyrazolo[1,5-a][1,3,5]benzotriazepine-9-carboxylic acid (Example 40), following the synthetic procedure as described in the synthesis of 3-chloro-5-(2,6-difluorophenyl)-6H-pyrazolo[1,5-a][1,3,5]benzotriazepine-9-carboxamide (Example 41), except that 2-fluoroethanamine was used in the place of NH₄Cl, and the reaction was conducted in DMF at rt for 12 h. The crude final product was purified by preparative HPLC (column: YMC Triart 30×150 mm×7 μm; mobile phase: [water (ammonia hydroxide v/v)-ACN]; B %: 24%-64%, 9 min) to provide the title compound as a yellow solid (13.3 mg, 43.9%). ¹HNMR (400 MHz, DMSO-d₆) δ ppm 3.48-3.61 (m, 2H), 4.39-4.65 (m, 1H), 4.42-4.66 (m, 1H), 6.88 (d, J=8.3 Hz, 1H), 7.27 (t, J=8.2 Hz, 2H), 7.52-7.67 (m, 2H), 7.74 (s, 1H), 8.16 (d, J=1.8 Hz, 1H), 8.70 (br t, J=5.4 Hz, 1H), 9.83 (br s, 1H); LCMS (ESI) m/z 420.1 [M+H]⁺.

### Example 43

### Synthesis of 3-chloro-5-(2,6-difluorophenyl)-N-(2-hydroxyethyl)-6H-pyrazolo[1,5-a][1,3,5]benzotriazepine-9-carboxamide

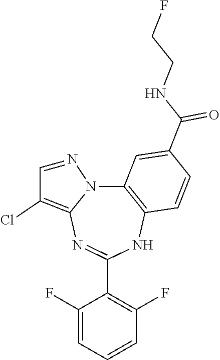 [SMILES: O=C(NCCF)c1ccc2c(c1)-n1ncc(Cl)c1N=C(c1c(F)cccc1F)N2]

The title compound was made from 3-chloro-5-(2,6-difluorophenyl)-6H-pyrazolo[1,5-a][1,3,5]benzotriazepine-9-carboxylic acid (Example 40), following the synthetic procedure as described in the synthesis of 3-chloro-5-(2,6-difluorophenyl)-6H-pyrazolo[1,5-a][1,3,5]benzotriazepine-9-carboxamide (Example 41), except that 2-aminoethanol was used in the place of NH₄Cl, and the reaction was conducted in DMF at 25 °C for 2 h. The crude final product was purified by prep-HPLC (YMC-Triart Prep C18 150×40 mm×7 μm; mobile phase: [water (NH₃H₂O+NH₄HCO₃)-MeCN]; B %: 50%-90%, 9 min) to provide the title compound as a yellow solid (17.5 mg, 52.3%). ¹HNMR (400 MHz, DMSO-d₆) b ppm 3.30 (d, J=5.9 Hz, 2H), 3.48-3.52 (m, 2H), 4.73 (t, J=5.6 Hz, 1H), 6.87 (d, J=8.3 Hz, 1H), 7.27 (t, J=8.1 Hz, 2H), 7.47-7.67 (m, 2H), 7.74 (s, 1H), 8.14 (d, J=2.1 Hz, 1H), 8.43 (t, J=5.4 Hz, 1H), 9.82 (br d, J=5.5 Hz, 1H); LCMS (ESI) m/z: 418.0 [M+H]⁺.

### Example 44

### Synthesis of 3-chloro-5-(2,6-difluorophenyl)-6H-pyrazolo[1,5-a][1,3,5]benzotriazepine-9-carbohydroxamic acid

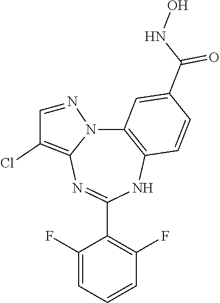 [SMILES: O=C(NO)c1ccc2c(c1)-n1ncc(Cl)c1N=C(c1c(F)cccc1F)N2]

The title compound was made from 3-chloro-5-(2,6-difluorophenyl)-6H-pyrazolo[1,5-a][1,3,5]benzotriazepine-9-carboxylic acid (Example 40), following the synthetic procedure as described in the synthesis of 3-chloro-5-(2,6-difluorophenyl)-6H-pyrazolo[1,5-a][1,3,5]benzotriazepine-9-carboxamide (Example 41), except that hydroxylamine was used in the place of NH₄Cl, and the reaction was conducted in DMF at 25 °C for 2 h. The crude final product was purified by prep-HPLC (column: Phenomenex C18 75×30 mm×3 μm; mobile phase: [water (FA)-MeCN]; B %: 12%-52%, 9 min) to provide the title compound as a yellow solid (8.5 mg, 27.2%). ¹HNMR (400 MHz, DMSO-d₆) δ ppm 6.86 (d, J=8.3 Hz, 1H), 7.27 (t, J=8.2 Hz, 2H), 7.42 (dd, J=8.4, 1.9 Hz, 1H), 7.57-7.68 (m, 1H), 7.74 (s, 1H), 8.07 (d, J=2.0 Hz, 1H), 9.04 (br s, 1H), 9.82 (br s, 1H); LCMS (ESI) m/z: 390.0 [M+H]⁺.

### Example 45

### Synthesis of 3-chloro-5-(2,6-difluorophenyl)-N-(2-methoxyethyl)-6H-pyrazolo[1,5-a][1,3,5]benzotriazepine-9-carboxamide

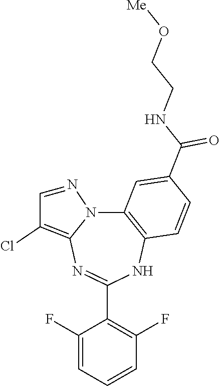 [SMILES: COCCNC(=O)c1ccc2c(c1)-n1ncc(Cl)c1N=C(c1c(F)cccc1F)N2]

To 3-chloro-5-(2,6-difluorophenyl)-6H-pyrazolo[1,5-a][1,3,5]benzotriazepine-9-carbonyl chloride (Intermediate 3) (50 mg, 0.133 mmol, 1 eq) in THF (2 mL) was added anhydrous Et₃N (40.5 mg, 0.40 mmol, 3 eq) and 2-methoxyethanamine (15.0 mg, 0.20 mmol, 1.5 eq). The mixture was stirred at 25 °C for 2 h and the volatiles was removed under reduced pressure. The remaining residue was purified by preparative HPLC (column: Boston Prime C18 150×30 mm×5 m; mobile phase: [water (ammonia hydroxide v/v)-MeCN]; B %: 29%-59%, 9 min) to afford the title compound as (10.3 mg, 17.8%) as yellow solid. ¹HNMR (400 MHz, DMSO-d₆) δ ppm 3.26 (s, 3H), 3.37-3.44 (m, 4H), 7.27 (t, J=8.2 Hz, 2H), 7.50-7.66 (m, 2H), 7.74 (s, 1H), 8.13 (d, J=1.8 Hz, 1H), 8.53 (br t, J=5.1 Hz, 1H), 9.82 (s, 1H); LCMS (ESI) m/z 432.1 [M+H]⁺.

### Example 46

### Synthesis of 3-chloro-5-(2,6-difluorophenyl)-N-methoxy-6H-pyrazolo[1,5-a][1,3,5]benzotriazepine-9-carboxamide

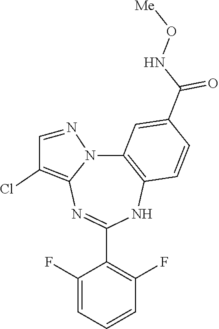 [SMILES: CONC(=O)c1ccc2c(c1)-n1ncc(Cl)c1N=C(c1c(F)cccc1F)N2]

The title compound was made from 3-chloro-5-(2,6-difluorophenyl)-6H-pyrazolo[1,5-a][1,3,5]benzotriazepine-9-carbonyl chloride (Intermediate 3), following the synthetic procedure as described in the synthesis of 3-chloro-5-(2,6-difluorophenyl)-N-(2-methoxyethyl)-6H-pyrazolo[1,5-a][1,3,5]benzotriazepine-9-carboxamide (Example 45), except that O-methylhydroxylamine was used in the place of methoxyethanamine, The crude mixture was purified by preparative HPLC (column: YMC-Actus Triart C18 150×30 mm×7 μm; mobile phase: [water (ammonia hydroxide v/v)-MeCN]; B %: 16%-56%, 9 min) to afford 3-chloro-5-(2,6-difluorophenyl)-N-methoxy-6H-pyrazolo[1,5-a][1,3,5]benzotriazepine-9-carboxamide (9.6 mg, 18.7%) as yellow solid. ¹HNMR (400 MHz, DMSO-d₆) b ppm 3.68 (s, 3H), 6.87 (d, J=8.3 Hz, 1H), 7.27 (t, J=8.1 Hz, 2H), 7.42 (br d, J=8.4 Hz, 1H), 7.56-7.67 (m, 1H), 7.74 (s, 1H), 8.05 (d, J=1.6 Hz, 1H), 9.85 (br s, 1H), 11.75 (br s, 1H); LCMS (ESI) m/z: 404.0 [M+H]⁺.

### Example 47

### Synthesis of 3-chloro-N-cyclopentyl-5-(2,6-difluorophenyl)-6H-pyrazolo[1,5-a][1,3,5]benzotriazepine-9-carboxamide

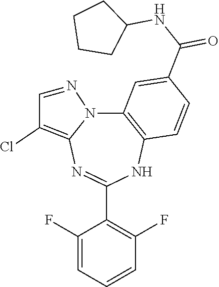 [SMILES: O=C(NC1CCCC1)c1ccc2c(c1)-n1ncc(Cl)c1N=C(c1c(F)cccc1F)N2]

The title compound was made from 3-chloro-5-(2,6-difluorophenyl)-6H-pyrazolo[1,5-a][1,3,5]benzotriazepine-9-carboxylic acid (Example 40), following the synthetic procedure as described in the synthesis of 3-chloro-5-(2,6-difluorophenyl)-6H-pyrazolo[1,5-a][1,3,5]benzotriazepine-9-carboxamide (Example 41), except that cyclopentanamine was used in the place of O-methylhydroxylamine, and the reaction was conducted with THF as solvent (25 °C, 2 h). The crude final product was purified by prep-HPLC (YMC-Triart Prep C18 150×40 mm×7 μm; mobile phase: [water (NH₃H₂O+NH₄HCO₃)-MeCN]; B %: 50%-90%, 9 min) to provide the title compound as a yellow solid (6.2 mg, 11.0%). ¹HNMR (400 MHz, DMSO-d₆) δ ppm 1.51 (m, 4H), 1.68 (m, 2H), 1.86 (m, 2H), 4.05-4.26 (m, 1H), 6.86 (d, J=8.2 Hz, 1H), 7.26 (t, J=8.1 Hz, 2H), 7.45-7.66 (m, 2H), 7.74 (s, 1H), 8.11 (d, J=1.5 Hz, 1H), 8.29 (br d, J=7.1 Hz, 1H), 9.80 (br s, 1H); LCMS (ESI) m/z: 442.0 [M+H]⁺.

### Example 48

### Synthesis of 3-chloro-5-(2,6-difluorophenyl)-N-methyl-6H-pyrazolo[1,5-a][1,3,5]benzotriazepine-9-carboxamide

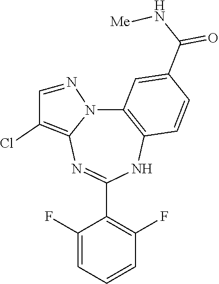 [SMILES: CNC(=O)c1ccc2c(c1)-n1ncc(Cl)c1N=C(c1c(F)cccc1F)N2]

The title compound was made from 3-chloro-5-(2,6-difluorophenyl)-6H-pyrazolo[1,5-a][1,3,5]benzotriazepine-9-carbonyl chloride (Intermediate 3), following the synthetic procedure as described in the synthesis of 3-chloro-5-(2,6-difluorophenyl)-N-(2-methoxyethyl)-6H-pyrazolo[1,5-a][1,3,5]benzotriazepine-9-carboxamide (Example 45), except that methylamine was used in the place of methoxyethanamine, and the reaction was conducted with THF as solvent (25 °C, 2 h). The crude final product was purified by prep-HPLC (YMC-Triart Prep C18 150×40 mm×7 m; mobile phase: [water (NH₃H₂O+NH₄HCO₃)-MeCN]; B %: 50%-90%, 9 min) to provide the title compound as a yellow solid (9.8 mg, 19.8%). ¹HNMR (400 MHz, DMSO-d₆) δ ppm 2.75 (d, J=4.3 Hz, 3H), 6.86 (d, J=8.3 Hz, 1H), 7.27 (br t, J=8.2 Hz, 2H), 7.45-7.67 (m, 2H), 7.73 (s, 1H), 8.12 (s, 1H), 8.42 (br d, J=4.0 Hz, 1H), 9.81 (s, 1H); LCMS (ESI) m/z: 387.9 [M+H]⁺.

### Example 49

### Synthesis of 3-chloro-5-(2,6-difluorophenyl)-N-isopropyl-6H-pyrazolo[1,5-a][1,3,5]benzotriazepine-9-carboxamide

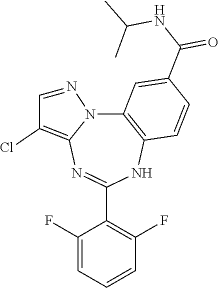 [SMILES: CC(C)NC(=O)c1ccc2c(c1)-n1ncc(Cl)c1N=C(c1c(F)cccc1F)N2]

The title compound was made from 3-chloro-5-(2,6-difluorophenyl)-6H-pyrazolo[1,5-a][1,3,5]benzotriazepine-9-carbonyl chloride (Intermediate 3), following the synthetic procedure as described in the synthesis of 3-chloro-5-(2,6-difluorophenyl)-N-(2-methoxyethyl)-6H-pyrazolo[1,5-a][1,3,5]benzotriazepine-9-carboxamide (Example 45), except that propan-2-amine was used in the place of methoxyethanamine. The crude final product was purified by prep-HPLC (YMC-Triart Prep C18 150×40 mm×7 m; mobile phase: [water (NH₃H₂O+NH₄HCO₃)-MeCN]; B %: 50%-90%, 9 min) to provide the title compound as a yellow solid (11.3 mg, 21.3%). ¹HNMR (400 MHz, DMSO-d₆) δ ppm 1.15 (br d, J=6.5 Hz, 6H), 3.95-4.17 (m, 1H), 6.87 (br d, J=8.3 Hz, 1H), 7.27 (brt, J=8.1 Hz, 2H), 7.49-7.80 (m, 3H), 8.07-8.31 (m, 2H), 9.81 (br s, 1H). LCMS (ESI) m/z: 416.0 [M+H]⁺.

### Example 50

### Synthesis of 3-chloro-N-(cyclopropylmethyl)-5-(2,6-difluorophenyl)-6H-pyrazolo[1,5-a][1,3,5]benzotriazepine-9-carboxamide

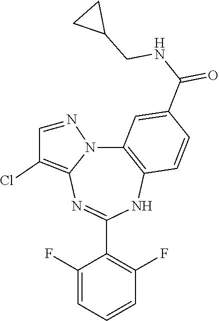 [SMILES: O=C(NCC1CC1)c1ccc2c(c1)-n1ncc(Cl)c1N=C(c1c(F)cccc1F)N2]

The title compound was made from 3-chloro-5-(2,6-difluorophenyl)-6H-pyrazolo[1,5-a][1,3,5]benzotriazepine-9-carbonyl chloride (Intermediate 3), following the synthetic procedure as described in the synthesis of 3-chloro-5-(2,6-difluorophenyl)-N-(2-methoxyethyl)-6H-pyrazolo[1,5-a][1,3,5]benzotriazepine-9-carboxamide (Example 45), except that cyclopropylmethanamine was used in the place of methoxyethanamine. The crude mixture product was purified by prep-HPLC (YMC-Triart Prep C18 150×40 mm×7 m; mobile phase: [water (NH₃H₂O+NH₄HCO₃)-MeCN]; B %: 50%-90%, 9 min) to provide the title compound as a yellow solid (11.1 mg, 20.4%). ¹HNMR (400 MHz, DMSO-d₆) δ ppm 0.22 (br d, J=4.2 Hz, 2H), 0.43 (br d, J=6.8 Hz, 2H), 1.02 (br s, 1H), 3.11 (br t, J=6.2 Hz, 2H), 6.88 (d, J=8.4 Hz, 1H), 7.27 (br t, J=8.2 Hz, 2H), 7.49-7.68 (m, 2H), 7.75 (s, 1H), 8.15 (s, 1H), 8.57 (brt, J=5.4 Hz, 1H), 9.82 (s, 1H); LCMS (ESI) m/z: 428.0 [M+H]⁺.

### Example 51

### Synthesis of 3-chloro-N-cyclobutyl-5-(2,6-difluorophenyl)-6H-pyrazolo[1,5-a][1,3,5]benzotriazepine-9-carboxamide

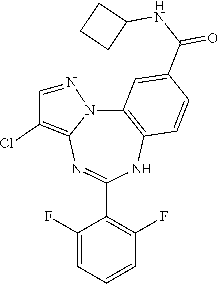 [SMILES: O=C(NC1CCC1)c1ccc2c(c1)-n1ncc(Cl)c1N=C(c1c(F)cccc1F)N2]

The title compound was made from 3-chloro-5-(2,6-difluorophenyl)-6H-pyrazolo[1,5-a][1,3,5]benzotriazepine-9-carbonyl chloride (Intermediate 3), following the synthetic procedure as described in the synthesis of 3-chloro-5-(2,6-difluorophenyl)-N-(2-methoxyethyl)-6H-pyrazolo[1,5-a][1,3,5]benzotriazepine-9-carboxamide (Example 45), except that cyclobutanamine was used in the place of methoxyethanamine. The crude product was purified by prep-HPLC (YMC-Triart Prep C18 150×40 mm×7 m; mobile phase: [water (NH₃H₂O+NH₄HCO₃)-MeCN]; B %: 50%-90%, 9 min) to provide the title compound as a yellow solid (11.6 mg, 21.3%). ¹HNMR (400 MHz, DMSO-d₆) δ ppm 1.61-1.71 (m, 2H), 2.00-2.12 (m, 2H), 2.14-2.26 (m, 2H), 4.39 (sxt, J=8.1 Hz, 1H), 6.87 (d, J=8.3 Hz, 1H), 7.27 (t, J=8.2 Hz, 2H), 7.51-7.67 (m. 2H), 7.75 (s, 1H), 8.13 (d, J=2.0 Hz, 1H), 8.62 (d, J=7.5 Hz, 1H), 9.82 (s, 1H); LCMS (ESI) m/z: 428.0 [M+H]⁺.

### Example 52

### Synthesis of N-(tert-butyl)-3-chloro-5-(2,6-difluorophenyl)-6H-benzo[f]pyrazolo[1,5-a][1,3,5]triazepine-9-carboxamide

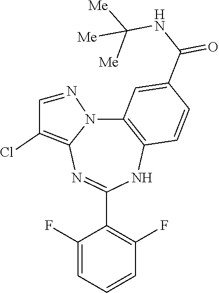 [SMILES: CC(C)(C)NC(=O)c1ccc2c(c1)-n1ncc(Cl)c1N=C(c1c(F)cccc1F)N2]

The title compound was made from 3-chloro-5-(2,6-difluorophenyl)-6H-pyrazolo[1,5-a][1,3,5]benzotriazepine-9-carbonyl chloride (Intermediate 3), following the synthetic procedure as described in the synthesis of 3-chloro-5-(2,6-difluorophenyl)-N-(2-methoxyethyl)-6H-pyrazolo[1,5-a][1,3,5]benzotriazepine-9-carboxamide (Example 45), except that 2-methylpropan-2-amine was used in the place of methoxyethanamine. The crude product was purified by prep-HPLC (YMC-Triart Prep C18 150×40 mm×7 m; mobile phase: [water (NH₃H₂O+NH₄HCO₃)-MeCN]; B %: 50%-90%, 9 min) to provide the title compound as a yellow solid (13.4 mg, 24.5%). ¹HNMR (400 MHz, DMSO-d₆) δ ppm 1.36 (s, 9H), 6.85 (d, J=8.3 Hz, 1H), 7.27 (t, J=8.1 Hz, 2H), 7.50 (dd, J=8.4, 2.0 Hz, 1H), 7.56-7.66 (m, 1H), 7.72-0.81 (m, 2H), 8.05 (d, J=2.0 Hz, 1H), 9.79 (s, 1H); LCMS (ESI) m/z: 430.1 [M+H]⁺.

### Example 53

### Synthesis of 3-chloro-5-(2,6-difluorophenyl)-N-(oxetan-3-yl)-6H-benzo[f]pyrazolo[1,5-a][1,3,5]triazepine-9-carboxamide

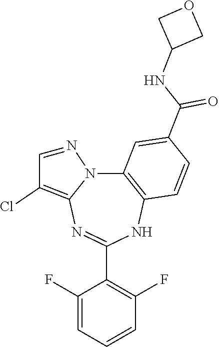 [SMILES: O=C(NC1COC1)c1ccc2c(c1)-n1ncc(Cl)c1N=C(c1c(F)cccc1F)N2]

The title compound was made from 3-chloro-5-(2,6-difluorophenyl)-6H-pyrazolo[1,5-a][1,3,5]benzotriazepine-9-carbonyl chloride (Intermediate 3), following the synthetic procedure as described in the synthesis of 3-chloro-5-(2,6-difluorophenyl)-N-(2-methoxyethyl)-6H-pyrazolo[1,5-a][1,3,5]benzotriazepine-9-carboxamide (Example 45), except that oxetan-3-amine was used in the place of methoxyethanamine. The crude product was purified by prep-HPLC (YMC-Triart Prep C18 150×40 mm×7 μm; mobile phase: [water (NH₃H₂O+NH₄HCO₃)-MeCN]; B %: 50%-90%, 9 min) to provide the title compound as a yellow solid (18.2 mg, 33.3%). ¹HNMR (400 MHz, DMSO-d₆) δ ppm 4.58 (t, J=6.4 Hz, 2H), 4.75 (t, J=6.9 Hz, 2H), 4.89-5.08 (m, 1H), 6.88 (d. J=8.4 Hz, 1H), 7.26 (t, J=8.1 Hz, 2H), 7.50-7.66 (m, 2H), 7.75 (s, 1H), 8.17 (s, 1H), 9.02-9.19 (m, 1H), 9.84 (s, 1H); LCMS (ESI) m/z: 430.0 [M+H]⁺.

### Example 54

### Synthesis of 3-chloro-5-(2,6-difluorophenyl)-N,N-dimethyl-6H-pyrazolo[1,5-a][1,3,5]benzotriazepine-9-carboxamide

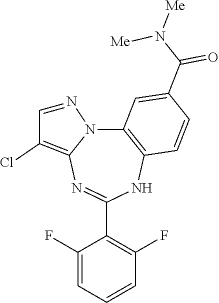 [SMILES: CN(C)C(=O)c1ccc2c(c1)-n1ncc(Cl)c1N=C(c1c(F)cccc1F)N2]

The title compound was made from 3-chloro-5-(2,6-difluorophenyl)-6H-pyrazolo[1,5-a][1,3,5]benzotriazepine-9-carbonyl chloride (Intermediate 3), following the synthetic procedure as described in the synthesis of 3-chloro-5-(2,6-difluorophenyl)-N-(2-methoxyethyl)-6H-pyrazolo[1,5-a][1,3,5]benzotriazepine-9-carboxamide (Example 45), except that N,N-dimethyl amine was used in the place of methoxyethanamine. The crude product was purified by prep-HPLC (YMC-Triart Prep C18 150×40 mm×7 m; mobile phase: [water (NH₃H₂O+NH₄HCO₃)-MeCN]; B %: 50%-90%, 9 min) to provide the title compound as a yellow solid (7.2 mg, 13.4%). ¹HNMR (400 MHz, DMSO-d₆) δ ppm 2.95 (s, 6H), 6.86 (d, J=8.2 Hz, 1H), 7.13 (dd, J=8.1, 1.7 Hz, 1H), 7.27 (t, J=8.1 Hz, 2H), 7.56-7.77 (m, 3H), 9.79 (br s, 1H). LCMS (ESI) m/z: 402.0 [M+H]⁺.

### Example 55

### Synthesis of Methyl 3-chloro-5-(2,6-difluorophenyl)-N-isopropylsulfonyl-6H-pyrazolo[1,5-a][1,3,5]benzotriazepine-9-carboxamide

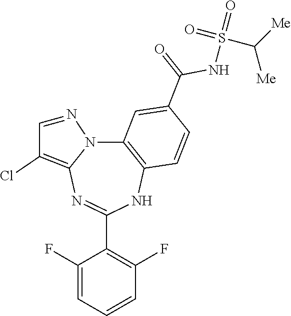 [SMILES: CC(C)S(=O)(=O)NC(=O)c1ccc2c(c1)-n1ncc(Cl)c1N=C(c1c(F)cccc1F)N2]

To a solution of 3-chloro-5-(2,6-difluorophenyl)-6H-pyrazolo[1,5-a][1,3,5]benzotriazepine-9-carboxylic acid (Example 40) (50 mg, 0.133 mmol, 1 eq) in DCM (2 mL) was added 4-N,N-dimethylamino-pyridine (8.15 mg, 66.7 μmol, 0.5 eq), 2-Chloro-1-methylpyridinium iodide (40.9 mg, 0.16 mmol, 1.2 eq), triethylamine (40.5 mg, 0.40 mmol, 3 eq) and propane-2-sulfonamide (32.9 mg, 0.266 mmol, 2 eq). The resulting mixture was stirred at 25 °C for 2 h and the volatiles were removed under reduced pressure. The remaining residue was purified by preparative HPLC (column: Boston Prime C18 150×30 mm×5 μm; mobile phase: [water (ammonia hydroxide v/v)-MeCN]; B %: 5%-35%, 9 min) to afford the title product as yellow solid (29 mg, 45.3%). ¹HNMR (400 MHz, DMSO-d₆) δ ppm 1.20 (br d, J=5.0 Hz, 6H), 3.54-3.79 (m, 1H), 6.82 (br d, J=8.4 Hz, 1H), 6.92-7.11 (m, 1H), 7.26 (t, J=8.2 Hz, 2H), 7.54-7.64 (m, 2H), 7.71 (s, 1H), 8.23 (d, J=1.8 Hz, 1H), 9.81 (br s, 1H); LCMS (ESI) m/z 480.2 [M+H]⁺.

### Example 56

### Synthesis of 4-[5-(2,6-difluorophenyl)-6H-pyrazolo[1,5-a][1,3,5]benzotriazepin-9-yl]morpholine

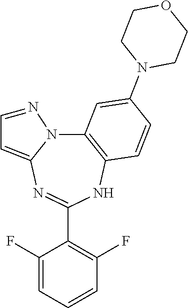 [SMILES: Fc1cccc(F)c1C1=Nc2ccnn2-c2cc(N3CCOCC3)ccc2N1]

A rt mixture of 4-[3-chloro-5-(2,6-difluorophenyl)-6H-pyrazolo[1,5-a][1,3,5]benzotriazepin-9-yl]morpholine (Example 2) (30 mg, 72.2 mol, 1 eq) and Pd/C (16.7 mg, 10 wt % Pd with 50 wt % water) in MeOH (2 mL) was degassed and purged with H₂ for 3 times. The resulting mixture was stirred under H₂ atmosphere (15 psi) for 2 h. The reaction mixture was filtered through a small pad of celite and the celite was washed with MeOH (2 mL). The combined organic solutions was concentrated under reduced pressure. The remaining residue was purified by column chromatography (ISCO®; 4 g SepaFlash® Silica Flash Column, petroleum ether/EtOAc with EtOAc from 0~25%, 20 mL/min, 254 nm) to afford the title compound as a yellow solid (13.5 mg, 48.1%). ¹HNMR (400 MHz, DMSO-d₆) δ ppm 3.06-2.98 (m, 4H), 3.76-3.67 (m, 4H), 5.89 (d, J=1.6 Hz, 1H), 6.73-6.62 (m, 2H), 7.26-7.17 (m, 3H), 7.44 (d, J=1.6 Hz, 1H), 7.61-7.51 (m, 1H), 9.18 (s, 1H); LCMS (ESI) m/z: 382.1 [M+H]⁺.

### Example 57

### Synthesis of 4-[5-chloro-8-(2,6-difluorophenyl)-2,3,7,9,11-pentazatricyclo[8.4.0.02,6]tetradeca-1(10),3,5,11,13-pentaen-13-yl]morpholine

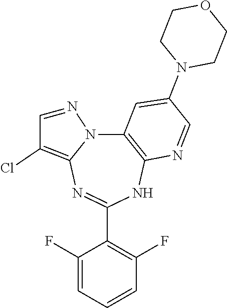 [SMILES: Fc1cccc(F)c1C1=Nc2c(Cl)cnn2-c2cc(N3CCOCC3)cnc2N1]

### Step 1

A mixture of morpholine (5.49 g, 63.1 mmol, 5.5 eq) and 5-bromo-2-nitro-pyridin-3-amine (2.5 g, 11.5 mmol, 1 eq) was degassed and purged with N₂ for 3 times. The mixture was stirred at 140 °C under N₂ atmosphere for 30 min, cooled to rt, and treated with H₂O (30 mL). The precipitate was collected via filtration, washed with EtOH (30 mL), and dried over reduced pressure to provide a crude product of 5-morpholino-2-nitro-pyridin-3-amine as a yellow solid (5.17 g). LCMS (ESI) m/z: 225.1 [M+H]⁺.

### Step 2

A solution of CuCl (1.06 g, 10.7 mmol, 1.2 eq) and isopentyl nitrite (1.36 g, 11.6 mmol, 1.3 eq) in MeCN (35 mL) was warmed to 55 °C, and stirred for 0.5 h. A solution of 5-morpholino-2-nitro-pyridin-3-amine (2 g, 8.92 mmol, 1 eq) in MeCN (25 mL) was slowly added to the resulting reaction mixture, while gas evolution was observed. Once the addition was completed, the reaction temperature was raised to 65 °C, and the mixture was stirred for additional 1.0 h. The reaction mixture was cooled to rt, diluted with H₂O (10 mL) and extracted with EtOAc (20 mL×3). The combined organic layers were washed with brine (10 mL), dried over Na₂SO₄, filtered and concentrated under reduced pressure. The remaining residue was purified by flash silica gel chromatography (ISCO®; 40 g SepaFlash® Silica Flash Column, Eluent of 0~50% petroleum ether/EtOAc @80 mL/min) to provide 4-(5-chloro-6-nitro-3-pyridyl)morpholine as yellow solid (740 mg, 32.0%). LCMS (ESI) m/z: 244.0 [M+H]⁺.

### Step 3

A mixture of 4-(5-chloro-6-nitro-3-pyridyl)morpholine (2.7 g, 11.0 mmol), N₂H₄·H₂O (2.61 g, 44.3 mmol, 4 eq) in dioxane (20 mL) was degassed and purged with N₂ for 3 times, and then the mixture was stirred at 100 °C under N₂ atmosphere for 3 h. The reaction mixture was cooled to rt, diluted with H₂O (30 mL) and extracted with EtOAc (30 mL×3). The combined organic layers were washed with brine (30 mL×2), dried with anhydrous Na₂SO₄, filtered and concentrated under reduced pressure. The remaining residue was purified by flash chromatography (ISCO®; 12 g SepaFlash® Silica Flash Column, petroleum ether/EtOAc with EtOAc from 0~5%, 45 mL/min, 254 nM) to provide (5-morpholino-2-nitro-3-pyridyl)hydrazine as yellow solid (1.7 g, 29.5%). LCMS (ESI) m/z: 240.1 [M+H]⁺.

### Step 4

To a solution of (5-morpholino-2-nitro-3-pyridyl)hydrazine (900 mg. 3.76 mmol, 1 eq) in EtOH (3 mL) was added AcOH (451 mg, 7.52 mmol, 2 eq) and ethyl (E)-2-cyano-3-ethoxy-prop-2-enoate (700 mg, 4.14 mmol, 1.1 eq). The mixture was stirred at 80 °C for 16 h. The reaction mixture was cooled to rt, diluted with H₂O (30 mL) and extracted with EtOAc (30 mL×3). The combined organic layers were washed with brine (30 mL×2), dried with anhydrous Na₂SO₄, filtered and concentrated under reduced pressure to give a crude product of ethyl 5-amino-1-(5-morpholino-2-nitro-3-pyridyl)pyrazole-4-carboxylate as brown syrup (1 g).

### Step 5

To a solution of crude ethyl 5-amino-1-(5-morpholino-2-nitro-3-pyridyl)pyrazole-4-carboxylate (900 mg, 2.48 mmol, 1 eq) in mixture solvent of EtOH (9 mL) and H₂O (2 mL). was added Fe (1.39 g, 24.8 mmol, 1 eq) and NH₄Cl (1.33 g, 24.8 mmol, 10 eq). After stirring at 50 °C for 2 h, the reaction mixture was cooled to rt, diluted with H₂O (30 mL), and extracted with EtOAc (30 mL×3). The organic layers were combined, washed with brine (30 mL×2), dried with anhydrous Na₂SO₄, filtered and concentrated under reduced pressure. The remaining residue was purified by flash chromatography (ISCO®; 40 g SepaFlash® Silica Flash Column, petroleum ether/EtOAc with EtOAc from 0-100%, 45 mL/min, 254 nM) to provide ethyl 5-amino-1-(2-amino-5-morpholino-3-pyridyl)pyrazole-4-carboxylate as brown oil (700 mg, 82.2%). LCMS (ESI) m/z: 333.1 [M+H]⁺.

### Step 6

A mixture of ethyl 5-amino-1-(2-amino-5-morpholino-3-pyridyl)pyrazole-4-carboxylate (700 mg, 2.11 mmol, 1 eq) in H₃PO₄ (7 mL, 85 wt %) was stirred at 160 °C for 4 h. The resulting reaction mixture was cooled to rt, diluted with H₂O (30 mL) and extracted with EtOAc (30 mL×3). The combined organic layers were washed with brine (30 mL×2), dried with anhydrous Na₂SO₄, filtered, and concentrated under reduced pressure. The remaining residue was purified by flash chromatography (ISCO®; 12 g SepaFlash® Silica Flash Column, petroleum ether/EtOAc with EtOAc from 0~100%, 45 mL/min, 254 mn) to provide 3-(5-aminopyrazol-1-yl)-5-morpholino-pyridin-2-amine as brown oil (400 mg, 65.6%). LCMS (ESI) m/z: 261.0 [M+H]⁺.

### Step 7

To a solution of 3-(5-aminopyrazol-1-yl)-5-morpholino-pyridin-2-amine (400 mg, 1.54 mmol, 1 eq) in DCM (1 mL) was added NCS (205 mg, 1.54 mmol, 1 eq). After stirring at rt for 2 h, the reaction mixture was diluted with H₂O (30 mL) and extracted with EtOAc (30 mL×3). The combined organic layers were washed with brine (30 mL×2), dried with anhydrous Na₂SO₄, filtered and concentrated under reduced pressure. The remaining residue was triturated with 10:1 petroleum ether/EtOAc at 25 °C for 2 h, to provide 3-(5-amino-4-chloro-pyrazol-1-yl)-5-morpholino-pyridin-2-amine as a brown oil (320 mg, 42.3%).

### Step 8

To a solution of 3-(5-amino-4-chloro-pyrazol-1-yl)-5-morpholino-pyridin-2-amine (250 mg, 0.848 mmol, 1 eq) in THF (3 mL) was slowly added 2,6-Difluorobenzaldehyde (120 mg, 0.848 mmol, 1 eq) in THF (3 mL). After stirring at 60 °C for 16 h, the reaction solution was cooled to rt, diluted with H₂O (30 mL) and extracted with EtOAc (30 mL×3). The combined organic layers were washed with brine (30 mL×2), dried with anhydrous Na₂SO₄, and concentrated under reduced pressure to removed volatiles. To the remaining residue was added DDQ (130 mg, 0.573 mmol, 1.2 eq) followed by DCM (1 mL). The mixture was stirred at 25 °C for 48 h and concentrated under reduced pressure. The remaining residue was purified by flash silica gel chromatography (ISCO®; 4 g SepaFlash® Silica Flash Column, Eluent of 0~45% petroleum ether/EtOAc @35 mL/min) to afford the desired product of 4-[5-chloro-8-(2,6-difluorophenyl)-2,3,7,9,11-pentazatricyclo[8.4.0.02,6]tetradeca-1(10),3,5,7,11,13-hexaen-13-yl]morpholine as yellow solid (7.86 mg, 3.8%). ¹HNMR (400 MHz, DMSO-d₆) δ 3.15-3.09 (m, 4H), 3.80-3.61 (m, 4H), 7.19 (t, J=8.1 Hz, 2H), 7.62-7.49 (m, 2H), 7.82-7.67 (m, 2H), 10.22-9.99 (m, 1H). LCMS (ESI) m/z: 417.0 [M+H]⁺.

### Example 58

### Synthesis of Methyl 5-chloro-8-(2,6-difluorophenyl)-2,3,7,9,11-pentazatricyclo[8.4.0.02,6]tetradeca-1(10),3,5,7,11,13-hexaene-13-carboxylate

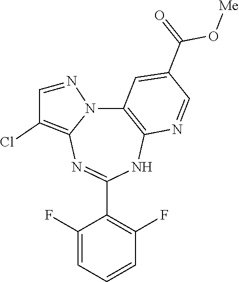 [SMILES: COC(=O)c1cnc2c(c1)-n1ncc(Cl)c1N=C(c1c(F)cccc1F)N2]

### Step 1

To a solution of 5-bromo-3-fluoro-2-nitro-pyridine (5 g, 22.6 mmol, 1 eq) in EtOH (50 mL) was added hydrazine (1.28 g, 33.9 mmol, 85 wt %, 1.5 eq). After stirring at 80 °C for 6 h, the reaction mixture was cooled to rt, treated with water (150 mL) and extracted with EtOAc (100 mL×3). The combined organic layers were washed with brine (100 mL), dried with anhydrous Na₂SO₄, filtered and concentrated under reduced pressure. The remaining residue was purified by flash silica gel chromatography (ISCO®; 20 g SepaFlash® Silica Flash Column, petroleum ether/EtOAc with EtOAc from 0~30%, 35 mL/min, 254 nm) to provide (5-bromo-2-nitro-3-pyridyl) hydrazine yellow solid (4.75 g, 89.2%). LCMS (ESI) m/z: 233.0 [M+H]⁺.

### Step 2

To a solution of (5-bromo-2-nitro-3-pyridyl)hydrazine (4.75 g, 20.3 mmol, 1 eq) in EtOH (60 mL) was added ethyl (E)-2-cyano-3-ethoxy-prop-2-enoate (4.14 g, 24.4 mmol, 1.2 eq). After stirring at 80 °C for 5 h, the reaction mixture was cooled to rt, filtered and concentrated under reduced pressure. The remaining residue was purified by flash silica gel chromatography (ISCO®; 120 g SepaFlash® Silica Flash Column, petroleum ether/EtOAc with EtOAc from 0~30%, 85 mL/min, 254 nm) to provide ethyl 5-amino-1-(5-bromo-2-nitro-3-pyridyl) pyrazole-4-carboxylate as a yellow solid (5.9 g, 80.9%). LCMS (ESI) m/z: 355.9 [M+H]⁺.

### Step 3

To a solution of ethyl 5-amino-1-(5-bromo-2-nitro-3-pyridyl) pyrazole-4-carboxylate (5.9 g, 16.5 mmol, 1 eq) in H₂O (15 mL) and EtOH (45 mL) was added Fe (9.25 g, 0.165 mol, 10 eq) and NH₄Cl (8.86 g, 0.165 mol, 10 eq). After stirring at 50 °C for 3 h, the reaction mixture was cooled to rt, filtered and concentrated under reduced pressure to give crude product of ethyl 5-amino-1-(2-amino-5-bromo-3-pyridyl) pyrazole-4-carboxylate as white solid (4.68 g, 81.4%). LCMS (ESI) m/z: 327.9 [M+H]⁺.

### Step 4

A mixture of ethyl 5-amino-1-(2-amino-5-bromo-3-pyridyl) pyrazole-4-carboxylate (4.6 g, 14.1 mmol, 1 eq) and H₃PO₄ (15 mL) was stirred at 160 °C for 3 h, the resulting solution was cooled to rt, and treated with aqueous aqueous NaHCO₃ solution (40 mL). The resulting mixture was extracted with EtOAc (30 mL×3), and the combined organic layers were washed with brine (20 mL), dried with anhydrous Na₂SO₄, filtered and concentrated under reduced pressure. The remaining residue was purified by flash silica gel chromatography (ISCO®; 12 g SepaFlash® Silica Flash Column, petroleum ether/EtOAc with EtOAc from 0~50%, 35 mL/min, 254 nm) to provide 3-(5-aminopyrazol-1-yl)-5-bromo-pyridin-2-amine as yellow solid (2.65 g, 72.8%). LCMS (ESI) m/z: 253.9 [M+H]⁺.

### Step 5

A mixture of 3-(5-aminopyrazol-1-yl)-5-bromo-pyridin-2-amine (2.6 g, 10.2 mmol, 1 eq), Pd(dppf)Cl₂ (748 mg, 1.02 mmol, 0.1 eq) and TEA (3.11 g, 30.7 mmol, 3 eq) in MeOH (70 mL) was degassed and purged with CO for 3 times. The resulting mixture under CO (50 psi) was stirred at 80 °C atmosphere for 48 h, cooled to rt, filtered and concentrated under reduced pressure. The remaining residue was purified by flash silica gel chromatography (ISCO®; 12 g SepaFlash® Silica Flash Column, petroleum ether/EtOAc with EtOAc from 0~50%, 35 mL/min, 254 nm) to provide methyl 6-amino-5-(5-aminopyrazol-1-yl) pyridine-3-carboxylate as a yellow solid (2.23 g, 83.5%). LCMS (ESI) m/z: 234.0 [M+H]⁺.

### Step 6

To a solution of methyl 6-amino-5-(5-aminopyrazol-1-yl)pyridine-3-carboxylate (1 g, 4.29 mmol, 1 eq) in MeCN (5 mL) was added NCS (629.8 mg, 4.72 mmol, 1.1 eq). After stirring at 0 °C for 3 h, the reaction mixture was filtered and concentrated under reduced pressure. The remaining residue was purified by flash silica gel chromatography (ISCO®; 12 g SepaFlash® Silica Flash Column, Eluent of 0~50% EtOAc/petroleum ether, flow rate: 35 mL/min) to afford methyl 6-amino-5-(5-amino-4-chloro-pyrazol-1-yl)pyridine-3-carboxylate as a yellow solid (1.1 g, 93.6%). LCMS (ESI) m/z: 268.0 [M+H]⁺.

### Step 7

A solution of methyl 6-amino-5-(5-amino-4-chloro-pyrazol-1-yl)pyridine-3-carboxylate (200 mg, 0.747 mmol, 1 eq) and 2,6-difluorobenzaldehyde (127 mg, 0.90 mmol, 1.2 eq) in toluene (1 mL) was stirred at 100 °C for 2 h. The reaction mixture was cooled to rt, filtered and concentrated under reduced pressure to give crude product of methyl 5-chloro-8-(2,6-difluorophenyl)-2,3,7,9,11-pentazatricyclo[8.4.0.02,6]tetradeca-1(10),3,5,11,13-pentaene-13-carboxylate as yellow solid (230 mg), which was directly used in next step without further purification. LCMS (ESI) [M+H]⁺ m/z: 392.0.

### Step 8

To a solution of crude methyl 5-chloro-8-(2,6-difluorophenyl)-2,3,7,9,11-pentazatricyclo[8.4.0.02,6]tetradeca-1(10),3,5,11,13-pentaene-13-carboxylate (230 mg, 0.587 mmol, 1 eq) in CH₂Cl₂ (3 mL) was added DDQ (199 mg, 0.880 mmol, 1.5 eq). After stirring at 25 °C for 2 h, the resulting reaction mixture was filtered and concentrated under reduced pressure. The remaining residue was purified by flash silica gel chromatography (ISCO®; 4 g SepaFlash® Silica Flash Column, petroleum ether/EtOAc with EtOAc from 0~30%, 20 mL/min, 254 nm) to afford the desired title compound of methyl 5-chloro-8-(2,6-difluorophenyl)-2,3,7,9,11-pentazatricyclo[8.4.0.02,6]tetradeca-1(10),3,5,7,11,13-hexaene-13-carboxylate as yellow solid (50 mg, 21.9%). ¹HNMR (400 MHz, DMSO-d₆) δ ppm 3.88 (s, 3H), 7.15-7.19 (m, 2H), 7.54-7.58 (m, 1H), 7.76 (s, 1H), 8.40 (s, 1H), 8.46 (s, 1H), 10.59 (brs, 1H). LCMS (ESI) m/z: 390.0 [M+H]⁺.

### Example 59

### Synthesis of 5-chloro-8-(2,6-difluorophenyl)-N-(2-fluoroethyl)-2,3,7,9,11-pentazatricyclo[8.4.0.02,6]tetradeca-1(10),3,5,7,11,13-hexaene-13-carboxamide

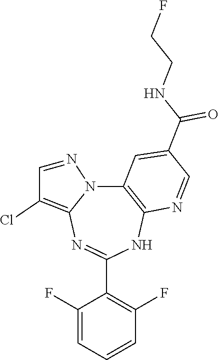 [SMILES: O=C(NCCF)c1cnc2c(c1)-n1ncc(Cl)c1N=C(c1c(F)cccc1F)N2]

The title compound was made from 5-Chloro-8-(2,6-difluorophenyl)-2,3,7,9,11-pentazatricyclo[8.4.0.02,6]tetradeca-1(10),3,5,7,11,13-hexaene-13-carboxylic acid (Intermediate 8), following the synthetic procedure as described in the synthesis of 3-chloro-5-(2,6-difluorophenyl)-6H-pyrazolo[1,5-a][1,3,5]benzotriazepine-9-carboxamide (Example 41), except that 2-fluoroethanamine was used in the place of NH₄Cl, and the reaction was conducted in CH₂Cl₂ at 25 °C for 3 h. The crude final product was purified by flash silica gel chromatography (ISCO®; 4 g SepaFlash® Silica Flash Column, petroleum ether/EtOAc with EtOAc from 0-40%, flow rate=20 mL/min, 254 nm) to provide the desired title compound of 5-chloro-8-(2,6-difluorophenyl)-N-(2-fluoroethyl)-2,3,7,9,11-pentazatricyclo[8.4.0.02,6]tetradeca-1(10),3,5,7,11,13-hexaene-13-carboxamide as a yellow solid (11.2 mg, 24.1%). ¹HNMR (400 MHz, DMSO-d₆) δ ppm 3.49-3.64 (m, 2H), 4.48 (t, J=5.0 Hz, 1H), 4.59 (t, J=5.0 Hz, 1H), 7.20 (t, J=8.2 Hz, 2H), 7.57 (quin, J=7.5 Hz, 1H), 7.82 (s, 1H), 8.42 (dd, J=14.8, 1.8 Hz, 2H), 8.89 (br t, J=5.4 Hz, 1H), 10.65 (br s, 1H); LCMS (ESI) m/z 421.0 [M+H]⁺.

### Example 60

### Synthesis of 4-(3-Chloro-5-(2,6-difluorophenyl)-6H-pyrazolo[1,5-a]pyrido[3,4-f][1,3,5]triazepin-9-yl)morpholine

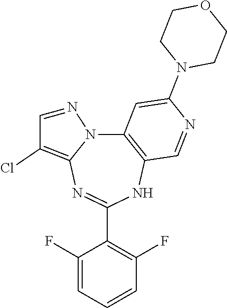 [SMILES: Fc1cccc(F)c1C1=Nc2c(Cl)cnn2-c2cc(N3CCOCC3)ncc2N1]

### Step 1

To a solution of 4-(5-amino-4-chloro-pyrazol-1-yl)-6-morpholino-pyridin-3-amine (Intermediate 6) (300 mg, 1.02 mmol) and K₂CO₃ (422 mg, 3.05 mmol) in THF (2 mL) and t-BuOH (8 mL) was added 2,6-difluorobenzaldehyde (174 mg. 1.22 mmol). After stirring at 50 °C for 16 h, the resulting mixture was cooled to rt, filtered and concentrated under reduced pressure. The remaining residue was purified by flash silica gel chromatography (ISCO®; 12 g SepaFlash® Silica Flash Column, petroleum ether/EtOAc with EtOAc from 0~50%, flow rate=40 mL/min, 254 nm) to provide crude product of 4-[5-chloro-8-(2,6-difluorophenyl)-2,3,7,9,12-pentazatricyclo[8.4.0.02,6]tetradeca-1(10),3,5,11,13-pentaen-13-yl]morpholine as a yellow solid (340 mg, 79.7%). LCMS (ESI) m/z 419.0 [M+H]⁺.

### Step 2

To a solution of 4-[5-chloro-8-(2,6-difluorophenyl)-2,3,7,9,12-pentazatricyclo[8.4.0.02,6]tetradeca-1(10),3,5,11,13-pentaen-13-yl]morpholine (340 mg, 0.812 mmol) in CH₂Cl₂ (10 mL) was added DDQ (203 mg, 0.893 mmol). After stirring at 25 °C for 3 h, the resulting mixture was diluted with EtOAc (100 mL) and washed with saturated aqueous NaHCO₃ solution (25 mL) and brine (50 mL) subsequently. The organic solution was dried over anhydrous Na₂SO₄, filtered and concentrated under reduced pressure. The remaining residue was purified by flash silica gel chromatography (ISCO®; 12 g SepaFlash® Silica Flash Column, petroleum ether/EtOAc with EtOAc from 0~40%, flow rate=40 mL/min, 254 nm) to provide the desired product of 4-[5-chloro-8-(2,6-difluorophenyl)-2,3,7,9,12-pentazatricyclo[8.4.0.02,6]tetradeca-1(10),3,5,7,11,13-hexaen-13-yl]morpholine as a yellow solid (321.53 mg, 93.6%). ¹HNMR (400 MHz, CD₃OD) δ ppm 3.39-3.46 (m, 4H), 3.76-3.80 (m, 4H), 7.05-7.16 (m, 3H), 7.48-7.59 (m, 3H); LCMS (ESI) m/z 417.1 [M+H]⁺.

### Example 61

### Synthesis of 3-(3-Chloro-5-(2,6-difluorophenyl)-6H-pyrazolo[1,5-a]pyrido[3,4-f][1,3,5]triazepin-9-yl)-8-oxa-3-azabicyclo[3.2.1]octane

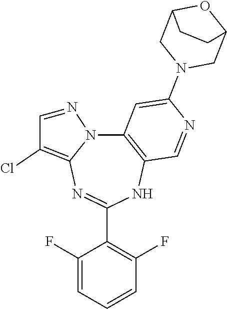 [SMILES: Fc1cccc(F)c1C1=Nc2c(Cl)cnn2-c2cc(N3CC4CCC(C3)O4)ncc2N1]

### Step 1

A mixture of ethyl 5-amino-1-(2-chloro-5-nitropyridin-4-yl)-1H-pyrazole-4-carboxylate (Intermediate 4) (2.5 g, 8.02 mmol), TEA (2.43 g, 24.1 mmol) and 8-oxa-3-azabicyclo[3.2.1]octane (1.20 g, 8.01 mmol) in DCM (25 mL) was stirred at 25 °C for 3 h. The reaction mixture was concentrated under reduced pressure and the remaining residue was purified by flash silica gel chromatography (ISCO®; 12 g SepaFlash® Silica Flash Column, petroleum ether/EtOAc with EtOAc from 0~70%, flow rate=50 mL/min, 254 nm) to provide ethyl 5-amino-1-[5-nitro-2-(8-oxa-3-azabicyclo[3.2.1]octan-3-yl)-4-pyridyl]pyrazole-4-carboxylate as a yellow solid (2.2 g, 70.6%). LCMS (ESI) m/z 389.0 [M+H]⁺.

### Step 2

A solution of ethyl 5-amino-1-[5-nitro-2-(8-oxa-3-azabicyclo[3.2.1]octan-3-yl)-4-pyridyl]pyrazole-4-carboxylate (2.3 g, 5.92 mmol) in H₃PO₄ (15 mL) was stirred at 160 °C for 4 h. The reaction mixture was cooled to rt and treated with H₂O (10 mL). The resulting mixture was neutralized with saturated aqueous NaHCO₃ to pH=7-8 and extracted with EtOAc (50 mL×3). The combined organic layers were washed with brine (20 mL), dried with anhydrous Na₂SO₄, filtered and concentrated under reduced pressure. The remaining residue was purified by flash silica gel chromatography (ISCO®; 20 g SepaFlash® Silica Flash Column, petroleum ether/EtOAc with EtOAc from 0~70%, flow rate=50 mL/min, 254 nm) to provide 2-[5-nitro-2-(8-oxa-3-azabicyclo[3.2.1]octan-3-yl)-4-pyridyl]pyrazol-3-amine as a yellow solid (1.5 g, 64.0%). LCMS (ESI) m/z 317.1 [M+H]⁺.

### Step 3

A solution of 2-[5-nitro-2-(8-oxa-3-azabicyclo[3.2.1]octan-3-yl)-4-pyridyl]pyrazol-3-amine (1.4 g, 4.43 mmol) and NCS (591 mg, 4.43 mmol) in DCM (20 mL) was stirred at 25 °C for 2 h. The resulting mixture was treated with H₂O (20 mL) and extracted with EtOAc (50 mL×3). The combined organic layers were washed with brine (20 mL), dried with anhydrous Na₂SO₄, filtered and concentrated under reduced pressure. The remaining residue was purified by flash silica gel chromatography (ISCO®; 12 g SepaFlash® Silica Flash Column, petroleum ether/EtOAc with EtOAc from 0~50%, flow rate=50 mL/min, 254 nm) to provide 4-chloro-2-[5-nitro-2-(8-oxa-3-azabicyclo[3.2.1]octan-3-yl)-4-pyridyl]pyrazol-3-amine as a brown solid (1.32 g, 81.6%). LCMS (ESI) m/z 351.0 [M+H]⁺.

### Step 4

To a 0 °C solution of 4-chloro-2-[5-nitro-2-(8-oxa-3-azabicyclo[3.2.1]octan-3-yl)-4-pyridyl]pyrazol-3-amine (1.22 g, 3.48 mmol) in THF (15 mL) under N₂ atmosphere was added NiCl₂₋₆H₂O (2.48 g, 10.4 mmol) and NaBH₄ (790 mg, 20.9 mmol) at. The mixture was stirred at 0 °C for 1 h, and quenched by addition H₂O (1 mL). The resulting mixture was filtered through a small pad of celite and the filtrate was concentrated under reduced pressure. The remaining residue was purified by flash silica gel chromatography (ISCO®; 12 g SepaFlash® Silica Flash Column, petroleum ether/EtOAc with EtOAc from 0~100%, flow rate=50 mL/min, 254 nm) to provide 4-(5-amino-4-chloro-pyrazol-1-yl)-6-(8-oxa-3-azabicyclo[3.2.1]octan-3-yl)pyridin-3-amine as a brown solid (550 mg, 49.3%). LCMS (ESI) m/z 321.0 [M+H]⁺.

### Step 5

A solution of 4-(5-amino-4-chloro-pyrazol-1-yl)-6-(8-oxa-3-azabicyclo[3.2.1]octan-3-yl)pyridin-3-amine (100 mg, 0.312 mmol), 2,6-difluorobenzaldehyde (44.3 mg, 0.312 mmol) and K₂CO₃ (129 mg, 0.935 mmol) in THF (1 mL) and t-BuOH (4 mL) was stirred at 50 °C for 16 h. The reaction mixture was cooled to rt, filtered and concentrated under reduced pressure. The remaining residue was purified by flash silica gel chromatography (ISCO®; 4 g SepaFlash® Silica Flash Column, petroleum ether/EtOAc with EtOAc from 0~40%, flow rate=35 mL/min, 254 nm) to provide 3-[5-chloro-8-(2,6-difluorophenyl)-2,3,7,9,12-pentazatricyclo[8.4.0.02,6]tetradeca-1(10),3,5,11,13-pentaen-13-yl]-8-oxa-3-azabicyclo[3.2.1]octane as a yellow solid (119 mg, 70.3%). LCMS (ESI) m/z 445.1 [M+H]⁺.

### Step 6

A solution of 3-[5-chloro-8-(2,6-difluorophenyl)-2,3,7,9,12-pentazatricyclo[8.4.0.02,6]tetradeca-1(10),3,5,11,13-pentaen-13-yl]-8-oxa-3-azabicyclo[3.2.1]octane (119 mg, 268 mmol) and DDQ (72.9 mg, 0.321 mmol) in DCM (4 mL) was stirred at 25 °C for 3 h. The resulting mixture was diluted with EtOAc (80 mL) and washed with aqueous NaHCO₃ solution. The organic layers were washed with brine (50 mL), dried with anhydrous Na₂SO₄, filtered and concentrated under reduced pressure. The remaining residue was purified by flash silica gel chromatography (ISCO®; 12 g SepaFlash® Silica Flash Column, petroleum ether/EtOAc with EtOAc from 0~50%, flow rate=40 mL/min, 254 nm) to provide desired product of 3-[5-chloro-8-(2,6-difluorophenyl)-2.3,7,9,12-pentazatricyclo[8.4.0.02.6]tetradeca-1(10),3,5,7,11,13-hexaen-13-yl]-8-oxa-3-azabicyclo[3.2.1]octane as a yellow solid (82.07 mg, 68.1%). ¹HNMR (400 MHz, CD₃OD) δ ppm 1.82-1.98 (m, 4H), 3.03 (dd, J=12.4, 2.4 Hz, 2H), 3.73 (d, J=12.4 Hz, 2H), 4.46 (br s, 2H), 7.03-7.14 (m, 3H), 7.48-7.60 (m, 3H); LCMS (ESI) m/z 443.1 [M+H]⁺.

### Example 62

### Synthesis of (1R,4R)-5-[5-chloro-8-(2,6-difluorophenyl)-2,3,7,9,12-pentazatricyclo[8.4.0.02,6]tetradeca-1(10),3,5,7,11,13-hexaen-13-yl]-2-oxa-5-azabicyclo[2.2.1]heptane

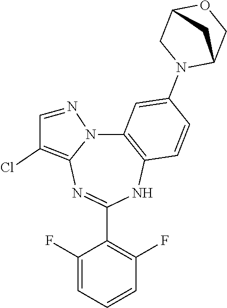 [SMILES: Fc1cccc(F)c1C1=Nc2c(Cl)cnn2-c2cc(N3C[C@H]4C[C@@H]3CO4)ccc2N1]

The title compound was made from ethyl 5-amino-1-(2-chloro-5-nitropyridin-4-yl)-1H-pyrazole-4-carboxylate (Intermediate 4), following the synthetic procedure as described in the synthesis of 3-(3-Chloro-5-(2,6-difluorophenyl)-6H-pyrazolo[1,5-a]pyrido[3,4-f][1,3,5]triazepin-9-yl)-8-oxa-3-azabicyclo[3.2.1]octne (Example 61), except that (1R,4R)-2-oxa-5-azabicyclo[2.2.1]heptane was used in the place of 8-oxa-3-azabicyclo[3.2.1]octane (step 1). The final crude product (step 6) was purified by flash chromatography (ISCOC; 4 g SepaFlash® Silica Flash Column, petroleum ether/EtOAc with EtOAc from 0~45%, flow rate=45 mL/min, 254 nm) to afford the title product of (1R,4R)-5-[5-chloro-8-(2,6-difluorophenyl)-2,3,7,9,12-pentazatricyclo[8.4.0.02,6]tetradeca-1(10),3,5,7,11,13-hexaen-13-yl]-2-oxa-5-azabicyclo[2.2.1]heptane as a yellow solid (35.09 mg, 10.3%, 98.1% purity). ¹HNMR (400 MHz, DMSO-d₆) δ ppm 1.84-1.78 (m, 1H), 1.91-1.85 (m, 1H), 3.17 (d, J=9.8 Hz, 1H), 3.43 (d, J=8.8 Hz, 1H), 3.63 (d, J=7.3 Hz, 1H), 3.74 (d, J=6.5 Hz, 1H), 4.62 (s, 1H), 4.74 (s, 1H), 6.71 (s, 1H), 7.25 (t, J=8.0 Hz, 2H), 7.64-7.53 (m, 2H), 7.68 (s, 1H), 9.70 (s, 1H); LCMS (ESI) m/z 429.0 [M+H]⁺.

### Example 63

### Synthesis of 4-(5-(2,6-difluorophenyl)-6H-pyrazolo[1,5-a]pyrido[3,4-f][1,3,5]triazepin-9-yl)morpholine

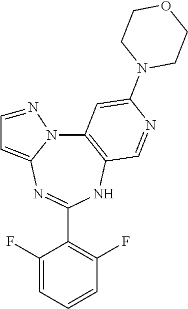 [SMILES: Fc1cccc(F)c1C1=Nc2ccnn2-c2cc(N3CCOCC3)ncc2N1]

### Step 1

To a solution of 2-(2-morpholino-5-nitro-4-pyridyl)pyrazol-3-amine (Intermediate 5) (0.7 g, 2.41 mmol) in THF (15 mL) was added Pd/C (200 mg, 10 wt % Pd with 50 wt % water). After stirring under H₂ atmosphere (15 psi) at 25 °C for 6 h, the reaction mixture was filtered and the solution was concentrated under reduced pressure to provide crude product of 4-(5-aminopyrazol-1-yl)-6-morpholino-pyridin-3-amine as brown oil (550 mg, 82.3%). LCMS (ESI) m/z 261.2 [M+H]⁺.

### Step 2

A solution of 4-(5-aminopyrazol-1-yl)-6-morpholino-pyridin-3-amine (400 mg, 1.54 mmol) and 2,6-difluorobenzaldehyde (262 mg, 1.84 mmol) and K₂CO₃ (637 mg, 4.61 mmol) in THF (4 mL) and t-BuOH (12 mL) was stirred at 50 °C for 16 h. The reaction mixture was cooled to rt, filtered and concentrated under reduced pressure. The remaining residue was purified by flash silica gel chromatography (ISCO®; 12 g SepaFlash® Silica Flash Column, petroleum ether/EtOAc with EtOAc from 0~40%, flow rate=30 mL/min, 254 nm) to provide 4-[8-(2,6-difluorophenyl)-2,3,7,9,12-pentazatricyclo[8.4.0.02,6]tetradeca-1(10),3,5,11,13-pentaen-13-yl]morpholine as a yellow solid (560 mg, 66.3%). LCMS (ESI) m/z 385.0 [M+H]⁺.

### Step 3

A solution of 4-[8-(2,6-difluorophenyl)-2,3,7,9,12-pentazatricyclo[8.4.0.02,6]tetradeca-1(10),3,5,11,13-pentaen-13-yl]morpholine (560 mg, 1.46 mmol) and DDQ (397 mg, 1.75 mmol) in DCM (15 mL) was stirred at 25 °C for 3 h. The resulting solution was diluted with EtOAc (80 mL×2), and washed with saturated aqueous NaHCO₃ solution. The combined organic layers were washed with brine (50 mL), dried with anhydrous Na₂SO₄, filtered and concentrated under reduced pressure. The remaining residue was purified by flash silica gel chromatography (ISCO®; 12 g SepaFlash® Silica Flash Column, petroleum ether/EtOAc with EtOAc from 0~50%, flow rate=50 mL/min, 254 nm) to provide the title product of 4-[8-(2,6-difluorophenyl)-2,3,7,9,12-pentazatricyclo[8.4.0.02,6]tetradeca-1(10),3,5,7,11,13-hexaen-13-yl]morpholine as a yellow solid (400 mg, 69.3%). ¹HNMR (400 MHz, CD₃OD) δ ppm 3.42-3.54 (m, 4H), 3.78-3.83 (m, 4H), 5.95 (s, 1H), 7.06-7.22 (m, 3H), 7.45-7.69 (m, 3H); LCMS (ESI) m/z 383.0 [M+H]⁺.

### Example 64

### Synthesis of 4-(3-bromo-5-(2,6-difluorophenyl)-6H-pyrazolo[1,5-a]pyrido[3,4-f][1,3,5]triazepin-9-yl)morpholine

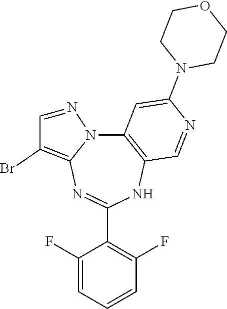 [SMILES: Fc1cccc(F)c1C1=Nc2c(Br)cnn2-c2cc(N3CCOCC3)ncc2N1]

A solution of 4-[8-(2,6-difluorophenyl)-2,3,7,9,12-pentazatricyclo[8.4.0.02,6]tetradeca-1(10),3,5,7,11,13-hexaen-13-yl]morpholine (Example 63) (100 mg, 0.262 mmol) and NBS (46.6 mg, 0.262 mmol) in DCM (3 mL) was stirred at 25 °C for 2 h. The reaction mixture was quenched with H₂O (10 mL) and extracted with EtOAc (30 mL×3). The combined organic layers were washed with brine (20 mL), dried with anhydrous Na₂SO₄, filtered and concentrated under reduced pressure. The remaining residue was purified by flash silica gel chromatography (ISCO®; 4 g SepaFlash® Silica Flash Column, petroleum ether/EtOAc with EtOAc from 0-35%, flow rate=35 mL/min, 254 nm) to provide the title compound as a yellow solid (28.79 mg, 23.6%). ¹HNMR (400 MHz, CD₃OD) δ ppm 3.39-3.47 (m, 4H), 3.74-3.82 (m, 4H), 7.05-7.15 (m, 3H), 7.47-7.59 (m, 3H); LCMS (ESI) m/z 461.0 [M+H]⁺.

### Example 65

### Synthesis of Synthesis of 5-(2,6-difluorophenyl)-9-morpholino-6H-pyrazolo[1,5-a]pyrido[3,4-f][1,3,5]triazepine-3-carbonitrile

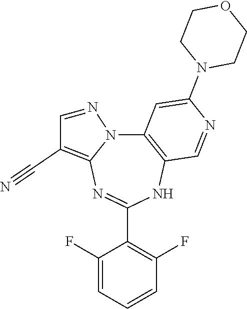 [SMILES: N#Cc1cnn2c1N=C(c1c(F)cccc1F)Nc1cnc(N3CCOCC3)cc1-2]

To a solution of 4-[5-bromo-8-(2,6-difluorophenyl)-2,3,7,9,12-pentazatricyclo[8.4.0.02,6]tetradeca-1(10),3,5,7,11,13-hexaen-13-yl]morpholine (Example 64) (200 mg, 0.434 mmol, 1 eq.), Zn(CN)₂ (153 mg, 1.30 mmol, 3 eq.) in dioxane (5 mL) was added BrettPhos-Pd-G₃ (118 mg, 0.130 mmol, 0.3 eq.) and Cs₂CO₃ (424 mg, 1.30 mmol, 3 eq.). The mixture was stirred at 110 °C for 2 h under microwave reactor. The resulting reaction mixture was cooled to rt, filtered and concentrated under reduced pressure. The remaining residue was purified by prep-HPLC (column: 2_Phenomenex Gemini C18 75×40 mm×3 μm; mobile phase: [water (NH₄HCO₃)-MeCN]; B %: 35%-65%, 9.5 min, 254 nm) to provide the desired title product as a red solid (44.8 mg, 24.2%). ¹HNMR (400 MHz, DMSO-d₆) δ ppm 3.33-3.42 (m, 4H), 3.59-3.73 (m, 4H), 6.97 (s, 1H), 7.27 (t, J=8.16 Hz, 2H), 7.57-7.67 (m, 2H), 7.99 (s, 1H), 9.41-10.58 (m, 1H); ¹⁹F NMR (377 MHz, DMSO-d₆) ppm −113.24; LCMS (ESI) m/z 408.1 [M+H]⁺.

### Example 66

### Synthesis of 4-(5-(2,6-difluorophenyl)-3-methyl-6H-pyrazolo[1,5-a]pyrido[3,4-f][1,3,5]triazepin-9-yl)morpholine

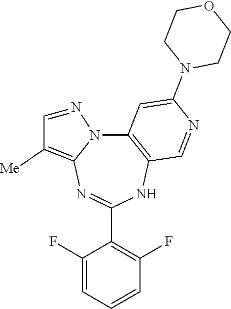 [SMILES: Cc1cnn2c1N=C(c1c(F)cccc1F)Nc1cnc(N3CCOCC3)cc1-2]

A mixture of 4-[5-bromo-8-(2,6-difluorophenyl)-2,3,7,9,12-pentazatricyclo[8.4.0.02,6]tetradeca-1(10),3,5,7,11,13-hexaen-13-yl]morpholine (Example 64) (80 mg, 0.173 mmol), potassium;trifluoro(methyl)boranuide (63.5 mg, 0.520 mmol), K₃PO₄ (110 mg, 0.520 mmol), Pd(OAc)₂ (3.89 mg, 17.3 μmol) and RuPhos (16.2 mg, 34.7 μmol) in dioxane (0.5 mL) and H₂O (0.1 mL) was degassed and purged with N₂ for 3 times. After stirring at 110 °C under N₂ atmosphere for 12 h, the reaction mixture was cooled to rt, diluted with THF (5 mL), and filtered through a small pad of celite. The resulting solution was concentrated under reduced pressure, and remaining residue was purified by prep-HPLC (column: 2_Phenomenex Gemini C18 75×40 mm×3 μm; mobile phase: [water (NH₄HCO₃)-MeCN]; B %: 35%-65%, 9.5 min) to provide 4-(5-(2,6-difluorophenyl)-3-methyl-6H-pyrazolo[1,5-a]pyrido[3,4-f][1,3,5]triazepin-9-yl)morpholine as a yellow solid (4.53 mg, 6.6%). ¹HNMR (400 MHz, CD₃OD) δ ppm 1.93 (s, 3H), 3.40-3.46 (m, 4H), 3.77-3.84 (m, 41H), 7.07-7.16 (m, 31H), 7.40 (s, 1H), 7.47-7.61 (m, 2H); LCMS (ESI) m/z 397.0 [M+H]⁺.

### Example 67

### Synthesis of 4-(5-(2,6-difluorophenyl)-3-fluoro-6H-pyrazolo[1,5-a]pyrido[3,4-f][1,3,5]triazepin-9-yl)morpholine

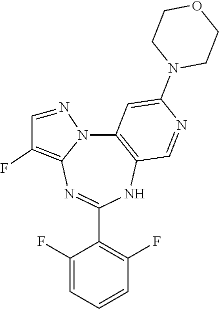 [SMILES: Fc1cccc(F)c1C1=Nc2c(F)cnn2-c2cc(N3CCOCC3)ncc2N1]

### Step 1

A solution of 1-(2-morpholino-5-nitropyridin-4-yl)-1H-pyrazol-5-amine (Intermediate 5) (500 mg, 1.72 mmol) and Select F (610 mg, 1.72 mmol) in MeCN (10 mL) was added stirred at 25 °C for 16 h. The resulting mixture was concentrated under reduced pressure. The remaining residue was purified by flash silica gel chromatography (ISCO®; 12 g SepaFlash® Silica Flash Column, petroleum ether/EtOAc with EtOAc from 0~50%, flow rate=50 mL/min, 254 nm) to provide 4-fluoro-2-(2-morpholino-5-nitro-4-pyridyl)pyrazol-3-amine as a yellow solid (150 mg, 22.0%). ¹HNMR (400 MHz, CDCl₃) ppm 3.28 (br s, 1H), 3.79 (br dd, J=16.1, 5.0 Hz, 8H), 6.61 (s, 1H), 7.53 (d. J=4.3 Hz, 1H), 8.93-9.05 (m, 1H); LCMS (ESI) m/z 308.9 [M+H]⁺.

### Step 2

To a 0 °C solution of 4-fluoro-2-(2-morpholino-5-nitro-4-pyridyl)pyrazol-3-amine (130 mg, 0.422 mmol) in THF (1 mL) under N₂ atmosphere was added NaBH₄ (95.7 mg, 2.53 mmol) and NiCl₂-6H₂O (200 mg, 0.843 mmol). After stirring at 0 °C for 1 h, the resulting mixture was treated with H₂O (10 mL) and extracted with EtOAc (10 mL×3). The combined organic layers were washed with brine (10 mL), dried with anhydrous Na₂SO₄, filtered and concentrated under reduced pressure. The remaining residue was purified by flash silica gel chromatography (ISCO®; 4 g SepaFlash® Silica Flash Column, petroleum ether/EtOAc with EtOAc from 0~100%, flow rate=60 mL/min, 254 nm) to provide 4-(5-amino-4-fluoro-pyrazol-1-yl)-6-morpholino-pyridin-3-amine as a white solid (60 mg, 51.1%). LCMS (ESI) m/z 279.0 [M+H]⁺.

### Step 3

A mixture of 4-(5-Amino-4-fluoro-pyrazol-1-yl)-6-morpholino-pyridin-3-amine (60 mg, 0.216 mmol), 2,6-difluorobenzaldehyde (36.8 mg, 0.259 mmol) and K₂CO₃ (89.4 mg, 0.647 mmol) in THF (0.5 mL) and t-BuOH (1 mL) was stirred at 50 °C for 16 h. The reaction mixture was cooled to rt, diluted with THF (5 mL), filtered and concentrated under reduced pressure. The remaining residue was purified by flash silica gel chromatography (ISCO®; 4 g SepaFlash® Silica Flash Column, petroleum ether/EtOAc with EtOAc from 0~45%, flow rate=40 mL/min, 254 nm) to provide 4-[8-(2,6-difluorophenyl)-5-fluoro-2,3,7.9,12-pentazatricyclo[8.4.0.02,6]tetradeca-1(10),3,5,11,13-pentaen-13-yl]morpholine as a white solid (50 mg 42.0%). LCMS (ESI) m/z 403.1 [M+H]⁺.

### Step 4

A solution of 4-[8-(2,6-difluorophenyl)-5-fluoro-2,3,7,9,12-pentazatricyclo[8.4.0.02,6]tetradeca-1(10),3,5,11,13-pentaen-13-yl]morpholine (45 mg, 0.112 mmol) and DDQ (27.9 mg, 0.123 mmol) in DCM (3 mL) was stirred at 25 °C for 3 h. The resulting solution was diluted with EtOAc (30 mL) and washed with saturated aqueous NaHCO₃ solution and brine (10 mL) subsequentially. The resulting organic solution was dried over anhydrous Na₂SO₄, filtered and concentrated under reduced pressure. The remaining residue was purified by flash silica gel chromatography (ISCO®; 4 g SepaFlash® Silica Flash Column, petroleum ether/EtOAc with EtOAc from 0~40%, flow rate=40 mL/min, 254 nm) to provide the desired title product of 4-[8-(2,6-difluorophenyl)-5-fluoro-2,3,7,9,12-pentazatricyclo[8.4.0.02,6]tetradeca-1(10),3,5,7,11,13-hexaen-13-yl]morpholine as a yellow solid (19.75 mg, 41.2%). ¹HNMR (400 MHz, CD₃OD) δ ppm 3.38-3.47 (m, 4H), 3.76-3.80 (m, 4H), 7.07-7.14 (m, 2H), 7.17 (s, 1H), 7.49-7.56 (m, 21H), 7.57-7.60 (m, 1H); LCMS (ESI) m/z 401.1 [M+H]⁺.

### Example 68

### Synthesis of 4-(3-chloro-5-(2,4,6-trifluorophenyl)-6H-pyrazolo[1,5-a]pyrido[3,4-f][1,3,5]triazepin-9-yl)morpholine

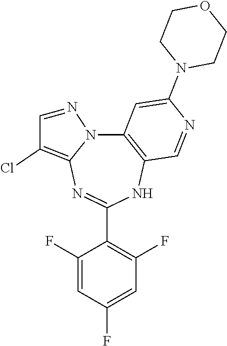 [SMILES: Fc1cc(F)c(C2=Nc3c(Cl)cnn3-c3cc(N4CCOCC4)ncc3N2)c(F)c1]

### Step 1

A mixture of 4-(5-amino-4-chloro-pyrazol-1-yl)-6-morpholino-pyridin-3-amine (Intermediate 6) (100 mg, 0.339 mmol), 2,4,6-trifluorobenzaldehyde (54.3 mg, 0.339 mmol) and K₂CO₃ (141 mg, 1.02 mmol) in THF (1.5 mL) and t-BuOH (6 mL) was stirred at 50 °C for 16 h. The reaction mixture was cooled to rt, filtered and concentrated under reduced pressure. The remaining residue was purified by flash silica gel chromatography (ISCOC; 4 g SepaFlash® Silica Flash Column, petroleum ether/EtOAc with EtOAc from 0~45%, flow rate=35 mL/min, 254 nm) to provide 4-[5-chloro-8-(2,4,6-trifluorophenyl)-2,3,7,9,12-pentazatricyclo[8.4.0.02,6]tetradeca-1(10),3,5,11,13-pentaen-13-yl]morpholine as a yellow solid (83 mg, 45.3%). LCMS (ESI) m/z 437.0 [M+H]⁺.

### Step 2

A mixture of 4-[5-chloro-8-(2,4,6-trifluorophenyl)-2,3,7,9,12-pentazatricyclo[8.4.0.02,6]tetradeca-1(10),3,5,11,13-pentaen-13-yl]morpholine (83 mg, 0.190 mmol) and DDQ (51.8 mg, 0.228 mmol) in DCM (5 mL) was stirred at 25 °C for 3 h. The reaction mixture was diluted with EtOAc (20) and washed with saturated aqueous NaHCO₃ solution. The resulting organic solution was washed with brine (50 mL), dried with anhydrous Na₂SO₄, filtered and concentrated under reduced pressure. The remaining residue was purified by flash silica gel chromatography (ISCO®; 4 g SepaFlash® Silica Flash Column, petroleum ether/EtOAc with EtOAc from 0~45%, flow rate=40 mL/min, 254 nm) to provide the desired title product of 4-[5-chloro-8-(2,4,6-trifluorophenyl)-2,3,7,9,12-pentazatricyclo[8.4.0.02,6]tetradeca-1(10),3,5,7,11,13-hexaen-13-yl]morpholine as a yellow solid (50.83 mg, 61.5%). ¹HNMR (400 MHz, DMSO-d₆) δ ppm 3.33-3.36 (m, 4H), 3.64-3.69 (m, 4H), 7.00 (s, 1H), 7.61 (s, 1H), 7.38 (t, J=8.7 Hz, 2H), 7.70 (s, 1H), 9.74 (s, 1H); LCMS (ESI) m/z 435.0 [M+H]⁺.

### Example 69

### Synthesis of 4-(3-chloro-5-(2-chloro-6-fluorophenyl)-6H-pyrazolo[1,5-a]pyrido[3,4-f][1,3,5]triazepin-9-yl)morpholine

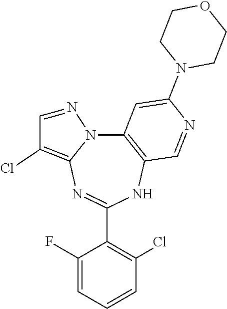 [SMILES: Fc1cccc(Cl)c1C1=Nc2c(Cl)cnn2-c2cc(N3CCOCC3)ncc2N1]

### Step 1

Into a rt mixture of 4-(5-amino-4-chloro-pyrazol-1-yl)-6-morpholino-pyridin-3-amine (Intermediate 6) (60 mg, 0.204 mmol) and K₂CO₃ (84.4 mg, 0.611 mmol) in t-BuOH (3 mL) and THF (1 mL) was added 2-Chloro-6-fluoro-benzaldehyde (32.3 mg, 0.204 mmol) in THF (0.4 mL) dropwise. After stirring at rt for 12 h, the resulting mixture was diluted with THF (10 mL) and the solution was filtered through a small pad of celite. The filtrate was concentrated under reduced pressure to give crude product of 4-(3-chloro-5-(2-chloro-6-fluorophenyl)-5,6-dihydro-4H-pyrazolo[1,5-a]pyrido[3,4-f][1,3,5]triazepin-9-yl)morpholine (30 mg, 30.5%, 90% purity) as a light yellow solid, which was used for the next step without further purification. LCMS (ESI) m/z 435.0 [M+H]⁺.

### Step 2

A mixture of 4-(3-chloro-5-(2-chloro-6-fluorophenyl)-5,6-dihydro-4H-pyrazolo[1,5-a]pyrido[3,4-f][1,3,5]triazepin-9-yl)morpholine (50 mg, 0.115 mmol) and DDQ (39.1 mg, 0.172 mmol) in DCM (2 mL) was stirred at 25 °C for 3 h. The resulting reaction mixture was treated with saturated aqueous NaHCO₃ (5 mL) solution, followed by extraction with DCM (15 mL×3). The combined organic layers were washed with brine (15 mL), dried over anhydrous Na₂SO₄, filtered and concentrated under reduced pressure. The remaining residue was purified by flash chromatography (ISCO®; 4 g SepaFlash® Silica Flash Column, petroleum ether/EtOAc with EtOAc from 0~60%, flow rate=30 mL/min, 254 nm) to give the desired title product of 4-(3-chloro-5-(2-chloro-6-fluorophenyl)-6H-pyrazolo[1,5-a]pyrido[3,4-f][1,3,5]triazepin-9-yl)morpholine as a yellow solid (20.5 mg, 38.8%). ¹HNMR (400 MHz, DMSO-d₆) δ ppm 3.39 (brs, 4H), 3.63-3.69 (m, 4H), 7.00 (s, 1H), 7.35-7.42 (m, 1H), 7.44-7.49 (m, 1H), 7.52-7.60 (m, 1H), 7.63 (s, 1H), 7.70 (s, 1H), 9.74 (s, 1H); LCMS (ESI) m/z 433.0 [M+H]⁺.

### Example 70

### Synthesis of (2S,6S)-4-[5-chloro-8-(2,6-difluorophenyl)-2,3,7,9,12-pentazatricyclo[8.4.0.02,6]tetradeca-1(10),3,5,7,11,13-hexaen-13-yl]-2,6-dimethyl-morpholine

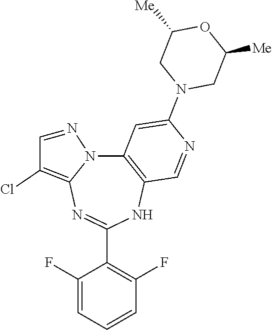 [SMILES: C[C@H]1CN(c2cc3c(cn2)NC(c2c(F)cccc2F)=Nc2c(Cl)cnn2-3)C[C@H](C)O1]

### Step 1

A solution of ethyl 5-amino-1-(2-bromo-5-nitro-4-pyridyl)pyrazole-4-carboxylate (Intermediate 7) (1.5 g, 4.21 mmol), (2S,6S)-2,6-dimethylmorpholine (485 mg, 4.21 mmol) and TEA (1.28 g, 12.6 mmol) in DCM (10 mL) was stirred at 25 °C for 16 h. The resulting mixture was poured into water (80 mL) and extracted with EtOAc (80 mL×3). The organic layers were combined, washed with water (50 mL) and brine (20 mL) subsequentially. The resulting organic solution was dried over anhydrous Na₂SO₄ and concentrated under reduced pressure. The remaining residue was purified by column chromatography (silica, petroleum ether/EtOAc=1/1, 254 nm) to afford ethyl 5-amino-1-[2-[(2S,6S)-2,6-dimethylmorpholin-4-yl]-5-nitro-4-pyridyl]pyrazole-4-carboxylate as a yellow solid (1.5 g, 90.3%). LCMS (ESI) m/z 391.1 [M+H]⁺.

### Step 2

A mixture of ethyl 5-amino-1-[2-[(2S,6S)-2,6-dimethylmorpholin-4-yl]-5-nitro-4-pyridyl]pyrazole-4-carboxylate (1.5 g, 3.84 mmol) and H₃PO₄ (7 mL, 85% purity) was stirred at 160 °C for 4.0 h. The reaction mixture was cooled to rt, treated with H₂O (30 mL), and 2.0 N NaOH (50 mL). The resulting mixture was extracted with EtOAc (30 mL×3). The combined organic layers were washed with brine (15 mL×2), dried with anhydrous Na₂SO₄, filtered and concentrated under reduced pressure. The remaining residue was purified by flash chromatography (ISCO®; 12 g SepaFlash® Silica Flash Column, petroleum ether/EtOAc with EtOAc from 0~100%, flow rate=45 mL/min, 254 nm) to afford 2-[2-[(2S,6S)-2,6-dimethylmorpholin-4-yl]-5-nitro-4-pyridyl]pyrazol-3-amine as yellow solid (840 mg, 68.6%). LCMS (ESI) m/z 319.0 [M+H]⁺.

### Step 3

A mixture of 2-[2-[(2S,6S)-2,6-dimethylmorpholin-4-yl]-5-nitro-4-pyridyl]pyrazol-3-amine (840 mg, 2.64 mmol) and NCS (387 mg, 2.90 mmol) in DCM (1 mL) was stirred at 25 °C for 2 h. The resulting mixture was treated with water (10 mL), and extracted with DCM (20 mL×2). The combined organic layers were washed with brine (10 mL), dried over anhydrous Na₂SO₄, filtered and concentrated under reduced pressure. The remaining residue was purified by flash chromatography (ISCO®; 12 g SepaFlash® Silica Flash Column, petroleum ether/EtOAc with EtOAc from 0~78%, flow rate=45 mL/min, 254 nm) to afford 4-chloro-2-[2-[(2S,6S)-2,6-dimethylmorpholin-4-yl]-5-nitro-4-pyridyl]pyrazol-3-amine as yellow solid (860 mg, 75.7%, 82% purity). LCMS (ESI) m/z 353.0 [M+H]⁺.

### Step 4

To a solution of 4-chloro-2-[2-[(2S,6S)-2,6-dimethylmorpholin-4-yl]-5-nitro-4-pyridyl]pyrazol-3-amine (860 mg, 2.44 mmol) in THF (8 mL) was added NaBH₄ (553 mg, 14.6 mmol) and NiCl₂.6H₂O (3.48 g, 14.6 mmol). After stirring at 25 °C for 2 h, and the resulting mixture was concentrated under reduced pressure to remove the volatiles, and the rest residue was redissolved in DCM (40 mL). The organic solution was washed with brine (15 mL), dried over anhydrous Na₂SO₄, filtered and concentrated under reduced pressure. The remaining residue was purified by flash chromatography (ISCO®; 12 g SepaFlash® Silica Flash Column, petroleum ether/EtOAc with EtOAc from 0~100%, flow rate=45 mL/min, 254 nm) to afford 4-(5-amino-4-chloro-pyrazol-1-yl)-6-[(2S,6S)-2,6-dimethylmorpholin-4-yl]pyridin-3-amine as brown solid (520 mg, 58.1%, 88% purity). LCMS (ESI) m/z 323.0 [M+H]⁺.

### Step 5

Into a rt solution of crude 2,6-difluorobenzaldehyde (88.0 mg, 619 umol) and K₂CO₃ (221 mg, 1.61 mmol) in THF (3 mL) and t-BuOH (7 mL) was slowly added a solution of 4-(5-amino-4-chloro-pyrazol-1-yl)-6-[(2S,6S)-2,6-dimethylmorpholin-4-yl]pyridin-3-amine (200 mg, 0.619 mmol) in THF (0.5 mL). After stirring at 60 °C for 16 h, the resulting reaction mixture was cooled to rt, treated with water (15 mL), and concentrated under reduced pressure to remove the organic volatiles. The remaining residue was extracted with DCM (20 mL×2). The combined organic layers were washed with brine (10 mL), dried over anhydrous Na₂SO₄, filtered and concentrated under reduced pressure to afford (2S,6S)-4-[5-chloro-8-(2,6-difluorophenyl)-2,3,7,9,12-pentazatricyclo[8.4.0.02,6]tetradeca-1(10),3,5,11,13-pentaen-13-yl]-2,6-dimethyl-morpholine (200 mg, crude) was obtained as brown oil. LCMS (ESI) m/z 323.0 [M+H]⁺.

### Step 6

A solution of (2S,6S)-4-[5-chloro-8-(2,6-difluorophenyl)-2,3,7,9,12-pentazatricyclo[8.4.0.02,6]tetradeca-1(10),3,5,11,13-pentaen-13-yl]-2,6-dimethyl-morpholine (200 mg, 0.447 mmol) and DDQ (121 mg, 0.537 mmol) in DCM (1 mL) was stirred at 25 °C for 48 h. The reaction mixture was treated with water (15 mL), and extracted with DCM (20 mL×2). The combined organic layers were washed with brine (20 mL), dried over anhydrous Na₂SO₄, filtered and concentrated under reduced pressure. The remaining residue was purified by flash chromatography (ISCO®; 12 g SepaFlash® Silica Flash Column, petroleum ether/EtOAc with EtOAc from 0~45%, flow rate=45 mL/min, 254 nm) to afford the title product of (2S,6S)-4-[5-chloro-8-(2,6-difluorophenyl)-2,3,7,9,12-pentazatricyclo[8.4.0.02,6]tetradeca-1(10),3,5,7,11,13-hexaen-13-yl]-2,6-dimethyl-morpholine as yellow solid (83 mg, 40.7%). ¹HNMR (400 MHz, DMSO-d₆) δ ppm 1.14 (d, J=6.3 Hz, 6H), 3.11 (dd, J=6.3, 12.5 Hz, 2H), 3.47 (dd, J=3.3, 12.5 Hz, 2H), 4.06-3.92 (m, 2H), 6.97 (s, 1H), 7.30-7.20 (m, 2H), 7.64-7.54 (m, 2H), 7.69 (s, 1H), 9.73 (s, 1H); LCMS (ESI) m/z 445.2 [M+H]⁺.

### Example 71

### Synthesis of (S)-4-(5-(2,6-difluorophenyl)-6H-pyrazolo[1,5-a]pyrido[3,4-f][1,3,5]triazepin-9-yl)-2-methylmorpholine

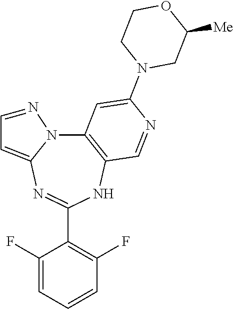 [SMILES: C[C@H]1CN(c2cc3c(cn2)NC(c2c(F)cccc2F)=Nc2ccnn2-3)CCO1]

### Step 1

A solution of ethyl 5-amino-1-(2-bromo-5-nitro-4-pyridyl)pyrazole-4-carboxylate (Intermediate 7) (5 g, 14.0 mmol), TEA (4.26 g, 42.1 mmol) and (2S)-2-methylmorpholine (1.93 g, 14.0 mmol) in DCM (30 mL) was stirred at 25 °C for 16 h. The resulting mixture was treated with H₂O (30 mL), and extracted with EtOAc (60 mL×3). The combined organic layers were washed with brine (30 mL), dried with anhydrous Na₂SO₄, filtered and concentrated under reduced pressure. The remaining residue was purified by flash silica gel chromatography (ISCO®; 40 g SepaFlash® Silica Flash Column, petroleum ether/EtOAc with EtOAc from 0~50%, flow rate=50 mL/min, 254 nm) to provide ethyl 5-amino-1-[2-[(2S)-2-methylmorpholin-4-yl]-5-nitro-4-pyridyl]pyrazole-4-carboxylate as a yellow solid (5 g, 94.6%). LCMS (ESI) m/z 377.1 [M+H]⁺.

### Step 2

A mixture of ethyl 5-amino-1-[2-[(2S)-2-methylmorpholin-4-yl]-5-nitro-4-pyridyl]pyrazole-4-carboxylate (2.45 g, 6.51 mmol) in H₃PO₄ (20 mL, 85 wt %) was stirred at 160 °C for 4 h. The reaction mixture was cooled to rt, and treated with H₂O (50 mL). The resulting mixture was neutralized to pH=78 with saturated aqueous NaHCO₃ solution, and extracted with EtOAc (100 mL×3). The combined organic layers were washed with brine (50 mL), dried with anhydrous Na₂SO₄, filtered and concentrated under reduced pressure. The remaining residue was purified by flash silica gel chromatography (ISCO®; 20 g SepaFlash® Silica Flash Column, petroleum ether/EtOAc with EtOAc from 0~50%, flow rate=50 mL/min, 254 nm) to provide 2-[2-[(2S)-2-methylmorpholin-4-yl]-5-nitro-4-pyridyl]pyrazol-3-amine as a yellow solid (1.9 g, 45.5%). LCMS (ESI) m/z 304.9 [M+H]⁺.

### Step 3

A mixture of 2-[2-[(2S)-2-methylmorpholin-4-yl]-5-nitro-4-pyridyl]pyrazol-3-amine (350 mg, 1.15 mmol) and Pd/C (50 mg, 10 wt % Pd with 50 wt % water) in THF (5 mL) was purged with H₂ three time. After stirring under H₂ atmosphere (15 psi) at 25 °C for 4 h, the resulting reaction mixture was filtered and concentrated under reduced pressure to provide 4-(5-aminopyrazol-1-yl)-6-[(2S)-2-methylmorpholin-4-yl]pyridin-3-amine as brown oil (300 mg, 90.3%) which was used into next step without purification. LCMS (ESI) m/z 274.9 [M+H]⁺.

### Step 4

A mixture of 4-(5-aminopyrazol-1-yl)-6-[(2S)-2-methylmorpholin-4-yl]pyridin-3-amine (300 mg, 1.09 mmol), K₂CO₃ (151 mg, 1.09 mmol) and 2,6-difluorobenzaldehyde (171 mg, 1.20 mmol) in THF (1 mL) and t-BuOH (3 mL) was stirred at 50 °C for 16 h. The reaction mixture was cooled to rt, diluted with THF (10 mL), and filtered through a small pad of celite. The filtrate was concentrated under reduced pressure. The remaining residue was purified by flash silica gel chromatography (ISCO®; 12 g SepaFlash® Silica Flash Column, petroleum ether/EtOAc with EtOAc from 0~50%, flow rate=50 mL/min, 254 nm) to provide (2S)-4-[8-(2,6-difluorophenyl)-2,3,7,9,12-pentazatricyclo[8.4.0.02,6]tetradeca-1(10),3,5,11,13-pentaen-13-yl]-2-methyl-morpholine as a yellow solid (400 mg, 61.5%). LCMS (ESI) m/z 399.2 [M+H]⁺.

### Step 5

A mixture of (2S)-4-[8-(2,6-difluorophenyl)-2,3,7,9,12-pentazatricyclo[8.4.0.02,6]tetradeca-1(10),3,5,11,13-pentaen-13-yl]-2-methyl-morpholine (200 mg, 0.502 mmol) and DDQ (125 mg, 0.552 mmol) in DCM (8 mL) was stirred at 25 °C for 3 h. The resulting reaction mixture was diluted with EtOAc (60 mL) and washed with saturated aqueous NaHCO₃ solution. The organic solution were further washed with brine (12 mL), dried with anhydrous Na₂SO₄, filtered and concentrated under reduced pressure. The remaining residue was purified by flash silica gel chromatography (ISCO®; 20 g SepaFlash® Silica Flash Column, petroleum ether/EtOAc with EtOAc from 0~50%, flow rate=50 mL/min, 254 nm) to provide the title product of (2S)-4-[8-(2,6-difluorophenyl)-2,3,7,9,12-pentazatricyclo[8.4.0.02,6]tetradeca-1(10),3,5,7,11,13-hexaen-13-yl]-2-methyl-morpholine as a yellow solid (54.76 mg, 27.5%). ¹HNMR (400 MHz, CD₃OD) δ ppm 1.23 (d, J=6.3 Hz, 3H), 2.55 (brt, J=11.4 Hz, 1H), 2.83-2.99 (m, 1H), 3.60-3.74 (m, 2H), 3.85-4.11 (m, 3H), 5.93 (s, 1H), 7.06-7.20 (m, 3H), 7.44-7.60 (m, 3H); LCMS (ESI) m/z 397.0 [M+H]V.

### Example 72

### Synthesis of (2S,6S)-4-(5-(2,6-difluorophenyl)-3-fluoro-6H-pyrazolo[1,5-a]pyrido[3,4-f][1,3,5]triazepin-9-yl)-2,6-dimethylmorpholine

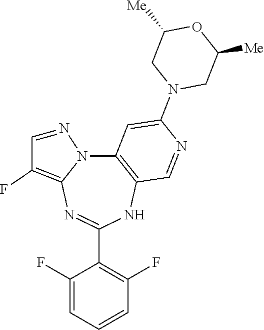 [SMILES: C[C@H]1CN(c2cc3c(cn2)NC(c2c(F)cccc2F)=Nc2c(F)cnn2-3)C[C@H](C)O1]

### Step 1

A solution of ethyl 5-amino-1-(2-bromo-5-nitro-4-pyridyl)pyrazole-4-carboxylate (Intermediate 7) (3.09 g, 8.68 mmol), TEA (2.64 g, 26.1 mmol) and (2S,6S)-2,6-dimethylmorpholine (1 g, 8.68 mmol) in DCM (30 mL) was stirred at 25 °C for 16 h. The reaction mixture was treated with H₂O (30 mL) and extracted with EtOAc (50 mL×3). The combined organic layers were washed with brine (30 mL), dried with anhydrous Na₂SO₄, filtered and concentrated under reduced pressure. The remaining residue was purified by flash silica gel chromatography (ISCO®; 20 g SepaFlash® Silica Flash Column, petroleum ether/EtOAc with EtOAc from 0~60%, flow rate=50 mL/min, 254 nm) to provide ethyl 5-amino-1-[2-[(2S,6S)-2,6-dimethylmorpholin-4-yl]-5-nitro-4-pyridyl]pyrazole-4-carboxylate as a yellow solid (3.15 g, 92.9%). LCMS (ESI) m/z 391.1 [M+H]⁺.

### Step 2

A solution of ethyl 5-amino-1-[2-[(2S,6S)-2,6-dimethylmorpholin-4-yl]-5-nitro-4-pyridyl]pyrazole-4-carboxylate (1.5 g, 3.84 mmol) in H₃PO₄ (10 mL, 85 wt %) was stirred at 160 °C for 4 h. The reaction mixture was cooled to it, treated with H₂O (50 mL) and neutralized to pH=7-8 with saturated aqueous NaHCO₃ solution. The resulting mixture was extracted with EtOAc (100 mL×3). The combined organic layers were washed with brine (50 mL), dried with anhydrous Na₂SO₄, filtered and concentrated under reduced pressure. The remaining residue was purified by flash silica gel chromatography (ISCO®; 20 g SepaFlash® Silica Flash Column, petroleum ether/EtOAc with EtOAc from 0~60%, flow rate=60 mL/min, 254 nm) to provide 2-[2-[(2S,6S)-2,6-dimethylmorpholin-4-yl]-5-nitro-4-pyridyl]pyrazol-3-amine as a white solid (1.5 g, 57.6%). LCMS (ESI) m/z 319.0 [M+H]⁺.

### Step 3

A mixture of 2-[2-[(2S,6S)-2,6-dimethylmorpholin-4-yl]-5-nitro-4-pyridyl]pyrazol-3-amine (500 mg, 1.57 mmol) and Select F (556 mg, 1.57 mmol) in THF (10 mL) was stirred at 60 °C for 16 h. The reaction mixture was cooled to it, treated with H₂O (50 mL) and extracted with EtOAc (100 mL×3). The combined organic layers were washed with brine (20 mL), dried with anhydrous Na₂SO₄, filtered and concentrated under reduced pressure. The remaining residue was purified by flash silica gel chromatography (ISCO®; 12 g SepaFlash® Silica Flash Column, petroleum ether/EtOAc with EtOAc from 0~50%, flow rate=50 mL/min, 254 nm) to provide 2-[2-[(2S,6S)-2,6-dimethylmorpholin-4-yl]-5-nitro-4-pyridyl]-4-fluoro-pyrazol-3-amine as a yellow solid (350 mg, 58.3%). ¹HNMR (400 MHz, CDCl₃) δ ppm 1.26 (d, J=6.4 Hz, 6H), 3.29 (br s, 2H), 3.41-3.59 (m, 2H), 3.83 (br d, J=11.0 Hz, 2H), 4.06-4.21 (m, 2H), 6.57 (s, 1H), 7.53 (d, J=4.3 Hz, 1H), 8.94-9.02 (m, 1H); LCMS (ESI) m/z 337.0 [M+H]⁺.

### Step 4

Into a 0 °C solution of 2-[2-[(2S,6S)-2,6-dimethylmorpholin-4-yl]-5-nitro-4-pyridyl]-4-fluoro-pyrazol-3-amine (300 mg, 0.892 mmol) in THF (1 mL) under N₂ atmosphere was added NiCl₂-6H₂O (42 mg, 1.78 mmol) and NaBH₄ (135 mg, 3.57 mmol). After stirring at 0 °C for 1.0 h, the reaction mixture was treated with H₂O (10 mL) and extracted with EtOAc (50 mL×3). The combined organic layers were washed with brine (30 mL), dried with anhydrous Na₂SO₄, filtered and concentrated under reduced pressure to provide crude product of 4-(5-amino-4-fluoro-pyrazol-1-yl)-6-[(2S,6S)-2,6-dimethylmorpholin-4-yl]pyridin-3-amine as a brown solid (230 mg, 79.9%) that was carried to next step without further purification. LCMS (ESI) m/z 307.1 [M+H]⁺.

### Step 5

Into a solution of crude 4-(5-amino-4-fluoro-pyrazol-1-yl)-6-[(2S,6S)-2,6-dimethylmorpholin-4-yl]pyridin-3-amine (230 mg, 0.751 mmol) and K₂CO₃ (104 mg, 0.751 mmol) in THF (2.5 mL) and t-BuOH (3 mL) was added a solution of 2,6-difluorobenzaldehyde (117 mg, 0.826 mmol) in THF (0.3 mL). After stirring at 50 °C for 16 h, the resulting mixture was cooled to rt, diluted with THF (10 mL), filtered through a small pad of celite. The filtrate was concentrated under reduced pressure. The remaining residue was purified by flash silica gel chromatography (ISCO®; 12 g SepaFlash® Silica Flash Column, petroleum ether/EtOAc with EtOAc from 0~50%, flow rate=50 mL/min, 254 nm) to provide (2S,6S)-4-[8-(2,6-difluorophenyl)-5-fluoro-2,3,7,9,12-pentazatricyclo[8.4.0.02,6]tetradeca-1(10),3,5,11,13-pentaen-13-yl]-2,6-dimethyl-morpholine as a yellow solid (300 mg, 70.5%). LCMS (ESI) m/z 431.2 [M+H]⁺.

### Step 6

A solution of (2S,6S)-4-[8-(2,6-difluorophenyl)-5-fluoro-2,3,7,9,12-pentazatricyclo[8.4.0.02,6]tetradeca-1(10),3,5,11,13-pentaen-13-yl]-2,6-dimethyl-morpholine (300 mg, 0.697 mmol) and DDQ (174 mg, 0.767 mmol) in DCM (6 mL) was stirred at 25 °C for 3 h. The reaction mixture was diluted with EtOAc (100 mL) and washed with saturated aqueous NaHCO₃ solution. The resulting solution were washed with brine (20 mL), dried with anhydrous Na₂SO₄, filtered and concentrated under reduced pressure. The remaining residue was purified by flash silica gel chromatography (ISCO®; 12 g SepaFlash® Silica Flash Column, petroleum ether/EtOAc with EtOAc from 0~50%, flow rate=50 mL/min, 254 nm) to provide the desired product of (2S,6S)-4-[8-(2,6-difluorophenyl)-5-fluoro-2,3,7,9,12-pentazatricyclo[8.4.0.02,6]tetradeca-1(10),3,5,7,11,13-hexaen-13-yl]-2,6-dimethyl-morpholine as a yellow solid (183.18 mg, 58.9%). ¹HNMR (400 MHz, DMSO-d₆) δ ppm 1.14 (d, J=6.3 Hz, 6H), 3.10 (br dd, J=12.6, 6.3 Hz, 2H), 3.47 (br dd, J=12.4, 3.1 Hz, 2H), 3.94-4.05 (m, 2H), 7.00 (s, 1H), 7.24 (br t, J=8.2 Hz, 2H), 7.54-7.61 (m, 1H), 7.63 (s, 1H), 7.71 (d, J=4.3 Hz, 1H), 9.66 (s, 1H); LCMS (ESI) m/z 429.1 [M+H]⁺.

### Example 73

### Synthesis of (S)-4-(5-(2,6-difluorophenyl)-3-fluoro-6H-pyrazolo[1,5-a]pyrido[3,4-f][1,3,5]triazepin-9-yl)-2-methylmorpholine

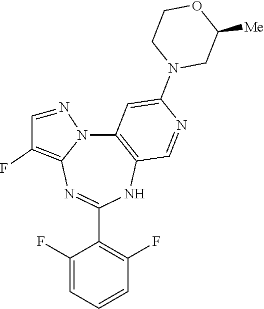 [SMILES: C[C@H]1CN(c2cc3c(cn2)NC(c2c(F)cccc2F)=Nc2c(F)cnn2-3)CCO1]

The title compound was made from ethyl 5-amino-1-(2-chloro-5-nitropyridin-4-yl)-1H-pyrazole-4-carboxylate (Intermediate 4), following a 6-step synthetic procedure as described for the synthesis of (2S,6S)-4-(5-(2,6-difluorophenyl)-3-fluoro-6H-pyrazolo[1,5-a]pyrido[3,4-f][1,3,5]triazepin-9-yl)-2,6-dimethylmorpholine (Example 72), except that (2S)-2-methylmorpholine was used in the place of (2S, 6S)-2,6-dimethylmorpholine (step 1). The crude final product (step 6) was purified by flash silica gel chromatography (ISCO®; 12 g SepaFlash® Silica Flash Column, petroleum ether/EtOAc with EtOAc from 0~50%, flow rate=50 mL/min, 254 nm) to provide the desired product of (S)-4-(5-(2,6-difluorophenyl)-3-fluoro-6H-pyrazolo[1,5-a]pyrido[3,4-f][1,3,5]triazepin-9-yl)-2-methylmorpholine as a yellow solid (50.74 mg, 26.9%, 95% purity). ¹HNMR (400 MHz, CD₃OD) δ ppm 1.21 (d, J=6.3 Hz, 3H), 2.53 (dd, J=12.6, 10.5 Hz, 1H), 2.87 (td, J=12.2, 3.5 Hz, 1H), 3.58-3.71 (m, 2H), 3.85-4.00 (m, 3H), 7.06-7.13 (m, 2H), 7.15 (s, 1H), 7.47-7.55 (m, 2H), 7.56 (s, 1H); LCMS (ESI) m/z 415.0 [M+H]⁺.

### Example 74

### Synthesis of (2S,6S)-4-(5-(2,6-difluorophenyl)-6H-pyrazolo[1,5-a]pyrido[3,4-f][1,3,5]triazepin-9-yl)-2,6-dimethylmorpholine

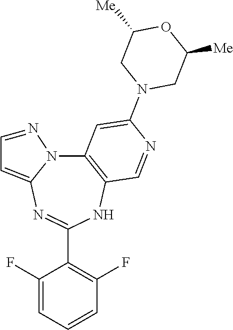 [SMILES: C[C@H]1CN(c2cc3c(cn2)NC(c2c(F)cccc2F)=Nc2ccnn2-3)C[C@H](C)O1]

A suspension of (2S,6S)-4-[5-chloro-8-(2,6-difluorophenyl)-2,3,7,9,12-pentazatricyclo [8.4.0.02,6]tetradeca-1(10),3,5,7,11,13-hexaen-13-yl]-2,6-dimethyl-morpholine (Example 70) (53 mg, 0.119 mmol) and Pd/C (126 mg, 10 wt % Pd with 50 wt % water) in THF (1 mL) was degassed and purged with H₂ for 3 times. The resulting mixture was stirred under H₂ atmosphere (15 psi) at 25 °C for 16 h. The reaction mixture was treated with H₂O (10 mL) and extracted with EtOAc (12 mL×3). The combined organic layers were washed with brine (12 mL), dried with anhydrous Na₂SO₄, filtered and concentrated under reduced pressure. The remaining residue was purified by preparative TLC (silica, PE/EA=1/1, 254 nm) to provide the title compound as a yellow solid (19.17 mg, 38.8%, 99% purity). ¹HNMR (400 MHz, DMSO-d₆) δ ppm 1.15 (br d, J=6.3 Hz, 6H), 3.07-3.12 (br dd, J=6.1, 12.4 Hz, 2H), 3.55 (br d, J=12.0 Hz, 3H), 4.00 (br, 2H), 5.92 (s, 1H), 7.01 (s, 1H), 7.22 (br t, J=8.0 Hz, 2H), 7.72-7.44 (m, 3H), 9.51 (s, 1H); LCMS (ESI) m/z 411.2 [M+H]⁺.

### Example 75

The compounds listed in Table 2 were tested for their inhibitory effect on LRRK2 kinase according to the following procedures.

### Biochemical Assays:

The protocol for basic TR-FRET LanthaScreen Tb Kinase Activity Assay inhibitor studies were as follows. LanthaScreen Kinase Activity Assays (ThermoFisher/USA) to evaluate inhibitors were performed by addition of 100 nl of test compound in corresponding DMSO dilutions/5 μl of kinase/fluorescein-ERM(LRRKtide) peptide mixture, Compound & kinase were pre-incubated for 15 minutes, then 5 μl of ATP into 384 well small volume plates. After incubation for 120 minutes at room temperature, the detection reagents containing Tb-anti-pLRRKtide antibody were added to monitor phosphorylation level of peptide. Then, after 60 min minutes incubation at room temperature plates were read in Envision. Data analysis of emission ratios was according to LanthaScreen Tb Kinase Activity Assay protocol.

Kinase and assay components were adjusted to final concentrations according to the kit protocol. For LRRK2: 2 nM wt human LRRK2, catalytic site, catalytic site (ThermoFisher/USA), 400 nM peptide, 38 M ATP in 1× Kinase Buffer A.

Basic protocol for TR-FRET LanthaScreen Tb Kinase Activity Assay inhibitor studies involved two steps:

Enzymatic step: Addition of 100 μL of test compound in corresponding DMSO dilutions, 5 μL of kinase/substrate mixture, 5 μL of ATP into 384 well small volume plates. Incubation for 120 minutes at room temperature.

Detection step: Addition of 10 μL EDTA & antibody, read plate after 60 minutes. Data analysis of emission ratios according to KinEASE assay protocol.

Kinase and assay components were adjusted to final concentrations according to the kit protocol.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification, and guidance that can be adapted to the practice of this invention in its various embodiments and equivalents thereof.

1. A compound of Formula (I): 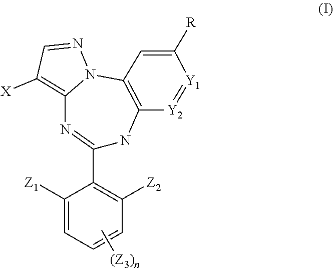
or an enantiomer, mixture of enantiomers, tautomer, or pharmaceutically acceptable salt thereof,
wherein:
n is 1, 2, or 3;
Y₁ and Y₂ are independently N or CH;
Z₁, Z₂, and Z₃ are independently selected from H, —OH, halo, C₁-C₆ alkyl, C₁-C₆ heteroalkyl, haloalkyl, alkoxy, haloalkxoxy, —CH(OH)-alkyl, hydroxyalkyl, hydroxyalkoxy, amino, cyano, or morpholino;
X is H, —OH, halo, cyano, alkyl, haloalkyl, cyanoalkyl, or cycloalkyl with one or more heteroatoms, wherein the heteroatoms are selected from a group consisting of N, O, or S;
R is H, halo, substituted or unsubstituted aryl, substituted or unsubstituted C₁-C₆-alkyl, branched alkyl, alkenyl, alkynyl, haloalkyl, alkoxy, cycloalkoxy, haloalkoxy, -cyano, —CH₂-cycloalkyl, —CF₂-cycloalky, —CH(CH₃)-cycloalkyl, —CH₂-aryl, —CF₂-aryl, —CH(—CH₃)-aryl, C(═O)-alkyl, —C(═O)cycloalkyl, —C(═O)—NH-alkyl, —COOH (and esters and carboxamides thereof), C(═O)—NH—S(═O)₂alkyl, C(═O)—NH—S(═O)₂aryl, —C(═O)-morpholine, —C(═O)-heterocycles, —C(—CH₃)₂—OH, —CH₂—C(═O)—NH₂; -hydroxy, alkylhydroxy, alkyl-COOH (and esters and carboxamides thereof), substituted or unsubstituted alkylsulfonyl, arylsulfonyl, amino, —NHC(═O)alkyl, —N(alkyl)C(═O)alkyl, —NHC(═O)aryl, —N(alkyl)C(═O)aryl, —N(H)S(═O)₂alkyl, —N(H)S(═O)₂aryl, —N(alkyl)S(═O)₂alkyl, cyanoalkyl, haloalkyl, —S═(O)₂-alkyl, —S═(O)₂-cycloalkyl, —S═(O)₂-aryl, —S(═O)₂N(H)alkyl, substituted or unsubstituted morpholine, 3-7 membered heterocycle, any of which may have one or more substituents, 3-7 membered cycloalkyl or heterocycle, wherein the 3-7 membered cycloalkyl or heterocycle is fused with another 3-7 cycloalkyl or heterocycle, wherein the rings are spiro, bridged bicyclic, or spiro, wherein the at least one heteroatom in the heterocycle rings are independently selected from O, S, and N;
one or more of hydrogens atom is optionally deuterium; and
when Y₁ is CH and Y₂ is CH; R is not H, fluoro, chloro, or methyl.

2. The compound of claim 1, wherein Y₁ is N and Y₂ is CH.

3. The compound of claim 1, wherein Y₁ is CH and Y₂ is N.

4. The compound of claim 1, wherein Z₁ and Z₂ are fluoro and Z₃ is H.

5. The compound of claim 1, wherein Z₁ and Z₂ are fluoro and chloro respectively, and Z₃ is H.

6. The compound of claim 1, wherein Z₁, Z₂, and Z₃ are fluoro.

7. The compound of claim 1, wherein R is selected from a group consisting of: chloro, hydrogen, bromo, fluoro, methyl, methylcyano, and morpholine.

8. The compound of claim 1, wherein X is H, bromo, chloro, fluoro, or methyl.

9. The compound of claim 1, wherein R is selected from a group consisting of substituted or unsubstituted morpholines, amides, piperazines, alkyls, azetidine, heterocycle, or heteroaryl.

10. The compound of claim 1, wherein R is not any of H, fluoro, chloro, or methyl.

11. The compound of claim 1, wherein X is not any of H, fluoro, chloro, or cyano.

12. The compound of claim 1, wherein R is selected from a group consisting of: H, bromo, amide, ester, carboxylic acid,

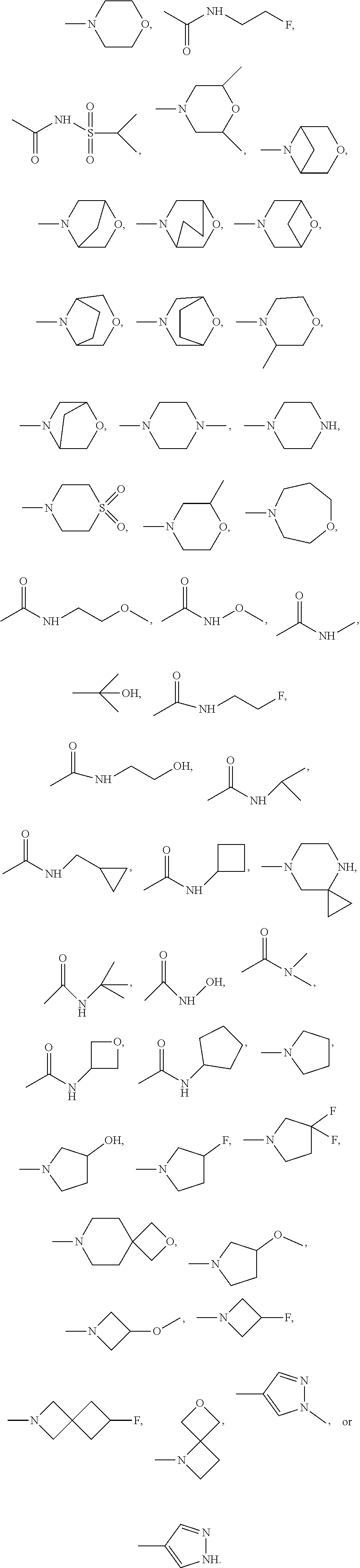

13. The compound of claim 1, wherein the compound is selected from:

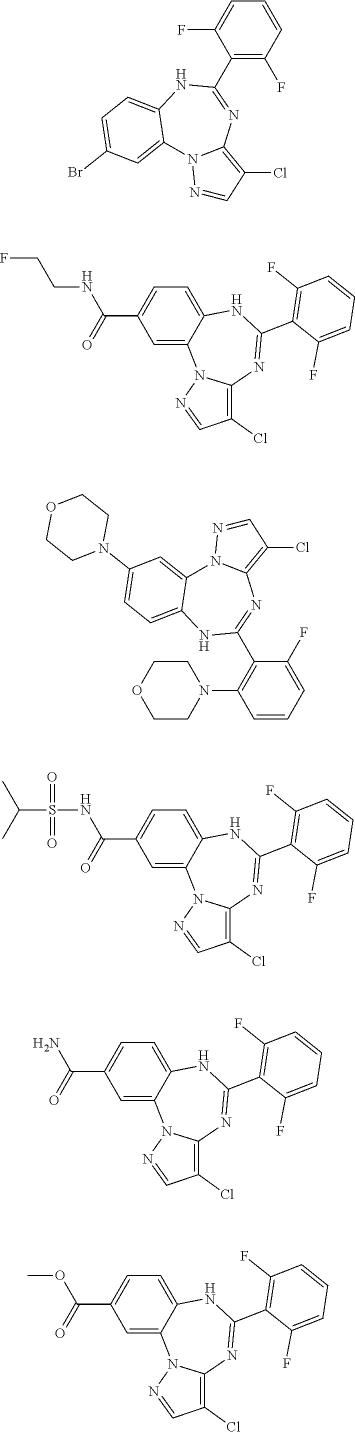 [SMILES: Fc1cccc(F)c1C1=Nc2c(Cl)cnn2-c2cc(Br)ccc2N1 ; O=C(NCCF)c1ccc2c(c1)-n1ncc(Cl)c1N=C(c1c(F)cccc1F)N2 ; Fc1cccc(N2CCOCC2)c1C1=Nc2c(Cl)cnn2-c2cc(N3CCOCC3)ccc2N1 ; CC(C)S(=O)(=O)NC(=O)c1ccc2c(c1)-n1ncc(Cl)c1N=C(c1c(F)cccc1F)N2 ; NC(=O)c1ccc2c(c1)-n1ncc(Cl)c1N=C(c1c(F)cccc1F)N2 ; COC(=O)c1ccc2c(c1)-n1ncc(Cl)c1N=C(c1c(F)cccc1F)N2]

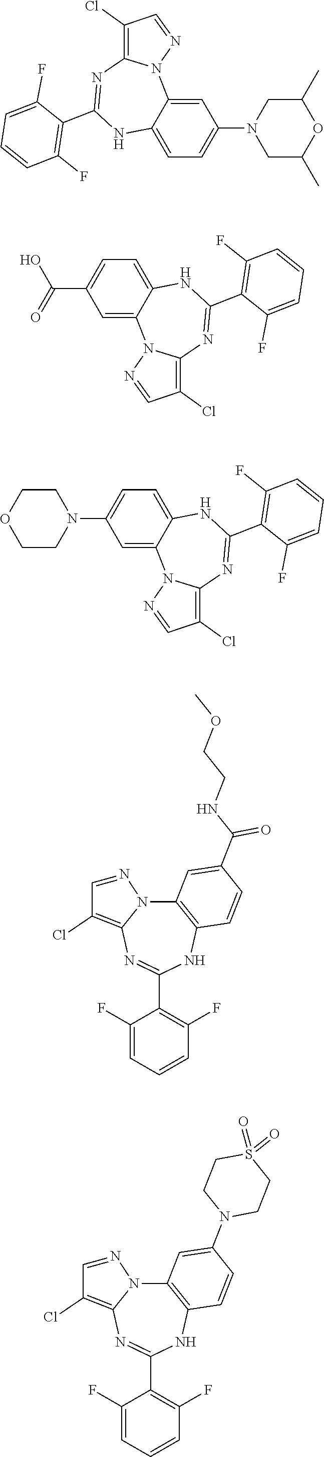 [SMILES: CC1CN(c2ccc3c(c2)-n2ncc(Cl)c2N=C(c2c(F)cccc2F)N3)CC(C)O1 ; O=C(O)c1ccc2c(c1)-n1ncc(Cl)c1N=C(c1c(F)cccc1F)N2 ; Fc1cccc(F)c1C1=Nc2c(Cl)cnn2-c2cc(N3CCOCC3)ccc2N1 ; COCCNC(=O)c1ccc2c(c1)-n1ncc(Cl)c1N=C(c1c(F)cccc1F)N2 ; O=S1(=O)CCN(c2ccc3c(c2)-n2ncc(Cl)c2N=C(c2c(F)cccc2F)N3)CC1]

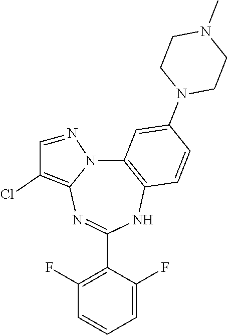 [SMILES: CN1CCN(c2ccc3c(c2)-n2ncc(Cl)c2N=C(c2c(F)cccc2F)N3)CC1]

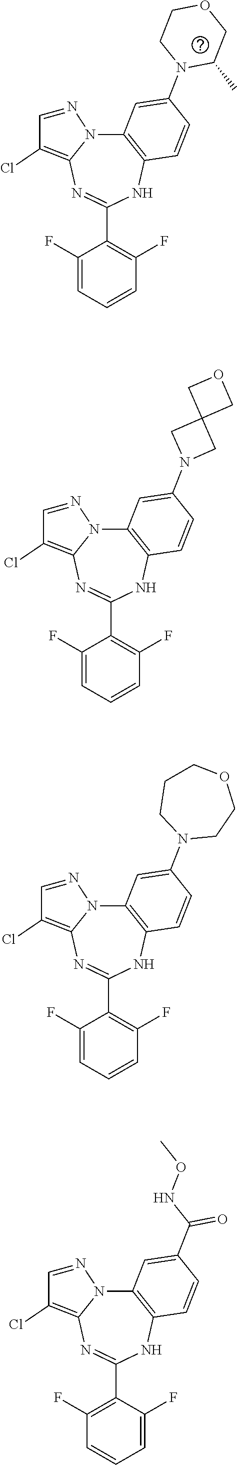 [SMILES: C[C@H]1COCCN1c1ccc2c(c1)-n1ncc(Cl)c1N=C(c1c(F)cccc1F)N2 ; Fc1cccc(F)c1C1=Nc2c(Cl)cnn2-c2cc(N3CC4(COC4)C3)ccc2N1 ; Fc1cccc(F)c1C1=Nc2c(Cl)cnn2-c2cc(N3CCCOCC3)ccc2N1 ; CONC(=O)c1ccc2c(c1)-n1ncc(Cl)c1N=C(c1c(F)cccc1F)N2]

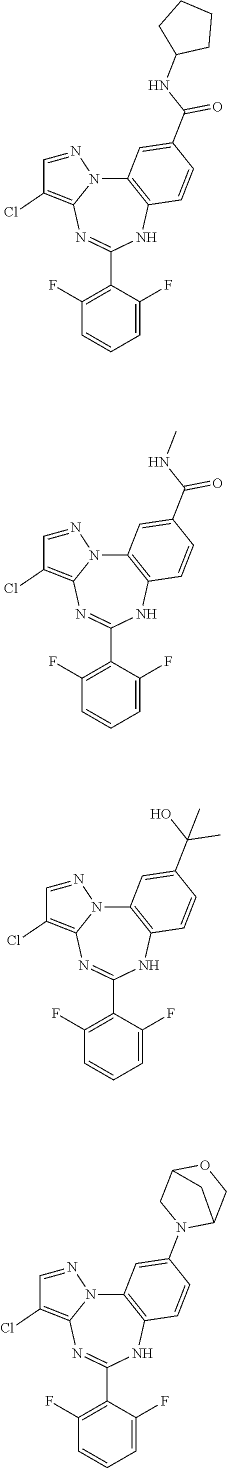 [SMILES: O=C(NC1CCCC1)c1ccc2c(c1)-n1ncc(Cl)c1N=C(c1c(F)cccc1F)N2 ; CNC(=O)c1ccc2c(c1)-n1ncc(Cl)c1N=C(c1c(F)cccc1F)N2 ; CC(C)(O)c1ccc2c(c1)-n1ncc(Cl)c1N=C(c1c(F)cccc1F)N2 ; Fc1cccc(F)c1C1=Nc2c(Cl)cnn2-c2cc(N3CC4CC3CO4)ccc2N1]

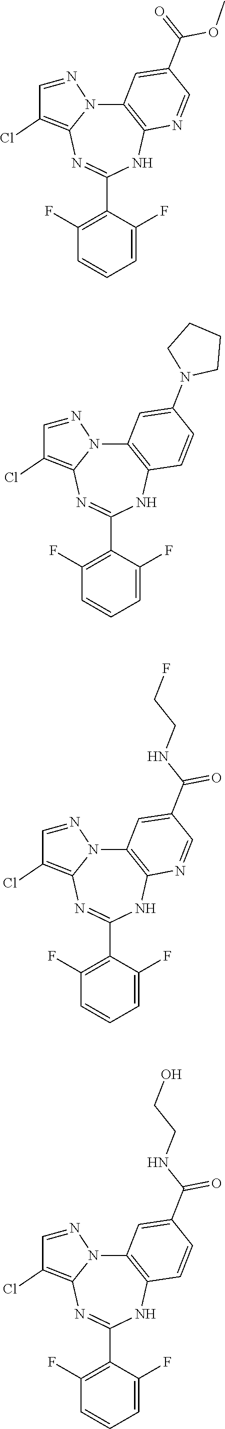 [SMILES: COC(=O)c1cnc2c(c1)-n1ncc(Cl)c1N=C(c1c(F)cccc1F)N2 ; Fc1cccc(F)c1C1=Nc2c(Cl)cnn2-c2cc(N3CCCC3)ccc2N1 ; O=C(NCCF)c1cnc2c(c1)-n1ncc(Cl)c1N=C(c1c(F)cccc1F)N2 ; O=C(NCCO)c1ccc2c(c1)-n1ncc(Cl)c1N=C(c1c(F)cccc1F)N2]

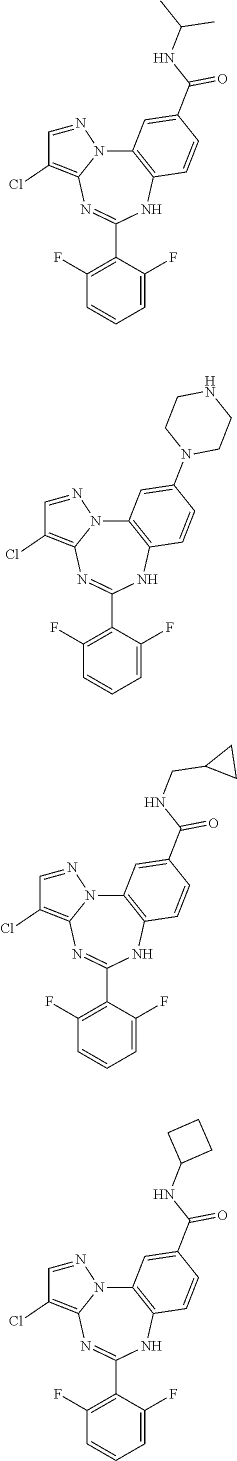 [SMILES: CC(C)NC(=O)c1ccc2c(c1)-n1ncc(Cl)c1N=C(c1c(F)cccc1F)N2 ; Fc1cccc(F)c1C1=Nc2c(Cl)cnn2-c2cc(N3CCNCC3)ccc2N1 ; O=C(NCC1CC1)c1ccc2c(c1)-n1ncc(Cl)c1N=C(c1c(F)cccc1F)N2 ; O=C(NC1CCC1)c1ccc2c(c1)-n1ncc(Cl)c1N=C(c1c(F)cccc1F)N2]

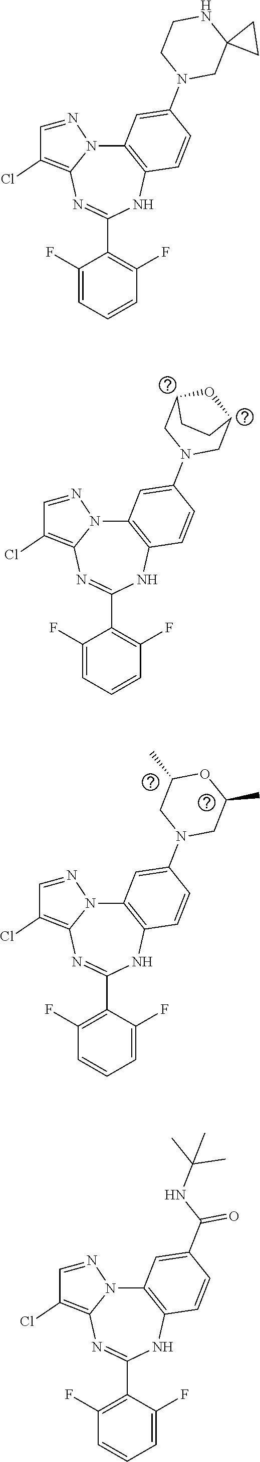 [SMILES: Fc1cccc(F)c1C1=Nc2c(Cl)cnn2-c2cc(N3CCNC4(CC4)C3)ccc2N1 ; Fc1cccc(F)c1C1=Nc2c(Cl)cnn2-c2cc(N3C[C@H]4CC[C@@H](C3)O4)ccc2N1 ; C[C@H]1CN(c2ccc3c(c2)-n2ncc(Cl)c2N=C(c2c(F)cccc2F)N3)C[C@H](C)O1 ; CC(C)(C)NC(=O)c1ccc2c(c1)-n1ncc(Cl)c1N=C(c1c(F)cccc1F)N2]

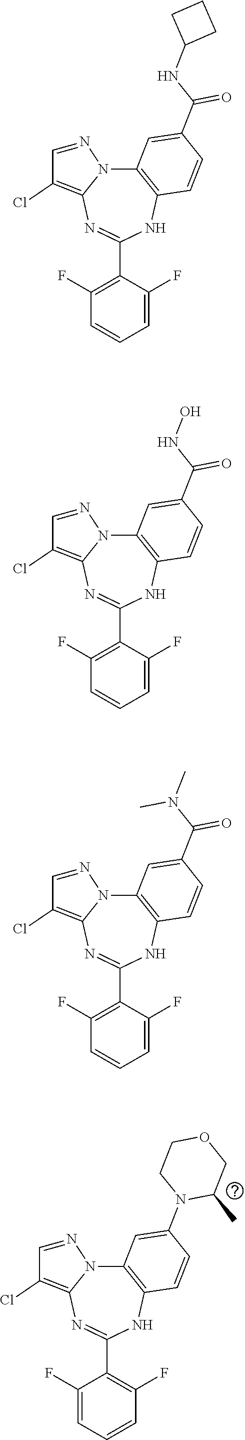 [SMILES: O=C(NC1CCC1)c1ccc2c(c1)-n1ncc(Cl)c1N=C(c1c(F)cccc1F)N2 ; O=C(NO)c1ccc2c(c1)-n1ncc(Cl)c1N=C(c1c(F)cccc1F)N2 ; CN(C)C(=O)c1ccc2c(c1)-n1ncc(Cl)c1N=C(c1c(F)cccc1F)N2 ; C[C@@H]1COCCN1c1ccc2c(c1)-n1ncc(Cl)c1N=C(c1c(F)cccc1F)N2]

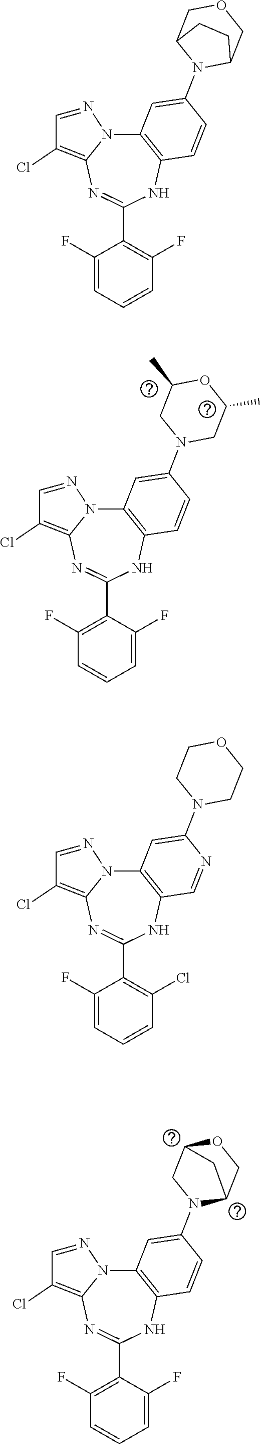 [SMILES: Fc1cccc(F)c1C1=Nc2c(Cl)cnn2-c2cc(N3C4CCC3COC4)ccc2N1 ; C[C@@H]1CN(c2ccc3c(c2)-n2ncc(Cl)c2N=C(c2c(F)cccc2F)N3)C[C@@H](C)O1 ; Fc1cccc(Cl)c1C1=Nc2c(Cl)cnn2-c2cc(N3CCOCC3)ncc2N1 ; Fc1cccc(F)c1C1=Nc2c(Cl)cnn2-c2cc(N3C[C@@H]4C[C@H]3CO4)ccc2N1]

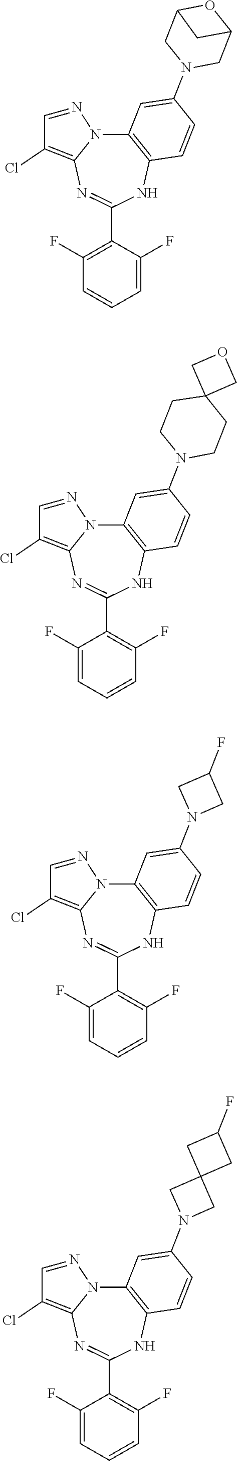 [SMILES: Fc1cccc(F)c1C1=Nc2c(Cl)cnn2-c2cc(N3CC4CC(C3)O4)ccc2N1 ; Fc1cccc(F)c1C1=Nc2c(Cl)cnn2-c2cc(N3CCC4(CC3)COC4)ccc2N1 ; Fc1cccc(F)c1C1=Nc2c(Cl)cnn2-c2cc(N3CC(F)C3)ccc2N1 ; Fc1cccc(F)c1C1=Nc2c(Cl)cnn2-c2cc(N3CC4(CC(F)C4)C3)ccc2N1]

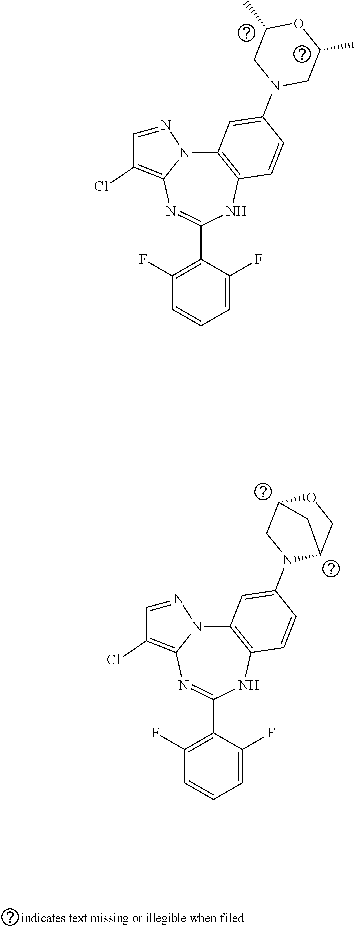 [SMILES: C[C@@H]1CN(c2ccc3c(c2)-n2ncc(Cl)c2N=C(c2c(F)cccc2F)N3)C[C@H](C)O1 ; Fc1cccc(F)c1C1=Nc2c(Cl)cnn2-c2cc(N3C[C@H]4C[C@@H]3CO4)ccc2N1]

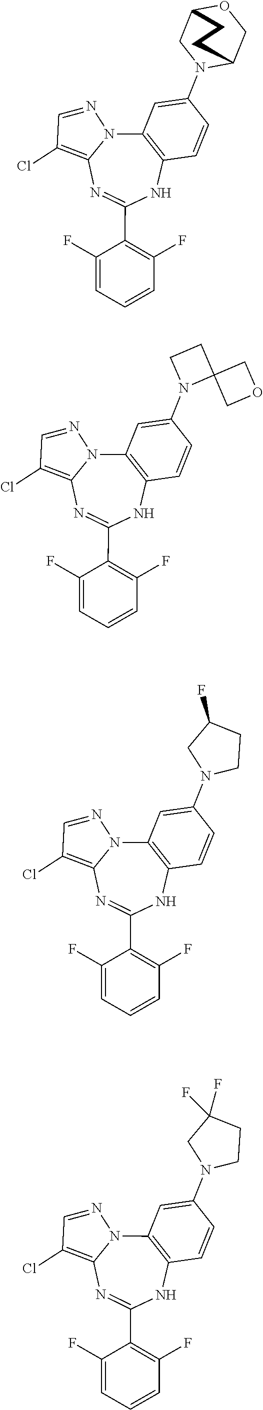

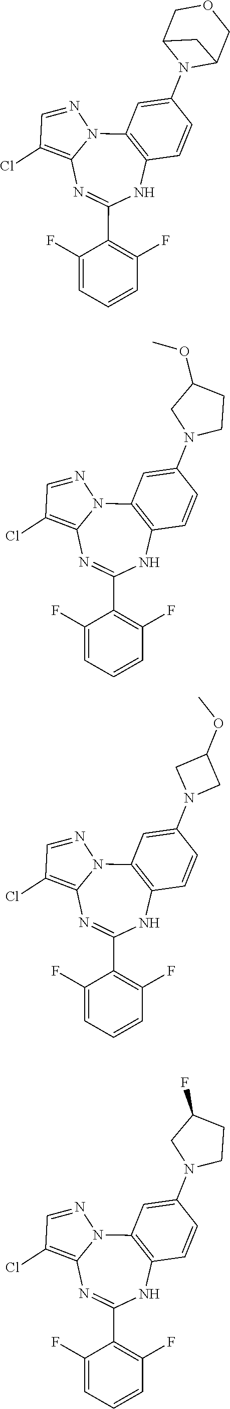 [SMILES: Fc1cccc(F)c1C1=Nc2c(Cl)cnn2-c2cc(N3C4COCC3C4)ccc2N1 ; COC1CCN(c2ccc3c(c2)-n2ncc(Cl)c2N=C(c2c(F)cccc2F)N3)C1 ; COC1CN(c2ccc3c(c2)-n2ncc(Cl)c2N=C(c2c(F)cccc2F)N3)C1 ; Fc1cccc(F)c1C1=Nc2c(Cl)cnn2-c2cc(N3CC[C@H](F)C3)ccc2N1]

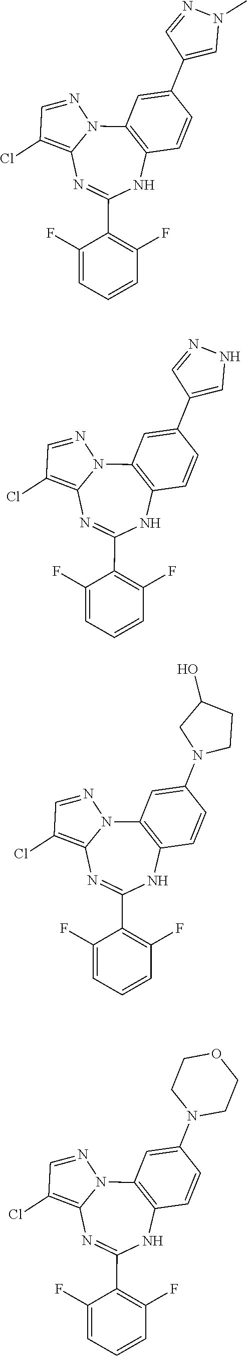 [SMILES: Cn1cc(-c2ccc3c(c2)-n2ncc(Cl)c2N=C(c2c(F)cccc2F)N3)cn1 ; Fc1cccc(F)c1C1=Nc2c(Cl)cnn2-c2cc(-c3cn[nH]c3)ccc2N1 ; OC1CCN(c2ccc3c(c2)-n2ncc(Cl)c2N=C(c2c(F)cccc2F)N3)C1 ; Fc1cccc(F)c1C1=Nc2c(Cl)cnn2-c2cc(N3CCOCC3)ccc2N1]

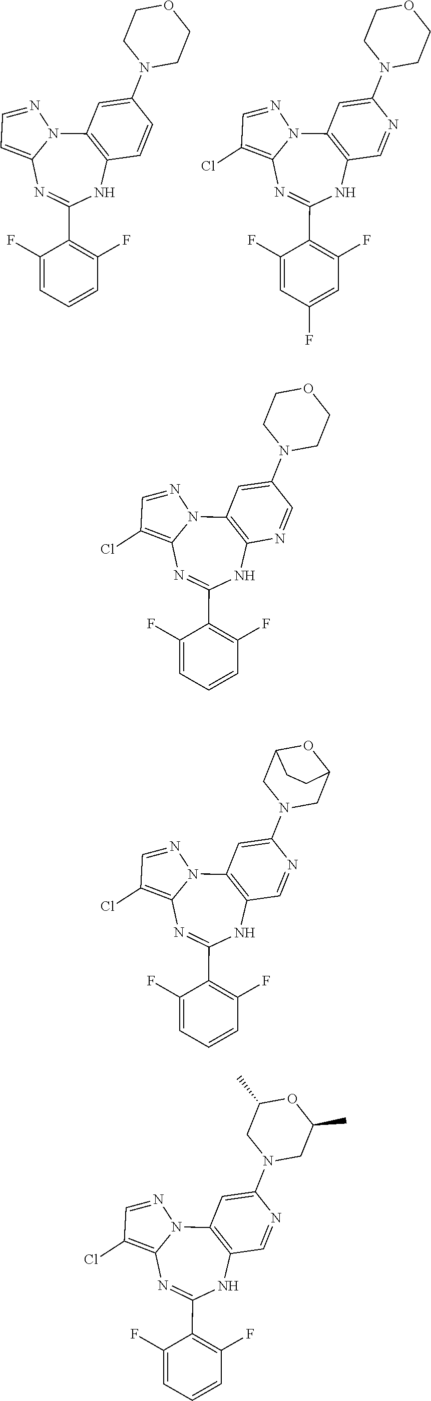

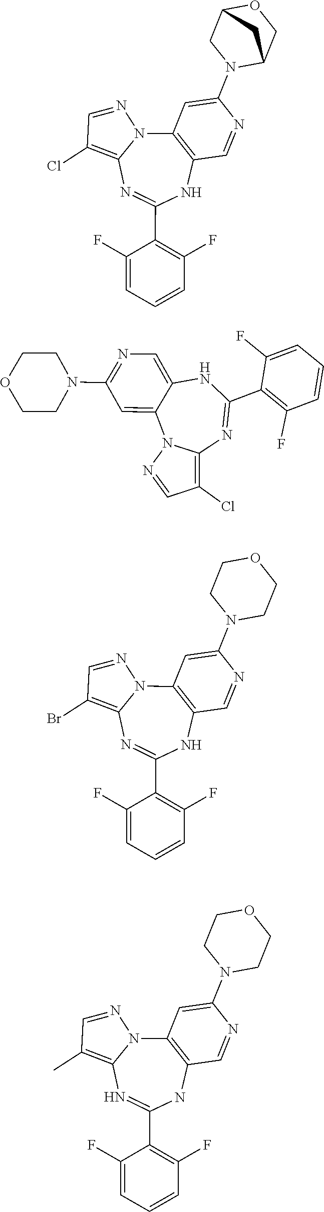 [SMILES: Fc1cccc(F)c1C1=Nc2c(Cl)cnn2-c2cc(N3C[C@H]4C[C@@H]3CO4)ncc2N1 ; Fc1cccc(F)c1C1=Nc2c(Cl)cnn2-c2cc(N3CCOCC3)ncc2N1 ; Fc1cccc(F)c1C1=Nc2c(Br)cnn2-c2cc(N3CCOCC3)ncc2N1 ; Cc1cnn2c1N=C(c1c(F)cccc1F)Nc1cnc(N3CCOCC3)cc1-2]

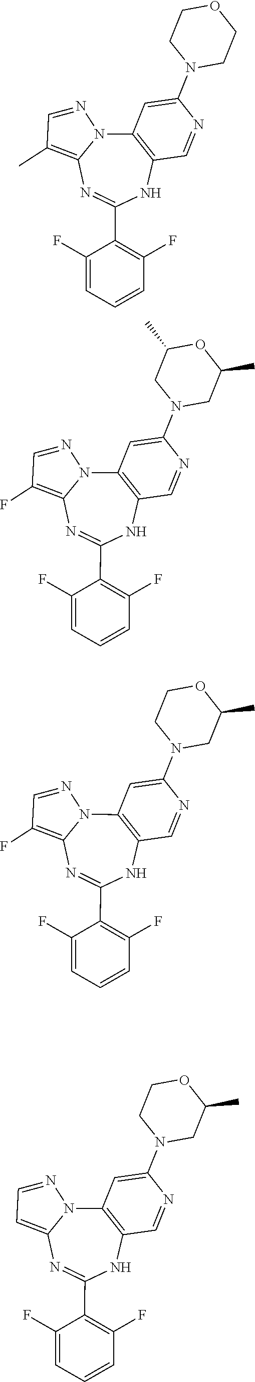

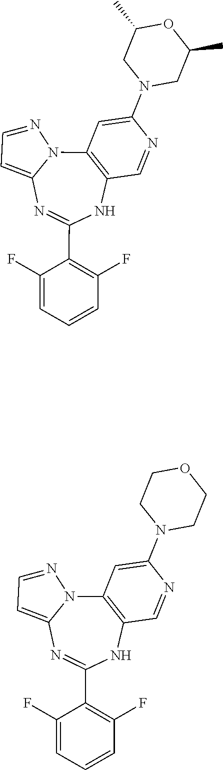 [SMILES: C[C@H]1CN(c2cc3c(cn2)NC(c2c(F)cccc2F)=Nc2ccnn2-3)C[C@H](C)O1 ; Fc1cccc(F)c1C1=Nc2ccnn2-c2cc(N3CCOCC3)ncc2N1]

14. A method of treatment of a patient suffering from a neurological condition, wherein the method comprises administering a therapeutically effective amount of a compound in any of claims 1-13.
